(19) United States
(12) Patent Application Publication
(10) Pub. No.: US 2025/0186566 A1
(43) Pub. Date: Jun. 12, 2025

(54) CELL-BASED VACCINES

(71) Applicant: Arkea Bio Corp., Charlestown, MA (US)

(72) Inventors: Matthew Dunn, Louisville, CO (US); Brigid Arciero, Boulder, CO (US); Colin South, Reno, NV (US); Kristin Adolfsen, Burlington, MA (US); Jeff Bickmeier, Arlington, MA (US); John Sullivan, Lexington, MA (US); Sara Kimler, Boston, MA (US); Lauren Fitch, Malden, MA (US); Joseph Koos, Somerville, MA (US); Laura Holland, Exeter, RI (US); Taylor Falk, Boston, MA (US); James Spoonamore, Somerville, MA (US); Zeljko Radulovic, Cambridge, MA (US); Caitlin Allen, Somerville, MA (US); Anna Li, Boston, MA (US); Alexander Hsu, Belmont, MA (US); Sara Tabandeh, Cambridge, MA (US); Frederick Richard Kearney, Walpole, MA (US)

(21) Appl. No.: 18/977,008

(22) Filed: Dec. 11, 2024

Related U.S. Application Data
(60) Provisional application No. 63/645,276, filed on May 10, 2024.
(60) Provisional application No. 63/608,497, filed on Dec. 11, 2023.

(51) Int. Cl. A61K 39/00; A61P 37/04

(52) U.S. Cl. CPC A61K 39/0001; A61P 37/04; A61K 2039/521; A61K 2039/552; A61K 2039/55505; A61K 2039/55522; A61K 2039/55544; A61K 2039/55555; A61K 2039/55566; A61K 2039/55577; A61K 2039/6081

(57) ABSTRACT

The present invention relates to cell-based vaccine compositions and methods that reduce methane and/or hydrogen production in animals. The present invention relates to the treatment of diseases that are associated with methanogens. The present invention also relates to methods of growing hydrogenotrophs in safe anaerobic conditions.

### CROSS-REFERENCE TO RELATED APPLICATIONS

This application claims the benefit of U.S. Provisional Application No. 63/608,497, filed on Dec. 11, 2023; and U.S. Provisional Application No. 63/645,276, filed on May 10, 2024, the entire contents of each of said applications are incorporated herein in their entirety by this reference.

### BACKGROUND OF THE INVENTION

Methane (CH₄) is the world's second most abundant greenhouse gas after carbon dioxide (CO₂), accounting for ˜16% of total greenhouse gas emissions. Livestock emissions, in particular, account for ˜32% of all anthropogenic CH₄ emissions—equating to ˜6% of all CO₂ equivalents (CO₂e) of greenhouse gas emissions and ˜3 billion Tonnes/yr of CO₂e. CH₄ is a powerful greenhouse gas with a potential global warming effect ˜28-fold higher than that of CO₂ over a 100 year period and ˜80-fold higher than that of CO₂ over a 20 year period. Furthermore, CH₄ has an atmospheric half-life of ˜10 years, thus reducing enteric CH₄ emissions could have an immediate and dramatic effect on limiting the rate of global warming, which would be of great significance to efforts to reduce global greenhouse gas emissions. Furthermore, CH₄ emissions also represent energy losses during ruminant production. On average, approximately 2-12% of the energy consumed in feed is lost in the form of CH₄ emissions.

For ruminants, CH₄ is predominantly formed in the ruminant fore-stomach (rumen) by methanogens, a subgroup of the Archaea. During normal rumen function, plant material is broken down by fiber-degrading microorganisms and fermented mainly to volatile fatty acids, ammonia, H₂ and CO₂. Ruminal methanogens principally use H₂ to reduce CO₂ to CH₄ in a series of reactions that are coupled to ATP synthesis. Additional biochemical pathways to produce CH₄ from acetate and methanol/methylamine substrates are also present, but represent a lower fraction of the total CH₄ biosynthesis in the rumen.

Attempts have been made to inhibit the action of methanogens in the rumen using a variety of interventions but most have failed, or were met with only limited success, due to low efficacy, poor selectivity, toxicity of compounds against the host, and/or build-up of resistance to anti-methanogen compounds. Further most solutions, e.g., feed additives and antibiotics, are only applicable to intensive production environments where the animal's diet can be effectively controlled to ensure precise daily dosages of the intervention. These solutions are, thus, not effective in extensive, grass-fed production environments where the animals a rarely handled and/or operations where feed cannot readily be mixed daily.

Accordingly, there is a great need in the art for effective, selective, and safe compositions and methods for inhibiting methanogens in ruminant populations.

### SUMMARY OF THE INVENTION

The present invention is based, at least in part, on the discovery that vaccines of the present disclosure (e.g., cell-based vaccines comprising a cell and/or a cell part) against at least one cell surface antigen or a fragment thereof (e.g., antigenic fragment, epitope) of at least one methanogen, when administered to a subject (e.g., animal, ruminant), are surprisingly effective in inducing immune response and antibody production against the methanogen, and reducing the CH₄ production in ruminants.

Previous attempts to vaccinate ruminants and reduce CH₄ production have been largely unsuccessful. Research on a vaccine targeting methanogen(s) has cost between $4 million to $5 million a year for more than 20 years. However, it has been very challenging because of the mechanism used (see World Wide Web at dairyherd.com/news/dairy-production/vaccine-could-provide-cattle-ghg-solution). Specifically, the vaccine must elicit an immune response that results in the production of antibodies. At least a portion of the produced antibodies are present in the ruminant's saliva, which then pass to the ruminant's rumen and bind with the methanogens, e.g., those that convert H₂ and CO₂ into CH₄. The antibody must then impair the methanogen thereby reducing total CH₄ production in the rumen and subsequent emission. As of current knowledge, the rumen is a relatively isolated organ lacking an adaptive immune response similar to that found systemically, thereby requiring the produced antibodies to have a direct effect on methanogens in the rumen.

While previous attempts at vaccination were able to induce an immune response in sheep resulting in production of antibodies that bind to methanogens, it was unfortunately not possible to induce production of a consistently large amount of antibodies introduced to the rumen via saliva; and to produce effective antibodies that can neutralize the growth of the methanogen and/or the production of CH₄.

Wright et al (2004) Vaccine 22:29-30 was able to immunize sheep with a whole-cell preparation from a mixture of 3 methanogens and tentatively reduce CH₄ production (per kg/DMI) by 7.7%. However, when the study was repeated with a mixture of 5 methanogens, vaccination failed to demonstrate any CH₄ abatement, although it changed the microbial fauna in the rumen (Williams et al. (2009) Appl Environ Microb 75(7):1860-1866). Accordingly, there has been a long-felt need that could not be resolved due to failure of others.

Compounding the failed attempts to reproducibly reduce CH₄ emissions in sheep following vaccination, there has yet to be any successful demonstration of reduced CH₄ emission in other ruminants following vaccination. This is even more important in cattle, which contribute the majority of ruminant greenhouse gas emissions.

In contrast to the failures of others, the vaccines of the present disclosure yielded at least 17% reduction in the emission of CH₄ by ruminants following treatment. Furthermore, the ruminants vaccinated with a vaccine of the present disclosure resulted in continued and sustained reductions of CH₄ emissions, and over the course of 5 weeks have mitigated ˜1.1 kg of CH₄ emissions per treated ruminant, which over the course of a year equates to ˜0.3-0.4 tonnes (1000 kg) of mitigated CH₄ per ruminant per year. These results are unprecedented in the field, even more so in cattle. Treated ruminants also showed continued in sustained reduction of H₂ emissions. Accordingly, the vaccines of the present disclosure provide a surprising and unexpected means of reducing CH₄ and/or H₂ emission in ruminants, which could not have been achieved by others despite diligent efforts.

The vaccine compositions and methods of the present disclosure are useful beyond reducing the CH₄ emission in ruminants.

In addition to CH₄ emission reductions, vaccine compositions and methods of the present disclosure have shown surprising and unexpected reductions in emitted H₂ following treatment. These results are both surprising and unexpected as reductions in CH₄ following treatment with small molecule inhibitors and feed additives have contrarily shown increases in H₂ emissions. The reduction in both CH₄ and H₂ emissions suggest that the vaccine compositions and methods of the present disclosure have utility in improving the feed conversion efficiency and thereby the productivity of treated animals, e.g., increasing the production and/or ruminal concentration of one or more volatile fatty acids (e.g., propionate, butyrate, acetate) in the rumen of the animal, increasing the average daily gain of the animal, reducing the dry matter intake of the animal, reducing the feed requirements of the animal during lactation, and/or increasing the milk production of the animal. Increasing productivity of treated animals can further reduce the carbon intensity of resultant animal products (e.g., milk and meat) as treated animals are not only emit less CH₄ but also produce more product per animal. Thus, the total reduction in carbon intensity of animal-derived products from animals treated with a vaccine composition of the present disclosure may can be calculated as the composite of the CH₄ reduction of the animal following treatment in combination with the reduced carbon footprint associated with growing and maintaining those animals (e.g., less total feed, manure, urine, etc.).

Additionally, it is well documented that methanogens are associated with various diseases, including periodontal disease, inflammatory bowel disease (IBD), irritable bowel syndrome (ISB), e.g., IBS-C, SIBO, colorectal cancer, obesity and metabolic syndrome, diverticulosis and diverticulitis, gingivitis, and bloat. Thus, the vaccine compositions and methods of the present disclosure have utility in treating these diseases in animals including humans. The vaccines of the present disclosure also provide a surprising and unexpected effect on lactic acidosis (e.g., reducing rumen lactate, increasing pH, or combination thereof). Therefore, the vaccine compositions methods of the present disclosure have utility in treating diseases associated with elevated, increased, or severe lactic acidosis, e.g., liver abscess.

Further provided herein are compositions, systems, and methods of growing hydrogenotrophs (e.g., methanogens) without explosive and flammable concentrations of H₂ under high pressure.

### BRIEF DESCRIPTION OF FIGURES

FIG. 1A-FIG. 1B show schematic diagrams representing the relative abundance of methanogen species across geographies and herds. The diagrams indicate that methanogen populations are relatively stable across geographies and herds. For example, Methanobrevibacter gottschalkii comprises ˜30-40% of ruminal methanogens globally; thus, a vaccine comprising cells and/or cell parts of at least one methanogen could provide an effective global solution for reducing the amount of CH₄ generated by ruminants.

FIG. 2 shows a representative graph demonstrating the reduction of CH₄ emissions by cows vaccinated with a cell-based vaccine of the present disclosure. The graph represents data from the first 40 days of vaccination and indicates a significant reduction (˜17%) in emitted CH₄ after boosting.

FIG. 3 shows an increased antigen-specific antibody titer in the sera of vaccinated ruminants in FIG. 2.

FIG. 4 shows a schematic diagram that illustrates vaccination and post-vaccination testing of a ruminant (e.g., a cow).

FIG. 5A-FIG. 5C show schematic diagrams that illustrate exemplary tests performed for a ruminant vaccinated with a cell-based vaccine of the present disclosure. FIG. 5A shows quantifying Ig antibody in serum and saliva of a vaccinated ruminant. The antibody is evaluated for methanogen binding to methanogens (e.g., affinity, specificity, etc.) and/or its effect on the fitness of methanogens (e.g., proliferation, CH₄ production, etc.) upon binding. FIG. 5B shows measuring the amount of CH₄ produced by a vaccinated ruminant using the GreenFeed system (C-lock Inc., Rapid City, South Dakota). FIG. 5C shows evaluation of the changes in the rumen microbiome in a vaccinated ruminant. The 16S rRNA sequencing identifies the presence and the amount of specific methanogens.

FIG. 6A-FIG. 6B show an exemplary vaccine preparation workflow. FIG. 6A shows growth of methanogens, subsequent fixation (e.g., with formaldehyde) and, washing (using e.g., Phosphate-Buffered Saline (PBS)). FIG. 6B shows how cells are counted and cell titer is normalized. Adjuvant is added to the mixture of cells to prepare cell-based methanogen vaccines.

FIG. 7 shows a schematic diagram of an exemplary instrument (e.g., GreenFeed) that measures the CH₄ produced from the rumen of a ruminant.

FIG. 8 shows an exemplary vaccination schedule. The bottom panel shows the measurements of animal temperature taken during the vaccinated period. No adverse events were reported for the vaccinated ruminants.

FIG. 9-FIG. 10 show CH₄ emissions analysis performed using the GreenFeed system. FIG. 9 and FIG. 10 demonstrate statistically significant reductions in CH₄ emitted by vaccinated cows after boosting.

FIG. 11 shows a representative analysis of sera from vaccinated cows by whole-cell methanogen ELISA. The analysis showed increased binding following consecutive vaccinations, a specific binding response that correlates to the vaccinated strain of methanogen, and binding to both strains in the blended vaccine.

FIG. 12 shows the CH₄ produced by the cows vaccinated with M. gottschalkii. A significant reduction (˜17% reduction) in emitted CH₄ was observed for 4 out of 5 ruminants vaccinated with M. gottschalkii.

FIG. 13 shows a representative H₂ emissions analysis (using GreenFeed system). A statistically significant reduction in emitted H₂ was observed after boosting, especially in cows vaccinated with M. gottschalkii.

FIG. 14 is a schematic of a biochemical pathway and enzymes for the production of CH₄ from acetate (i.e., the acetoclastic pathway), H₂ and CO₂ (i.e., the hydrogenotrophic pathway), and methanol and derivatives thereof (i.e., the methylotrophic pathway).

FIG. 15 is a schematic describing the reduction of methyl-CoM and coenzyme B into CH₄ by methyl-coenzyme M reductase (MCR), a key enzyme present in CH₄ production via the acetoclastic, hydrogenotrophic, and methylotrophic pathways.

FIG. 16 is a schematic describing the application of small molecules that affect MCR activity to modulate CH₄ production.

FIG. 17 is a cross-sectional diagram of the application of a composition for reduction of deleterious atmospheric gases and/or precursors thereof to a water source, such as a trough or a pond. The water source (401) comprises the composition (402). While the composition (402) is shown to be at the bottom of the water source (401), the composition (402) can be at any suitable position, for example floating at the top, admixed within, or dissolved within the water source (401).

FIG. 18 shows aerial delivery of a composition for reduction of deleterious atmospheric gases and/or precursors thereof to a water source. A aerial delivery device (501) delivers (502) a composition (within the device) to a water source (503), such as a trough or a pond.

FIG. 19 shows 3NOP concentration (mM) v. adsorbent (˜20 mM stock solution).

FIG. 20 shows 3NOP concentration (mM) v. adsorbent (˜8 mM stock solution).

FIG. 21 shows exemplary multilayer polyelectrolyte coatings of 15% activated carbon tablets.

FIG. 22 shows exemplary multilayer polyelectrolyte coatings of 25% activated carbon tablets.

FIG. 23 shows exemplary multilayer polyelectrolyte coatings of 15% activated carbon tablets with 5% sodium lignosulfonate.

FIG. 24 shows exemplary multilayer polyelectrolyte coatings of 15% activated carbon with 5% hydroxypropyl cellulose.

FIG. 25 is a graph showing the release profiled of a silica v. activated carbon adsorbent.

FIG. 26 is a table showing the composition of various polycaprolactone-based formulations according to some embodiments of the invention.

FIG. 27 is a bar graph showing 3NOP release in mM for exemplary polycaprolactone-based formulations.

FIG. 28 is a graph showing normalized 3NOP concentration v. release time in days of exemplary polybutylene succinate-based formulations.

FIG. 29 is a bar graph showing 3NOP release (%) from exemplary PEC microcapsules. Samples are prepared with a 3NOP concentration of 100 μM. Final pH solution ˜7. K, L, F, and E refer to lysine, leucine, phenylalanine, and glutamic acid, respectively. PSS refers to polystyrene sulfonate.

FIG. 30 is bar graph showing 3NOP release (%) from exemplary PEC microcapsules. Samples are prepared with a 3NOP concentration of 100 μM. Final pH solution ˜7. PLR refers to poly (L-arginine), and PLK refers to poly (L-lysine). SLS and PSS refer to sodium lignosulfonate and polystyrene sulfonate.

FIG. 31 shows the sera antibody binding to M. gottschalkii in the ELISA assay.

FIG. 32 shows the sera antibody binding to M. ruminantium in the ELISA assay.

FIG. 33 shows the sera antibody (from sera collected on d49) binding to M. gottschalkii in the FACS analysis.

FIG. 34 shows the binding of antibodies in clarified sera, collected from Animal 432K, to M. ruminantium and M. gottschalkii, observed using Western blot.

FIG. 35 shows the mitigated CH₄ emissions per treatment group.

FIG. 36 shows the mitigated H₂ emissions per treatment group.

FIG. 37A shows the mitigated CO₂-normalized CH₄ emissions per treatment group.

FIG. 37B shows the increased CO₂ emissions per treatment group.

FIG. 38 shows the results of in vitro CH₄ production using the d63 sera from ruminants vaccinated with cell-based vaccines.

FIG. 39 shows mandatory components of system for growing hydrogenotrophic organisms.

FIG. 40 shows a process flow diagram of the continuous flow methanogen (e.g., M. gottschalkii) culturing system. Gas is sparged through anaerobic water for humidification prior to entering the methanogen culture bottle, to prevent evaporation. The methanogen converts H₂ and CO₂ into CH₄ during growth. The H₂, CO₂, N₂, CH₄ mixture then passes through a trap before entering a flow meter to minimize fouling risk to the flow meter.

FIG. 41 shows an example of a gas diffusion system for the improvement of gas transfer within the system. In this example, a sintered stainless steel gas diffuser is used. However, other instruments, e.g., spargers, baffles, impellers, etc., that are used in culturing microorganisms may be used. Optionally, a humidifying water bottle can be placed upstream to minimize volume loss of the culture. Alternatively, a condenser or other volume capture or addition system can be used.

FIG. 42 shows a labeled picture of the water bottle used in the water bath. The culture bottle followed the same setup.

FIG. 43 shows cell titer within an M. gottschalkii culture grown with continuously flowing 4% H₂, 10% CO₂, and 86% N₂ (“continuous”) compared to an M. gottschalkii culture grown with over pressurization of the headspace at multiple time points throughout growth with 80% H₂, 20% CO₂ to 15 psi overpressure. Cell titering was performed on a SONY SH800 FACS using the protocol described in Example 1.

FIG. 44 shows an exemplary process flow diagram for manufacturing of a whole cell vaccine. A vial of the Master Cell Bank is thawed and expanded to produce a Working Cell Bank (1), which is then used to inoculate the seed train (2-3) and production fermenter (4). Cells are then killed and buffer exchanged (5), formulated (6), and then filled into vials for distribution (7).

FIG. 45 shows CH₄ produced from a M. gottschalkii culture grown in BY medium (bottom curve) or semi-defined media SD0001 (top curve) in Hungate tubes with ˜25 psi 80% H₂/20% CO₂ initial batched headspace. CH₄ measurements were performed using a Gazomat Gazoscan using a custom jig for consistent positioning of the tubes relative to the measurement device. Data shown is the average of 2 duplicate tubes with error bars representing 1 standard deviation.

FIG. 46 shows reduction in CH₄ production in vitro following treatment with sera before and after vaccination with a vaccine as disclosed herein.

FIG. 47A shows reduction in methanogen growth and FIG. 47B shows reduction in CH₄ production in vitro following treatment with sera before and after vaccination.

### DETAILED DESCRIPTION OF THE INVENTION

Provided herein are vaccines (e.g., cell-based vaccines comprising a cell and/or a cell part) against at least one cell surface antigen or a fragment thereof (e.g., antigenic fragment, epitope) of at least one methanogen, which are effective in inducing immune response and antibody production against the methanogen antigen, and reducing the CH₄ production in subjects. The vaccines of the present disclosure are also useful in treating diseases in subjects (e.g., animals, mammals, ruminants, humans) that are associated with methanogens (e.g., periodontal disease, Inflammatory Bowel Disease (IBD), gingivitis, and/or bloat). The vaccines of the present disclosure are also useful in treating diseases in subjects (e.g., animals, mammals, ruminants, humans) that are associated with elevated, increased, or severe lactic acidosis, e.g., liver abscess.

Further provided herein are compositions, systems, and methods of growing hydrogenotrophs (e.g., methanogens), e.g., without explosive and flammable concentrations of H₂ under high pressure.

### Definitions

The articles “a” and “an” are used herein to refer to one or to more than one (i.e., to at least one) of the grammatical object of the article. By way of example, “an element” means one element or more than one element.

As used herein, the term “about” when used before a numerical designation, e.g., temperature, time, amount, concentration, and such other, including a range, indicates approximations which may vary by (+) or (−) 10%, 9%, 8%, 7%, 6%, 5%, 4%, 3%, 2%, or 1%.

The term “administering” is intended to include routes of administration which allow an agent (e.g., a vaccine composition, an agent that reduces CH₄ production in a subject) to perform its intended function. Examples of routes of administration which can be used include injection (subcutaneous, intravenous, parenteral, intraperitoneal, intrathecal, intradermal, intramuscular, etc.), oral, inhalation, and transdermal routes. The injections can be bolus injections or can be continuous infusion. Depending on the route of administration, the agent (e.g., a vaccine composition, an agent that reduces CH₄ production in a subject) can be coated with or disposed in a selected material to protect it from natural conditions which may detrimentally affect its ability to perform its intended function. The agent may be administered alone, or in conjunction with a pharmaceutically acceptable carrier or adjuvant. The agent (e.g., a vaccine composition, an agent that reduces CH₄ production in a subject) also may be administered as a prodrug, which is converted to its active form in vivo.

As used herein, “anaerobic conditions” are conditions with reduced levels of oxygen compared to normal atmospheric conditions. For example, in some embodiments anaerobic conditions are conditions wherein the oxygen levels are partial pressure of oxygen (pO₂) no more than 8%. In some instances, anaerobic conditions are conditions wherein the pO₂ is no more than 2%. In some instances, anaerobic conditions are conditions wherein the pO₂ is no more than 0.5%. In certain embodiments, anaerobic conditions may be achieved by purging a growth chamber and/or a bioreactor with a gas other than oxygen such as, for example, N₂, H₂, and/or CO₂.

The term “cell parts,” as used herein encompasses any and all that is less than a whole cell. In some embodiments, cell parts of the present disclosure comprise the cell membrane with the membrane-bound proteins. In some embodiments, the cell parts comprise an antigenic part of the cell. Such antigenic part may comprise at least one epitope that binds to the antibody. In preferred embodiments, the cell parts of the present disclosure are effective in eliciting immune response and/or inducing antibody production when administered to a subject. In some embodiments, the vaccine composition of the present disclosure comprises at least about 10, 15, 20, 25, 30, 35, 40, 45, 50, 55, 60, 65, 70, 75, 80, 85, 90, 95, 96, 97, 98, 99, 99.9, 99.99 or 100% whole cells. In some embodiments, the vaccine composition of the present disclosure comprises at least about 10, 15, 20, 25, 30, 35, 40, 45, 50, 55, 60, 65, 70, 75, 80, 85, 90, 95, 96, 97, 98, 99, 99.9, 99.99 or 100% cell parts.

The term “conjoint” or “combination” administration, as used herein, refers to the administration of two or more agents that aid in reducing CH₄ production in a subject. The different agents comprising the combination may be administered concomitant with, prior to, or following the administration of one or more agents.

The term “fragment,” as used herein encompasses any and all that is less than the full length. In some embodiments, a fragment of a polypeptide of the present disclosure is an antigenic fragment of the polypeptide. Such antigenic fragment may comprise at least one epitope that binds to the antibody. In preferred embodiments, a fragment of a polypeptide of the present disclosure is a fragment of the polypeptide that is effective in eliciting immune response and/or inducing antibody production when administered to a subject.

The term “methanogen,” as used herein, refers to a microorganism that produces CH₄ as a metabolic byproduct. Methanogens belong to the domain Archaea, and include, but are not limited to those of a family Methanobacteriaceae, e.g., those of genera Methanobrevibacter, Methanosphaera, Methanomassiliicoccaceae, Methanomicrobium, Methanobacterium, Methanocorpusulum, Methanosaeta, Methanoculleus, Methanosarcina, and Thermoplasmatales. Specific methanogens include, but are not limited to, Methanobrevibacter ruminantium (e.g., the M1 strain or strain DSM 1093 (see e.g., World Wide Web at dsmz.de/microorganisms/html/strains/strain.dsm001093.htm) and Methanobrevibacter gottschalkii. Additional relevant species are further described below.

The term “ruminant” refers to a hoofed herbivorous grazing or browsing mammal that is able to acquire nutrients from plant-based food by fermenting it in a specialized stomach prior to digestion, principally through microbial actions. The process, which takes place in the front part of the digestive system and therefore is called foregut fermentation, typically requires the fermented ingesta (known as cud) to be regurgitated and chewed again. The roughly 200 species of ruminants include both domestic and wild species. Ruminants include, but are not limited to, cattle (e.g., large domesticated ruminant animals, e.g., cows (including dairy cattle), bulls), all domesticated and wild bovines (i.e., those belonged to the family Bovidae; e.g., cows, bulls, bisons, yaks, African buffalos, water buffalos, antelopes), goats, sheep, giraffes, deer, caribou, and gazelles. In preferred embodiments, ruminants are domesticated. As used herein, the term “ruminant” includes ruminant-like animals or pseudo-ruminant animals such as macropods, llamas, camels, and alpacas. In some embodiments, a ruminant has not been administered with an agent that reduces CH₄. In other embodiments, a ruminant has been administered or is being administered with an agent that reduces CH₄.

The term “subject” refers to any healthy or diseased animal, including any mammal, ruminant, canine, feline, or human.

### Methanogens

The diversity of ruminal methanogens is much lower than that of rumen bacteria, with archaeal SSU rRNA only accounting for 6.8% of rumen total SSU rRNA. Archaea in the rumen is represented by <3.3% of the total rRNA (both 16S and 18S) therein. Representative family of methanogens includes Methanobacteriaceae. Rumen methanogens typically comprises 2-3% of the total microbial biomass in the rumen.

Representative genera of methanogens include Methanobrevibacter, Methanosphaera, Methanomassiliicoccaceae, Methanomicrobium, Methanobacterium, Methanocorpusulum, Methanosaeta, Methanoculleus, Methanosarcina, and Thermoplasmatales.

Certain species of ruminal methanogens have been isolated into pure cultures: Methanobacterium formicicum, Methanobacterium bryantii, Methanobrevibacter ruminantium, Methanobrevibacter gottschalkii, Methanobrevibacter millerae, Methanobrevibacter olleyae, Methanomicrobium mobile, Methanoculleus olentangyi, and Methanosarcina barkeri. Additional species have been recently isolated, including Methanobrevibacter boviskoreani (isolated from the rumen of Korean native cattle), Methanobacterium beijingense (isolated from the rumen of goat), Methanoculleus marisnigri (isolated from the rumen of Indian crossbred cattle), Methanoculleus bourgensis (isolated from the rumen of Holstein cattle), and Methanosarcina mazei (isolated from the rumen of Korean Hanwoo cattle) (based on the RDP database). A Thermoplasmatales-like pyrrolysine-dependent archaeon BRNA1 was also isolated from bovine (GenBank access number: CP002916).

Collectively, 16S rRNA gene sequences from cultured methanogens only accounted for approximately 0.7% of the total archaeal sequences of rumen origin, and several taxa do not have a single cultured representative. Most of the isolates are members of the family Methanobacteriaceae. Compared to other anaerobic habitats where >100 species of methanogens of 28 genera have been isolated, the diversity and species richness of ruminal methanogens are quite low, reflecting the highly selective ruminal environment for methanogens. In addition, sequenced ruminal 16S rRNA gene clones shared >95% sequence similarity with that of Methanobrevibacter gottschalkii, Methanobrevibacter thaueri, Methanobrevibacter smithii and Methanosphaera stadtmanae, indicating that these species may be common ruminal methanogens.

Much of the ruminal methanogen diversity was characterized by 16S rRNA gene sequences. The RDP Release 11 (Update 3) contains 8,623 archaeal 16S rRNA gene sequences of rumen origin. These sequences were generated using the Sanger sequencing technology, which produces higher sequence accuracy than NGS technologies, in 96 separate studies including 48 unpublished studies. About 90% of these sequences were assigned to methanogens. These sequences were classified to 10 known genera, with Methanobrevibacter being represented by 63.2% of all the sequences followed by Methanosphaera (9.8%), Methanomicrobium (7.7%), and Methanobacterium (1.2%). The order Thermoplasmatales, which was previously referred to as the rumen cluster C (RCC) group, is represented by 7.4% of the total archaeal sequences.

### Cell Surface Proteins of Methanogens

At least one cell surface protein or a fragment thereof that is present on the cell surface of at least one methanogen may be effective in eliciting immune response, antibody production, and antibody-mediated neutralization of the growth of methanogens and/or production of CH₄. The representative cell surface antigens of a methanogen, Methanobrevibacter ruminantium, are listed in Table 1. The representative cell surface antigens of various methanogens, and their nucleic acid sequences and amino acid sequences are provided in the U.S. application Ser. No. 18/350,526 (e.g., Table 2A, Table 2B, Table 3, Table 19, Table 20, and Table 21), U.S. Application No. 63/359,978, or U.S. Application No. 63/524,513, the entire contents of each of which are incorporated herein by reference in their entirety.

TABLE 1
Representative cell surface antigens of a methanogen,
Methanobrevibacter ruminantium (M1 (DSM 1093))

| | | | | No. of |
|---|---|---|---|---|
| Locus | | Nucleic Acid | Signal | transmembrane |
| tag | Annotation | Size (bp) | peptide (SP) | domains |
| mru0019 | adhesin-like protein | 1220 | SP | 2 |
| mru0327 | adhesin-like protein | 2090 | SP | 2 |
| mru0687 | adhesin-like protein | 2963 | SP | 2 |
| mru1210 | adhesin-like protein | 7250 | SP | 2 |
| mru1222 | adhesin-like protein | 4055 | SP | 2 |
| mru1506 | adhesin-like protein | 857 | SP | 2 |
| mru2053 | adhesin-like protein | 3494 | SP | 2 |
| mru2134 | adhesin-like protein | 17957 | SP | 2 |
| mru2147 | adhesin-like protein | 16955 | SP | 4 |
| mru2178 | adhesin-like protein | 9239 | SP | 2 |
| mru0031 | adhesin-like protein | 4415 | Expressed on | 1 |
| | | | cell wall | |
| mru0704 | adhesin-like protein | 2858 | SP | 1 |
| mru0963 | adhesin-like protein | 8159 | Expressed on | 0 |
| | | | cell wall | |
| mru0976/0977 | adhesin-like protein | 4775 | SP | 2 |
| mru0020 | adhesin-like protein with cysteine | 6014 | SP | 1 |
| | protease domain | | | |
| mru0064 | adhesin-like protein | 3536 | SP | 1 |
| mru0072 | adhesin-like protein | 2918 | SP | 1 |
| mru0076 | adhesin-like protein | 6605 | SP | 1 |
| mru0077 | adhesin-like protein | 9161 | SP | 1 |
| mru0079 | adhesin-like protein | 3560 | SP | 1 |
| mru0083 | adhesin-like protein | 839 | SP | 1 |
| mru0084 | adhesin-like protein | 14477 | SP | 1 |
| mru0085 | adhesin-like protein | 8030 | SP | 1 |
| mru0086 | adhesin-like protein | 10175 | SP | 1 |
| mru0143 | adhesin-like protein with cysteine | 3284 | SP | 0 |
| | protease domain | | | |
| mru0160 | adhesin-like protein | 3176 | SP | 1 |
| mru0222 | adhesin-like protein with cysteine | 3302 | SP | 1 |
| | protease domain | | | |
| mru0327 | adhesin-like protein | 2060 | SP | 1 |
| mru0338 | adhesin-like protein | 6929 | SP | 1 |
| mru0417/0418 | adhesin-like protein | 1391 | SP | 1 |
| mru0419 | adhesin-like protein | 4175 | SP | 1 |
| mru0727 | adhesin-like protein with cysteine | 3788 | SP | 0 |
| | protease domain | | | |
| mru0772 | adhesin-like protein with cysteine | 3281 | SP | 1 |
| | protease domain | | | |
| mru0839 | adhesin-like protein with cysteine | 8639 | SP | 1 |
| | protease domain | | | |
| mru0842 | adhesin-like protein with cysteine | 3977 | SP | 1 |
| | protease domain | | | |
| mru0978 | adhesin-like protein | 6606 | SP | 0 |
| mru0979 | adhesin-like protein | 8753 | SP | 1 |
| mru1076 | adhesin-like protein | 2681 | SP | 1 |
| mru1077 | adhesin-like protein | 2273 | SP | 1 |
| mru1246 | adhesin-like protein | 4619 | SP | 1 |
| mru1247 | adhesin-like protein | 5060 | SP | 1 |
| mru1465 | adhesin-like protein | 2882 | SP | 1 |
| mru1513 | adhesin-like protein | 1853 | SP | 1 |
| mru1650 | adhesin-like protein | 9161 | SP | 1 |
| mru1726 | adhesin-like protein | 6767 | SP | 1 |
| mru1971 | adhesin-like protein | 1937 | SP | 1 |
| mru1996 | adhesin-like protein | 4496 | SP | 1 |
| mru2043 | adhesin-like protein | 9530 | SP | 1 |
| mru2048 | adhesin-like protein | 5417 | SP | 1 |
| mru2049 | adhesin-like protein | 10355 | SP | 1 |
| mru2052 | adhesin-like protein | 4112 | SP | 1 |
| mru2054 | adhesin-like protein | 5054 | SP | 1 |
| mru2055 | adhesin-like protein | 8906 | SP | 1 |
| mru2059 | adhesin-like protein | 4415 | SP | 0 |
| mru2090 | adhesin-like protein | 15200 | SP | 1 |
| mru0004 | adhesin-like protein | 2237 | SP | 1 |
| mru0331 | adhesin-like protein | 1622 | SP | 1 |
| mru0843 | adhesin-like protein with cysteine | 6197 | SP | 1 |
| | protease domain | | | |
| mru0015 | adhesin-like protein with cysteine | 3845 | SP | 1 |
| | protease domain | | | |
| mru0090 | adhesin-like protein | 2063 | SP | 1 |
| mru0255 | adhesin-like protein | 4187 | SP | 1 |
| mru0450 | adhesin-like protein | 803 | SP | 1 |
| mru0723 | adhesin-like protein | 7727 | SP | 1 |
| mru0962 | adhesin-like protein | 14789 | SP | 1 |
| mru0970 | adhesin-like protein | 2483 | SP | 1 |
| mru1263 | adhesin-like protein | 2585 | SP | 1 |
| mru1358 | adhesin-like protein | 2243 | SP | 1 |
| mru1386 | adhesin-like protein | 1841 | SP | 1 |
| mru1387 | adhesin-like protein with cysteine | 2957 | SP | 1 |
| | protease domain | | | |
| mru1424 | adhesin-like protein | 1445 | SP | 1 |
| mru1500 | adhesin-like protein | 3896 | SP | 1 |
| mru0493 | adhesin-like protein | 2447 | SP | 1 |
| mru0824 | adhesin-like protein with | 2027 | SP | 1 |
| | transglutaminase domain | | | |
| mru1499 | adhesin-like protein with | 3032 | SP | 0 |
| | transglutaminase domain | | | |
| mru1604 | adhesin-like protein with | 2996 | SP | 0 |
| | transglutaminase domain | | | |

| | |
|---|---|
| mru1919 | tetrahydromethanopterin S-methyltransferase subunit A MtrA1 |
| mru0441 | tetrahydromethanopterin S-methyltransferase subunit A MtrA2 |
| mru1920 | tetrahydromethanopterin S-methyltransferase subunit B MtrB |
| mru1921 | tetrahydromethanopterin S-methyltransferase subunit C MtrC |
| mru1922 | tetrahydromethanopterin S-methyltransferase subunit D MtrD |
| mru1923 | tetrahydromethanopterin S-methyltransferase subunit E MtrE |
| mru1918 | tetrahydromethanopterin S-methyltransferase subunit F MtrF |
| mru1917 | tetrahydromethanopterin S-methyltransferase subunit G MtrG |
| mru1916 | tetrahydromethanopterin S-methyltransferase subunit H MtrH |

### Growing Hydrogenotrophs (e.g., Methanogens)

### Hydrogenotrophic Organisms

In certain aspects provided herein are compositions, systems, and methods of growing at least one hydrogenotrophic organism or hydrogenotroph (e.g., bacteria, e.g., methanogen), which are able to metabolize molecular hydrogen as a source of energy. Additional information regarding hydrogenotrophs or their growth conditions can be found in He et al. (2019) Chapter 3.09 Biogas, Comprehensive Biotechnology (Third Edition), pages 110-127 (ISBN 9780444640475; World Wide Web at doi.org/10.1016/B978-0-444-64046-8.00154-3); and Kim and Whitman (2014) Methanogens, Encyclopedia of Food Microbiology (Second Edition), pages 602-606 (ISBN 9780123847331; World Wide Web at doi.org/10.1016/B978-0-12-384730-0.00204-4), each of which is incorporated herein by reference. These composition, systems, and methods for growing hydrogenotrophs, e.g., methanogens, have broad utility included but not limited to production of antigenic material for vaccines as disclosed herein.

In some embodiments, the at least one hydrogenotroph comprises at least one methanogen selected from those known in the art or described herein.

In some embodiments, the at least one hydrogenotroph comprises a methanogen of a genus of Methanobrevibacter.

In some embodiments, the at least one hydrogenotroph comprises Methanobrevibacter ruminantium.

In some embodiments, the at least one hydrogenotroph comprises Methanobrevibacter gottschalkii.

The compositions and methods disclosed herein are suitable for coculturing hydrogenotrophs. In some embodiments, the at least one hydrogenotroph comprises at least 2, 3, 4, 5, 6, 7, 8, 9, or 10 different hydrogenotrophs. In some embodiments, at least one of the hydrogenotrophs comprises Methanobrevibacter ruminantium. In some embodiments, at least one of the hydrogenotrophs comprises Methanobrevibacter gottschalkii. In some embodiments, the compositions, systems, and methods disclosed herein are suitable for coculturing Methanobrevibacter ruminatium and Methanobrevibacter gottschalkii, optionally with one or more additional hydrogenotrophs.

In some embodiments, the at least one hydrogenotroph comprises an organism selected from Table A. Table A lists organisms that contain the 5,10-methenyltetrahydromethanopterin hydrogenase gene, which catalyzes the addition of hydrogen with 5,10-methenyl-5,6,7,8-tetrahydromethanopterin to form 5,10-methylenetetrahydromethanopterin. Source: UniProt (World Wide Web at uniprot.org). However, a skilled artisan would understand that this list is not meant to be limiting and that any suitable hydrogenotroph can be used.

In some embodiments, the at least one hydrogenotroph comprises an organism selected from Table B. Table B lists organisms that have been reported to utilize H₂ according to NCBI Taxonomy (World Wide Web at ncbi.nlm.nih.gov/taxonomy). However, a skilled artisan would understand that this list is not meant to be limiting and any suitable hydrogenotroph can be used.

TABLE A
Exemplary hydrogenotrophs

| Organism | Organism |
|---|---|
| Methanobacterium aarhusense | Methanoplanus sp. enrichment culture clone |
| | CIBANICRA-64 |
| Methanobacterium aggregans | Methanoplanus sp. enrichment culture clone |
| | CIBANICRA-65 |
| Methanobacterium alcaliphilum | Methanoplanus sp. enrichment culture clone |
| | CIBANICRA-66 |
| Methanobacterium alkalithermotolerans | uncultured Methanoplanus sp. |
| Methanobacterium arcticum | Candidatus Methanoculleus |
| | thermohydrogenotrophicum |
| Methanobacterium beijingense | Methanoculleus bourgensis |
| Methanobacterium bryantii | Methanoculleus bourgensis MS2 |
| Methanobacterium cahuitense | Methanoculleus chikugoensis |
| Methanobacterium congolense | Methanoculleus chikugoensis JCM 10825 |
| Methanobacterium curvum | Methanoculleus horonobensis |
| Methanobacterium espanolae | Methanoculleus hydrogenitrophicus |
| Methanobacterium ferruginis | Methanoculleus marisnigri |
| Methanobacterium flexile | Methanoculleus marisnigri JR1 |
| Methanobacterium formicicum | Methanoculleus palmolei |
| Methanobacterium formicicum DSM 3637 | Methanoculleus receptaculi |
| Methanobacterium formicicum JCM 10132 | Methanoculleus sediminis |
| Methanobacterium ivanovii | Methanoculleus submarinus |
| Methanobacterium kanagiense | Methanoculleus taiwanensis |
| Methanobacterium lacus | Methanoculleus thermophilus |
| Methanobacterium movens | Methanoculleus thermophilus DSM 2373 |
| Methanobacterium movilense | unclassified Methanoculleus |
| Methanobacterium oryzae | Methanoculleus sp. |
| Methanobacterium paludis | Methanoculleus sp. 10 |
| Methanobacterium palustre | Methanoculleus sp. 12X3c12 |
| Methanobacterium petrolearium | Methanoculleus sp. 1H2c2 |
| Methanobacterium spitsbergense | Methanoculleus sp. 20 |
| Methanobacterium subterraneum | Methanoculleus sp. 22 |
| Methanobacterium thermaggregans | Methanoculleus sp. 25XMc2 |
| Methanobacterium uliginosum | Methanoculleus sp. 7T |
| Methanobacterium veterum | Methanoculleus sp. Afa-1 |
| Methanobacterium sp. | Methanoculleus sp. Annu2 |
| Methanobacterium sp. 0372-D1 | Methanoculleus sp. Annu3 |
| Methanobacterium sp. 25 | Methanoculleus sp. Annu6 |
| Methanobacterium sp. 28 | Methanoculleus sp. Annu7 |
| Methanobacterium sp. 3Ac | Methanoculleus sp. Annu8 |
| Methanobacterium sp. 3H2 | Methanoculleus sp. BA1 |
| Methanobacterium sp. 42_16 | Methanoculleus sp. CAG: 1088 |
| Methanobacterium sp. 8-1 | Methanoculleus sp. CWC-02 |
| Methanobacterium sp. A39 | Methanoculleus sp. dm2 |
| Methanobacterium sp. AH1 | Methanoculleus sp. DTU007 |
| Methanobacterium sp. AS1 | Methanoculleus sp. EBM-46 |
| Methanobacterium sp. BAmetb5 | Methanoculleus sp. F27 |
| Methanobacterium sp. BRmetb2 | Methanoculleus sp. FWC-SCC1 |
| Methanobacterium sp. C5/51 | Methanoculleus sp. FWC-SCC3 |
| Methanobacterium sp. Ch | Methanoculleus sp. HC-1 |
| Methanobacterium sp. CM1 | Methanoculleus sp. IIE1 |
| Methanobacterium sp. CWC-01 | Methanoculleus sp. LH |
| Methanobacterium sp. CX10MB1 | Methanoculleus sp. LH2 |
| Methanobacterium sp. DP | Methanoculleus sp. M06 |
| Methanobacterium sp. ER19 | Methanoculleus sp. M07 |
| Methanobacterium sp. ERen5 | Methanoculleus sp. M11 |
| Methanobacterium sp. F | Methanoculleus sp. MAB1 |
| Methanobacterium sp. G8 | Methanoculleus sp. MAB2 |
| Methanobacterium sp. GRAU-8 | Methanoculleus sp. MAB3 |
| Methanobacterium sp. HD-1 | Methanoculleus sp. MCMB-578 |
| Methanobacterium sp. IM1 | Methanoculleus sp. MCMB-579 |
| Methanobacterium sp. M03 | Methanoculleus sp. MCMB-580 |
| Methanobacterium sp. Maddingley MBC34 | Methanoculleus sp. MCMB-889 |
| Methanobacterium sp. MB1 | Methanoculleus sp. MH98A |
| Methanobacterium sp. Mb10 | Methanoculleus sp. MQ-4 |
| Methanobacterium sp. Mb2 | Methanoculleus sp. RPS4 |
| Methanobacterium sp. Mb3 | Methanoculleus sp. SDB |
| Methanobacterium sp. MB4 | Methanoculleus sp. SLH121 |
| Methanobacterium sp. Mb5 | Methanoculleus sp. T02 |
| Methanobacterium sp. Mb6 | Methanoculleus sp. T03 |
| Methanobacterium sp. Mb7 | Methanoculleus sp. T05 |
| Methanobacterium sp. Mb8 | Methanoculleus sp. T14 |
| Methanobacterium sp. Mb9 | Methanoculleus sp. T6-6.21 |
| Methanobacterium sp. Mba6 | Methanoculleus sp. T6-6.68 |
| Methanobacterium sp. Mg38 | Methanoculleus sp. T6-7.27 |
| Methanobacterium sp. MH | Methanoculleus sp. T6-7.90 |
| Methanobacterium sp. MZ-A1 | Methanoculleus sp. T6-8.48 |
| Methanobacterium sp. NBRC 105039 | Methanoculleus sp. T6-8.7 |
| Methanobacterium sp. OM15 | Methanoculleus sp. UBA208 |
| Methanobacterium sp. Ps21 | Methanoculleus sp. UBA291 |
| Methanobacterium sp. PtaB.Bin024 | Methanoculleus sp. UBA300 |
| Methanobacterium sp. PtaU1.Bin097 | Methanoculleus sp. UBA303 |
| Methanobacterium sp. PtaU1.Bin242 | Methanoculleus sp. UBA307 |
| Methanobacterium sp. R40H9 | Methanoculleus sp. UBA312 |
| Methanobacterium sp. SA-12 | Methanoculleus sp. UBA320 |
| Methanobacterium sp. SMA-27 | Methanoculleus sp. UBA326 |
| Methanobacterium sp. T01 | Methanoculleus sp. UBA331 |
| Methanobacterium sp. T11 | Methanoculleus sp. UBA334 |
| Methanobacterium sp. Tc3 | Methanoculleus sp. UBA340 |
| Methanobacterium sp. TM-8 | Methanoculleus sp. UBA374 |
| Methanobacterium sp. UBA176 | Methanoculleus sp. UBA377 |
| Methanobacterium sp. UBA279 | Methanoculleus sp. UBA389 |
| Methanobacterium sp. UBA283 | Methanoculleus sp. UBA406 |
| Methanobacterium sp. UBA290 | Methanoculleus sp. UBA413 |
| Methanobacterium sp. UBA294 | Methanoculleus sp. UBA416 |
| Methanobacterium sp. UBA295 | Methanoculleus sp. UBA430 |
| Methanobacterium sp. UBA297 | Methanoculleus sp. UBA45 |
| Methanobacterium sp. UBA299 | Methanoculleus sp. UBA77 |
| Methanobacterium sp. UBA302 | Methanoculleus sp. Wushi-C6 |
| Methanobacterium sp. UBA305 | Methanoculleus sp. YWC-01 |
| Methanobacterium sp. UBA310 | Methanoculleus sp. enrichment culture |
| Methanobacterium sp. UBA311 | Methanoculleus sp. enrichment culture clone 01-49 |
| Methanobacterium sp. UBA316 | Methanoculleus sp. enrichment culture clone 01-50 |
| Methanobacterium sp. UBA322 | Methanoculleus sp. enrichment culture clone 01-60 |
| Methanobacterium sp. UBA324 | Methanoculleus sp. enrichment culture clone 1 |
| Methanobacterium sp. UBA327 | Methanoculleus sp. enrichment culture clone A14111 |
| Methanobacterium sp. UBA330 | Methanoculleus sp. enrichment culture clone A2290 |
| Methanobacterium sp. UBA336 | Methanoculleus sp. enrichment culture clone A2294 |
| Methanobacterium sp. UBA339 | Methanoculleus sp. enrichment culture clone A5_10 |
| Methanobacterium sp. UBA341 | Methanoculleus sp. enrichment culture clone A5_11 |
| Methanobacterium sp. UBA350 | Methanoculleus sp. enrichment culture clone A5_12 |
| Methanobacterium sp. UBA351 | Methanoculleus sp. enrichment culture clone A5_14 |
| Methanobacterium sp. UBA353 | Methanoculleus sp. enrichment culture clone A5_15 |
| Methanobacterium sp. UBA355 | Methanoculleus sp. enrichment culture clone A5_16 |
| Methanobacterium sp. UBA357 | Methanoculleus sp. enrichment culture clone A5_18 |
| Methanobacterium sp. UBA368 | Methanoculleus sp. enrichment culture clone A5_19 |
| Methanobacterium sp. UBA373 | Methanoculleus sp. enrichment culture clone A5_2 |
| Methanobacterium sp. UBA375 | Methanoculleus sp. enrichment culture clone A5_21 |
| Methanobacterium sp. UBA379 | Methanoculleus sp. enrichment culture clone A5_22 |
| Methanobacterium sp. UBA380 | Methanoculleus sp. enrichment culture clone A5_23 |
| Methanobacterium sp. UBA384 | Methanoculleus sp. enrichment culture clone A5_25 |
| Methanobacterium sp. UBA385 | Methanoculleus sp. enrichment culture clone A5_26 |
| Methanobacterium sp. UBA388 | Methanoculleus sp. enrichment culture clone A5_27 |
| Methanobacterium sp. UBA390 | Methanoculleus sp. enrichment culture clone A5_28 |
| Methanobacterium sp. UBA391 | Methanoculleus sp. enrichment culture clone A5_3 |
| Methanobacterium sp. UBA397 | Methanoculleus sp. enrichment culture clone A5_30 |
| Methanobacterium sp. UBA405 | Methanoculleus sp. enrichment culture clone A5_31 |
| Methanobacterium sp. UBA410 | Methanoculleus sp. enrichment culture clone A5_33 |
| Methanobacterium sp. UBA418 | Methanoculleus sp. enrichment culture clone A5_35 |
| Methanobacterium sp. UBA419 | Methanoculleus sp. enrichment culture clone A5_36 |
| Methanobacterium sp. UBA42 | Methanoculleus sp. enrichment culture clone A5_4 |
| Methanobacterium sp. UBA426 | Methanoculleus sp. enrichment culture clone A5_40 |
| Methanobacterium sp. UBA428 | Methanoculleus sp. enrichment culture clone A5_42 |
| Methanobacterium sp. UBA44 | Methanoculleus sp. enrichment culture clone A5_47 |
| Methanobacterium sp. UBA455 | Methanoculleus sp. enrichment culture clone A5_52 |
| Methanobacterium sp. UBA479 | Methanoculleus sp. enrichment culture clone A5_53 |
| Methanobacterium sp. UBA74 | Methanoculleus sp. enrichment culture clone A5_56 |
| Methanobacterium sp. UBA8 | Methanoculleus sp. enrichment culture clone A5_57 |
| Methanobacterium sp. XJ-3a | Methanoculleus sp. enrichment culture clone A5_6 |
| Methanobacterium sp. YCM1 | Methanoculleus sp. enrichment culture clone A5_61 |
| Methanobacterium sp. YSL | Methanoculleus sp. enrichment culture clone A5_62 |
| Methanobacterium sp. enrichment culture clone | Methanoculleus sp. enrichment culture clone A5_8 |
| A1ER16 | |
| Methanobacterium sp. enrichment culture clone | Methanoculleus sp. enrichment culture clone |
| E4ER19 | AD_mcrA_17 |
| uncultured Methanobacterium sp. | Methanoculleus sp. enrichment culture clone |
| | AD_mcrA_24 |
| Methanothermobacter crinale | Methanoculleus sp. enrichment culture clone |
| | AD_mcrA_44 |
| Methanothermobacter defluvii | Methanoculleus sp. enrichment culture clone AP10 |
| Methanothermobacter marburgensis | Methanoculleus sp. enrichment culture clone AP17 |
| Methanothermobacter marburgensis str. Marburg | Methanoculleus sp. enrichment culture clone AP18 |
| Methanothermobacter tenebrarum | Methanoculleus sp. enrichment culture clone AP19 |
| Methanothermobacter thermautotrophicus | Methanoculleus sp. enrichment culture clone AP20 |
| Methanothermobacter thermautotrophicus str. | Methanoculleus sp. enrichment culture clone Arz- |
| Delta H | Arch-1 |
| Methanothermobacter thermautotrophicus str. | Methanoculleus sp. enrichment culture clone Arz- |
| Winter | ArchMG-1 |
| Methanothermobacter thermoflexus | Methanoculleus sp. enrichment culture clone BAMC-1 |
| Methanothermobacter thermophilus | Methanoculleus sp. enrichment culture clone BAMC-2 |
| Methanothermobacter wolfeii | Methanoculleus sp. enrichment culture clone gang 12 |
| Methanothermobacter sp. | Methanoculleus sp. enrichment culture clone gang 13 |
| Methanothermobacter sp. CaT2 | Methanoculleus sp. enrichment culture clone HA1_1 |
| Methanothermobacter sp. EMTCatA1 | Methanoculleus sp. enrichment culture clone HA1_10 |
| Methanothermobacter sp. K4 | Methanoculleus sp. enrichment culture clone HA1_11 |
| Methanothermobacter sp. KEPCO-1 | Methanoculleus sp. enrichment culture clone HA1_12 |
| Methanothermobacter sp. MT-2 | Methanoculleus sp. enrichment culture clone HA1_16 |
| Methanothermobacter sp. RY3 | Methanoculleus sp. enrichment culture clone HA1_18 |
| Methanothermobacter sp. TCHS-010 | Methanoculleus sp. enrichment culture clone HA1_19 |
| Methanothermobacter sp. THM-1 | Methanoculleus sp. enrichment culture clone HA1_2 |
| Methanothermobacter sp. THM-2 | Methanoculleus sp. enrichment culture clone HA1_20 |
| Methanothermobacter sp. THUT3 | Methanoculleus sp. enrichment culture clone HA1_21 |
| Methanothermobacter sp. enrichment clone M2 | Methanoculleus sp. enrichment culture clone HA1_22 |
| Methanothermobacter sp. enrichment clone PY1 | Methanoculleus sp. enrichment culture clone HA1_26 |
| Methanothermobacter sp. enrichment clone PY2 | Methanoculleus sp. enrichment culture clone HA1_29 |
| Methanothermobacter sp. enrichment clone SA11 | Methanoculleus sp. enrichment culture clone HA1_3 |
| Methanothermobacter sp. enrichment clone SA2 | Methanoculleus sp. enrichment culture clone HA1_30 |
| Methanothermobacter sp. enrichment culture | Methanoculleus sp. enrichment culture clone HA1_31 |
| Methanothermobacter sp. enrichment culture clone | Methanoculleus sp. enrichment culture clone HA1_32 |
| 02-52 | |
| Methanothermobacter sp. enrichment culture clone | Methanoculleus sp. enrichment culture clone HA1_33 |
| 12 | |
| Methanothermobacter sp. enrichment culture clone | Methanoculleus sp. enrichment culture clone HA1_37 |
| 2 | |
| Methanothermobacter sp. enrichment culture clone | Methanoculleus sp. enrichment culture clone HA1_39 |
| 3 | |
| Methanothermobacter sp. enrichment culture clone | Methanoculleus sp. enrichment culture clone HA1_5 |
| ASK_11 | |
| Methanothermobacter sp. enrichment culture clone | Methanoculleus sp. enrichment culture clone HA1_7 |
| ASK_15 | |
| Methanothermobacter sp. enrichment culture clone | Methanoculleus sp. enrichment culture clone HA1_8 |
| ASK_21 | |
| Methanothermobacter sp. enrichment culture clone | Methanoculleus sp. enrichment culture clone HA_1 |
| ASK_25 | |
| Methanothermobacter sp. enrichment culture clone | Methanoculleus sp. enrichment culture clone HA_10 |
| ASK_3 | |
| Methanothermobacter sp. enrichment culture clone | Methanoculleus sp. enrichment culture clone HA_11 |
| ASK_30 | |
| Methanothermobacter sp. enrichment culture clone | Methanoculleus sp. enrichment culture clone HA_15 |
| ASK_31 | |
| Methanothermobacter sp. enrichment culture clone | Methanoculleus sp. enrichment culture clone HA_17 |
| ASK_33 | |
| Methanothermobacter sp. enrichment culture clone | Methanoculleus sp. enrichment culture clone HA_18 |
| ASK_34 | |
| Methanothermobacter sp. enrichment culture clone | Methanoculleus sp. enrichment culture clone HA_19 |
| ASK_37 | |
| Methanothermobacter sp. enrichment culture clone | Methanoculleus sp. enrichment culture clone HA_2 |
| ASK_38 | |
| Methanothermobacter sp. enrichment culture clone | Methanoculleus sp. enrichment culture clone HA_20 |
| ASK_42 | |
| Methanothermobacter sp. enrichment culture clone | Methanoculleus sp. enrichment culture clone HA_23 |
| ASK_43 | |
| Methanothermobacter sp. enrichment culture clone | Methanoculleus sp. enrichment culture clone HA_26 |
| ASK_6 | |
| Methanothermobacter sp. enrichment culture clone | Methanoculleus sp. enrichment culture clone HA_27 |
| ASK_8 | |
| Methanothermobacter sp. enrichment culture clone | Methanoculleus sp. enrichment culture clone HA_28 |
| ASK_9 | |
| Methanothermobacter sp. enrichment culture clone | Methanoculleus sp. enrichment culture clone HA_29 |
| B11-A-16 | |
| Methanothermobacter sp. enrichment culture clone | Methanoculleus sp. enrichment culture clone HA_3 |
| B11-A-76 | |
| Methanothermobacter sp. enrichment culture clone | Methanoculleus sp. enrichment culture clone HA_30 |
| B13-A-210 | |
| Methanothermobacter sp. enrichment culture clone | Methanoculleus sp. enrichment culture clone HA_32 |
| B13-A-297 | |
| Methanothermobacter sp. enrichment culture clone | Methanoculleus sp. enrichment culture clone HA_35 |
| C1 | |
| Methanothermobacter sp. enrichment culture clone | Methanoculleus sp. enrichment culture clone HA_36 |
| C13 | |
| Methanothermobacter sp. enrichment culture clone | Methanoculleus sp. enrichment culture clone HA_37 |
| C2 | |
| Methanothermobacter sp. enrichment culture clone | Methanoculleus sp. enrichment culture clone HA_38 |
| C3 | |
| Methanothermobacter sp. enrichment culture clone | Methanoculleus sp. enrichment culture clone HA_39 |
| C4 | |
| Methanothermobacter sp. enrichment culture clone | Methanoculleus sp. enrichment culture clone HA_4 |
| CIBANICRA-MBT1 | |
| Methanothermobacter sp. enrichment culture clone | Methanoculleus sp. enrichment culture clone HA_40 |
| L55A_12 | |
| Methanothermobacter sp. enrichment culture clone | Methanoculleus sp. enrichment culture clone HA_41 |
| L55A_24 | |
| Methanothermobacter sp. enrichment culture clone | Methanoculleus sp. enrichment culture clone HA_42 |
| L55A_25 | |
| Methanothermobacter sp. enrichment culture clone | Methanoculleus sp. enrichment culture clone HA_44 |
| L55A_26 | |
| Methanothermobacter sp. enrichment culture clone | Methanoculleus sp. enrichment culture clone HA_46 |
| L55A_35 | |
| Methanothermobacter sp. enrichment culture clone | Methanoculleus sp. enrichment culture clone HA_6 |
| L55A_37 | |
| Methanothermobacter sp. enrichment culture clone | Methanoculleus sp. enrichment culture clone HA_7 |
| MTY2_1 | |
| Methanothermobacter sp. enrichment culture clone | Methanoculleus sp. enrichment culture clone HA_8 |
| MTY2_2 | |
| Methanothermobacter sp. enrichment culture clone | Methanoculleus sp. enrichment culture clone |
| MTY2_3 | L35A3_27 |
| Methanothermobacter sp. enrichment culture clone | Methanoculleus sp. enrichment culture clone |
| MTY2_4 | L35A3_28 |
| Methanothermobacter sp. enrichment culture clone | Methanoculleus sp. enrichment culture clone |
| MTY2_5 | L35A3_29 |
| Methanothermobacter sp. enrichment culture clone | Methanoculleus sp. enrichment culture clone |
| MTY2_6 | L35A3_44 |
| Methanothermobacter sp. enrichment culture clone | Methanoculleus sp. enrichment culture clone L35A_10 |
| MTY2_7 | |
| Methanothermobacter sp. enrichment culture clone | Methanoculleus sp. enrichment culture clone L35A_2 |
| MTY2_8 | |
| Methanothermobacter sp. enrichment culture clone | Methanoculleus sp. enrichment culture clone L35A_26 |
| MTYC_1 | |
| Methanothermobacter sp. enrichment culture clone | Methanoculleus sp. enrichment culture clone L35A_8 |
| MTYC_2 | |
| Methanothermobacter sp. enrichment culture clone | Methanoculleus sp. enrichment culture clone L55A_11 |
| MTYC_4 | |
| Methanothermobacter sp. enrichment culture clone | Methanoculleus sp. enrichment culture clone L55A_14 |
| MTYC_6 | |
| Methanothermobacter sp. enrichment culture clone | Methanoculleus sp. enrichment culture clone L55A_15 |
| MTYC_8 | |
| Methanothermobacter sp. enrichment culture clone | Methanoculleus sp. enrichment culture clone L55A_28 |
| MTYC_9 | |
| uncultured Methanothermobacter sp. | Methanoculleus sp. enrichment culture clone L55A_36 |
| Methanobrevibacter acididurans | Methanoculleus sp. enrichment culture clone L55A_5 |
| Methanobrevibacter arboriphilus | Methanoculleus sp. enrichment culture clone L55A_9 |
| Methanobrevibacter arboriphilus ANOR1 | Methanoculleus sp. enrichment culture clone LA_11 |
| Methanobrevibacter arboriphilus JCM 13429 = | Methanoculleus sp. enrichment culture clone LA_14 |
| DSM 1125 | |
| Methanobrevibacter arboriphilus JCM 9315 | Methanoculleus sp. enrichment culture clone LA_16 |
| Methanobrevibacter boviskoreani | Methanoculleus sp. enrichment culture clone LA_2 |
| Methanobrevibacter boviskoreani JH1 | Methanoculleus sp. enrichment culture clone LA_22 |
| Methanobrevibacter curvatus | Methanoculleus sp. enrichment culture clone LA_45 |
| Methanobrevibacter cuticularis | Methanoculleus sp. enrichment culture clone LA_46 |
| Methanobrevibacter filiformis | uncultured Methanoculleus sp. |
| Methanobrevibacter gottschalkii | Methanofollis aquaemaris |
| Methanobrevibacter gottschalkii DSM 11977 | Methanofollis ethanolicus |
| Methanobrevibacter millerae | Methanofollis fontis |
| Methanobrevibacter olleyae | Methanofollis formosanus |
| Methanobrevibacter oralis | Methanofollis liminatans |
| Methanobrevibacter oralis JMR01 | Methanofollis liminatans DSM 4140 |
| Methanobrevibacter ruminantium | Methanofollis propanolicus |
| Methanobrevibacter ruminantium | Methanofollis tationis |
| Methanobrevibacter smithii | unclassified Methanofollis |
| Methanobrevibacter smithii ATCC 35061 | Methanofollis sp. |
| Methanobrevibacter smithii CAG: 186 | Methanofollis sp. UBA420 |
| Methanobrevibacter smithii DSM 11975 | Methanofollis sp. W23 |
| Methanobrevibacter smithii DSM 2374 | Methanofollis sp. YCM2 |
| Methanobrevibacter smithii DSM 2375 | Methanofollis sp. YCM3 |
| Methanobrevibacter smithii TS145A | Methanofollis sp. YCM4 |
| Methanobrevibacter smithii TS145B | Methanofollis sp. enrichment culture |
| Methanobrevibacter smithii TS146A | uncultured Methanofollis sp. |
| Methanobrevibacter smithii TS146B | Methanospirillum hungatei |
| Methanobrevibacter smithii TS146C | Methanospirillum hungatei JF-1 |
| Methanobrevibacter smithii TS146D | Methanospirillum lacunae |
| Methanobrevibacter smithii TS146E | Methanospirillum psychrodurum |
| Methanobrevibacter smithii TS147A | Methanospirillum stamsii |
| Methanobrevibacter smithii TS147B | unclassified Methanospirillum |
| Methanobrevibacter smithii TS147C | Methanospirillum sp. |
| Methanobrevibacter smithii TS94A | Methanospirillum sp. AJ_1 |
| Methanobrevibacter smithii TS94B | Methanospirillum sp. AJ_10 |
| Methanobrevibacter smithii TS94C | Methanospirillum sp. AJ_11 |
| Methanobrevibacter smithii TS95A | Methanospirillum sp. AJ_2 |
| Methanobrevibacter smithii TS95B | Methanospirillum sp. AJ_3 |
| Methanobrevibacter smithii TS95C | Methanospirillum sp. AJ_4 |
| Methanobrevibacter smithii TS95D | Methanospirillum sp. AJ_5 |
| Methanobrevibacter smithii TS96A | Methanospirillum sp. AJ_7 |
| Methanobrevibacter smithii TS96B | Methanospirillum sp. AJ_8 |
| Methanobrevibacter smithii TS96C | Methanospirillum sp. J.3.6.1-F.2.7.3 |
| Methanobrevibacter thaueri | Methanospirillum sp. JGI 0000059-J12 |
| Methanobrevibacter woesei | Methanospirillum sp. TM20-1 |
| Methanobrevibacter wolinii | Methanospirillum sp. enrichment culture |
| Methanobrevibacter wolinii SH | Methanospirillum sp. enrichment culture clone |
| | AD_Archaea_16 |
| Methanobrevibacter sp. | Methanospirillum sp. enrichment culture clone |
| | AD_Archaea_19 |
| Methanobrevibacter sp. 110 | Methanospirillum sp. enrichment culture clone |
| | AD_Archaea_25 |
| Methanobrevibacter sp. 1Y | Methanospirillum sp. enrichment culture clone |
| | AD_Archaea_43 |
| Methanobrevibacter sp. 229/11 | Methanospirillum sp. enrichment culture clone |
| | AD_Archaea_5 |
| Methanobrevibacter sp. 229/14JGI 0000059-119 | Methanospirillum sp. enrichment culture clone |
| | AD_Archaea_6 |
| Methanobrevibacter sp. 229/4 | Methanospirillum sp. enrichment culture clone |
| | D2CL_Arch_16S_clone2A |
| Methanobrevibacter sp. 229/5 | Methanospirillum sp. enrichment culture clone |
| | D2CL_Arch_16S_clone2B |
| Methanobrevibacter sp. 30Y | Methanospirillum sp. enrichment culture clone |
| | D2CL_mvrD_Clone1 |
| Methanobrevibacter sp. 31A | uncultured Methanospirillum sp. |
| Methanobrevibacter sp. 62 | Candidatus Methanocorpusculum equi |
| Methanobrevibacter sp. 87.7 | Candidatus Methanocorpusculum faecipullorum |
| Methanobrevibacter sp. A27 | Methanocorpusculum aggregans |
| Methanobrevibacter sp. A54 | Methanocorpusculum bavaricum |
| Methanobrevibacter sp. AbM1 | Methanocorpusculum bavaricum DSM 4179 |
| Methanobrevibacter sp. AbM23 | Methanocorpusculum labreanum |
| Methanobrevibacter sp. AbM4 | Methanocorpusculum labreanum Z |
| Methanobrevibacter sp. AK-87 | Methanocorpusculum parvum |
| Methanobrevibacter sp. Alpaca | Methanocorpusculum petauri |
| Methanobrevibacter sp. CIRG-GMbb01 | Methanocorpusculum sinense |
| Methanobrevibacter sp. CIRG-GMbb02 | Methanocorpusculum vombati |
| Methanobrevibacter sp. D5 | unclassified Methanocorpusculum |
| Methanobrevibacter sp. FM1 | Metopus contortus archaeal symbiont |
| Methanobrevibacter sp. FMB1 | Metopus palaeformis endosymbiont |
| Methanobrevibacter sp. FMB2 | Methanocorpusculum sp. |
| Methanobrevibacter sp. FMB3 | Methanocorpusculum sp. GPch4 |
| Methanobrevibacter sp. FMBK1 | Methanocorpusculum sp. MCE |
| Methanobrevibacter sp. FMBK2 | Methanocorpusculum sp. MSP |
| Methanobrevibacter sp. FMBK3 | Methanocorpusculum sp. T07 |
| Methanobrevibacter sp. FMBK4 | Methanocorpusculum sp. T08 |
| Methanobrevibacter sp. FMBK5 | Methanocorpusculum sp. UBA362 |
| Methanobrevibacter sp. FMBK6 | Methanocorpusculum sp. UBA424 |
| Methanobrevibacter sp. FMBK7 | Methanocorpusculum sp. UBA592 |
| Methanobrevibacter sp. G16 | Trimyema sp. archaeal symbiont |
| Methanobrevibacter sp. HW23 | uncultured Methanocorpusculum sp. |
| Methanobrevibacter sp. KB01 | Methanocalculus alkaliphilus |
| Methanobrevibacter sp. LRsD4 | Methanocalculus chunghsingensis |
| Methanobrevibacter sp. Mc30 | Methanocalculus halotolerans |
| Methanobrevibacter sp. MCTS 1-B | Methanocalculus natronophilus |
| Methanobrevibacter sp. MCTS 2-G | Methanocalculus pumilus |
| Methanobrevibacter sp. MD101 | Methanocalculus taiwanensis |
| Methanobrevibacter sp. MD102 | unclassified Methanocalculus |
| Methanobrevibacter sp. MD103 | Methanocalculus sp. |
| Methanobrevibacter sp. MD104 | Methanocalculus sp. 1H1Hc7 |
| Methanobrevibacter sp. MD105 | Methanocalculus sp. 52_23 |
| Methanobrevibacter sp. MO-MVB | Methanocalculus sp. AMF-A1 |
| Methanobrevibacter sp. N13 | Methanocalculus sp. AMF-B2M |
| Methanobrevibacter sp. N17 | Methanocalculus sp. AMF-Bu2 |
| Methanobrevibacter sp. N17A | Methanocalculus sp. AMF-Cr1 |
| Methanobrevibacter sp. N17G | Methanocalculus sp. AMF-Pr1 |
| Methanobrevibacter sp. N30 | Methanocalculus sp. AMF10 |
| Methanobrevibacter sp. N51 | Methanocalculus sp. AMF3 |
| Methanobrevibacter sp. N58 | Methanocalculus sp. AMF4 |
| Methanobrevibacter sp. N58C | Methanocalculus sp. AMF5 |
| Methanobrevibacter sp. N58T | Methanocalculus sp. AMF6 |
| Methanobrevibacter sp. N70 | Methanocalculus sp. AMF7 |
| Methanobrevibacter sp. NOE | Methanocalculus sp. CA100 |
| Methanobrevibacter sp. OCP | Methanocalculus sp. CC-3 |
| Methanobrevibacter sp. OttesenSCG-928-108 | Methanocalculus sp. LA1 |
| Methanobrevibacter sp. OttesenSCG-928-K11 | Methanocalculus sp. LA2 |
| Methanobrevibacter sp. R4C | Methanocalculus sp. LA3 |
| Methanobrevibacter sp. RsI3 | Methanocalculus sp. LA4 |
| Methanobrevibacter sp. RsW3 | Methanocalculus sp. LA5 |
| Methanobrevibacter sp. SM9 | Methanocalculus sp. LA6 |
| Methanobrevibacter sp. TLL-48-HuF1 | Methanocalculus sp. LA7 |
| Methanobrevibacter sp. TMH8 | Methanocalculus sp. MSAO_Arc1 |
| Methanobrevibacter sp. UBA187 | Methanocalculus sp. MSAO_Arc2 |
| Methanobrevibacter sp. UBA188 | Methanocalculus sp. O1F9702c |
| Methanobrevibacter sp. UBA189 | Methanocalculus sp. enrichment culture clone 01-23 |
| Methanobrevibacter sp. UBA190 | Methanocalculus sp. enrichment culture clone 01-28 |
| Methanobrevibacter sp. UBA212 | Methanocalculus sp. enrichment culture clone 01-67 |
| Methanobrevibacter sp. UBA313 | Methanocalculus sp. enrichment culture clone 02-69 |
| Methanobrevibacter sp. UBA318 | Methanocalculus sp. enrichment culture clone A2-23 |
| Methanobrevibacter sp. UBA325 | Methanocalculus sp. enrichment culture clone A22101 |
| Methanobrevibacter sp. UBA337 | Methanocalculus sp. enrichment culture clone A3-40 |
| Methanobrevibacter sp. UBA352 | Methanocalculus sp. enrichment culture clone A3-53 |
| Methanobrevibacter sp. UBA365 | Methanocalculus sp. enrichment culture clone A4-3 |
| Methanobrevibacter sp. UBA395 | Methanocalculus sp. enrichment culture clone AF10 |
| Methanobrevibacter sp. UBA401 | Methanocalculus sp. enrichment culture clone AF12 |
| Methanobrevibacter sp. UBA403 | Methanocalculus sp. enrichment culture clone AF13 |
| Methanobrevibacter sp. UBA412 | Methanocalculus sp. enrichment culture clone AF14 |
| Methanobrevibacter sp. UBA417 | Methanocalculus sp. enrichment culture clone AF16 |
| Methanobrevibacter sp. UBA46 | Methanocalculus sp. enrichment culture clone AF17 |
| Methanobrevibacter sp. UBA586 | Methanocalculus sp. enrichment culture clone AF18 |
| Methanobrevibacter sp. UBA594 | Methanocalculus sp. enrichment culture clone AF19 |
| Methanobrevibacter sp. V14 | Methanocalculus sp. enrichment culture clone AF2 |
| Methanobrevibacter sp. V74 | Methanocalculus sp. enrichment culture clone AF20 |
| Methanobrevibacter sp. WBY1 | Methanocalculus sp. enrichment culture clone AF3 |
| Methanobrevibacter sp. XT106 | Methanocalculus sp. enrichment culture clone AF4 |
| Methanobrevibacter sp. XT108 | Methanocalculus sp. enrichment culture clone AF5 |
| Methanobrevibacter sp. XT109 | Methanocalculus sp. enrichment culture clone AF7 |
| Methanobrevibacter sp. YE286 | Methanocalculus sp. enrichment culture clone AF8 |
| Methanobrevibacter sp. YE287 | Methanocalculus sp. enrichment culture clone AF9 |
| Methanobrevibacter sp. YE288 | Methanocalculus sp. enrichment culture clone |
| | L35A_25 |
| Methanobrevibacter sp. YE296 | Methanocalculus sp. enrichment culture clone |
| | L35A_27 |
| Methanobrevibacter sp. YE300 | Methanocalculus sp. enrichment culture clone L35A_3 |
| Methanobrevibacter sp. YE301 | Methanocalculus sp. enrichment culture clone L35A_9 |
| Methanobrevibacter sp. YE302 | Methanocalculus sp. enrichment culture clone SA2-91 |
| Methanobrevibacter sp. YE303 | uncultured Methanocalculus sp. |
| Methanobrevibacter sp. YE304 | Methanocella arvoryzae |
| Methanobrevibacter sp. YE315 | Methanocella arvoryzae MRE50 |
| Methanobrevibacter sp. YLM1 | Methanocella conradii |
| Methanobrevibacter sp. Z4 | Methanocella conradii HZ254 |
| Methanobrevibacter sp. Z6 | Methanocella paludicola |
| Methanobrevibacter sp. Z8 | Methanocella paludicola SANAE |
| unclassified Methanobrevibacter (miscellaneous) | unclassified Methanocella |
| endosymbiont ‘TS1’ of Trimyema compressum | Methanocella sp. |
| methanogenic endosymbiont of Nyctotherus | Methanocella sp. CWC-04 |
| cordiformis | |
| methanogenic endosymbiont of Nyctotherus ovalis | Methanocella sp. PtaU1.Bin125 |
| methanogenic endosymbiont of Nyctotherus velox | uncultured Methanocella sp. |
| methanogenic symbiont RS104 | Methanolinea mesophila |
| methanogenic symbiont RS105 | Methanolinea tarda |
| methanogenic symbiont RS208 | Methanolinea tarda NOBI-1 |
| methanogenic symbiont RS301 | unclassified Methanolinea |
| methanogenic symbiont RS404 | Methanolinea sp. |
| methanogenic symbiont RS801 | Methanolinea sp. G10 |
| methanogenic symbiont RS802 | Methanolinea sp. JGI 0000059-I19 |
| methanogenic endosymbiont of Scuticociliatia sp. | Methanolinea sp. SDB |
| GW7 | |
| Methanobrevibacter sp. enrichment culture clone | Methanolinea sp. UBA144 |
| 10 | |
| Methanobrevibacter sp. enrichment culture clone 9 | Methanolinea sp. UBA145 |
| Methanobrevibacter sp. enrichment culture clone | Methanolinea sp. UBA155 |
| IZQ1_CE | |
| Methanobrevibacter sp. enrichment culture clone | Methanolinea sp. UBA245 |
| IZQ2_CE | |
| Methanobrevibacter sp. HI1 | Methanolinea sp. UBA275 |
| Methanobrevibacter sp. HI26 | Methanolinea sp. UBA277 |
| Methanobrevibacter sp. HI28 | Methanolinea sp. UBA286 |
| Methanobrevibacter sp. HW1 | Methanolinea sp. UBA437 |
| Methanobrevibacter sp. HW2 | Methanolinea sp. UBA451 |
| Methanobrevibacter sp. HW3 | Methanolinea sp. UBA473 |
| Methanobrevibacter sp. LHD12 | Methanolinea sp. UBA477 |
| Methanobrevibacter sp. LHD2 | Methanolinea sp. enrichment culture |
| Methanobrevibacter sp. LHM8 | Methanolinea sp. enrichment culture clone B1-A-15 |
| Methanobrevibacter sp. LRsD2 | Methanolinea sp. enrichment culture clone B1-A-23 |
| Methanobrevibacter sp. LRsD3 | Methanolinea sp. enrichment culture clone HA_24 |
| Methanobrevibacter sp. LRsM1 | Methanolinea sp. enrichment culture clone HA_31 |
| Methanobrevibacter sp. R1 | Methanolinea sp. enrichment culture clone HA_9 |
| Methanobrevibacter sp. R2 | Methanolinea sp. enrichment culture clone L35A3_11 |
| Methanobrevibacter sp. R3 | Methanolinea sp. enrichment culture clone L35A3_14 |
| Methanobrevibacter sp. R4 | Methanolinea sp. enrichment culture clone L35A3_2 |
| Methanobrevibacter sp. R5 | Methanolinea sp. enrichment culture clone L35A3_21 |
| Methanobrevibacter sp. RsI12 | Methanolinea sp. enrichment culture clone L35A3_22 |
| Methanobrevibacter sp. RsI17 | Methanolinea sp. enrichment culture clone L35A3_23 |
| Methanobrevibacter sp. RsI4 | Methanolinea sp. enrichment culture clone L35A3_26 |
| Methanobrevibacter sp. RsW10 | Methanolinea sp. enrichment culture clone L35A3_3 |
| Methanobrevibacter sp. RsW2 | Methanolinea sp. enrichment culture clone L35A3_30 |
| uncultured archaeon Ar40 | Methanolinea sp. enrichment culture clone L55A_18 |
| uncultured Methanobrevibacter sp. | Methanolinea sp. enrichment culture clone L55A_3 |
| uncultured termite gut bacterium Cd30 | Methanolinea sp. enrichment culture clone L55A_7 |
| unidentified methanogen ARC1 | Methanolinea sp. enrichment culture clone SA2-93 |
| unidentified methanogen ARC12 | uncultured Methanolinea sp. |
| unidentified methanogen ARC13 | Methanoregula boonei |
| unidentified methanogen ARC15 | Methanoregula boonei 6A8 |
| unidentified methanogen ARC19 | Methanoregula formicica |
| unidentified methanogen ARC20 | Methanoregula formicica SMSP |
| unidentified methanogen ARC23 | unclassified Methanoregula |
| unidentified methanogen ARC24 | Methanoregula sp. |
| unidentified methanogen ARC25 | Methanoregula sp. PtaB.Bin085 |
| unidentified methanogen ARC26 | Methanoregula sp. PtaU1.Bin006 |
| unidentified methanogen ARC27 | Methanoregula sp. PtaU1.Bin051 |
| unidentified methanogen ARC28 | Methanoregula sp. SKADARSKE-2 |
| unidentified methanogen ARC32 | Methanoregula sp. UBA143 |
| unidentified methanogen ARC33 | Methanoregula sp. UBA154 |
| unidentified methanogen ARC40 | Methanoregula sp. UBA244 |
| unidentified methanogen ARC41 | Methanoregula sp. UBA247 |
| unidentified methanogen ARC44 | Methanoregula sp. UBA274 |
| unidentified methanogen ARC50 | Methanoregula sp. UBA276 |
| unidentified methanogen ARC51 | Methanoregula sp. UBA278 |
| unidentified methanogen ARC52 | Methanoregula sp. UBA434 |
| unidentified methanogen ARC53 | Methanoregula sp. UBA450 |
| unidentified methanogen ARC61 | Methanoregula sp. UBA452 |
| unidentified methanogen ARC65 | Methanoregula sp. UBA469 |
| unidentified methanogen ARC66 | Methanoregula sp. UBA471 |
| Methanosphaera cuniculi | Methanoregula sp. UBA64 |
| Methanosphaera stadtmanae | uncultured Methanoregula sp. |
| Methanosphaera stadtmanae DSM 3091 | Methanosphaerula palustris |
| unclassified Methanosphaera | Methanosphaerula palustris E1-9c |
| Methanosphaera sp. | Methanosphaerula sp. enrichment culture clone |
| | MGHR |
| Methanosphaera sp. A4 | uncultured Methanosphaerula sp. |
| Methanosphaera sp. A6 | Methanosarcina acetivorans |
| Methanosphaera sp. BMS | Methanosarcina acetivorans C2A |
| Methanosphaera sp. DEW79 | Methanosarcina baltica |
| Methanosphaera sp. ISO3-F5 | Methanosarcina baltica GS1-A |
| Methanosphaera sp. r00010 | Methanosarcina barkeri |
| Methanosphaera sp. rholeuAM130 | Methanosarcina barkeri 227 |
| Methanosphaera sp. rholeuAM270 | Methanosarcina barkeri 3 |
| Methanosphaera sp. rholeuAM6 | Methanosarcina barkeri CM1 |
| Methanosphaera sp. rholeuAM74 | Methanosarcina barkeri JCM 10043 |
| Methanosphaera sp. SHI1033 | Methanosarcina barkeri MS |
| Methanosphaera sp. SHI613 | Methanosarcina barkeri str. Fusaro |
| Methanosphaera sp. TY-2 | Methanosarcina barkeri str. Wiesmoor |
| Methanosphaera sp. Vir-13MRS | Methanosarcina calensis |
| Methanosphaera sp. WGK6 | Methanosarcina calensis str. Cali |
| Methanosphaera sp. R6 | Methanosarcina flavescens |
| uncultured Methanosphaera sp. | Methanosarcina horonobensis |
| unidentified methanogen ARC14 | Methanosarcina horonobensis HB-1 = JCM 15518 |
| unidentified methanogen ARC17 | Methanosarcina lacustris |
| unidentified methanogen ARC18 | Methanosarcina lacustris Z-7289 |
| unidentified methanogen ARC21 | Methanosarcina lacustris ZS |
| unidentified methanogen ARC29 | Methanosarcina mazei |
| unidentified methanogen ARC30 | Methanosarcina mazei C16 |
| unidentified methanogen ARC39 | Methanosarcina mazei Go1 |
| unidentified methanogen ARC43 | Methanosarcina mazei JCM 9314 |
| unidentified methanogen ARC49 | Methanosarcina mazei LYC |
| unidentified methanogen ARC62 | Methanosarcina mazei S-6 |
| unidentified methanogen ARC8 | Methanosarcina mazei SarPi |
| Methanothermus fervidus | Methanosarcina mazei TMA |
| Methanothermus fervidus DSM 2088 | Methanosarcina mazei Tuc01 |
| Methanothermus jannaschii | Methanosarcina mazei WWM610 |
| Methanothermus sociabilis | Methanosarcina semesiae |
| uncultured Methanothermus sp. | Methanosarcina siciliae |
| Methanopyrus kandleri | Methanosarcina siciliae C2J |
| Methanopyrus kandleri AV19 | Methanosarcina siciliae HI350 |
| unclassified Methanopyrus | Methanosarcina siciliae T4/M |
| Methanopyrus sp. | Methanosarcina soligelidi |
| Methanopyrus sp. Dodo7_105P | Methanosarcina spelaei |
| Methanopyrus sp. Fe97_1 | Methanosarcina subterranea |
| Methanopyrus sp. KOL6 | Methanosarcina thermophila |
| Methanopyrus sp. SMT4-100P | Methanosarcina thermophila CHTI-55 |
| Methanopyrus sp. SNP6 | Methanosarcina thermophila MST-A1 |
| uncultured Methanopyrus sp. | Methanosarcina thermophila TM-1 |
| Methanococcus aeolicus | Methanosarcina vacuolata |
| Methanococcus aeolicus Nankai-3 | Methanosarcina vacuolata Z-761 |
| Methanococcus maripaludis | unclassified Methanosarcina |
| Methanococcus maripaludis C5 | Methanosarcina sp. |
| Methanococcus maripaludis C6 | Methanosarcina sp. 1.H.A.2.2 |
| Methanococcus maripaludis C7 | Methanosarcina sp. 1.H.T.1A.1 |
| Methanococcus maripaludis KA1 | Methanosarcina sp. 13XMc1 |
| Methanococcus maripaludis OS7 | Methanosarcina sp. 1H1 |
| Methanococcus maripaludis S2 | Methanosarcina sp. 2.H.A.1B.4 |
| Methanococcus maripaludis X1 | Methanosarcina sp. 2.H.T.1A.15 |
| Methanococcus vannielii | Methanosarcina sp. 2.H.T.1A.3 |
| Methanococcus vannielii SB | Methanosarcina sp. 2.H.T.1A.6 |
| Methanococcus voltae | Methanosarcina sp. 2.H.T.1A.8 |
| Methanococcus voltae A3 | Methanosarcina sp. 2214B |
| Methanococcus voltae PS | Methanosarcina sp. 48 |
| unclassified Methanococcus | Methanosarcina sp. 795 |
| Methanococcus sp. | Methanosarcina sp. A14 |
| Methanococcus sp. Dex60a43 | Methanosarcina sp. AbM25 |
| Methanococcus sp. Fe85_1_1 | Methanosarcina sp. AK-6 |
| Methanococcus sp. Mc55_1 | Methanosarcina sp. Ant1 |
| Methanococcus sp. Mc55_19 | Methanosarcina sp. CM2 |
| Methanococcus sp. Mc55_2 | Methanosarcina sp. DH1 |
| Methanococcus sp. Mc55_20 | Methanosarcina sp. DH2 |
| Methanococcus sp. Mc70_1 | Methanosarcina sp. DSM 11855 |
| Methanococcus sp. Mc70_2 | Methanosarcina sp. DTU009 |
| Methanococcus sp. Mc85_2 | Methanosarcina sp. ERenArc_MAG2 |
| Methanococcus sp. Ms33_19 | Methanosarcina sp. FR |
| Methanococcus sp. Ms33_20 | Methanosarcina sp. GRAU-10 |
| Methanococcus sp. Ms55_19 | Methanosarcina sp. JL01 |
| Methanococcus sp. Ms55_20 | Methanosarcina sp. JM-1 |
| Methanococcus sp. P2F9701a | Methanosarcina sp. Kolksee |
| Methanococcus sp. enrichment culture clone | Methanosarcina sp. KYL-1 |
| CIBANICRA-21 | |
| Methanococcus sp. enrichment culture clone | Methanosarcina sp. M15 |
| CIBANICRA-22 | |
| Methanococcus sp. enrichment culture clone | Methanosarcina sp. M37 |
| CIBANICRA-23 | |
| Methanococcus sp. enrichment culture clone | Methanosarcina sp. MET5BHJ |
| CIBANICRA-24 | |
| Methanococcus sp. enrichment culture clone | Methanosarcina sp. MO-MS1 |
| CIBANICRA-25 | |
| Methanococcus sp. enrichment culture clone | Methanosarcina sp. MSH10X1 |
| CIBANICRA-26 | |
| Methanococcus sp. enrichment culture clone | Methanosarcina sp. MSS35 |
| CIBANICRA-27 | |
| Methanococcus sp. enrichment culture clone | Methanosarcina sp. MTP4 |
| CIBANICRA-28 | |
| Methanococcus sp. enrichment culture clone | Methanosarcina sp. Naples 100 |
| CIBANICRA-29 | |
| Methanococcus sp. enrichment culture clone | Methanosarcina sp. Pr1 |
| CIBANICRA-30 | |
| Methanococcus sp. enrichment culture clone | Methanosarcina sp. Pr2 |
| CIBANICRA-31 | |
| Methanococcus sp. enrichment culture clone | Methanosarcina sp. RPS13 |
| CIBANICRA-32 | |
| Methanococcus sp. enrichment culture clone | Methanosarcina sp. SMA-17 |
| CIBANICRA-33 | |
| Methanococcus sp. enrichment culture clone | Methanosarcina sp. T36 |
| CIBANICRA-34 | |
| Methanococcus sp. enrichment culture clone | Methanosarcina sp. TMA3RMK |
| CIBANICRA-35 | |
| Methanococcus sp. enrichment culture clone | Methanosarcina sp. UBA135 |
| CIBANICRA-36 | |
| Methanococcus sp. enrichment culture clone | Methanosarcina sp. UBA289 |
| CIBANICRA-37 | |
| Methanococcus sp. enrichment culture clone | Methanosarcina sp. UBA293 |
| CIBANICRA-38 | |
| Methanococcus sp. enrichment culture clone | Methanosarcina sp. UBA301 |
| CIBANICRA-MBT10 | |
| Methanococcus sp. enrichment culture clone | Methanosarcina sp. UBA304 |
| CIBANICRA-MBT4 | |
| uncultured Methanococcus sp. | Methanosarcina sp. UBA323 |
| Methanothermococcus okinawensis | Methanosarcina sp. UBA338 |
| Methanothermococcus okinawensis IH1 | Methanosarcina sp. UBA342 |
| Methanothermococcus thermolithotrophicus | Methanosarcina sp. UBA361 |
| Methanothermococcus thermolithotrophicus DSM | Methanosarcina sp. UBA363 |
| 2095 | |
| unclassified Methanothermococcus | Methanosarcina sp. UBA369 |
| Methanothermococcus sp. | Methanosarcina sp. UBA376 |
| Methanothermococcus sp. BW11 | Methanosarcina sp. UBA383 |
| Methanothermococcus sp. Dodo7_55M | Methanosarcina sp. UBA392 |
| Methanothermococcus sp. E1855-M | Methanosarcina sp. UBA398 |
| Methanothermococcus sp. Ep55 | Methanosarcina sp. UBA402 |
| Methanothermococcus sp. Ep70 | Methanosarcina sp. UBA411 |
| Methanothermococcus sp. JdFR-03 | Methanosarcina sp. UBA423 |
| Methanothermococcus sp. KM5-1nC | Methanosarcina sp. UBA43 |
| Methanothermococcus sp. Mc-1-55 | Methanosarcina sp. UBA47 |
| Methanothermococcus sp. Mc37 | Methanosarcina sp. UBA5 |
| Methanothermococcus sp. Mc55 | Methanosarcina sp. UBA591 |
| Methanothermococcus sp. Mc70 | Methanosarcina sp. UBA7 |
| Methanothermococcus sp. Mc70_19 | Methanosarcina sp. WH-1 |
| Methanothermococcus sp. Pal55-Mc | Methanosarcina sp. WH1 |
| Methanothermococcus sp. SCGC AD-155-C09 | Methanosarcina sp. WWM596 |
| Methanothermococcus sp. SCGC AD-155-E23 | Methanosarcina sp. Z-7115 |
| Methanothermococcus sp. SCGC AD-155-K20 | Methanosarcina sp. enrichment culture |
| Methanothermococcus sp. SCGC AD-155-M21 | Methanosarcina sp. enrichment culture clone 01-27 |
| Methanothermococcus sp. SCGC AD-155-N22 | Methanosarcina sp. enrichment culture clone 4 |
| Methanothermococcus sp. enrichment clone M11 | Methanosarcina sp. enrichment culture clone 5 |
| Methanothermococcus sp. enrichment clone M37 | Methanosarcina sp. enrichment culture clone 6 |
| uncultured Methanothermococcus sp. | Methanosarcina sp. enrichment culture clone A02 |
| Methanocaldococcus bathoardescens | Methanosarcina sp. enrichment culture clone A03 |
| Methanocaldococcus fervens | Methanosarcina sp. enrichment culture clone A04 |
| Methanocaldococcus fervens AG86 | Methanosarcina sp. enrichment culture clone A05 |
| Methanocaldococcus indicus | Methanosarcina sp. enrichment culture clone A06 |
| Methanocaldococcus infernus | Methanosarcina sp. enrichment culture clone A07 |
| Methanocaldococcus infernus ME | Methanosarcina sp. enrichment culture clone A08 |
| Methanocaldococcus jannaschii | Methanosarcina sp. enrichment culture clone A09 |
| Methanocaldococcus jannaschii DSM 2661 | Methanosarcina sp. enrichment culture clone A1-10 |
| Methanocaldococcus lauensis | Methanosarcina sp. enrichment culture clone A1-14 |
| Methanocaldococcus villosus | Methanosarcina sp. enrichment culture clone A1-18 |
| Methanocaldococcus villosus KIN24-T80 | Methanosarcina sp. enrichment culture clone A1-24 |
| Methanocaldococcus vulcanius | Methanosarcina sp. enrichment culture clone A1-30 |
| Methanocaldococcus vulcanius M7 | Methanosarcina sp. enrichment culture clone A1-6 |
| unclassified Methanocaldococcus | Methanosarcina sp. enrichment culture clone A10 |
| Methanocaldococcus sp. | Methanosarcina sp. enrichment culture clone A11 |
| Methanocaldococcus sp. 70-8-3 | Methanosarcina sp. enrichment culture clone A12 |
| Methanocaldococcus sp. Dodo7_85M | Methanosarcina sp. enrichment culture clone A2-50 |
| Methanocaldococcus sp. E1885-M | Methanosarcina sp. enrichment culture clone A2-7 |
| Methanocaldococcus sp. FS406-22 | Methanosarcina sp. enrichment culture clone A2-9 |
| Methanocaldococcus sp. Mc-1-85 | Methanosarcina sp. enrichment culture clone A4-11 |
| Methanocaldococcus sp. Mc-2-70 | Methanosarcina sp. enrichment culture clone A4-19 |
| Methanocaldococcus sp. Mc-2-85 | Methanosarcina sp. enrichment culture clone A4-2 |
| Methanocaldococcus sp. Mc-365-70 | Methanosarcina sp. enrichment culture clone A4-23 |
| Methanocaldococcus sp. Mc-365-85 | Methanosarcina sp. enrichment culture clone A4-49 |
| Methanocaldococcus sp. Mc-I-85 | Methanosarcina sp. enrichment culture clone |
| | AD_Archaea_44 |
| Methanocaldococcus sp. Mc-S-85 | Methanosarcina sp. enrichment culture clone AM11 |
| Methanocaldococcus sp. SLH | Methanosarcina sp. enrichment culture clone AM12 |
| Methanocaldococcus sp. SMT4-70M | Methanosarcina sp. enrichment culture clone AM17 |
| uncultured Methanocaldococcus sp. | Methanosarcina sp. enrichment culture clone AM18 |
| Methanotorris formicicus | Methanosarcina sp. enrichment culture clone AM19 |
| Methanotorris formicicus Mc-S-70 | Methanosarcina sp. enrichment culture clone AM4 |
| Methanotorris igneus | Methanosarcina sp. enrichment culture clone AM6 |
| Methanotorris igneus Kol 5 | Methanosarcina sp. enrichment culture clone B01 |
| unclassified Methanotorris | Methanosarcina sp. enrichment culture clone B02 |
| Methanotorris sp. Mc-I-70 | Methanosarcina sp. enrichment culture clone B03 |
| uncultured Methanotorris sp. | Methanosarcina sp. enrichment culture clone B04 |
| Methanomicrobium antiquum | Methanosarcina sp. enrichment culture clone B05 |
| Methanomicrobium mobile | Methanosarcina sp. enrichment culture clone B06 |
| Methanomicrobium mobile BP | Methanosarcina sp. enrichment culture clone B07 |
| unclassified Methanomicrobium | Methanosarcina sp. enrichment culture clone B08 |
| Methanomicrobium sp. | Methanosarcina sp. enrichment culture clone B09 |
| Methanomicrobium sp. W14 | Methanosarcina sp. enrichment culture clone B10 |
| Methanobacterium sp. enrichment culture | Methanosarcina sp. enrichment culture clone B11 |
| Methanobacterium sp. enrichment culture clone 01- | Methanosarcina sp. enrichment culture clone B12 |
| 01 | |
| Methanobacterium sp. enrichment culture clone 01- | Methanosarcina sp. enrichment culture clone BAMC-6 |
| 20 | |
| Methanobacterium sp. enrichment culture clone 01- | Methanosarcina sp. enrichment culture clone |
| 21 | BER1_CD |
| Methanobacterium sp. enrichment culture clone 01- | Methanosarcina sp. enrichment culture clone |
| 31 | BER2_CD |
| Methanobacterium sp. enrichment culture clone 01- | Methanosarcina sp. enrichment culture clone |
| 42 | BER3_CD |
| Methanobacterium sp. enrichment culture clone 01- | Methanosarcina sp. enrichment culture clone |
| 66 | BER4_CD |
| Methanobacterium sp. enrichment culture clone 02- | Methanosarcina sp. enrichment culture clone C02 |
| 01 | |
| Methanobacterium sp. enrichment culture clone 02- | Methanosarcina sp. enrichment culture clone C03 |
| 15 | |
| Methanobacterium sp. enrichment culture clone 02- | Methanosarcina sp. enrichment culture clone C04 |
| 35 | |
| Methanobacterium sp. enrichment culture clone 02- | Methanosarcina sp. enrichment culture clone C05 |
| 56 | |
| Methanobacterium sp. enrichment culture clone 1 | Methanosarcina sp. enrichment culture clone C06 |
| Methanobacterium sp. enrichment culture clone 3 | Methanosarcina sp. enrichment culture clone C07 |
| Methanobacterium sp. enrichment culture clone 8 | Methanosarcina sp. enrichment culture clone C08 |
| Methanobacterium sp. enrichment culture clone | Methanosarcina sp. enrichment culture clone C09 |
| A1499 | |
| Methanobacterium sp. enrichment culture clone | Methanosarcina sp. enrichment culture clone C10 |
| B13-A-141 | |
| Methanobacterium sp. enrichment culture clone | Methanosarcina sp. enrichment culture clone C11 |
| CIBANICRA-16 | |
| Methanobacterium sp. enrichment culture clone | Methanosarcina sp. enrichment culture clone C12 |
| CIBANICRA-17 | |
| Methanobacterium sp. enrichment culture clone | Methanosarcina sp. enrichment culture clone |
| CIBANICRA-18 | CIBANICRA-15 |
| Methanobacterium sp. enrichment culture clone | Methanosarcina sp. enrichment culture clone |
| CIBANICRA-19 | CIBANICRA-MBT2 |
| Methanobacterium sp. enrichment culture clone | Methanosarcina sp. enrichment culture clone |
| CIBANICRA-20 | CIBANICRA-MBT5 |
| Methanobacterium sp. enrichment culture clone | Methanosarcina sp. enrichment culture clone |
| CIBANICRA-MBT3 | CIBANICRA-MBT6 |
| Methanobacterium sp. enrichment culture clone | Methanosarcina sp. enrichment culture clone D01 |
| CIBANICRA-MBT7 | |
| Methanobacterium sp. enrichment culture clone | Methanosarcina sp. enrichment culture clone D02 |
| CIBANICRA-MBT8 | |
| Methanobacterium sp. enrichment culture clone | Methanosarcina sp. enrichment culture clone D03 |
| CIBANICRA-MBT9 | |
| Methanobacterium sp. enrichment culture clone | Methanosarcina sp. enrichment culture clone D04 |
| MBT-1 | |
| Methanobacterium sp. enrichment culture clone | Methanosarcina sp. enrichment culture clone D05 |
| MBT-10 | |
| Methanobacterium sp. enrichment culture clone | Methanosarcina sp. enrichment culture clone D06 |
| MBT-12 | |
| Methanobacterium sp. enrichment culture clone | Methanosarcina sp. enrichment culture clone D07 |
| MBT-2 | |
| Methanobacterium sp. enrichment culture clone | Methanosarcina sp. enrichment culture clone D08 |
| MBT-3 | |
| Methanobacterium sp. enrichment culture clone | Methanosarcina sp. enrichment culture clone D09 |
| MBT-5 | |
| Methanobacterium sp. enrichment culture clone | Methanosarcina sp. enrichment culture clone D10 |
| MBT-6 | |
| Methanobacterium sp. enrichment culture clone | Methanosarcina sp. enrichment culture clone D11 |
| MBT-7 | |
| Methanobacterium sp. enrichment culture clone | Methanosarcina sp. enrichment culture clone D12 |
| MBT-8 | |
| Methanobacterium sp. enrichment culture clone | Methanosarcina sp. enrichment culture clone E01 |
| MBT-9 | |
| Methanobacterium sp. enrichment culture clone | Methanosarcina sp. enrichment culture clone E02 |
| No15 | |
| Methanobacterium sp. enrichment culture clone | Methanosarcina sp. enrichment culture clone E03 |
| No36 | |
| Methanobacterium sp. enrichment culture clone | Methanosarcina sp. enrichment culture clone E04 |
| No4 | |
| Methanobacterium sp. enrichment culture clone | Methanosarcina sp. enrichment culture clone E05 |
| No51 | |
| Methanobacterium sp. enrichment culture clone | Methanosarcina sp. enrichment culture clone E06 |
| No55 | |
| Methanobacterium sp. enrichment culture clone | Methanosarcina sp. enrichment culture clone E07 |
| No7 | |
| Methanobacterium sp. enrichment culture clone | Methanosarcina sp. enrichment culture clone E08 |
| T01 | |
| Methanobacterium sp. enrichment culture clone | Methanosarcina sp. enrichment culture clone E09 |
| T02 | |
| Methanobacterium sp. enrichment culture clone | Methanosarcina sp. enrichment culture clone E10 |
| T029 | |
| Methanobacterium sp. enrichment culture clone | Methanosarcina sp. enrichment culture clone E12 |
| TES21 | |
| Methanobacterium sp. enrichment culture clone | Methanosarcina sp. enrichment culture clone F01 |
| TES3 | |
| Methanobacterium sp. enrichment culture clone | Methanosarcina sp. enrichment culture clone F02 |
| TES30 | |
| Methanobacterium sp. enrichment culture clone | Methanosarcina sp. enrichment culture clone F03 |
| TES38 | |
| Methanobacterium sp. enrichment culture clone | Methanosarcina sp. enrichment culture clone F04 |
| TES4 | |
| Methanobacterium sp. enrichment culture clone | Methanosarcina sp. enrichment culture clone F05 |
| TES65 | |
| Methanoculleus sp. enrichment culture clone | Methanosarcina sp. enrichment culture clone F06 |
| CIBANICRA-MBT11 | |
| Methanoculleus sp. enrichment culture clone | Methanosarcina sp. enrichment culture clone F07 |
| CIBANICRA-MBT12 | |
| Methanomicrobium sp. enrichment culture clone | Methanosarcina sp. enrichment culture clone F08 |
| MBT-4 | |
| uncultured Methanomicrobium sp. | Methanosarcina sp. enrichment culture clone F09 |
| Methanolacinia paynteri | Methanosarcina sp. enrichment culture clone F10 |
| Methanolacinia paynteri G-2000 | Methanosarcina sp. enrichment culture clone F11 |
| Methanolacinia petrolearia | Methanosarcina sp. enrichment culture clone G01 |
| Methanolacinia petrolearia DSM 11571 | Methanosarcina sp. enrichment culture clone G02 |
| uncultured Methanolacinia sp. | Methanosarcina sp. enrichment culture clone G03 |
| Methanogenium boonei | Methanosarcina sp. enrichment culture clone G04 |
| Methanogenium cariaci | Methanosarcina sp. enrichment culture clone G05 |
| Methanogenium cariaci JCM 10550 | Methanosarcina sp. enrichment culture clone G06 |
| Methanogenium frigidum | Methanosarcina sp. enrichment culture clone G07 |
| Methanogenium frigidum Ace-2 | Methanosarcina sp. enrichment culture clone G08 |
| Methanogenium marinum | Methanosarcina sp. enrichment culture clone G09 |
| Methanogenium organophilum | Methanosarcina sp. enrichment culture clone G10 |
| unclassified Methanogenium | Methanosarcina sp. enrichment culture clone G11 |
| Methanogenium sp. | Methanosarcina sp. enrichment culture clone G12 |
| Methanogenium sp. AK-8 | Methanosarcina sp. enrichment culture clone gang 14 |
| Methanogenium sp. M3 | Methanosarcina sp. enrichment culture clone gang 15 |
| Methanogenium sp. MK-MG | Methanosarcina sp. enrichment culture clone H01 |
| Methanogenium sp. S4BF | Methanosarcina sp. enrichment culture clone H02 |
| uncultured archaeon ACE1_A | Methanosarcina sp. enrichment culture clone H03 |
| uncultured archaeon SCALE-14 | Methanosarcina sp. enrichment culture clone H04 |
| uncultured Methanogenium sp. | Methanosarcina sp. enrichment culture clone H06 |
| Methanoplanus endosymbiosus | Methanosarcina sp. enrichment culture clone H07 |
| Methanoplanus limicola | Methanosarcina sp. enrichment culture clone H08 |
| Methanoplanus limicola DSM 2279 | Methanosarcina sp. enrichment culture clone H09 |
| unclassified Methanoplanus | Methanosarcina sp. enrichment culture clone H10 |
| Methanoplanus sp. 6TMc1 | Methanosarcina sp. enrichment culture clone H11 |
| Methanoplanus sp. FWC-SCC4 | Methanosarcina sp. enrichment culture clone H12 |
| Methanoplanus sp. M7 | Methanosarcina sp. enrichment culture clone MGAHR |
| Methanoculleus sp. enrichment culture clone | Methanosarcina sp. enrichment culture clone MSC-1 |
| CIBANICRA-39 | |
| Methanoculleus sp. enrichment culture clone | Methanosarcina sp. enrichment culture clone MSC-2 |
| CIBANICRA-40 | |
| Methanoculleus sp. enrichment culture clone | Methanosarcina sp. enrichment culture clone MSC-3 |
| CIBANICRA-41 | |
| Methanoculleus sp. enrichment culture clone | Methanosarcina sp. enrichment culture clone MSC-4 |
| CIBANICRA-42 | |
| Methanoculleus sp. enrichment culture clone | Methanosarcina sp. enrichment culture clone MSC-5 |
| CIBANICRA-43 | |
| Methanoculleus sp. enrichment culture clone | Methanosarcina sp. enrichment culture clone MSC-6 |
| CIBANICRA-44 | |
| Methanoculleus sp. enrichment culture clone | Methanosarcina sp. enrichment culture clone SA3-106 |
| CIBANICRA-45 | |
| Methanoculleus sp. enrichment culture clone | Methanosarcina sp. enrichment culture clone SA4-12 |
| CIBANICRA-46 | |
| Methanoculleus sp. enrichment culture clone | Methanosarcina sp. enrichment culture clone SA4-24 |
| CIBANICRA-47 | |
| Methanoculleus sp. enrichment culture clone | Methanosarcina sp. enrichment culture clone SA4-8 |
| CIBANICRA-48 | |
| Methanoculleus sp. enrichment culture clone | Methanosarcina sp. enrichment culture clone T-RF52 |
| CIBANICRA-49 | |
| Methanoculleus sp. enrichment culture clone | Methanosarcina sp. enrichment culture DGGE gel |
| CIBANICRA-50 | band Met1 |
| Methanoculleus sp. enrichment culture clone | Methanosarcina sp. enrichment culture DGGE gel |
| CIBANICRA-51 | band Met2 |
| Methanoculleus sp. enrichment culture clone | Methanosarcina sp. enrichment culture DGGE gel |
| CIBANICRA-52 | band Met3 |
| Methanoplanus sp. enrichment culture clone | Methanosarcina sp. enrichment culture DGGE gel |
| CIBANICRA-53 | band Met4 |
| Methanoplanus sp. enrichment culture clone | Methanosarcina sp. enrichment culture DGGE gel |
| CIBANICRA-54 | band Met5 |
| Methanoplanus sp. enrichment culture clone | Methanosarcina sp. mixed culture AL10 |
| CIBANICRA-55 | |
| Methanoplanus sp. enrichment culture clone | uncultured archaeon ACE2_A |
| CIBANICRA-56 | |
| Methanoplanus sp. enrichment culture clone | uncultured Methanosarcina sp. |
| CIBANICRA-57 | |
| Methanoplanus sp. enrichment culture clone | Methanimicrococcus blatticola |
| CIBANICRA-58 | |
| Methanoplanus sp. enrichment culture clone | unclassified Methanimicrococcus |
| CIBANICRA-59 | |
| Methanoplanus sp. enrichment culture clone | Methanimicrococcus sp. |
| CIBANICRA-60 | |
| Methanoplanus sp. enrichment culture clone | Methanimicrococcus sp. At1 |
| CIBANICRA-61 | |
| Methanoplanus sp. enrichment culture clone | Methanimicrococcus sp. Es2 |
| CIBANICRA-62 | |
| Methanoplanus sp. enrichment culture clone | Methanimicrococcus sp. Hf6 |
| CIBANICRA-63 | |
| uncultured Methanimicrococcus sp. | Methanimicrococcus sp. OttesenSCG-928-J09 |

TABLE B
Additional exemplary hydrogenotrophs

| Organism | Organism |
|---|---|
| Candidatus Bathyarchaeota archaeon | Methanocaldococcus infernus (strain DSM 11812/ |
| | JCM 15783/ME) |
| Methanobacteriaceae archaeon | Methanocaldococcus jannaschii |
| Methanobacteriales archaeon | Methanocaldococcus jannaschii (strain ATCC |
| | 43067/DSM 2661/JAL-1/JCM 10045/NBRC |
| | 100440) (Methanococcus jannaschii) |
| Methanobacteriales archaeon HGW- | Methanocaldococcus lauensis |
| Methanobacteriales-1 | |
| Methanobacterium alkalithermotolerans | Methanocaldococcus sp. (strain FS406-22) |
| Methanobacterium bryantii | Methanocaldococcus villosus KIN24-T80 |
| Methanobacterium formicicum (strain DSM 3637/ | Methanocaldococcus vulcanius (strain ATCC |
| PP1) | 700851/DSM 12094/M7) (Methanococcus |
| | vulcanius) |
| Methanobacterium sp. | Methanococcaceae archaeon |
| Methanobacterium sp. A39 | Methanococcus aeolicus (strain ATCC BAA-1280/ |
| | DSM 17508/OCM 812/Nankai-3) |
| Methanobacterium sp. BRmetb2 | Methanococcus maripaludis (Methanococcus |
| | deltae) |
| Methanobacterium sp. Maddingley MBC34 | Methanococcus maripaludis (strain C5/ATCC |
| | BAA-1333) |
| Methanobacterium sp. PtaB.Bin024 | Methanococcus maripaludis (strain C6/ATCC |
| | BAA-1332) |
| Methanobacterium sp. PtaU1.Bin097 | Methanococcus maripaludis (strain C7/ATCC |
| | BAA-1331) |
| Methanobacterium sp. PtaU1.Bin242 | Methanococcus maripaludis (strain S2/LL) |
| Methanobacterium subterraneum | Methanococcus maripaludis KA1 |
| Methanobacterium veterum | Methanococcus maripaludis OS7 |
| Methanobrevibacter gottschalkii | Methanococcus maripaludis X1 |
| Methanobrevibacter gottschalkii DSM 11977 | Methanococcus vannielii (strain ATCC 35089/ |
| | DSM 1224/JCM 13029/OCM 148/SB) |
| Methanobrevibacter millerae | Methanococcus voltae |
| Methanobrevibacter olleyae | Methanococcus voltae (strain ATCC BAA-1334/A3) |
| Methanobrevibacter oralis | Methanocorpusculum labreanum (strain ATCC |
| | 43576/DSM 4855/Z) |
| Methanobrevibacter ruminantium (strain ATCC 35063/ | Methanofervidicoccus abyssi |
| DSM 1093/JCM 13430/OCM 146/M1) | |
| (Methanobacterium ruminantium) | |
| Methanobrevibacter smithii | Methanofervidicoccus sp. A16 |
| Methanobrevibacter smithii (strain ATCC 35061/DSM | Methanolacinia paynteri G-2000 |
| 861/OCM 144/PS) | |
| Methanobrevibacter smithii CAG: 186 | Methanolacinia petrolearia (strain DSM 11571/ |
| | OCM 486/SEBR 4847) (Methanoplanus |
| | petrolearius) |
| Methanobrevibacter smithii DSM 2374 | Methanomicrobiales archaeon |
| Methanobrevibacter smithii DSM 2375 | Methanopyri archaeon |
| Methanobrevibacter sp | Methanopyrus kandleri |
| Methanobrevibacter sp. 87.7 | Methanopyrus kandleri (strain AV19/DSM 6324/ |
| | JCM 9639/NBRC 100938) |
| Methanobrevibacter sp. A27 | Methanoregula formicica (strain DSM 22288/ |
| | NBRC 105244/SMSP) |
| Methanobrevibacter sp. A54 | Methanospirillum hungatei |
| Methanobrevibacter sp. AbM4 | Methanothermobacter defluvii |
| Methanobrevibacter sp. TLL-48-HuF1 | Methanothermobacter marburgensis (strain ATCC |
| | BAA-927/DSM 2133/JCM 14651/NBRC |
| | 100331/OCM 82/Marburg) (Methanobacterium |
| | thermoautotrophicum) |
| Methanobrevibacter sp. YE315 | Methanothermobacter sp |
| Methanobrevibacter thaueri | Methanothermobacter sp. CaT2 |
| Methanobrevibacter woesei | Methanothermobacter sp. EMTCatA1 |
| Methanocaldococcus bathoardescens | Methanothermobacter sp. KEPCO-1 |
| Methanocaldococcus fervens (strain DSM 4213/JCM | Methanothermobacter sp. MT-2 |
| 15782/AG86) (Methanococcus fervens) | |
| Methanothermobacter wolfeii (Methanobacterium | Methanothermobacter sp. THM-1 |
| wolfei) | |
| Methanothermococcus okinawensis | Methanothermobacter sp. THM-2 |
| Methanothermococcus okinawensis (strain DSM 14208/ | Methanothermobacter tenebrarum |
| JCM 11175/IH1) | |
| Methanothermococcus sp. SCGC AD-155-M21 | Methanothermobacter thermautotrophicus |
| | (Methanobacterium thermoformicicum) |
| Methanothermococcus thermolithotrophicus | Methanothermobacter thermautotrophicus (strain |
| (Methanococcus thermolithotrophicus) | ATCC 29096/DSM 1053/JCM 10044/NBRC |
| | 100330/Delta H) (Methanobacterium |
| | thermoautotrophicum) |
| Methanothermus fervidus (strain ATCC 43054/DSM | Methanothermobacter thermautotrophicus (strain |
| 2088/JCM 10308/V24 S) | Winter) (Methanobacterium |
| | thermoautotrophicum) |
| Methanotorris formicicus Mc-S-70 | Methanotorris igneus (strain DSM 5666/JCM |
| | 11834/Kol 5) |

### Systems, Compositions, and Methods for Growing Hydrogenotrophs

Hydrogenotrophs are typically strict anaerobes—they are sensitive to oxygen and oxidants and thus cannot survive with exposure to oxygen or air. Sensitivity to oxygen and oxidants does vary between hydrogenotrophs. Some hydrogenotrophs may require addition of medium additives such as methanol, ethanol, or other non-hydrogenotrophic substrates. Importantly, traditional techniques for culturing hydrogenotrophs require highly explosive gas mixes consisting of 80% H₂ and 20% CO₂ under high pressure (e.g., a range of 180 kPa to 276 kPa) for optimal growth. Thus, growing hydrogenotrophs, especially on a large scale, are difficult, pose significant risks of explosion, and require high capital investment in systems that can both withstand high pressure and operate anaerobically.

In certain aspects, provided herein are systems, compositions, and methods for growing at least one hydrogenotroph that are different from those understood to be required for growing hydrogenotrophs. In some embodiments, a system comprises lower pressure (less than 180 kPa), which is less than the pressure commonly used in the art. In some embodiments, the H₂ concentration in the growth chamber is lower than 80%, which is the concentration commonly used in the art. Such a lower H₂ concentration in the growth chamber allows the use of a lower H₂ concentration in a supply tank. For example, the H₂ concentration in the supply tank may be non-flammable, e.g., less than 4% (as anything higher than 4% is typically flammable). The systems of the present disclosure have not been previously utilized for growing hydrogenotrophs and were considered to provide an inadequate condition for growing hydrogenotrophs, especially at commercial scale.

### Systems

The systems of the present disclosure may comprise any container(s) or growth chamber(s) (e.g., flask, bottle, bioreactor, etc.) that may be adequate for growing hydrogenotrophs.

In certain aspects, provided herein are systems comprising at least one growth chamber. In certain aspects, provided herein are growth chambers of various sizes. In some embodiments, the growth chambers are at least 0.1 L in volume, at least 0.2 L in volume, at least 0.3 L in volume, at least 0.4 L in volume, at least 0.5 L in volume, at least 0.6 L in volume, at least 0.7 L in volume, at least 0.8 L in volume, at least 0.9 L in volume, at least 1 L in volume, at least 5 L in volume, at least 10 L in volume, at least 15 L in volume, at least 20 L in volume, at least 30 L in volume, at least 40 L in volume, at least 50 L in volume, at least 100 L in volume, at least 200 L in volume, at least 250 L in volume, at least 500 L in volume, at least 750 L in volume, at least 1000 L in volume, at least 1500 L in volume, at least 2000 L in volume, at least 2500 L in volume, at least 3000 L in volume, at least 3500 L in volume, at least 4000 L in volume, at least 5000 L in volume, at least 7500 L in volume, at least 10,000 L in volume, at least 15,000 L in volume, or at least 20,000 L in volume. In some embodiments, the bioreactors are about 1 L in volume, about 5 L in volume, about 10 L in volume, about 15 L in volume, about 20 L in volume, about 30 L in volume, about 40 L in volume, about 50 L in volume, about 100 L in volume, about 200 L in volume, about 250 L in volume, about 500 L in volume, about 750 L in volume, about 1000 L in volume, about 1500 L in volume, about 2000 L in volume, about 2500 L in volume, about 3000 L in volume, about 3500 L in volume, about 4000 L in volume, about 5000 L in volume, about 7500 L in volume, about 10,000 L in volume, about 15,000 L in volume, or about 20,000 L in volume.

In some embodiments, the at least one growth chamber comprises a flask, a bottle, or a suitable alternative. In some embodiments, the at least one growth chamber comprises a bioreactor, e.g., a chemostat, a turbidostat. A skilled artisan would understand how to select suitable alternatives.

### Culturing Methods

In certain aspects, provided herein are methods and/or compositions that facilitate the growth of at least one hydrogenotroph. The methods may comprise maintaining the temperature, substrate concentration, cell density, and pH of the growth media. The culturing may begin in a relatively small volume of growth media (e.g., 1 L) where the at least one hydrogenotroph is allowed to reach the log phase of growth. Such culture may be transferred to a larger volume of growth media (e.g., 20 L) for further growth to reach a larger biomass. Depending on the need of the final amount of biomass, such transfer may be repeated more than once.

In some embodiments, a culturing method of growing at least one hydrogenotroph comprises a batch process, in which no extra feeding (e.g., supply of a substrate for growth, e.g., mineral salt, sugar, etc.) of the hydrogenotrophs from beginning to end of the process.

In some embodiments, the culturing method comprises a fed-batch process, in which feeding with substrate and supplements can extend the duration of culture for higher cell densities or switch metabolism. The fed-batch process can comprise any suitable number of feeds, such as at least 1, 2, 3, 4, 5, 6, 7, 8, 9, 10, 11, 12, 13, 14, 15 16, 17, 18, or 19 feeds and/or not more than 2, 3, 4, 5, 6, 7, 8, 9, 10, 11, 12, 13, 14, 15 16, 17, 18, 19, or 20 feed, for example 2-20 feeds.

In some embodiments, the culturing method comprises a continuous process, where either the feed rate of a growth-limiting substance keeps cell density constant or cell density determines the feed rate of the substrate. Cell retention can offer an option of perfusion, which is a process that uses a method to keep cells in a bioreactor while continuously exchanging culture medium. Fresh medium replenishes nutrients and carbon sources, while cellular waste and medium depleted of nutrients are removed. The balanced nature of the feeding allows a steady state to be achieved which can last for days to months. This state is good for long-term production. In preferred embodiments, the continuous process comprises continuous harvesting of hydrogenotrophs (e.g., methanogens) through a liquid output while replacing new medium/substrate at the same rate. In some such embodiments, volume of the reactor may stay within a defined range.

### Pressure & Anaerobic Conditions

Anaerobic conditions may be established and/or maintained by inflow of anaerobic gas(es). In a system comprising a growth medium, gas may be introduced into the headspace and/or sparged through the growth medium. In some embodiments, a system comprising a growth medium (a) is filled with a gas mixture in the headspace; and (b) the headspace gas is sparged through the medium.

In some embodiments where the system comprises at least one methanogen, CH₄ is produced as methanogens grow in number and utilize H₂/CO₂. In some such embodiments, the headspace may be refreshed to provide inflow of new H₂/CO₂ to support growth. In some embodiments, H₂/CO₂ is supplied in batches periodically during culturing. In other embodiments, H₂/CO₂ is supplied continuously during culturing. In other embodiments, H₂/CO₂ is supplied to maintain a desired concentration in the growth vessel, for example using one or more sensors that measures one or more substrate or product of methanogenesis (e.g., H₂, CO₂, CH₄, bicarbonate, etc.) or a functional measure thereof (e.g., turbidity, OD, absorbance, fluorescence, transmittance), transmitting that information to a controller, and then adjusting the flow of H₂/CO₂ using an actuator (e.g., a valve).

In some embodiments, the system may comprise at least one auxiliary instrument that may facilitate the growth of at least one hydrogenotroph. Auxiliary instruments include but are not limited to analytical instruments (sensors, meters, detection devices), control instruments, and actuators. Exemplary auxiliary instruments are described herein and listed in Table C.

In some embodiments, the system may comprise at least one auxiliary (e.g., analytical) instrument that determines various parameters in the growth chamber (e.g., cell density, pH, level of any type of gas (e.g., CH₄, CO₂, H₂, etc.). The auxiliary instruments may include, e.g., a pH meter, a spectrophotometer, a turbimeter, and/or an instrument analyzing the gas content. For example, an auxiliary instrument may determine the gas content in the system (e.g., the level of CH₄, CO₂, H₂, or any other gas that is emitted or consumed by a particular hydrogenotroph). In some embodiments, a sampling valve may be connected to an auxiliary instrument to directly determine the gas composition in the headspace.

In some embodiments, the systems of the present disclosure further comprise at least one auxiliary (e.g., control or actuator) instrument, which triggers inflow of or controls the flow rate of (a) fresh medium, (b) any growth-limiting substance (e.g., nutrient(s) or additive(s)), (c) additional gas, (d) venting, or (e) any combination of two or more of (a)-(e). In some embodiments, the at least one auxiliary instrument alters the inflow or the flow rate of (a) fresh medium, (b) any growth-limiting substance (e.g., nutrient(s) or additive(s)), (c) additional gas, (d) venting, or (e) any combination of two or more of (a)-(e) based on the parameter(s) determined by the auxiliary instrument.

In some embodiments, the system comprises an auxostat, a culturing system in which while in operation, uses feedback from a measurement (e.g., cell density, pH, level of any type of gas (e.g., CH₄, CO₂, H₂, etc.)) taken on the growth chamber to control the flow rate of fresh medium, any growth-limiting substance (e.g., nutrient(s) or additive(s)), and/or at least one type of gas that enter the growth chamber, thereby maintaining the measurement at a constant.

In some embodiments, the system comprises a turbidostat, which has feedback between the turbidity of the culture vessel and the dilution rate. A turbidostat dynamically adjusts the flow rate (and therefore the dilution rate) to make the turbidity constant.

In some embodiments, the system comprises a chemostat, a culturing system in which fresh medium, any growth-limiting substance (e.g., nutrient(s)), and/or at least one type of gas are continuously added, while culture liquid containing left over nutrients, metabolic end products and hydrogenotrophs is continuously removed at the same rate to keep the culture volume constant.

In some embodiments, the system may comprise continuous inflow of at least one type of gas (e.g., CH₄, CO₂, H₂, or any other gas that is consumed by a particular hydrogenotroph), preferably at a constant rate. In some embodiments, the continuous inflow is independent of any one of the parameters determined by an auxiliary instrument.

Inflow gas may first be passed through an apparatus configured to reduced and/or scavenge any trace oxidants (e.g., oxygen) in the inflow gas, e.g., catalytic converter, a palladium catalyst, or the like.

In certain aspects, the culturing methods of the present disclosure comprise incubating at least one hydrogenotroph under anaerobic atmosphere.

In some embodiments, the culturing method comprises incubating at least one hydrogenotroph under anaerobic atmosphere comprising H₂.

In some embodiments, the anaerobic atmosphere comprises at least about, no more than about, less than about, or about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80% H₂. In preferred embodiments, the anaerobic atmosphere comprises no more than about 4% H₂, which is non-flammable and non-explosive.

In some embodiments, the anaerobic atmosphere comprises about 0.5%-79%, preferably about 0.5-50%, more preferably about 0.5-20%, even more preferably about 0.5-4%, still more preferably about 1-4%, yet still more preferably about 2-4% H₂.

In some embodiments, the culturing method comprises incubating at least one hydrogenotroph under anaerobic atmosphere comprising CO₂.

In some embodiments, the anaerobic atmosphere comprises at least about, no more than about, less than about, or about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% CO₂.

In preferred embodiments, the anaerobic atmosphere comprises at least about 1 part of CO₂ for every 4 parts of H₂. In some embodiments, it is preferable that the CO₂ concentration is non-limiting, for example to ensure maximal utilization of the H₂. Thus, some preferred embodiments have an anaerobic atmosphere comprising at least 2, 3, 4, 5, 6, 7, 8, 9, or 10 parts of CO₂ for every 4 parts of H₂.

In some embodiments, the culturing method comprises incubating at least one hydrogenotroph under anaerobic atmosphere comprising inert gas. Any inert gas can be used. An example of a cost-effective inert gas is N₂. Accordingly, in some embodiments, the inert gas is N₂.

In some embodiments, the anaerobic atmosphere comprises at least about, no more than about, less than about, or about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% inert gas.

In some embodiments, the anaerobic atmosphere comprises at least about, no more than about, less than about, or about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% N₂.

Thus, a gas composition of 0.5-4% H₂, 1-99.5% CO₂, and the remainder balanced with an inert gas such as N₂ is preferred. An exemplary gas composition is 1-4% H₂, 1-25% CO₂, and 71-98% N₂. Another exemplary cost-effective gas composition is about 4% H₂, about 10% CO₂, and about 86% N₂.

In some embodiments, the culturing method comprises incubating at least one hydrogenotroph under anaerobic atmosphere comprising a gaseous mixtures comprising H₂, CO₂, and inert gas.

In some embodiments, a combination of two or more anaerobic gases can be premixed as a blend (e.g., using a mixing device upstream of the system) before being supplied into the growth chamber. In some embodiments, each gas can be supplied separately to the growth chamber independently.

In some embodiments, an anaerobic gaseous mixture is continuously added to the growth chamber (e.g., flask, bioreactor) during culturing. In some embodiments, the continuously added anaerobic gaseous mixture is added at a gas flow rate of at least about, no more than about, less than about, or about 0.01 vvm, 0.02 vvm, 0.03 vvm, 0.04 vvm, 0.05 vvm, 0.06 vvm, 0.07 vvm, 0.08 vvm, 0.09 vvm, 0.1 vvm, 0.11 vvm, 0.12 vvm, 0.13 vvm, 0.14 vvm, 0.15 vvm, 0.16 vvm, 0.17 vvm, 0.18 vvm, 0.19 vvm, 0.2 vvm, 0.21 vvm, 0.22 vvm, 0.23 vvm, 0.24 vvm, 0.25 vvm, 0.26 vvm, 0.27 vvm, 0.28 vvm, 0.29 vvm, 0.3 vvm, 0.31 vvm, 0.32 vvm, 0.33 vvm, 0.34 vvm, 0.35 vvm, 0.36 vvm, 0.37 vvm, 0.38 vvm, 0.39 vvm, 0.4 vvm, 0.41 vvm, 0.42 vvm, 0.43 vvm, 0.44 vvm, 0.45 vvm, 0.46 vvm, 0.47 vvm, 0.48 vvm, 0.49 vvm, 0.5 vvm, 0.51 vvm, 0.52 vvm, 0.53 vvm, 0.54 vvm, 0.55 vvm, 0.56 vvm, 0.57 vvm, 0.58 vvm, 0.59 vvm, 0.6 vvm, 0.61 vvm, 0.62 vvm, 0.63 vvm, 0.64 vvm, 0.65 vvm, 0.66 vvm, 0.67 vvm, 0.68 vvm, 0.69 vvm, 0.7 vvm, 0.71 vvm, 0.72 vvm, 0.73 vvm, 0.74 vvm, 0.75 vvm, 0.76 vvm, 0.77 vvm, 0.78 vvm, 0.79 vvm, 0.8 vvm, 0.81 vvm, 0.82 vvm, 0.83 vvm, 0.84 vvm, 0.85 vvm, 0.86 vvm, 0.87 vvm, 0.88 vvm, 0.89 vvm, 0.9 vvm, 0.91 vvm, 0.92 vvm, 0.93 vvm, 0.94 vvm, 0.95 vvm, 0.96 vvm, 0.97 vvm, 0.98 vvm, 0.99 vvm, 1 vvm, 2 vvm, 3 vvm, 4 vvm, 5 vvm, 6 vvm, 7 vvm, 8 vvm, 9 vvm, 10 vvm, 11 vvm, 12 vvm, 13 vvm, 14 vvm, 15 vvm, 16 vvm, 17 vvm, 18 vvm, 19 vvm, 20 vvm, 21 vvm, 22 vvm, 23 vvm, 24 vvm, 25 vvm, 26 vvm, 27 vvm, 28 vvm, 29 vvm, 30 vvm, 31 vvm, 32 vvm, 33 vvm, 34 vvm, 35 vvm, 36 vvm, 37 vvm, 38 vvm, 39 vvm, 40 vvm, 41 vvm, 42 vvm, 43 vvm, 44 vvm, 45 vvm, 46 vvm, 47 vvm, 48 vvm, 49 vvm, 50 vvm, 51 vvm, 52 vvm, 53 vvm, 54 vvm, 55 vvm, 56 vvm, 57 vvm, 58 vvm, 59 vvm, 60 vvm, 61 vvm, 62 vvm, 63 vvm, 64 vvm, 65 vvm, 66 vvm, 67 vvm, 68 vvm, 69 vvm, 70 vvm, 71 vvm, 72 vvm, 73 vvm, 74 vvm, 75 vvm, 76 vvm, 77 vvm, 78 vvm, 79 vvm, 80 vvm, 81 vvm, 82 vvm, 83 vvm, 84 vvm, 85 vvm, 86 vvm, 87 vvm, 88 vvm, 89 vvm, 90 vvm, 91 vvm, 92 vvm, 93 vvm, 94 vvm, 95 vvm, 96 vvm, 97 vvm, 98 vvm, 99 vvm, or 100 vvm. In some embodiments, the gas flow rate is about 0.01 to about 100 volume per volume per minute (vvm). In preferred embodiments, the gas flow rate is about 0.1 to about 10 vvm. In even more preferred embodiments, the gas flow rate is about 0.5 to about 3 vvm. In some embodiments, the gas flow rate is about 0.01 to about 0.1 vvm. In some embodiments the continuously added anaerobic gaseous mixture is added at a gas flow rate of about 0.02 vvm. In some embodiments, the continuously added anaerobic gaseous mixture comprises any one of the gases described above or mixtures thereof. An exemplary use of a continuously flowing gas apparatus to grow methanogens is described in Bryant, et al. (1968) Hydrogen-oxidizing methane bacteria. Journal of Bacteriology, which is incorporated herein by reference in its entirety.

In certain aspects, the culturing methods of the present disclosure comprise incubating at least one hydrogenotroph under pressure.

In some embodiments, the system of growing at least one hydrogenotroph is under the pressure of at least about, no more than about, less than about, or about 100 kilopascal (kPa), 101 kPa, 102 kPa, 103 kPa, 104 kPa, 105 kPa, 106 kPa, 107 kPa, 108 kPa, 109 kPa, 110 kPa, 111 kPa, 112 kPa, 113 kPa, 114 kPa, 115 kPa, 116 kPa, 117 kPa, 118 kPa, 119 kPa, 120 kPa, 121 kPa, 122 kPa, 123 kPa, 124 kPa, 125 kPa, 126 kPa, 127 kPa, 128 kPa, 129 kPa, 130 kPa, 131 kPa, 132 kPa, 133 kPa, 134 kPa, 135 kPa, 136 kPa, 137 kPa, 138 kPa, 139 kPa, 140 kPa, 141 kPa, 142 kPa, 143 kPa, 144 kPa, 145 kPa, 146 kPa, 147 kPa, 148 kPa, 149 kPa, 150 kPa, 151 kPa, 152 kPa, 153 kPa, 154 kPa, 155 kPa, 156 kPa, 157 kPa, 158 kPa, 159 kPa, 160 kPa, 161 kPa, 162 kPa, 163 kPa, 164 kPa, 165 kPa, 166 kPa, 167 kPa, 168 kPa, 169 kPa, 170 kPa, 171 kPa, 172 kPa, 173 kPa, 174 kPa, 175 kPa, 176 kPa, 177 kPa, 178 kPa, 179 kPa, or 180 kPa.

In some embodiments, the pressure within the growth chamber (e.g., flask, bioreactor) is no more than about 180 kPa. In some embodiments, the pressure is at least 100 kPa but no more than 180 kPa. In some embodiments, the pressure is about 1 atm (101.325 kPa). In preferred embodiments, the pressure is about 105 kPa, about 110 kPa, about 120 kPa, or about 125 kPa.

It may be beneficial to culture the hydrogenotroph under pressure to prevent trace introductions of air into the system through minor leaks given the anaerobic nature of methanogens. Thus, a pressure of at least about 1 atm to about 120 kPa, preferably about 1 atm to about 115 kPa, more preferably about 1 atm to about 110 kPa is beneficial.

In some embodiments, inoculum can be prepared in flasks or in smaller bioreactors where growth is monitored. For example, the inoculum size may be between about 0.1% v/v and about 5% v/v of the total growth chamber (e.g., bioreactor) volume. In some embodiments, the inoculum is about 0.1-about 3% v/v, about 0.1-about 1% v/v, about 0.1-about 0.5% v/v, or about 0.5-about 1% v/v of the total final culture volume. In some embodiments, the inoculum is about 0.1% v/v, about 0.2% v/v, about 0.3% v/v, about 0.4%, v/v, about 0.5% v/v, about 0.6% v/v, about 0.7% v/v, about 0.8% v/v, about 0.9% v/v, about 1% v/v, about 1.5% v/v, about 2% v/v, about 2.5% v/v, about 3% v/v, about 4%, v/v, or about 5% v/v of the total final culture volume. In some embodiments, the inoculum size of the hydrogenotrophs may be between approximately 0.5 and 3% of the total final culture volume. In some embodiments, the inoculum size is at least about, no more than about, less than about, or about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% of the final culture volume. In some embodiments, the inoculum size is between about 0.001% to about 50% of the final volume. In preferred embodiments, the inoculum size is between about 0.1% to about 20% of the final volume. In even more preferred embodiments, the inoculum size is between about 1% to about 10% of the final volume.

In some embodiments, the culturing method comprises incubating at least one hydrogenotroph at a temperature of at least about, no more than about, less than about, or about 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., or 65° C. In some embodiments, the temperature is between about 25° C. and about 65° C. In some embodiments, the temperature is between about 25° C. and about 45° C. In preferred embodiments, the temperature is between about 35° C. to about 50° C. In even more preferred embodiments, the temperature is between about 37° C. to about 40° C. In yet other preferred embodiments, the temperature is at about 38° C.

### Table C: Exemplary Auxiliary Instrument

This Table provides exemplary auxiliary instruments (e.g., analytical, control, actuator) that may facilitate the growth of at least one hydrogenotroph. The at least one auxiliary instrument may be coupled with or be part of the systems of the present disclosure.

The systems of the present disclosure may comprise any one or more of the instruments or devices described herein.

1. Temperature control: In some embodiments, the temperature control system detects and maintains the temperature between about 25° C. and about 65° C. In some embodiments, the temperature control system detects and maintains the temperature between about 25° C. and about 45° C. In preferred embodiments, the temperature is between about 35° C. to about 50° C. In even more preferred embodiments, the temperature is between about 37° C. to about 40° C. In yet other preferred embodiments, the temperature control system detects and maintains the temperature at about 38° C., which is the suitable temperature for growing many methanogens.

2. Gas flow control: Gas flow sensor combined with one or more gas valves to control the input and/or output of gas into the growth chamber, which (a) controls flow rate of the gas, and/or (b) controls timing of gas input and/or output. A gas flow control may optionally further comprise or be coupled with gas supplies and/or a gas mixer that combines gases from one or more cylinders, wherein the flow rates determine the ratios of gases to be combined to achieve the desired gas mixture into the growth chamber. An exemplary use of a continuously flowing gas apparatus to grow methanogens is described in Bryant, et al. (1968) Hydrogen-oxidizing methane bacteria. Journal of Bacteriology, which is incorporated herein by reference in its entirety.

3. H₂/CO₂/CH₄ sensors: These sensors monitor the level of H₂/CO₂/CH₄ in growth chambers. The sensors may optionally be coupled with gas flow control and actuate gas input (of gas(es) that are required for growth, e.g., H₂, CO₂) and/or output of the emitted gas (e.g., CH₄), ensuring that the culture have sufficient gaseous substrate to grow. These sensors may also optionally be coupled with the gas supplies and/or a gas mixer (e.g., an impeller).

4. Spectrophotometer/turbimeter/fluorometer: An instrument to measures cell quantity/concentration in the growth chamber (e.g., turbidity or light absorbance). Certain hydrogenotrophs, e.g., methanogens, are fluorescent, thus a fluorometer may be used to detect the level of methanogens in the growth chamber. These instruments may be coupled with gas flow control, gas supplies, a gas mixer, or any combination thereof, to (a) increase the gas flow rate as the cell density increases; and/or (b) trigger gas inputs and/or outputs during a fed batch process at certain cell densities. These instruments may also be coupled with a shaker or a mixer to increase the mixing rate as the cell density increases.

5. pH sensor: It is well known in the art that the pH of the media changes in the course of cell growth, which indirectly indicates the cell density. The pH deviations may trigger addition of one or more buffering agents (e.g., acid, base) and/or reducing agents to maintain the correct solubility of CO₂ in the media. The pH sensor may be coupled with liquid flow control described below.

6. Oxygen sensor and potentially sacrificial electrode(s) in line with the gas supply

7. Liquid flow control: At least one liquid flow control may be coupled with the systems of the present disclosure. The liquid flow control may hold and actuate the inflow of any liquid necessary for growth of hydrogenotrophs. The liquid includes but is not limited to fresh media, any solution comprising any one or more of nutrients (e.g., carbon source, minerals, vitamins, additives (e.g., methanol)), buffering agents, reducing agents, and any other liquid known in the art that aids growth of hydrogenotrophs.

8. Vessel level control

9. Others

### Exemplary Sensors

pH meter: for example electrochemical, electrode, differential sensor, combination sensor
O₂ meter, liquid and/or gas phase: for example electrochemical, zirconia, titania, optical, clark, infrared, electro, ultrasonic, laser, paramagenetic, wideband, narrowband
H₂ meter, liquid and/or gas phase: for example FID, thermal conductivity, catalytic combustion, catalytic, electrochemical, thin film, thick film, microelectromechanical, chemochromic, diode, semiconductor, metal oxide semiconductor (MOS), non-resistive semiconductor, palladium based, optical, fiber Bragg grating (FBG) coated with a palladium layer, micromirror, tapered fibre coated with palladium, conductive, metallic La—Mg²—Ni, clark-type, any one of the above with or without Siloxane
CO₂ sensor, liquid and/or gas phase: for example catalytic, non-dispersive infrared, electrochemical, semiconductor, metal oxide semiconductor, optical, catalytic combustion
CH₄ meter, liquid and/or gas phase: for example laser, infrared, FID, catalytic, catalytic bead, optical, calorimetric, pyroelectric, semiconducting oxide, electrochemical, pellistor, non-dispersive infrared
Acetate sensor: for example bioelectrochemical, biosensor, electrochemical
Volatile fatty acid sensors: for example Isovaleric, Isobutyric, Valeric, 2-methylbutyric, 2-methylvaleric
Metabolite sensors: for example metabolic precursors, products and/or intermediates of methanogenesis
Cell density monitoring: for example absorbance, permittivity, in situ, biomass capacitance, optical
Fluorometer: for example filter fluorometer, spectrofluorometer
Temperature sensor: for example thermistor, thermocouple, semiconductor, RTD, contact temperature sensor, thermometer, infrared, NTC thermistor
Pressure sensor, gas and/or liquid phase: for example strain gauge, piezoelectric, capacitive, manometers, vacuum pressure, bourdon tube, aneroid barometer, resistive, optical, MEMS, potentiometric, inductive, variable reluctance
Foam sensor: for example conductivity switch, ultrasonic gas switch, ultrasonic gas switch, RF admittance, ultrasonic, infrared, camera, contact sensors, contactless sensors
H₂S sensor, liquid and/or gas phase: for example electrochemical, metal oxide semiconductor, semiconductor, solid electrolyte
Scale: for example spring scale, hydraulic, pneumatic, balance, torsion balance, roberval balance, strain gauge, beam balance, digital
Gas composition analytical system
Flow meter, liquid and/or gas phase: for example differential pressure, rotameter, variable area, orifice, venturi, pitot tube, positive displacement, reciprocating piston, oval-gear, nutating-disk, rotary-vane, volumetric, ultrasonic, mass flow, velocity, vortex, electromagnetic, turbine, paddlewheel, open channel, flow nozzle, sonic nozzle, thermal, target type, magnetic induction, scale
Camera
Level sensor: for example ultrasonic, float level, rotating paddle, diaphragm switch, radar, microwave, displacer switch, capacitance, optical, float switch, Ultrasound, Laser profiler, Inductive displacement, tuning fork, hydrostatic level transmitter, magnetic, optical, buoy level, level switch, scale
Redox sensor
Contamination sensor (e.g., spores, biosensor)

### Exemplary Actuators

Motor
Magnetic stirrer
pH regulation (e.g., with acid-base reservoir and pump)
Regulated recirculation of headspace (valve)
Unregulated recirculation of headspace (valve)
Recirculation of liquid
Pressure regulator upstream of tank
Pressure regulator downstream of tank
Condenser
Cooling water
Heating water
Cooling jacket
Heating jacket
Water bath
Hot plate
Burner at outlet
Overpressure release valve
Flow controller, liquid phase
Flow controller, gas phase
Automatic sampling port(s), can be triggered by time or sensor readout
Automated nutrient inlet (pump)
Automated antifoam addition (pump), can be triggered by foam sensor
Continuous/periodic culture removal (pump or valve), optionally triggered by sensor
Continuous/periodic liquid feed (pump or valve), optionally triggered by sensor

### Exemplary Hardware

Vessel type: stainless steel, glass, plastic, polycarbonate, others
Various culture vessel (growth chamber) geometries/shapes: round, conical, cylindrical, spherical, long and horizontal culture system (to maximize gas exchange), others
Impeller
Stirring paddles
Baffles
Sparger
Stainless steel gas diffuser
Filtration system
Humidifying tank upstream
Gas mixer upstream of tank
Multiple gas inlets to tank
Single gas inlet to tank
Sampling port(s), manual and/or automatic
Antifoam addition, manual and/or automatic
Injection port(s): septum, tubing with clamp/valve, triclamp
Harvest port
Sampling port, head space
Sampling port, dip tube: quick connects, tubing, triclamp
Viewing port
Antifoam addition, manual and/or automatic
Carbon filter
Exhaust system
Incubated room/environment

### Other

Electrolytic converter
CH₄ converter
CH₄ recovery
H₂S removal
Three phase system: e.g., gas, liquid, oil
CH₄ conversion system (converts CH₄ to CO₂ and H₂)

In some embodiments, a gas outlet comprises a CH₄ conversion system that converts CH₄ to CO₂ and H₂, which allows recycling of gas. An exemplary use of this system comprises a burner or catalyst that oxidizes the CH₄ with a suitable amount of air, the CO₂ and H₂ is then reintroduced into the culturing system.

In some embodiments, systems of the present disclosure comprises a H₂S scrubber, such as a catalytic scrubber, water scrubber, chemical scrubber, biological scrubber, biochemical scrubber and/or a carbon filter.

In some embodiments, the system of the present disclosure comprise an apparatus configured to oxidize any effluent CH₄ and/or capture produced CH₄ or oxidized derivatives thereof.

In some embodiments, the system may further comprise an apparatus configured to electrolyze water as a source of H₂. It would be understand that an H₂/O₂ separation system would be required as well to provide an anaerobic source of H₂ to the culture.

In some embodiments, the system may further comprise an oxygen impermeable membrane.

In some embodiments, the system may further comprise an apparatus configured to collect and optionally liquidize collected CH₄, thereby allowing transfer to suitable sites capable of using the collected CH₄.

Media Any culturing volume may be suitable for the culturing methods of the present disclosure. In some embodiments, the culturing volume or the amount of media is at least 0.01 L, at least 0.05 L, at least 0.1 L in volume, at least 0.2 L in volume, at least 0.3 L in volume, at least 0.4 L in volume, at least 0.5 L in volume, at least 0.6 L in volume, at least 0.7 L in volume, at least 0.8 L in volume, at least 0.9 L in volume, at least 1 L in volume, at least 5 L in volume, at least 10 L in volume, at least 15 L in volume, at least 20 L in volume, at least 30 L in volume, at least 40 L in volume, at least 50 L in volume, at least 100 L in volume, at least 200 L in volume, at least 250 L in volume, at least 500 L in volume, at least 750 L in volume, at least 1000 L in volume, at least 1500 L in volume, at least 2000 L in volume, at least 2500 L in volume, at least 3000 L in volume, at least 3500 L in volume, at least 4000 L in volume, at least 5000 L in volume, at least 7500 L in volume, at least 10,000 L in volume, at least 15,000 L in volume, or at least 20,000 L in volume.

In some embodiments, the culturing volume or the amount of media is about 0.01 L in volume, about 0.05 L in volume, about 0.1 L in volume, about 0.5 L in volume, about 1 L in volume, about 5 L in volume, about 10 L in volume, about 15 L in volume, about 20 L in volume, about 30 L in volume, about 40 L in volume, about 50 L in volume, about 100 L in volume, about 200 L in volume, about 250 L in volume, about 500 L in volume, about 750 L in volume, about 1000 L in volume, about 1500 L in volume, about 2000 L in volume, about 2500 L in volume, about 3000 L in volume, about 3500 L in volume, about 4000 L in volume, about 5000 L in volume, about 7500 L in volume, about 10,000 L in volume, about 15,000 L in volume, or about 20,000 L in volume.

In some embodiments, the culturing volume or the amount of media is about 100 L in volume, about 200 L in volume, about 300 L in volume, about 400 L in volume, about 500 L in volume, about 600 L in volume, about 700 L in volume, about 800 L in volume, about 900 L in volume, or about 1000 L in volume.

The systems, compositions, and/or methods of the present disclosure may use any media that are known in the art to facilitate growth of hydrogenotrophs may be used. In some embodiments, the media comprises BY medium (Joblin K. N. 2005. Methanogenic archaea.

Methods in Gut Microbila Ecology for Ruminants, which is incorporated herein by reference), BCYT media, SAB media (Khelaifia et at (2013) PS One, 8(4):e61563, which is incorporated herein by reference), and/or DSMZ media (e.g., DSMZ 119 media, DSMZ 322 media, DSMZ 334c media). In some embodiments, the systems, compositions, and/or methods of the present disclosure may use any media that has been developed and disclosed herein (e.g., the media in Tables D and E, or the media in working Examples).

TABLE D
An Exemplary Media Composition

| | Exemplary | | | |
|---|---|---|---|---|
| | concentration | Units for | Preferred range of | |
| | in the final | exemplary | concentration in | |
| Component | media | concentration | the final media | |
| Sodium hydrogen carbonate | 5.00E+00 | g/L | 0.5-50 | g/L |
| Yeast extract | 1.00E+00 | g/L | 0.1-10 | g/L |
| Rumen fluid | 3.00E+02 | mL/L | 100-600 | mL/L |
| Cysteine hydrochloride | 5.00E+02 | mg/L | 5-5000 | mg/L |
| Sodium chloride | 1.02E+00 | g/L | 0.1-10 | g/L |
| Potassium dihydrogen phosphate | 5.10E−01 | g/L | 0.05-5 | g/L |
| Calcium chloride dihydrate | 1.34E−01 | g/L | 0.01-1 | g/L |
| Magnesium sulfate heptahydrate | 2.04E−01 | g/L | 0.02-2 | g/L |
| Ammonium sulfate | 2.55E−01 | g/L | 0.02-3 | g/L |
| Dipotassium hydrogen phosphate | 1.02E+00 | g/L | 0.1-10 | g/L |
| Pyridoxine hydrochloride | 1.06E−01 | mg/L | 0.01-10 | mg/L |
| Ascorbic acid | 5.29E−02 | mg/L | 0.005-100 | mg/L |
| Calcium pantothenate | 5.29E−02 | mg/L | 0.005-100 | mg/L |
| Lipoic acid | 5.29E−02 | mg/L | 0.005-100 | mg/L |
| Nicotinamide | 5.29E−02 | mg/L | 0.005-100 | mg/L |
| Nicotinic acid (Niacin) | 5.29E−02 | mg/L | 0.005-100 | mg/L |
| 4-Aminobenzoic acid | 5.29E−02 | mg/L | 0.005-100 | mg/L |
| Pyridoxal hydrochloride | 5.29E−02 | mg/L | 0.005-100 | mg/L |
| Riboflavin (vitamin B2) | 5.29E−02 | mg/L | 0.005-100 | mg/L |
| Thiamine hydrochloride | 5.29E−02 | mg/L | 0.005-100 | mg/L |
| D-(+)-Biotin | 2.11E−02 | mg/L | 0.002-100 | mg/L |
| Folic acid | 2.11E−02 | mg/L | 0.002-100 | mg/L |
| Vitamin B12 (cyanocobalamin) | 1.06E−03 | mg/L | 0.0001-100 | mg/L |
| Sodium sulfide nonahydrate | 1.42E−03 | g/L | 0.0001-100 | g/L |
| Nitrilotriacetic acid | 1.50E−02 | g/L | 0.001-100 | g/L |
| Magnesium sulfate heptahydrate | 5.43E−02 | g/L | 0.005-100 | g/L |
| Manganese(II) sulfate monohydrate | 5.00E−03 | g/L | 0.0005-100 | g/L |
| Sodium chloride | 1.00E−02 | g/L | 0.001-100 | g/L |
| Iron(II) sulfate heptahydrate | 1.00E−03 | g/L | 0.0001-100 | g/L |
| Cobalt (II) chloride hexahydrate | 1.00E−03 | g/L | 0.0001-100 | g/L |
| Calcium chloride dihydrate | 1.32E−03 | g/L | 0.0001-100 | g/L |
| Zinc sulfate heptahydrate | 1.00E−03 | g/L | 0.0001-100 | g/L |
| Copper (II) sulfate pentahydrate | 1.00E−01 | mg/L | 0.01-100 | mg/L |
| Aluminium potassium sulfate dodecahydrate | 1.00E−01 | mg/L | 0.01-100 | mg/L |
| Boric acid | 1.00E−01 | mg/L | 0.01-100 | mg/L |
| Sodium molybdate(VI) dihydrate | 1.00E−01 | mg/L | 0.01-100 | mg/L |
| Nickel (II) sulfate hexahydrate | 3.00E−01 | mg/L | 0.03-100 | mg/L |
| Sodium selenite | 2.00E−01 | mg/L | 0.02-100 | mg/L |
| Sodium tungstate dihydrate | 2.00E−01 | mg/L | 0.02-100 | mg/L |
| Resazurin, sodium salt | 4.00E−04 | g/L | 0.000004-0.04 | g/L |

| | |
|---|---|
| water | balance |

* In the preferred range of concentrations above, endpoints are included. Furthermore, the values of the preferred range of concentrations above can assume any specific value or sub-range within the stated ranges, to the tenth of the unit of the lower limit of the range.

It should be appreciated that any suitable hydrated form of a listed component can be used, and the concentration should be adjusted to account for differences in the molecular weight of the alternative. Additionally, the rumen fluid may be dried or dehydrated. A skilled artisan would understand how to calculate the amount of powdered rumen fluid to add based on weight differences between the liquid and powdered preparations. Dried rumen fluid can be advantageous for consistent and reproducible productions of cell mass as it can be harvested, combined, checked for any transmissible elements, e.g., viruses, sterilized, and stored long term under suitable conditions. Thus, this disclosure provides compositions comprising dried, sterilized rumen fluid suitable for mass production of methanogens.

TABLE E
An Exemplary Media Composition

| | Exemplary | |
|---|---|---|
| | concentration | Preferred range of |
| | (g/L) in the | concentration in the |
| Component | final media | final media (g/L) |
| KH₂PO₄ | 5.00E−01 | 0.05-50 |
| MgSO₄, 7H₂O | 4.05E−01 | 0.04-40 |
| NaCl | 8.10E−01 | 0.08-80 |
| NH₄Cl | 4.00E−01 | 0.04-40 |
| CaCl 2H₂O | 6.62E−02 | 0.006-60 |
| Yeast Extract | 1.00E+00 | 0.1-20 |
| Na-acetate | 1.00E+00 | 0.1-20 |
| Na-formate | 2.00E+00 | 0.2-20 |
| Sodium resazurin | 5.00E−04 | 0.00005-0.5 |
| NaHCO₃ | 4.00E+00 | 0.4-40 |
| L-cysteine HCl H₂O | 5.00E−01 | 0.05-50 |
| Na₂S 9H₂O | 5.00E−01 | 0.05-50 |
| Trypticase | 2.00E+00 | 0.2-20 |
| L-Isoleucine | 1.31E−01 | 0.01-10 |
| L-leucine | 1.31E−01 | 0.01-10 |
| HCl (7.7M) | 1.00E−02 | 0.001-1 |
| FeCl₂ 4H₂O | 1.50E−03 | 0.0001-1 |
| ZnCl₂ | 7.00E−05 | 0.0000007-1 |
| MnC1₂ 4H₂O | 1.00E−04 | 0.000001-1 |
| H₃BO₃ | 1.06E−04 | 0.000001-1 |
| CoCl₂ 6H₂O | 1.19E−03 | 0.00001-1 |
| CuCl₂ 2H₂O | 2.00E−06 | 0.00000002-1 |
| NiCl 6H₂O | 2.40E−05 | 0.0000002-1 |
| Na₂MoO₄ 2H₂O | 1.36E−04 | 0.000001-1 |
| Isobutyric acid | 4.48E−01 | 0.004-10 |
| DL-2-Methylbutyric acid | 5.05E−01 | 0.005-10 |
| Valeric acid | 5.02E−01 | 0.005-10 |
| Isovaleric acid | 5.00E−01 | 0.005-10 |
| Nitrilotriacetic acid | 1.50E−02 | 0.0001-1 |
| MnSO₄ H₂O | 5.00E−03 | 0.00005-5 |
| FeSO₄ 7H₂O | 1.00E−03 | 0.00001-1 |
| ZnSO₄ 7H₂O | 1.00E−03 | 0.00001-1 |
| CuSO₄ 5H₂O | 1.00E−04 | 0.000001-1 |
| AlK(SO₄)₂ 12H₂O | 1.00E−04 | 0.000001-1 |
| NiSO₄ 6H₂O | 3.00E−04 | 0.00003-3 |
| Na₂SeO₃ | 2.00E−04 | 0.00002-2 |
| Na₂WO₄ 2H₂O | 2.00E−04 | 0.00002-2 |
| Pyridoxine Hydrochloride | 1.06E−04 | 0.00000001-01 |
| Ascorbic acid | 5.29E−05 | 0.00000004-0.4 |
| calcium pantothenate | 5.29E−05 | 0.00000004-0.4 |
| lipoic acid | 5.29E−05 | 0.00000004-0.4 |
| nicotinamide | 5.29E−05 | 0.00000004-0.4 |
| nicotinic acid | 5.29E−05 | 0.00000004-0.4 |
| 4-aminobenzoic acid | 5.29E−05 | 0.00000004-0.4 |
| pyridoxal hydrochloride | 5.29E−05 | 0.00000004-0.4 |
| riboflavin | 5.29E−05 | 0.00000004-0.4 |
| thiamine hydrochloride | 5.29E−05 | 0.00000004-0.4 |
| D-(+)-biotin | 2.11E−05 | 0.00000001-0.1 |
| folic acid | 2.11E−05 | 0.00000001-0.1 |
| vitamin b12 | 1.06E−06 | 0.000000001-0.1 |
| water | balance | |

* In the preferred range of concentrations above, endpoints are included. Furthermore, thevalues of the preferred range of concentrations above can assume any specific value or sub-range within the stated ranges, to the tenth of the unit of the lower limit of the range.

It should be appreciated that any suitable hydrated form of a listed component can be used, and the concentration should be adjusted to account for differences in the molecular weight of the alternative.

In some embodiments, any culture medium that facilitates the growth of a hydrogenotroph may be used for the methods of the present disclosure. In some embodiments, a culture medium comprises at least one component selected from those listed in Table D or Table E. In some embodiments, a culture medium comprises any combination of two or more components listed in Table D or Table E. In some embodiments, a culture medium comprises all components listed in Table D, but without at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 components. In some embodiments, a culture medium comprises all components listed in Table E, but without at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 components. In some embodiments, the culture medium comprises at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41 components listed in Table D. In some embodiments, the culture medium comprises at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51 components listed in Table E. In preferred embodiments, a culture medium comprises all components listed in Table D. In preferred embodiments, a culture medium comprises all components listed in Table E.

In preferred embodiments, where a culture medium comprises at least one component from Table D or Table E, the at least one component is present in the concentration listed in Table D or Table E; or in a range of concentrations, or any range in between, or any value in between, listed in Table D or Table E.

In some embodiments, a culture medium comprises one or more positively charged cations. In some embodiments, the positively charged cation is a transition metal, alkali metal, alkaline Earth metal, lanthanide, basic metal, semimetal, or nonmetal. Any suitable alkali metal could be used, for example lithium, cesium, rubidium, preferably potassium, more preferably sodium. Any suitable alkaline Earth metal could be used, for example beryllium, barium, strontium, preferably magnesium, more preferably calcium. Any suitable transition metal could be used, for example lead, palladium, gold, zirconium, chromium, rhenium, vanadium, or platinum, more preferably copper, cobalt, iron, manganese, nickel, or zinc. Any suitable lanthanide could be used, for example lanthanum, cerium. Any suitable semimetal could be used, such as antimony. Any suitable basic metal could be used, such as gallium. Alternatively or in combination, any suitable nonmetal can be used, for example H₂. In some embodiments, the cation is polyatomic, for example dihydrogen, guanidium, more preferably ammonium.

Additionally or alternatively, the culture medium also comprises one or more negatively charged anions. In some embodiments, the anion is a halogen or a nonmetal. Any suitable anion can be used, for example sulfur or chloride. In some embodiments, the anion is polyatomic, for example arsenate or iodate, preferably permanganate, more preferably nitrate, sulfate, phosphate, acetate, formate, carbonate, selenite, tungstate, borate, molybdate, or selenium trioxide.

These cations and anions can be added to the medium in the form of one or more salts. Any suitable combination of cation and anion species can comprise the salt, such as those listed in Table D or E. Any suitable concentration range can be used, such as those listed in Table D or E for example. The concentration of these salts can be determined based on the measured quantity added during media preparation.

In some embodiments, a culture medium comprises cofactors. In preferred embodiments, a culture medium comprises coenzyme M. Other cofactors include but are not limited to nicotineamideadenine dinucleotide (NAD), nicotineamide adenine dinucelotide phosphate (NADP), flavin adenine dinucleotide (FAD), and coenzyme A (CoA).

In some embodiments, a culture medium comprises an alcohol. In some embodiments, a culture medium comprises an alcohol selected from methanol, ethanol, propanol, butanol, and isopropanol.

In some embodiments, a culture medium comprises one or more sugars. In some embodiments, a culture medium comprises monosaccharide, disaccharide, polysaccharide, or any combination of two or more thereof.

In some embodiments, a culture medium comprises at least one monosaccharide. In some embodiments, a culture medium comprises at least one monosaccharide selected from glucose, mannose, fructose, ribose, galactose, xylose, and arabinose.

In some embodiments, a culture medium comprises at least one disaccharide. In some embodiments, a culture medium comprises at least one disaccharide selected from lactose, sucrose, and maltose.

In some embodiments, a culture medium comprises at least one polysaccharide. In some embodiments, a culture medium comprises at least one polysaccharide selected from starch, glucan, cellulose, dextran, and xanthan.

In some embodiments, it is preferable that the sugars are reducing sugars, such as glucose, fructose, galactose, mannose, ribose, xylose, arabinose, lactose, maltose, cellobiose, a suitable alternative, and/or a combination thereof. The reducing sugars may further act as scavengers of trace oxidizing agents, thereby improving hydrogenotroph culturing.

In some embodiments, a culture medium comprises one or more components which are themselves a combination of chemicals. In some embodiments, a culture medium comprises peptone, tryptone, casamino acids, trypticase, or any combination of two or more thereof. In some embodiments, the culture medium comprise yeast and/or yeast extract.

In some embodiments, a culture medium comprises at least one amino acid (natural or unnatural). Over 500 natural amino acids exist in nature. In some embodiments, a culture medium comprises at least one amino acid selected from 22 amino acids found in proteins (e.g., alanine, cysteine, aspartic acid, glutamic acid, phenylalanine, glycine, histidine, isoleucine, lysine, leucine, methionine, asparagine, pyrrolysine, proline, glutamine, arginine, serine, threonine, selenocysteine, valine, tryptophan, and tyrosine). In preferred embodiments, a culture medium comprises at least one amino acid selected from arginine, histidine, lysine, aspartic acid, glutamic acid, serine, threonine, asparagine, glutamine, selenocysteine, glycine, proline, alanine, valine, methionine, phenylalanine, tyrosine, tryptophan, citrulline, ornithine, pyroglutamate, 4-aminobutyrate, and β-alanine.

In some embodiments, a culture medium comprises at least one organic acid. In some embodiments, a culture medium comprises at least one organic acid selected from 3-hydroxybutyrate, 3-HPA, 3-phenylpropionate, 4-hydroxybutyrate, acetate, acetoacetate, benzoate, butyrate, ferulate, formate, fumarate, isobutyrate, isovalerate, lactate, nicotinate, phenylacetate, propionate, succinate, valerate, 2-hydroxyvalerate, 2-hydroxyisovalerate, 3-hydroxyphenylacetate, citrate, pyruvate, formate, benzoate, and fumarate.

In some embodiments, a culture medium comprises at least one vitamin. In some embodiments, a culture medium comprises at least one vitamin selected from vitamin A, niacin, choline, calciferol, vitamin E, vitamin K, pyridoxine, ascorbic acid, pantothenate, lipoic acid, nicotinamide, 4-aminobenzoic acid, pyridoxal, riboflavin, thiamine, biotin, folic acid, cyanocobalamin.

In some embodiments, a culture medium comprises at least one mineral. In some embodiments, a culture medium comprises at least one mineral selected from fluoride and iodine.

In some embodiments, a culture medium comprises at least one material of animal origin. In some embodiments, a culture medium comprises at least one material of animal origin is selected from rumen fluid, fetal bovine serum, bovine serum albumin, blood, bone, and fat. In some embodiments, the material of animal origin, for example rumen fluid, is delivered to the media preparation as a liquid, or in a frozen state. In other embodiments, the material of animal origin, for example rumen fluid, may be dried to a solid, and in some cases powdered. Where possible, the dried form may offer storage advantages over liquid. In some embodiments, the material of animal origin is sterilized and free of detectable amount of transmissible elements, e.g., viruses. This can be especially important to prevent disease transmission when producing antigenic material for a similar subject to that of subject from which the material of animal origin was isolated.

Thus, provided herein are powdered rumen fluid compositions prepared from at least about 1, 2, 3, 4, 5, 10, 20, 25, 50, or 100 different animals. In certain embodiments, the powdered rumen fluid is free of detectable transmissible disease elements, e.g., viruses. Any suitable method can be used to test for the transmissible disease elements, for example qPCR.

In some embodiments, a culture medium comprises at least one metabolite found in a ruminant's rumen. In some embodiments, a culture medium comprises at least one metabolite found in a bovine rumen. In some embodiments, a culture medium comprises at least one metabolite selected from cadaverine, nicotinate, acetone (or Propanone), 4-Hydroxy-3-methoxymandelate, imidazole, 1,3-DHA, cadaverine, caffeine, choline, dimethylamine, ethanolamine, glycerol, hypoxanthine, methylamine, N-nitrosodimethylamine, NADMA, PAG, thymine, uracil, xanthine, and endotoxin.

In some embodiments, a culture medium comprises at least one buffer. Any buffer known in the art can be used. In some embodiments, a culture medium comprises at least one buffer selected from phosphate buffer, HEPES, MOPS, MES, BES, MOPSO, ACES, TAPS, Bicine, and Tris.

In some embodiments, a culture medium comprises at least one trace element. In some embodiments, a culture medium comprises at least one trace element selected from copper, beryllium, boron, aluminum, thallium, zinc, chromium, molybdenum, cobalt, nickel, selenium, fluorine, iron, iodine, manganese, magnesium, rubidium, strontium, molybdenum, lead, arsenic, vanadium, and cadmium.

In some embodiments, a culture medium comprises at least one biogenic amine. In some embodiments, a culture medium comprises at least one biogenic amine selected from dimethylarginine, acetylornithine, carnosine, histamine, kynurenine, methioninesulfoxide, phenylethylamine, sarcosine, taurine, serotonin, and putrescine.

In some embodiments, a culture medium comprises at least one acylcarnitine. In some embodiments, a culture medium comprises at least one acylcarnitine selected from tetradecenoylcarnitine, tetradecadienylcarnitine, hydroxytetradecadienylcarnitine, hexadecanoylcarnitine, hydroxyhexadecanoylcarnitine, hexadecadienylcarnitine, hydroxyoctadecanoylcarnitine, octadecadienylcarnitine, propionylcarnitine, hydroxybutyrylcarnitine, hydroxypropionylcarnitine, butenylcarnitine, valerylcarnitinec, methylglutarylcarnitine, tiglylcarnitine, glutaconylcarnitine, hexenoylcarnitine, and pimelylcarnitine.

In some embodiments, a culture medium comprises at least one fatty acid. In some embodiments, a culture medium comprises at least one fatty acid selected from C6:0, C8:0, C10:0, C11:0, C12:0, C13:0, C14:0, C14:1n5, C15:0, C16:0, C16:1n7, C17:0, C18:0, C18:1n9, C18:2n6, C18:3n6, C20:0, C20:1n9, C20:2n6, C20:4n6, C22:0, C22:1n9, C22:2n6, C24:1n9, FFA16:1(9), FFA18:1(isomer), FFA18:1(9), FFA18:2(9,11), and FFA18:3(9,12,15).

In some embodiments, a culture medium comprises at least one cholesterol ester. In some embodiments, a culture medium comprises at least one cholesterol ester selected from CE12:0, CE14:0, CE15:0, CE16:0, CE16:1, CE18:0, CE18:1(isomer), CE18:2(9,11), CE20:0, CE22:0, CE22:1(13), and CE24:0.

In some embodiments, a culture medium comprises at least one lysophosphatidylcholine. In some embodiments, a culture medium comprises at least one lysophosphatidylcholine selected from lysoPC a C16:1, lysoPC a C17:0, lysoPC a C18:0, lysoPC a C18:1, lysoPC a C18:2, lysoPC a C20:4, lysoPC a C26:0, and lysoPC a C16:0.

In some embodiments, a culture medium comprises at least one phosphatidylcholine. In some embodiments, a culture medium comprises at least one phosphatidylcholine selected from PC aa C28:1, PC aa C30:2, PC aa C32:0, PC aa C32:2, PC aa C32:3, PC aa C34:1, PC aa C34:2, PC aa C34:3, PC aa C34:4, PC aa C36:1, PC aa C36:2, PC aa C36:3, PC aa C36:4, PC aa C36:5, PC aa C36:6, PC aa C38:1, PC aa C38:4, PC aa C38:5, PC aa C38:6, PC aa C40:3, PC aa C40:6, PC aa C42:1, PC aa C42:2, PC aa C42:4, PC aa C42:5, PC ae C30:2, PC ae C32:1, PC ae C32:2, PC ae C34:0, PC ae C34:1, PC ae C34:2, PC ae C34:3, PC ae C36:1, PC ae C36:2, PC ae C36:3, PC ae C36:4, PC ae C36:5, PC ae C38:1, PC ae C38:2, PC ae C38:3, PC ae C38:4, PC ae C38:5, PC ae C38:6, PC ae C40:2, PC ae C40:3, PC ae C42:1, PC ae C42:3, PC ae C44:3, PC ae C44:4, and PC ae C44:5.

In some embodiments, a culture medium comprises at least one sphingomyelin. In some embodiments, a culture medium comprises at least one sphingomyelin selected from SM (OH) (d18:1/14:1), SM (OH) (d18:1/C16:1), SM (OH) (d18:1/22:1), SM (OH) (d18:1/22:2), SM (OH) (d18:1/24:1), SM (d18:1/16:0), SM (d18:1/16:1), SM (d18:1/20:2), SM (d18:1/22:3), and SM (d18:1/24:1).

In some embodiments, a culture medium comprises at least one reducing agent. In some embodiments, a culture medium comprises at least one reducing agent selected from sodium thioglycolate, iron sulfide, dithiothreitol, sodium dithionite, lithium aluminum hydride, sodium borohydride, diisobutyl aluminum hydride, palladium, and platinum, more preferably sodium sulfide or cysteine.

In some embodiments, a culture medium comprises at least one solid phase within a liquid culture. In some embodiments, a culture medium comprises at least one solid phase selected from sand and aluminum oxide.

In some embodiments, a culture medium comprises at least one oil or oil phase. In some embodiments, a culture medium comprises at least one oil or oil phase is selected from mineral oil and halogenated oil. In some embodiments, the halogenated oil comprises a fluorinated oil. Fluorinated oils typically readily solubilize gases and thus could increase the availability of H₂ and CO₂ to cells in the culture In some embodiments, a culture medium comprises at least one component, ingredient, or compound described in Malheiros, et al. (2021) Comparative untargeted metabolome analysis of ruminal fluid and feces of Nelore steers (Bos indicus). Sci Rep; or Saleem, et al. (2013). The bovine ruminal fluid metabolome. Metabolomics, each of which is incorporated herein by reference.

Prior to inoculation, pH may be adjusted between pH 0-14, more preferably between 4-9, even more preferably between 5-8. During incubation or culturing, pH may be further controlled between pH 0-14, more preferably between 4-9, even more preferably between 5-8.

The media compositions of the present disclosure may be grown in liquid in various vessel types made of glass, stainless steel, plastic, polycarbonate, or other material and in shapes that may be cylindrical, spherical, conical, a long horizontal system, or other shape or combination of shapes. It may beneficial to select materials that are relatively impermeable to 02. It may also be beneficial to select materials that are relatively impermeable to H₂ to prevent substrate loss.

The media compositions of the present disclosure may optionally be converted to a solid medium by the addition of a gelling agent, such as agar, xantham gum, gellam gum, carrageenan, isubgol, and/or guar gum. Cells may be grown atop the solid medium or within the solid medium. Cells grown atop solid medium might improve gas substrate availability to the cells. Percentage of the gelling agent can range from 0.0001% to 99.9999%, or any range in between, or any value in between, preferably 0.1% to 10%, more preferably 0.5% to 2%. Biomass can be harvested from the plates by scraping, for example with a sterile “hockey stick.”

The biomass can then be prepared or processed using various downstream processes. In some embodiments, the biomass may be resuspended in a liquid solution. In some embodiments, the liquid solution comprises a cryoprotectant. In other embodiments, the biomass is converted to a powder by lyophilization or spray drying.

In some embodiments, the biomass of a single bacterial or archaeal strain may be prepared as a product (e.g., cell-based vaccine). In other embodiments, the biomass of multiple bacterial or archaeal strains may be combined before or after downstream processing, and be prepared as a product.

Culture may receive gas substrate (i.e., H₂ and CO₂) in a batched manner or via continuous flow.

### Cell-Based Vaccines

Vaccines of the present disclosure comprises at least one antigenic component comprising at least one cell surface antigen or a fragment thereof (e.g., cell and/or cell part) of at least one methanogen. In certain embodiments, the vaccine comprises at least one cell (e.g., whole cell) of at least one methanogen. In certain embodiments, the vaccine comprises cell part (e.g., fragment of a cell) of at least one methanogen. In certain embodiments, the vaccine is at least partially depleted of intracellular components. In some embodiments, the cell part or cell fragment is produced intentionally. In other embodiments, the cell part or cell fragment is produced during the preparation of a vaccine comprising a cell (e.g., whole cell).

Without being bound by theory, the vaccine presents one or more proteins, preferably cell surface proteins, that elicit an immune response. The vaccine of the present disclosure has the advantage of (a) presenting the native conformation of the cell surface protein that is recognized by an antibody in its natural context, (b) targeting multiple proteins present on a methanogen, and/or (c) targeting multiple methanogens, for example when a plurality of different methanogens have similar surface antigens and/or when a combination of methanogens is used.

In certain embodiments, the vaccine of the present disclosure comprises a cell and/or cell part of at least one hydrogenotroph, wherein the at least one hydrogenotroph comprises any one of the hydrogenotrophs listed in Table A or Table B.

In certain embodiments, the vaccine of the present disclosure comprises a cell and/or cell part of at least one hydrogenotroph, wherein the at least one hydrogenotroph comprises any two or more of the hydrogenotrophs listed in Table A or Table B.

In certain embodiments, the vaccine of the present disclosure comprises a cell and/or cell part at least one methanogen. In some embodiments, the at least one methanogen is of a genus Methanobrevibacter.

In some embodiments, the at least one methanogen comprises Methanobrevibacter ruminantium. In some embodiments, the at least one methanogen comprises Methanobrevibacter ruminantium M1.

In some embodiments, the at least one methanogen comprises Methanobrevibacter gottschalkii. In some embodiments, the at least one methanogen comprises Methanobrevibacter gottschalkii DSM11977.

In some embodiments, the at least one methanogen comprises Methanobrevibacter ruminantium and Methanobrevibacter gottschalkii. In some embodiments, the ratio of Methanobrevibacter ruminantium to Methanobrevibacter gottschalkii is at least about 1:1, 1.1:1, 1.2:1, 1.3:1, 1.4:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, or greater. In some embodiments, the ratio of Methanobrevibacter gottschalkii to Methanobrevibacter ruminantium is at least about 1:1, 1.1:1, 1.2:1, 1.3:1, 1.4:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, or greater.

In some such embodiments, the ratio of Methanobrevibacter ruminantium to Methanobrevibacter gottschalkii may be at least about 1:9, 2:8, 3:7, 4:6, 5:5, 6:4, 7:3, 8:2, or 9:1.

The relative dosage of cells and/or cell parts of each methanogen may be determined using any suitable technique, for example dry cell weight or a suitable cell counting technique. The relative dosage may be adjusted to account for batch differences, for example by using a potency assay, to ensure that consistency is maintained batch-to-batch.

In some embodiments, at least about 50%, 55%, 60%, 65%, 70%, 75%, 80%, 85%, 90%, 91%, 92%, 93%, 94%, 95%, 96%, 97%, 98%, 99%, or 100% of the cells and/or cell parts of the vaccine are cells (e.g., whole cells and/or fractions thereof, such as an extracellular fraction depleted in the quantity of one or more intracellular components).

In some embodiments, the vaccine composition comprises at least about 10⁶, 10⁷, 10⁸, 10⁹, 10¹⁰, or 10¹¹ cells.

In some embodiments, the cell(s) and/or cell part(s) of the vaccine composition are killed, fixed, and/or irradiated. Any suitable method can be used to kill and/or fix the cell(s) and/or cell part(s) of the vaccine's composition.

In some embodiments, the cell(s) and/or cell part(s) of the vaccine composition are killed by high temperature (heat), steam, or low temperature (freezing).

In some embodiments, the cell(s) and/or cell part(s) of the vaccine composition are fixed by formaldehyde or formalin.

In some embodiments, the cell(s) and/or cell part(s) of the vaccine composition are irradiated by UV or gamma irradiation.

In some embodiments, the cell(s) and/or cell part(s) of the vaccine composition are sonicated.

In some embodiments, the cell(s) and/or cell part(s) of the vaccine composition are crosslinked.

In some embodiments, the vaccine comprises cells and/or cell parts from at least 2, 3, 4, 5, 6, 7, 8, or 9 and/or not more than 20, 19, 18, 17, 16, 15, 14, 13, 12, 11, 10, 9, 8, 7, 6, 5, 4, or 3 different methanogen, for example cells and/or cell parts from 2-20 different methanogens, preferably cells and/or cell parts from 2-10 different methanogens. In embodiments, the vaccine comprises cells and/or cell parts from M. gottschalkii and M. ruminantium optionally in combination with cells and/or cell parts from 1-18 additional different methanogens.

In some embodiments, the cells and/or cell parts of the methanogens are endotoxin-free (e.g., less than or equal to about 100 EU as determined using a suitable test). In some embodiments, the production of the antigenic material does not require an endotoxin purification step.

In some embodiments, the vaccine composition comprises at least one adjuvant. Any suitable adjuvant can be used, for example a veterinary approved adjuvant. One of skill in the art would understand how to select an appropriate adjuvant.

In some embodiments, the at least one adjuvant comprises: oil emulsions, e.g., comprising at least (a) mineral oil lipid and (b) aqueous phase, (e.g., Freund's complete adjuvant, Freund's incomplete adjuvant, Montanide ISA series, e.g., ISA70, or Montanide ISA61, ISA206, ISA50), squaline-based emulsion, e.g., MF59 and AS03, saponins, (e.g., Quil-A, Spikoside, QS21, or ISCOMs, e.g., ISCOPREP 703), aluminum salts (e.g., aluminum hydroxide, aluminum phosphate, and potassium aluminum sulfate), also known to a skilled artisan as ‘alum’, (e.g., Imject Alum), dextran sulfate, chitosan thermogel, (e.g., monophosphoryl lipid A), Carbol, PLGA, MDP, CpG ODN, cytokine-based adjuvants such as IL-12 and/or GM-CSF, a lipid nanoparticle/cationic liposome adjuvant, an immune stimulating complex, or any combination of two or more thereof. In preferred embodiments, the at least one adjuvant comprises Freund's complete adjuvant and/or Freund's incomplete adjuvant.

In some embodiments, the at least one adjuvant comprises Emulsigen-D, Emulsigen, Emulsigen-P, and/or Polygen (MVP adjuvant, Omaha, NE). In some embodiments, the at least one adjuvant comprises ENABL 06 (HuvePharma, Peachtree City, GA). In some embodiments, the at least one adjuvant comprises Montainde ISA 201 and/or Montanide Gel 02 (Seppic Inc., New Jersey).

Without being bound by theory, the rumen may be considered a continuous flowing bioreactor with feed, water, saliva being introduced and gas, liquid, and solids exiting. Thus, any mechanism that yields a reduction in the activity and/or concentration of ruminal methanogens may be useful for a vaccine, for example (1) by binding to an impairing adhesion to the gastrointestinal tract and/or a component of feed, (2) by slowing growth rather and/or division, (3) by preventing adhesion to a microbial partner that provides an advantageous substrate thus lowering a thermodynamic benefit of the close proximity of the methanogen with its partner, (4) reducing the mobility of the methanogen, and/or (5) binding to and inhibiting a key protein/enzyme involved in methanogenesis either directly or indirectly.

In preferred embodiments, the vaccine composition induces immune response against at least one cell surface protein or a fragment thereof of the at least one methanogen. In some embodiments, the at least one cell surface protein or a fragment thereof is selected from an adhesin-like protein, adhesin-like protein with cysteine protease domain, a siderophore, a substrate/cofactor importer, a protein directly involved in methanogenesis (e.g., a bicarbonate transporter, tetrahydromethanopterin S-methyltransferase subunit), ATP generating enzymes, a fragment thereof, and/or any combination thereof.

In preferred embodiments, the vaccine composition induces production of an antibody that effectively neutralizes at least one methanogen and/or reduces the amount of CH₄ produced by the at least one methanogen.

In some embodiments, the vaccine composition is a pharmaceutical composition comprising at least one carrier and/or at least one excipient. Any suitable carrier and/or excipient can be used, for example a buffer (e.g., PBS, etc.), cryoprotectant (e.g., monosaccharides, polysaccharides, glycerol, etc.), etc.

For the cell-based vaccine composition of the present disclosure, methanogen cells (or cell parts derived from cells) can be administered at 1, 10, 1000, 10,000, 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, 1.0×10⁹, 1.0×10¹⁰, 1.0×10¹¹, 1.0×10¹² or more, or any range in between or any value in between, cells per kilogram of a subject body weight. The number of cells transplanted or injected may be adjusted based on the desired level of engraftment in a given amount of time. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be transplanted or injected. In some embodiments, transplantation or injection of at least about 100, 1000, 10,000, 0.1×10⁶, 0.5×10⁶, 1.0×10⁶, 2.0×10⁶, 3.0×10⁶, 4.0×10⁶, 5.0×10⁶, 1.0×10⁷, 1.0×10⁸, 1.0×10⁹, 1.0×10¹⁰, 1.0×10¹¹, or 1.0×10¹² total cells per dose for an average size subject is effective.

Alternatively, dosage may be held constant regardless of the body weight. In some embodiments of the cell-based vaccine composition of the present disclosure, methanogen cells (or cell parts derived from cells) can be administered at 1, 10, 1000, 10,000, 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, 1.0×10⁹, 1.0×10¹⁰, 1.0×10¹¹, 1.0×10¹² or more, or any range in between or any value in between, cells per dose.

In certain embodiments, antigenic material as disclosed herein is produced at a first location (e.g., manufacturing facility), transferred to a second location (e.g., kitting facility) for finishing (e.g., fill, finish, and kitting), and then transferred to a third location (e.g., animal site) for administration to the subject. The antigenic material may be stabilized before transfer from the first location to the second location, for example by inactivating, cross-linking, freezing, freeze-drying, or any suitable alternative. The antigen material may be transferred from the first location to the second location at any suitable temperature, for example at about environmental temperature, refrigerated, or frozen.

### Pharmaceutical Composition

Vaccines, antibodies, milk, animal feed, agents (e.g., an agent that reduces CH₄ production in a subject, a probiotic bacterial strain, etc.), or other compositions of the present disclosure may be in a pharmaceutical composition, and thus further comprise at least one excipient and/or carrier described herein or those known in the art.

The vaccine, antibody, milk, animal feed, or agent of the present disclosure (e.g., cell-based vaccines) may comprise at least one excipient that (1) increases stability; (2) permits the sustained or delayed release (e.g., from a depot formulation); and/or (3) alters the biodistribution (e.g., target to specific tissues or cell types). In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure include, without limitation, lipidoids, liposomes, lipid nanoparticles, polymers, lipoplexes, core-shell nanoparticles, peptides, proteins, hyaluronidase, nanoparticle mimics, and combinations thereof.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient (e.g., the vaccine). The amount of the active ingredient may be generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.01% and 99% (w/w) of the active ingredient. By way of example, the composition may comprise between 0.01% and 100%, e.g., between 0.05 and 50%, between 0.1-30%, between 5-80%, at least 80% (w/w) active ingredient.

Pharmaceutical compositions may comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients may optionally be included in the pharmaceutical formulations of the invention.

### Controlled/Sustained Release

In some embodiments, the compositions or agents of the present invention can be formulated for controlled release and/or targeted delivery. As used herein, “controlled release” refers to a pharmaceutical composition or compound release profile that conforms to a particular pattern of release to affect a specific outcome.

In some embodiments, the compositions or agents may be encapsulated into a delivery agent described herein and/or known in the art for controlled release and/or targeted delivery. As used herein, the term “encapsulate” means to enclose, surround or encase. As it relates to the formulation of the compounds of the invention, encapsulation may be substantial, complete or partial. The term “substantially encapsulated” means that at least greater than 50, 60, 70, 80, 85, 90, 95, 96, 97, 98, 99, 99.9, 99.9 or greater than 99.999% of the pharmaceutical composition or compound of the invention may be enclosed, surrounded or encased within the delivery agent. “Partially encapsulation” means that less than 10, 10, 20, 30, 40 50 or less of the pharmaceutical composition or compound of the invention may be enclosed, surrounded or encased within the delivery agent. Advantageously, encapsulation may be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, 99.99 or greater than 99.99% of the pharmaceutical composition or compound of the invention are encapsulated in the delivery agent.

In some embodiments, the controlled release formulation may include, but is not limited to, tri-block co-polymers. As a non-limiting example, the formulation may include two different types of tri-block co-polymers (International Pub. No. WO2012131104 and WO2012131106; the contents of each of which is herein incorporated by reference in its entirety).

### Excipients

Vaccines, antibodies, milk, animal feed, agents (e.g., an agent that reduces CH₄ production in a subject, a probiotic bacterial strain, etc.), or other compositions of the present disclosure may comprise at least one excipient and/or carrier described herein or those known in the art (e.g., a pharmaceutically acceptable excipient and/or carrier).

A pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, solid binders, lubricants, flavoring agents, stabilizers, antioxidants, osmolality adjusting agents, pH adjusting agents and the like, as suited to the particular dosage form desired.

In some embodiments, one or more excipients or accessory ingredients may make up greater than 50% of the total mass or volume of a pharmaceutical composition. In some embodiments, the one or more excipients or accessory ingredients may make up at least about 50%, 60%, 70%, 80%, 90%, or more of a pharmaceutical convention.

Various excipients for formulating pharmaceutical compositions and techniques for preparing the composition are known in the art (see Remington: The Science and Practice of Pharmacy, 21^{st} Edition, A. R. Gennaro (Lippincott, Williams & Wilkins, Baltimore, MD, 2006; incorporated herein by reference in its entirety).

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions. The composition may also include excipients such as cocoa butter and suppository waxes, coloring agents, coating agents, sweetening, flavoring, and/or perfuming agents.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ©45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRU®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g., cornstarch and starch paste); gelatin; sugars (e.g., sucrose, glucose, dextrose, dextrin, molasses, lactose, lactitol, mannitol); amino acids (e.g., glycine); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; etc.; and/or combinations thereof.

Exemplary preservatives may include, but are not limited to, antioxidants, chelating agents, antimicrobial preservatives, antifungal preservatives, alcohol preservatives, acidic preservatives, and/or other preservatives. Oxidation is a potential degradation pathway for many compounds. In order to prevent oxidation, antioxidants can be added to the formulation. Exemplary antioxidants include, but are not limited to, alpha tocopherol, ascorbic acid, acorbyl palmitate, benzyl alcohol, butylated hydroxyanisole, EDTA, m-cresol, methionine, butylated hydroxytoluene, monothioglycerol, potassium metabisulfite, propionic acid, propyl gallate, sodium ascorbate, sodium bisulfite, sodium metabisulfite, thioglycerol and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®, NEOLONE™, KATHON™, and/or EUXYL®.

In some embodiments, the pH of the vaccine solutions is maintained between pH 5 and pH 8 to improve stability. Exemplary buffers to control pH may include, but are not limited to sodium phosphate, sodium citrate, sodium succinate, histidine (or histidine-HCl), sodium carbonate, and/or sodium malate. In another embodiment, the exemplary buffers listed above may be used with additional monovalent counterions (including, but not limited to potassium). Divalent cations may also be used as buffer counterions.

Exemplary buffering agents may also include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and/or combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition.

Exemplary additives include physiologically biocompatible buffers (e.g., trimethylamine hydrochloride), addition of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). In addition, antioxidants and suspending agents can be used.

### Cryoprotectants

In some embodiments, the lipid nanoparticles and/or pharmaceutical compositions of the disclosure are refrigerated or frozen for storage and/or shipment (e.g., being stored at a temperature of 4° C. or lower, such as a temperature between about −150° C. and about 0° C. or between about −80° C. and about −20° C. (e.g., about −5° C., −10° C., −15° C., −20° C., −25° C., −30° C., −40° C., −50° C., −60° C., −70° C., −80° C., −90° C., −130° C. or −150° C.). For example, the pharmaceutical composition comprising one or more lipid nanoparticles is a solution or solid (e.g., via lyophilization) that is refrigerated for storage and/or shipment at, for example, about −20° C., −30° C., −40° C., −50° C., −60° C., −70° C., or −80° C. In certain embodiments, the disclosure also relates to a method of increasing stability of the lipid nanoparticles and by storing the lipid nanoparticles and/or pharmaceutical compositions thereof at a temperature of 4° C. or lower, such as a temperature between about −150° C. and about 0° C. or between about −80° C. and about −20° C., e.g., about −5° C., −10° C., −15° C., −20° C., −25° C., −30° C., −40° C., −50° C., −60° C., −70° C., −80° C., −90° C., −130° C. or −150° C.).

In some embodiments, vaccine formulations may comprise cryoprotectants. As used herein, there term “cryoprotectant” refers to one or more agent that when combined with a given substance, helps to reduce or eliminate damage to that substance that occurs upon freezing. In some embodiments, cryoprotectants are combined with vaccines in order to stabilize them during freezing. Frozen storage of vaccines between −20° C. and −80° C. may be advantageous for long term (e.g., 36 months) storage. In some embodiments, cryoprotectants are included in vaccine formulations through freeze/thaw cycles and under frozen storage conditions. Cryoprotectants of the present invention may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

### Inactive Ingredients

In some embodiments, vaccine formulations may comprise at least one excipient which is an inactive ingredient. As used herein, the term “inactive ingredient” refers to one or more inactive agents included in formulations. Exemplary non-exhaustive lists of inactive ingredients and the routes of administration the inactive ingredients may be formulated in are described in Tables 2 and 3.

TABLE 2
Exemplary inactive ingredients

| Inactive Ingredient | Route of Administration |
|---|---|
| Alpha-Terpineol | Topical |
| Alpha-Tocopherol | Intravenous; Topical |
| Alpha-Tocopherol Acetate, DI- | Topical |
| Alpha-Tocopherol, DI- | Intravenous; Topical |
| 1,2,6-Hexanetriol | Topical |
| 1,2-Dimyristoyl-Sn-Glycero-3-(Phospho-S- | Intravenous; Infusion (IV) |
| (1-Glycerol)) | |
| 1,2-Dimyristoyl-Sn-Glycero-3- | Intravenous; Infusion (IV) |
| Phosphocholine | |
| 1,2-Dioleoyl-Sn-Glycero-3-Phosphocholine | Epidural |
| 1,2-Dipalmitoyl-Sn-Glycero-3-(Phospho- | Epidural |
| Rac-(1-Glycerol)) | |
| 1,2-Distearoyl-Sn-Glycero-3-(Phospho-Rac- | Intravenous |
| (1-Glycerol)) | |
| 1,2-Distearoyl-Sn-Glycero-3-Phosphocholine | Intravenous |
| 1-O-Tolylbiguanide | Topical |

TABLE 3
Exemplary inactive ingredients

| Route of Administration | Inactive Ingredient |
|---|---|
| Intrathecal (AN, CNBLK) | Acetone Sodium Bisulfite; Citric Acid; ITydrochloric Acid; Sodium |
| | Chloride; Sodium Ilydroxide; Sodium Metabisulfite |
| Infiltration (AN) | Acetic Acid; Acetone Sodium Bisulfite; Ascorbic Acid; Benzyl |
| | Alcohol; Calcium Chloride; Carbon Dioxide; Chlorobutanol; Citric |
| | Acid; Citric Acid Monohydrate; Edetate Calcium Disodium; Edetate |
| | Disodium; Hydrochloric Acid; Hydrochloric Acid, Diluted; Lactic Acid; |
| | Methylparaben; Monothioglycerol; Nitrogen; Potassium Chloride; |
| | Potassium Metabisulfite; Potassium Phosphate, Monobasic; |
| | Propylparaben; Sodium Bisulfite; Sodium Carbonate; Sodium Chlorate; |
| | Sodium Chloride; Sodium Citrate; Sodium Hydroxide; Sodium Lactate; |
| | Sodium Metabisulfite; Sodium Phosphate, Dibasic, Heptahydrate |
| Sympathetic NBLK (AN) | Hydrochloric Acid; Sodium Chloride; Sodium Hydroxide |
| Auricular (Otic) | Acetic Acid; Aluminum Acetate; Aluminum Sulfate Anhydrous; |
| | Benzalkonium Chloride; Benzethonium Chloride; Benzyl Alcohol; |
| | Boric Acid; Calcium Carbonate; Cetyl Alcohol; Chlorobutanol; |
| | Chloroxylenol; Citric Acid; Creatinine; Cupric Sulfate; Cupric Sulfate |
| | Anhydrous; Edetate Disodium; Edetic Acid; Glycerin; Glyceryl |
| | Stearate; Hydrochloric Acid; Hydrocortisone; Hydroxyethyl Cellulose; |
| | Isopropyl Myristate; Lactic Acid; Lecithin. Hydrogenated; |
| | Methylparaben; Mineral Oil; Petrolatum; Petrolatum, White; |
| | Phenylethyl Alcohol; Polyoxyl 40 Stearate; Polyoxyl Stearate; |
| | Polysorbate 20; Polysorbate 80; Polyvinyl Alcohol; Potassium |
| | Metabisulfite; Potassium Phosphate, Monobasic; Povidone K901; |
| | Povidones; Propylene Glycol; Propylene Glycol Diacetate; |
| | Propylparaben; Sodium Acetate; Sodium Bisulfite; Sodium Borate; |
| | Sodium Chloride; Sodium Citrate; Sodium Hydroxide; Sodium |
| | Phosphate, Dibasic, Anhydrous; Sodium Phosphate, Dibasic, |
| | Heptahydrate; Sodium Phosphate, Monobasic, Anhydrous; Sodium |

In Table 3, “AN” means anesthetic, “CNBLK” means cervical nerve block, “NBLK” means nerve block, and “IV” means intravenous.

### Naked Delivery

The compositions or agents of the present invention may be delivered to a subject naked or in saline. The naked compositions or agents may be administered to an animal using routes of administration known in the art and described herein.

### Administration

Vaccines, antibodies, milk, animal feed, agents (e.g., an agent that reduces CH₄ production in a subject, a probiotic bacterial strain, a small molecule inhibitor, etc.), or other compositions of the present disclosure (e.g., those reducing CH₄ production in a subject) may be administered to a subject by any route which results in a therapeutically effective outcome. These include, but are not limited to, enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the auras media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique, ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis or spinal. In specific embodiments, compositions may be administered in a way which allows them to cross the blood-brain barrier, vascular barrier, or other epithelial barrier.

Non-limiting routes of administration for the compositions or agents of the present disclosure are described below.

### Parenteral and Injectable Administration

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

A pharmaceutical composition for parenteral administration may comprise at least one inactive ingredient. A non-exhaustive list of inactive ingredients for use in pharmaceutical compositions for parenteral administration includes hydrochloric acid, mannitol, N₂, sodium acetate, sodium chloride and sodium hydroxide. Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S. P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables. The sterile formulation may also comprise adjuvants such as local anesthetics, preservatives and buffering agents.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Injectable formulations may be for direct injection into a region of a tissue, organ and/or subject. As a non-limiting example, a tissue, organ and/or subject may be directly injected a formulation by intramyocardial injection into the ischemic region. (See e.g., Zangi et al. Nature Biotechnology 2013; the contents of which are herein incorporated by reference in its entirety).

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

Compositions for rectal or vaginal (e.g., transvaginal) administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

As a non-limiting example, the formulations for rectal and/or vaginal administration may be prepared by mixing the drug with a suitable non-irritating excipient that is solid at ordinary temperatures but liquid at the rectal temperature and will therefore melt in the rectum and/or vagina to release the drug. Such materials include cocoa butter and polyethylene glycols.

A pharmaceutical composition for rectal administration may comprise at least one inactive ingredient. A non-exhaustive list of inactive ingredients for use in pharmaceutical compositions for rectal administration includes alcohol, alcohol, dehydrated, aluminum subacetate, anhydrous citric acid, aniseed oil, ascorbic acid, ascorbyl palmitate, balsam peru, benzoic acid, benzyl alcohol, bismuth subgallate, butylated hydroxyanisole, butylated hydroxytoluene, butylparaben, caramel, carbomer 934, carbomer 934p, carboxypolymethylene, cerasynt-se, cetyl alcohol, cocoa butter, coconut oil, hydrogenated, coconut oil/palm kernel oil glycerides, hydrogenated, cola nitida seed extract, d&c yellow no. 10, dichlorodifluoromethane, dichlorotetrafluoroethane, dimethyldioctadecylammonium bentonite, edetate calcium disodium, edetate disodium, edetic acid, epilactose, ethylenediamine, fat, edible, fat, hard, fd&c blue no. 1, fd&c green no. 3, fd&c yellow no. 6, flavor fig 827118, flavor raspberry pfc-8407, fructose, galactose, glycerin, glyceryl palmitate, glyceryl stearate, glyceryl stearate/peg stearate, glyceryl stearate/peg-40 stearate, glycine, hydrocarbon, hydrochloric acid, hydrogenated palm oil, hypromelloses, lactose, lanolin, lecithin, light mineral oil, magnesium aluminum silicate, magnesium aluminum silicate hydrate, methylparaben, N₂, palm kernel oil, paraffin, petrolatum, white, polyethylene glycol 1000, polyethylene glycol 1540, polyethylene glycol 3350, polyethylene glycol 400, polyethylene glycol 4000, polyethylene glycol 6000, polyethylene glycol 8000, polysorbate 60, polysorbate 80, potassium acetate, potassium metabisulfite, propylene glycol, propylparaben, saccharin sodium, saccharin sodium anhydrous, silicon dioxide, colloidal, simethicone, sodium benzoate, sodium carbonate, sodium chloride, sodium citrate, sodium hydroxide, sodium metabisulfite, sorbitan monooleate, sorbitan sesquioleate, sorbitol, sorbitol solution, starch, steareth-10, steareth-40, sucrose, tagatose, d-, tartaric acid, dl-, trolamine, tromethamine, vegetable oil glyceride, hydrogenated, vegetable oil, hydrogenated, wax, emulsifying, white wax, xanthan gum and zinc oxide.

A pharmaceutical composition for vaginal administration may comprise at least one inactive ingredient. Any or none of the inactive ingredients used may have been approved by the US Food and Drug Administration (FDA). A non-exhaustive list of inactive ingredients for use in pharmaceutical compositions for vaginal administration includes adipic acid, alcohol, denatured, allantoin, anhydrous lactose, apricot kernel oil peg-6 esters, barium sulfate, beeswax, bentonite, benzoic acid, benzyl alcohol, butylated hydroxyanisole, butylated hydroxytoluene, calcium lactate, carbomer 934, carbomer 934p, cellulose, microcrystalline, ceteth-20, cetostearyl alcohol, cetyl alcohol, cetyl esters wax, cetyl palmitate, cholesterol, choleth, citric acid, citric acid monohydrate, coconut oil/palm kernel oil glycerides, hydrogenated, crospovidone, edetate disodium, ethylcelluloses, ethylene-vinyl acetate copolymer (28% vinyl acetate), ethylene-vinyl acetate copolymer (9% vinylacetate), fatty alcohols, fd&c yellow no. 5, gelatin, glutamic acid, dl-, glycerin, glyceryl isostearate, glyceryl monostearate, glyceryl stearate, guar gum, high density polyethylene, hydrogel polymer, hydrogenated palm oil, hypromellose 2208 (15000 mpa·s), hypromelloses, isopropyl myristate, lactic acid, lactic acid, dl-, lactose, lactose monohydrate, lactose, hydrous, lanolin, lanolin anhydrous, lecithin, lecithin, soybean, light mineral oil, magnesium aluminum silicate, magnesium aluminum silicate hydrate, magnesium stearate, methyl stearate, methylparaben, microcrystalline wax, mineral oil, nitric acid, octyldodecanol, peanut oil, peg 6-32 stearate/glycol stearate, peg-100 stearate, peg-120 glyceryl stearate, peg-2 stearate, peg-5 oleate, pegoxol 7 stearate, petrolatum, white, phenylmercuric acetate, phospholipon 90 g, phosphoric acid, piperazine hexahydrate, poly(dimethylsiloxane/methylvinylsiloxane/methylhydrogensiloxane) dimethylvinyl or dimethylhydroxy or trimethyl endblocked, polycarbophil, polyester, polyethylene glycol 1000, polyethylene glycol 3350, polyethylene glycol 400, polyethylene glycol 4000, polyethylene glycol 6000, polyethylene glycol 8000, polyglyceryl-3 oleate, polyglyceryl-4 oleate, polyoxyl palmitate, polysorbate 20, polysorbate 60, polysorbate 80, polyurethane, potassium alum, potassium hydroxide, povidone k29/32, povidones, promulgen d, propylene glycol, propylene glycol monopalmitostearate, propylparaben, quaternium-15 cis-form, silicon dioxide, silicon dioxide, colloidal, silicone, sodium bicarbonate, sodium citrate, sodium hydroxide, sodium lauryl sulfate, sodium metabisulfite, sodium phosphate, dibasic, anhydrous, sodium phosphate, monobasic, anhydrous, sorbic acid, sorbitan monostearate, sorbitol, sorbitol solution, spermaceti, stannous 2-ethylhexanoate, starch, starch 1500, pregelatinized, starch, corn, stearamidoethyl diethylamine, stearic acid, stearyl alcohol, tartaric acid, dl-, tert-butylhydroquinone, tetrapropyl orthosilicate, trolamine, urea, vegetable oil, hydrogenated, wecobee fs, white ceresin wax and white wax.

### Oral Administration

Liquid dosage forms for oral administration include, but are not limited to, pharmaceutically acceptable emulsions, microemulsions, solutions, suspensions, syrups, and/or elixirs. In addition to active ingredients, liquid dosage forms may comprise inert diluents and/or excipients commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

Suspensions for oral dosage may contain the active materials in a mixture with excipients suitable for the manufacture of aqueous suspensions. Such excipients may be suspending agents, as a non-limiting example the suspending agents may be sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate; or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions for oral dosage can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an antioxidant such as ascorbic acid.

The oral dosage may also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soybean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g., starches, lactose, sucrose, glucose, mannitol, and silicic acid), binders (e.g., carboxymethylcellulose, alginates, gelatin, polyvinylpyrrolidinone, sucrose, and acacia), humectants (e.g., glycerol), disintegrating agents (e.g., agar, calcium carbonate, potato or tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay), and lubricants (e.g., talc, calcium stearate, magnesium stearate, solid polyethylene glycols, sodium lauryl sulfate), and mixtures thereof. In the case of capsules, tablets and pills, the dosage form may comprise buffering agents. The solid dosage forms may also dissolve once they come in contact with liquid such as, but not limited to, salvia and bile.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations.

Solid dosage forms may be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Dosage forms for oral delivery may also be chewable. The chewable dosages forms may be sustained release formulations such as, but not limited to, the sustained release compositions described in International Publication No WO2013082470 and US Publication No US20130142876, each of which is herein incorporated by reference in its entirety. The chewable dosage forms may comprise amphipathic lipids such as, but not limited to, those described in International Publication No WO2013082470 and US Publication No US20130142876, each of which is herein incorporated by reference in its entirety.

### Topical or Transdermal Administration

As described herein, compositions of the present disclosure may be formulated for administration transdermally. The skin may be an ideal target site for delivery as it is readily accessible. Gene expression may be restricted not only to the skin, potentially avoiding nonspecific toxicity, but also to specific layers and cell types within the skin.

The site of cutaneous expression of the delivered compositions will depend on the route of delivery. Two routes are commonly considered to deliver compositions to the skin: (ii) intradermal injection; and (iii) systemic delivery (e.g., for treatment of dermatologic diseases that affect both cutaneous and extracutaneous regions). Compositions can be delivered to the skin by several different approaches known in the art.

In some embodiments, the invention provides for the compositions or agents to be delivered in more than one injection.

In some embodiments, before transdermal administration at least one area of tissue, such as skin, may be subjected to a device and/or solution which may increase permeability. In one embodiment, the tissue may be subjected to an abrasion device to increase the permeability of the skin (see U.S. Patent Publication No. 20080275468, herein incorporated by reference in its entirety). In another embodiment, the tissue may be subjected to an ultrasound enhancement device. An ultrasound enhancement device may include, but is not limited to, the devices described in U.S. Publication No. 20040236268 and U.S. Pat. Nos. 6,491,657 and 6,234,990; each of which are herein incorporated by reference in their entireties. Methods of enhancing the permeability of tissue are described in U.S. Publication Nos. 20040171980 and 20040236268 and U.S. Pat. No. 6,190,315; each of which are herein incorporated by reference in their entireties.

In some embodiments, a device may be used to increase permeability of tissue before delivering formulations of compositions described herein. The permeability of skin may be measured by methods known in the art and/or described in U.S. Pat. No. 6,190,315, herein incorporated by reference in its entirety. As a non-limiting example, a formulation may be delivered by the drug delivery methods described in U.S. Pat. No. 6,190,315, herein incorporated by reference in its entirety.

In another non-limiting example tissue may be treated with a eutectic mixture of local anesthetics (EMLA) cream before, during and/or after the tissue may be subjected to a device which may increase permeability. Katz et al. (Anesth Analg (2004); 98:371-76; herein incorporated by reference in its entirety) showed that using the EMLA cream in combination with a low energy, an onset of superficial cutaneous analgesia was seen as fast as 5 min. after a pretreatment with a low energy ultrasound.

In some embodiments, enhancers may be applied to the tissue before, during, and/or after the tissue has been treated to increase permeability. Enhancers include, but are not limited to, transport enhancers, physical enhancers, and cavitation enhancers. Non-limiting examples of enhancers are described in U.S. Pat. No. 6,190,315, herein incorporated by reference in its entirety.

In some embodiments, a device may be used to increase permeability of tissue before delivering formulations of compositions described herein, which may further contain a substance that invokes an immune response. In another non-limiting example, a formulation containing a substance to invoke an immune response may be delivered by the methods described in U.S. Publication Nos. 20040171980 and 20040236268; each of which are herein incorporated by reference in their entireties.

Dosage forms for transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required.

Additionally, compositions of the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

A pharmaceutical composition for transdermal administration may comprise at least one inactive ingredient. A non-exhaustive list of inactive ingredients for use in pharmaceutical compositions for transdermal administration includes acrylates copolymer, acrylic acid-isooctyl acrylate copolymer, acrylic adhesive 788, adcote 72a103, aerotex resin 3730, alcohol, alcohol, dehydrated, aluminum polyester, bentonite, butylated hydroxytoluene, butylene glycol, butyric acid, caprylic/capric triglyceride, carbomer 1342, carbomer 940, carbomer 980, carrageenan, cetylpyridinium chloride, citric acid, crospovidone, daubert 1-5 pestr (matte) 164z, diethylene glycol monoethyl ether, diethylhexyl phthalate, dimethicone copolyol, dimethicone mdx4-4210, dimethicone medical fluid 360, dimethylaminoethyl methacrylate-butyl methacrylate-methyl methacrylate copolymer, dipropylene glycol, duro-tak 280-2516, duro-tak 387-2516, duro-tak 80-1196, duro-tak 87-2070, duro-tak 87-2194, duro-tak 87-2287, duro-tak 87-2296, duro-tak 87-2888, duro-tak 87-2979, edetate disodium, ethyl acetate, ethyl oleate, ethylcelluloses, ethylene vinyl acetate copolymer, ethylene-propylene copolymer, fatty acid esters, gelva 737, glycerin, glyceryl laurate, glyceryl oleate, heptane, high density polyethylene, hydrochloric acid, hydrogenated polybutene 635-690, hydroxyethyl cellulose, hydroxypropyl cellulose, isopropyl myristate, isopropyl palmitate, lactose, lanolin anhydrous, lauryl lactate, lecithin, levulinic acid, light mineral oil, medical adhesive modified s-15, methyl alcohol, methyl laurate, mineral oil, N₂, octisalate, octyldodecanol, oleic acid, oleyl alcohol, oleyl oleate, pentadecalactone, petrolatum, white, polacrilin, polyacrylic acid (250000 mw), polybutene (1400 mw), polyester, polyester polyamine copolymer, polyester rayon, polyethylene terephthalates, polyisobutylene, polyisobutylene (1100000 mw), polyisobutylene (35000 mw), polyisobutylene 178-236, polyisobutylene 241-294, polyisobutylene 35-39, polyisobutylene low molecular weight, polyisobutylene medium molecular weight, polyisobutylene/polybutene adhesive, polypropylene, polyvinyl acetate, polyvinyl alcohol, polyvinyl chloride, polyvinyl chloride-polyvinyl acetate copolymer, polyvinylpyridine, povidone k29/32, povidones, propylene glycol, propylene glycol monolaurate, ra-2397, ra-3011, silicon, silicon dioxide, colloidal, silicone, silicone adhesive 4102, silicone adhesive 4502, silicone adhesive bio-psa q7-4201, silicone adhesive bio-psa q7-4301, silicone/polyester film strip, sodium chloride, sodium citrate, sodium hydroxide, sorbitan monooleate, stearalkonium hectorite/propylene carbonate, titanium dioxide, triacetin, trolamine, tromethamine, union 76 amsco-res 6038 and viscose/cotton.

A pharmaceutical composition for intradermal administration may comprise at least one inactive ingredient. A non-exhaustive list of inactive ingredients for use in pharmaceutical compositions for intradermal administration includes benzalkonium chloride, benzyl alcohol, carboxymethylcellulose sodium, creatinine, edetate disodium, glycerin, hydrochloric acid, metacresol, methylparaben, phenol, polysorbate 80, protamine sulfate, sodium acetate, sodium bisulfite, sodium chloride, sodium hydroxide, sodium phosphate, sodium phosphate, dibasic, sodium phosphate, dibasic, heptahydrate, sodium phosphate, monobasic, anhydrous and zinc chloride.

### Depot Administration

As described herein, in some embodiments, the composition is formulated in depots for extended release. Generally, a specific organ or tissue (a “target tissue”) is targeted for administration.

In some aspects, the compositions or agents are spatially retained within or proximal to a target tissue. Provided are method of providing a composition to a target tissue of a mammalian subject by contacting the target tissue (which contains one or more target cells) with the composition under conditions such that the composition is substantially retained in the target tissue, meaning that at least 10, 20, 30, 40, 50, 60, 70, 80, 85, 90, 95, 96, 97, 98, 99, 99.9, 99.99 or greater than 99.99% of the composition is retained in the target tissue.

Aspects of the invention are directed to methods of providing a composition to a target tissue of a subject, by contacting the target tissue (comprising one or more target cells) with the composition under conditions such that the composition is substantially retained in the target tissue.

In some embodiments, the compositions may be retained near target tissue using a small disposable drug reservoir, patch pump or osmotic pump. Non-limiting examples of patch pumps include those manufactured and/or sold by BD® (Franklin Lakes, NJ), Insulet Corporation (Bedford, MA), SteadyMed Therapeutics (San Francisco, CA), Medtronic (Minneapolis, MN) (e.g., MiniMed), UniLife (York, PA), Valeritas (Bridgewater, NJ), and SpringLeaf Therapeutics (Boston, MA). A non-limiting example of an osmotic pump include those manufactured by DURECT® (Cupertino, CA) (e.g., DUROS® and ALZET®).

### Pulmonary Administration

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are suitably in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50% to 99.9% (w/w) of the composition, and active ingredient may constitute 0.1% to 20% (w/w) of the composition. A propellant may further comprise additional ingredients such as a liquid non-ionic and/or solid anionic surfactant and/or a solid diluent (which may have a particle size of the same order as particles comprising the active ingredient). As a non-limiting example, the compositions described herein may be formulated for pulmonary delivery by the methods described in U.S. Pat. No. 8,257,685; herein incorporated by reference in its entirety.

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface-active agent, and/or a preservative such as methylhydroxybenzoate. Droplets provided by this route of administration may have an average diameter in the range from about 0.1 nm to about 200 nm.

The compositions and formulations provided herein which may be used for pulmonary delivery may further comprise one or more surfactants. Suitable surfactants or surfactant components for enhancing the uptake of the compositions of the invention include synthetic and natural as well as full and truncated forms of surfactant protein A, surfactant protein B, surfactant protein C, surfactant protein D and surfactant Protein E, di-saturated phosphatidylcholine (other than dipalmitoyl), dipalmitoylphosphatidylcholine, phosphatidylcholine, phosphatidylglycerol, phosphatidylinositol, phosphatidylethanolamine, phosphatidylserine; phosphatidic acid, ubiquinones, lysophosphatidylethanolamine, lysophosphatidylcholine, palmitoyl-lysophosphatidylcholine, dehydroepiandrosterone, dolichols, sulfatidic acid, glycerol-3-phosphate, dihydroxyacetone phosphate, glycerol, glycero-3-phosphocholine, dihydroxyacetone, palmitate, cytidine diphosphate (CDP) diacylglycerol, CDP choline, choline, choline phosphate; as well as natural and artificial lamellar bodies which are the natural carrier vehicles for the components of surfactant, omega-3 fatty acids, polyenic acid, polyenoic acid, lecithin, palmitinic acid, non-ionic block copolymers of ethylene or propylene oxides, polyoxypropylene, monomeric and polymeric, polyoxyethylene, monomelic and polymeric, poly(vinyl amine) with dextran and/or alkanoyl side chains, Brij 35, Triton X-100 and synthetic surfactants ALEC, Exosurf, Survan and Atovaquone, among others. These surfactants can be used either as single or part of a multiple component surfactant in a formulation, or as covalently bound to a component of a pharmaceutical composition herein.

### Intranasal, Nasal and Buccal Administration

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e., by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition for inhalation (respiratory) administration may comprise at least one inactive ingredient. A non-exhaustive list of inactive ingredients for use in pharmaceutical compositions for inhalation (respiratory) administration includes acetone sodium bisulfite, acetylcysteine, alcohol, alcohol, dehydrated, ammonia, apaflurane, ascorbic acid, benzalkonium chloride, calcium carbonate, CO₂, cetylpyridinium chloride, chlorobutanol, citric acid, d&c yellow no. 10, dichlorodifluoromethane, dichlorotetrafluoroethane, edetate disodium, edetate sodium, fd&c yellow no. 6, fluorochlorohydrocarbons, gelatin, glycerin, glycine, hydrochloric acid, hydrochloric acid, diluted, lactose, lactose monohydrate, lecithin, lecithin, hydrogenated soy, lecithin, soybean, lysine monohydrate, mannitol, menthol, methylparaben, nitric acid, N₂, norflurane, oleic acid, polyethylene glycol 1000, povidone k25, propylene glycol, propylparaben, saccharin, saccharin sodium, silicon dioxide, colloidal, sodium bisulfate, sodium bisulfite, sodium chloride, sodium citrate, sodium hydroxide, sodium lauryl sulfate, sodium metabisulfite, sodium sulfate anhydrous, sodium sulfite, sorbitan trioleate, sulfuric acid, thymol, titanium dioxide, trichloromonofluoromethane, tromethamine and zinc oxide.

A pharmaceutical composition for nasal administration may comprise at least one inactive ingredient. Any or none of the inactive ingredients used may have been approved by the US Food and Drug Administration (FDA). A non-exhaustive list of inactive ingredients for use in pharmaceutical compositions for nasal administration includes acetic acid, alcohol, dehydrated, allyl ·alpha·-ionone, anhydrous dextrose, anhydrous trisodium citrate, benzalkonium chloride, benzethonium chloride, benzyl alcohol, butylated hydroxyanisole, butylated hydroxytoluene, caffeine, CO₂, carboxymethylcellulose sodium, cellulose, microcrystalline, chlorobutanol, citric acid, citric acid monohydrate, dextrose, dichlorodifluoromethane, dichlorotetrafluoroethane, edetate disodium, glycerin, glycerol ester of hydrogenated rosin, hydrochloric acid, hypromellose 2910 (15000 mpa·s), methylcelluloses, methylparaben, N₂, norflurane, oleic acid, petrolatum, white, phenylethyl alcohol, polyethylene glycol 3350, polyethylene glycol 400, polyoxyl 400 stearate, polysorbate 20, polysorbate 80, potassium phosphate, monobasic, potassium sorbate, propylene glycol, propylparaben, sodium acetate, sodium chloride, sodium citrate, sodium hydroxide, sodium phosphate, sodium phosphate, dibasic, sodium phosphate, dibasic, anhydrous, sodium phosphate, dibasic, dihydrate, sodium phosphate, dibasic, dodecahydrate, sodium phosphate, dibasic, heptahydrate, sodium phosphate, monobasic, anhydrous, sodium phosphate, monobasic, dihydrate, sorbitan trioleate, sorbitol, sorbitol solution, sucralose, sulfuric acid, trichloromonofluoromethane and trisodium citrate dihydrate.

### Ophthalmic and Auricular (Otic) Administration

A pharmaceutical composition may be prepared, packaged, and/or sold in a formulation suitable for delivery to and/or around the eye and/or delivery to the ear (e.g., auricular (otic) administration). Non-limiting examples of route of administration for delivery to and/or around the eye include retrobulbar, conjuctival, intracorneal, intraocular, intravitreal, ophthlamic and subconjuctiva. Such formulations may, for example, be in the form of eye drops or ear drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this invention. A multilayer thin film device may be prepared to contain a pharmaceutical composition for delivery to the eye and/or surrounding tissue.

A pharmaceutical composition for ophthalmic administration may comprise at least one inactive ingredient. Any or none of the inactive ingredients used may have been approved by the US Food and Drug Administration (FDA). A non-exhaustive list of inactive ingredients for use in pharmaceutical compositions for ophthalmic administration includes acetic acid, alcohol, alcohol, dehydrated, alginic acid, amerchol-cab, ammonium hydroxide, anhydrous trisodium citrate, antipyrine, benzalkonium chloride, benzethonium chloride, benzododecinium bromide, boric acid, caffeine, calcium chloride, carbomer 1342, carbomer 934p, carbomer 940, carbomer homopolymer type b (allyl pentaerythritol crosslinked), carboxymethylcellulose sodium, castor oil, cetyl alcohol, chlorobutanol, chlorobutanol, anhydrous, cholesterol, citric acid, citric acid monohydrate, creatinine, diethanolamine, diethylhexyl phthalate, divinylbenzene styrene copolymer, edetate disodium, edetate disodium anhydrous, edetate sodium, ethylene vinyl acetate copolymer, gellan gum (low acyl), glycerin, glyceryl stearate, high density polyethylene, hydrocarbon gel, plasticized, hydrochloric acid, hydrochloric acid, diluted, hydroxyethyl cellulose, hydroxypropyl methylcellulose 2906, hypromellose 2910 (15000 mpa·s), hypromelloses, jelene, lanolin, lanolin alcohols, lanolin anhydrous, lanolin nonionic derivatives, lauralkonium chloride, lauroyl sarcosine, light mineral oil, magnesium chloride, mannitol, methylcellulose (4000 mpa·s), methylcelluloses, methylparaben, mineral oil, nitric acid, N₂, nonoxynol-9, octoxynol-40, octylphenol polymethylene, petrolatum, petrolatum, white, phenylethyl alcohol, phenylmercuric acetate, phenylmercuric nitrate, phosphoric acid, polidronium chloride, poloxamer 188, poloxamer 407, polycarbophil, polyethylene glycol 300, polyethylene glycol 400, polyethylene glycol 8000, polyoxyethylene-polyoxypropylene 1800, polyoxyl 35 castor oil, polyoxyl 40 hydrogenated castor oil, polyoxyl 40 stearate, polypropylene glycol, polysorbate 20, polysorbate 60, polysorbate 80, polyvinyl alcohol, potassium acetate, potassium chloride, potassium phosphate, monobasic, potassium sorbate, povidone k29/32, povidone k30, povidone k90, povidones, propylene glycol, propylparaben, soda ash, sodium acetate, sodium bisulfate, sodium bisulfite, sodium borate, sodium borate decahydrate, sodium carbonate, sodium carbonate monohydrate, sodium chloride, sodium citrate, sodium hydroxide, sodium metabisulfite, sodium nitrate, sodium phosphate, sodium phosphate dihydrate, sodium phosphate, dibasic, sodium phosphate, dibasic, anhydrous, sodium phosphate, dibasic, dihydrate, sodium phosphate, dibasic, heptahydrate, sodium phosphate, monobasic, sodium phosphate, monobasic, anhydrous, sodium phosphate, monobasic, dihydrate, sodium phosphate, monobasic, monohydrate, sodium sulfate, sodium sulfate anhydrous, sodium sulfate decahydrate, sodium sulfite, sodium thiosulfate, sorbic acid, sorbitan monolaurate, sorbitol, sorbitol solution, stabilized oxychloro complex, sulfuric acid, thimerosal, titanium dioxide, tocophersolan, trisodium citrate dihydrate, triton 720, tromethamine, tyloxapol and zinc chloride.

A pharmaceutical composition for retrobulbar administration may comprise at least one inactive ingredient. A non-exhaustive list of inactive ingredients for use in pharmaceutical compositions for retrobulbar administration includes hydrochloric acid and sodium hydroxide.

A pharmaceutical composition for intraocular administration may comprise at least one inactive ingredient. A non-exhaustive list of inactive ingredients for use in pharmaceutical compositions for intraocular administration includes benzalkonium chloride, calcium chloride, citric acid monohydrate, hydrochloric acid, magnesium chloride, polyvinyl alcohol, potassium chloride, sodium acetate, sodium chloride, sodium citrate and sodium hydroxide.

A pharmaceutical composition for intravitreal administration may comprise at least one inactive ingredient. A non-exhaustive list of inactive ingredients for use in pharmaceutical compositions for intravitreal administration includes calcium chloride, carboxymethylcellulose sodium, cellulose, microcrystalline, hyaluronate sodium, hydrochloric acid, magnesium chloride, magnesium stearate, polysorbate 80, polyvinyl alcohol, potassium chloride, sodium acetate, sodium bicarbonate, sodium carbonate, sodium chloride, sodium hydroxide, sodium phosphate dibasic heptahydrate, sodium phosphate monobasic monohydrate and trisodium citrate dehydrate.

A pharmaceutical composition for subconjunctival administration may comprise at least one inactive ingredient. A non-exhaustive list of inactive ingredients for use in pharmaceutical compositions for subconjunctival administration includes benzyl alcohol, hydrochloric acid and sodium hydroxide.

A pharmaceutical composition for auricular administration may comprise at least one inactive ingredient. A non-exhaustive list of inactive ingredients for use in pharmaceutical compositions for auricular administration includes acetic acid, aluminum acetate, aluminum sulfate anhydrous, benzalkonium chloride, benzethonium chloride, benzyl alcohol, boric acid, calcium carbonate, cetyl alcohol, chlorobutanol, chloroxylenol, citric acid, creatinine, cupric sulfate, cupric sulfate anhydrous, edetate disodium, edetic acid, glycerin, glyceryl stearate, hydrochloric acid, hydrocortisone, hydroxyethyl cellulose, isopropyl myristate, lactic acid, lecithin, hydrogenated, methylparaben, mineral oil, petrolatum, petrolatum, white, phenylethyl alcohol, polyoxyl 40 stearate, polyoxyl stearate, polysorbate 20, polysorbate 80, polyvinyl alcohol, potassium metabisulfite, potassium phosphate, monobasic, povidone k90f, povidones, propylene glycol, propylene glycol diacetate, propylparaben, sodium acetate, sodium bisulfite, sodium borate, sodium chloride, sodium citrate, sodium hydroxide, sodium phosphate, dibasic, anhydrous, sodium phosphate, dibasic, heptahydrate, sodium phosphate, monobasic, anhydrous, sodium sulfite, sulfuric acid and thimerosal.

### Dosing

Provided herein are methods comprising administering a vaccine composition to a subject. The specific dose level for any particular subject will depend upon a variety of factors including the species, the activity of the specific compound employed; the specific composition employed; the age, body weight, general health, sex, and diet of the subject; the time of administration, route of administration, and rate of excretion of the specific compound employed; drugs used in combination or coincidental with the specific compound employed; and like factors well known in the arts. Compositions in accordance with the present disclosure are typically formulated in dosage unit form for ease of administration and uniformity of dosage. It will be understood, however, that the total daily usage of the compositions of the present disclosure may be decided by the attending administrator within the scope of sound judgment.

In certain embodiments, compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect (see e.g., the range of unit doses described in International Publication No WO2013078199, herein incorporated by reference in its entirety).

For the cell-based vaccine composition of the present disclosure, methanogen cells (or cell parts derived from cells) can be administered at 1, 10, 1000, 10,000, 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, 1.0×10⁹, 1.0×10¹⁰, 1.0×10¹¹, 1.0×10¹² or more, or any range in between or any value in between, cells per kilogram of a subject body weight. The number of cells transplanted or injected may be adjusted based on the desired level of engraftment in a given amount of time. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be transplanted or injected. In some embodiment, transplantation or injection of at least about 100, 1000, 10,000, 0.1×10⁶, 0.5×10⁶, 1.0×10⁶, 2.0×10⁶, 3.0×10⁶, 4.0×10⁶, or 5.0×10⁶, 1.0×10⁷, 1.0×10⁸, 1.0×10⁹, 1.0×10¹⁰, 1.0×10¹¹, or 1.0×10¹² total cells per dose for an average size subject is effective.

For the cell-based vaccine composition of the present disclosure, methanogen cells (or cell parts derived from cells) can be administered at 1, 10, 1000, 10,000, 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, 1.0×10⁹, 1.0×10¹⁰, 1.0×10¹¹, 1.0×10¹² or more, or any range in between or any value in between, cells per dose irrespective of body weight.

The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, twenty, thirty, forty, or more administrations). When multiple administrations are employed, split dosing regimens such as those described herein may be used. In some embodiments, an animal is administered with a prime (initial dose), followed by a boost (second dose) at least about 1, 1.5, 2, 2.5, 3, 3.5, or 4 weeks after the prime. In some embodiments, at least one additional dose is given after the boost, optionally after about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months after the boost. In some embodiments, the at least one additional dose is repeated every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In preferred embodiments, an animal is administered with a prime, followed by a boost about 2 or 3 weeks after the prime. In yet other preferred embodiments, an animal is administered with (a) a prime, (b) a boost about 2 or 3 weeks after the prime, and (c) at least one additional dose every 3, 4, 5, or 6 months. Each dose in the dosing schedule may comprise the same or different number of cells. According to the present disclosure, the compositions of the present disclosure may be administered in split-dose regimens. As used herein, a “split dose” is the division of single unit dose or total daily dose into two or more doses, e.g., two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In some embodiments, the compositions of the present disclosure are administered to a subject in split doses. The compositions may be formulated in buffer only or in a formulation described herein.

### Dosage Forms

Vaccines, antibodies, milk, animal feed, agents (e.g., an agent that reduces CH₄ production in a subject, a probiotic bacterial strain, etc.), or other compositions of the present disclosure may be formulated into a dosage form described herein, such as an intranasal, intratracheal, or injectable (e.g., intravenous, intraocular, intravitreal, intramuscular, intracardiac, intraperitoneal, subcutaneous).

### Liquid Dosage Forms

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions may be mixed with solubilizing agents such as CREMOPHOR®, alcohols, oils, modified oils, glycols, polysorbates, cyclodextrins, polymers, and/or combinations thereof.

### Injectable

Injectable preparations, for example, sterile injectable aqueous or oleaginous suspensions may be formulated according to the known art and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S. P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it may be desirable to slow the absorption of the active ingredient from subcutaneous or intramuscular injection. This may be accomplished by the use of a liquid suspension of crystalline or amorphous material with poor water solubility. The rate of absorption of the compositions then depends upon its rate of dissolution which, in turn, may depend upon crystal size and crystalline form. Alternatively, delayed absorption of a parenterally administered compositions may be accomplished by dissolving or suspending the compositions in an oil vehicle. Injectable depot forms are made by forming microencapsule matrices of the compositions in biodegradable polymers such as polylactide-polyglycolide. Depending upon the ratio of compositions to polymer and the nature of the particular polymer employed, the rate of a compound release can be controlled. Examples of other biodegradable polymers include, but are not limited to, poly(orthoesters) and poly(anhydrides). Depot injectable formulations may be prepared by entrapping the compositions in liposomes or microemulsions which are compatible with body tissues.

### Pulmonary Formulation

Formulations described herein as being useful for pulmonary delivery may also be used for intranasal delivery of a pharmaceutical composition. Another formulation suitable for intranasal administration may be a coarse powder comprising the active ingredient and having an average particle from about 0.2 μm to 500 μm. Such a formulation may be administered in the manner in which snuff is taken, i.e., by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, contain about 0.1% to 20% (w/w) active ingredient, where the balance may comprise an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference in its entirety).

### Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### Multi-Dose and Repeat-Dose Administration

In some embodiments, vaccines, antibodies, milk, animal feed, agents (e.g., an agent that reduces CH₄ production in a subject, a probiotic bacterial strain, a small molecule inhibitor, etc.), or other compositions of the present disclosure may be administered in two or more doses (referred to herein as “multi-dose administration”). Such doses may comprise the same components or may comprise components not included in a previous dose. Such doses may comprise the same mass and/or volume of components or an altered mass and/or volume of components in comparison to a previous dose. In some embodiments, multi-dose administration may comprise repeat-dose administration. As used herein, the term “repeat-dose administration” refers to two or more doses administered consecutively or within a regimen of repeat doses comprising same or different components. In some embodiments, the repeat dose may comprise substantially the same components provided at substantially the same mass and/or volume. In other embodiments, the repeat dose may comprise different components (e.g., different adjuvant for a vaccine composition).

### Adjuvant

Adjuvants or immune potentiators, may also be administered with or in combination with one or more vaccine composition of the present disclosure.

The term “adjuvant” refers to an agent that when administered in conjunction with or as part of a composition described herein augments, enhances, and/or boosts the immune response to a methanogen, but when the agent is administered alone does not generate an immune response. In some embodiments, the adjuvant generates an immune response to a methanogen and does not produce an allergy or other adverse reaction. Adjuvants can enhance an immune response by several mechanisms including, e.g., lymphocyte recruitment, stimulation of B and/or T cells, and stimulation of macrophages.

In some embodiments, an adjuvant acts as a co-signal to prime T-cells and/or B-cells and/or NK cells as to the existence of the cell surface protein of a methanogen in a vaccine composition of the present disclosure.

Advantages of adjuvants include the enhancement of the immunogenicity of antigens, modification of the nature of the immune response, the reduction of the antigen amount needed for a successful immunization, the reduction of the frequency of booster immunizations needed and an improved immune response in elderly and immunocompromised vaccinees. These may be co-administered by any route, e.g., intramusculary, subcutaneous, IV or intradermal injections.

Adjuvants useful in the present invention may include, but are not limited to, natural or synthetic. They may be organic or inorganic.

When a vaccine or immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), ASO3 (Glaxo SmithKline), ASO4 (Glaxo SmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al, in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al, N. Engl. J. Med. 336, 86-91 (1997)).

Adjuvants may be selected from any of the classes (1) mineral salts, e.g., aluminium hydroxide and aluminium or calcium phosphate gels; (2) emulsions including: oil emulsions and surfactant based formulations, e.g., microfluidised detergent stabilised oil-in-water emulsion, purified saponin, oil-in-water emulsion, stabilised water-in-oil emulsion; (3) particulate adjuvants, e.g., virosomes (unilamellar liposomal vehicles incorporating influenza haemagglutinin), structured complex of saponins and lipids, polylactide co-glycolide (PLG); (4) microbial derivatives; (5) endogenous human immunomodulators; and/or (6) inert vehicles, such as gold particles; (7) microorganism derived adjuvants; (8) tensoactive compounds; (9) carbohydrates; or combinations thereof.

Other adjuvants which may be utilized in the vaccines of the present disclosure include any of those listed on the web-based vaccine adjuvant database, Vaxjo; World Wide Web at
violinet.org/vaxjo/and described in for example Sayers, et al., J. Biomedicine and Biotechnology, volume 2012 (2012), Article ID 831486, 13 pages, the content of which is incorporated herein by reference in its entirety.

Selection of appropriate adjuvants will be evident to one of ordinary skill in the art. Specific adjuvants may include, without limitation, cationic liposome-DNA complex JVRS-100, aluminum hydroxide vaccine adjuvant, aluminum phosphate vaccine adjuvant, aluminum potassium sulfate adjuvant, alhydrogel, ISCOM(s)™, Freund's complete adjuvant, Freund's incomplete adjuvant, CpG DNA Vaccine Adjuvant, Cholera toxin, Cholera toxin B subunit, Liposomes, Saponin Vaccine Adjuvant, DDA Adjuvant, Squalene-based Adjuvants, Etx B subunit Adjuvant, IL-12 Vaccine Adjuvant, LTK63 Vaccine Mutant Adjuvant, TiterMax Gold Adjuvant, Ribi Vaccine Adjuvant, Montanide ISA 720 Adjuvant, Corynebacterium-derb/ed P40 Vaccine Adjuvant, MPL™ Adjuvant, AS04, AS02, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumer™, Algal Glucan, Bay R1005, Theramide®, Stearyl Tyrosine, Specol, Algammulin, Avridine®, Calcium Phosphate Gel, CTA1-DD gene fusion protein, DOC/Alum Complex, Gamma Inulin, Gerbu Adjuvant, GM-CSF, GMDP, Recombinant hlFN-gamma/Interferon-g, Interleukin-ip, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(LC), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant El 12K of Cholera Toxin mCT-E1 12K, and/or Matrix-S.

In some embodiments, the at least one adjuvant comprises oil emulsions comprising at least (a) mineral oil lipid and (b) aqueous phase (e.g., Freund's complete adjuvant, Freund's incomplete adjuvant, Montanide ISA70, or Montanide ISA61), saponins, (e.g., Quil-A, Spikoside, QS21, or ISCOPREP 703), aluminum salts, also known to a skilled artisan as ‘alum’, (e.g., Imject Alum), dextran sulfate, chitosan thermogel, (e.g., monophosphoryl lipid A), a lipid nanoparticle/cationic liposome adjuvant, an immune stimulating complex, or any combination of two or more thereof. In preferred embodiments, the at least one adjuvant is Freund's complete adjuvant or Freund's incomplete adjuvant. See Spickler and Roth (2003) J Vet Intern Med, 17:273-281, which is incorporated herein by reference.

Other adjuvants which may be co-administered with the vaccine compositions of the invention include, but are not limited to interferons, TNF-alpha, TNF-beta, chemokines such as CCL21, eotaxin, HMGB1, SA100-8alpha, GCSF, GMCSF, granulysin, lactoferrin, ovalbumin, CD-40 L, CD28 agonists, PD-1, soluble PD1, LI or L2, or interleukins such as IL-1, IL-2, IL-4, IL-6, IL-7, IL-10, IL-12, IL-13, IL-21, IL-23, IL-15, IL-17, and IL-18. In some embodiments, the adjuvant comprises Glucopyranosyl Lipid Adjuvant (GLA), CpG oligodeoxynucleotides (e.g., Class A or B), poly(LC), aluminum hydroxide, or Pam3CSK4.

In some embodiments, the adjuvant comprises: (a) (±)-N-(3-aminopropyl)-N,N-dimethyl-2,3-bis(syn-9-tetradeceneyloxy)-1-propanaminium bromide (GAP-DMORIE) and a neutral lipid; (b) a cytokine; (c) mono-phosphoryl lipid A and trehalosedicorynomycolateAF (MPL+TDM); (d) a solubilized mono-phosphoryl lipid A formulation; and/or (e) CRL1005/BAK.

In some embodiments, the neutral lipid in (a) comprises (a) 1,2-dioleoyl-sn-glycero-3-phosphoethanolamine (DOPE); (b) 1,2-diphytanoyl-sn-glycero-3-phosphoethanolamine (DPyPE); and/or (c) 1,2-dimyristoyl-glycer-3-phosphoethanolamine (DMPE).

In some embodiments, the adjuvant comprises saponin, Montanide ISA61, a chitosan thermogel, a lipid nanoparticle/cationic liposome adjuvant, or any combination thereof. In preferred embodiments, the adjuvant comprises Montanide ISA61.

### Antibody

Unless otherwise specified here within, the terms “antibody” and “antibodies” broadly encompass naturally occurring forms of antibodies (e.g., IgG, IgA, IgM, IgE); and recombinant antibodies, such as single-chain antibodies, chimeric antibodies, and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., at least one cell surface protein or fragment thereof of at least one methanogen). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16:778). Such single chain antibodies are also intended to be encompassed within the term “antigen-binding portion” of an antibody. Any VH and VL sequences of specific scFv can be linked to immunoglobulin constant region cDNA or genomic sequences, in order to generate expression vectors encoding complete IgG polypeptides or other isotypes (e.g., IgGA). VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, biomarker peptide and a C-terminal polyhistidine tag to make bivalent and biotinylated scFv polypeptides (Kipriyanov et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g., chimeric, etc.). Antibodies may also be fully specific to the subject, e.g., the antibodies may be fully ruminant or fully human. The terms “monoclonal antibodies” and “monoclonal antibody composition,” as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

The present disclosure includes a monoclonal antibody that works particularly well in binding and neutralizing at least one methanogen. Upon immunizing a subject population (e.g., of milk-producing subject), the milk comprising the antibody can be screened for antibodies with superior activity (e.g., specific binding, neutralizing at least one methanogen, etc.). The amino acid sequence of such antibodies can be determined (e.g., mass spec-based sequencing, Next Gen Sequencing, or other methods known in the art), their expressing DNA vectors can be synthesized, and monoclonal antibodies can be produced. One or a combination of at least two or more antibodies can be added to the drinking water and/or animal feed and be given to a subject population.

Alternatively, such monoclonal antibodies can be generated by immunizing a vehicle animal (e.g., mouse, rabbit, etc.), and hybridomas expressing the animals can be recovered. Standard hybridoma methods for producing antibodies are described in, e.g., Harlow and Lane (eds.), Antibodies: A Laboratory Manual, CSH Press (1988), and CA. Janeway et al. (eds.), Immunobiology, 5^{th} Ed., Garland Publishing, New York, NY (2001)). Antibodies produced by hybridomas can be screened and utilized according to the methods described above and herein.

In some embodiments, the antibody is a polyclonal antibody. In some embodiments, the antibody is a monoclonal antibody. In some embodiments, the antibody is an IgG or IgA. In some embodiments, the antibody is IgA. The IgA isoform, at least in cattle, may be more stable in the rumen. For example, IgA levels in cattle saliva were reduced by only 40% after 8 h exposure to rumen contents while IgG levels were reduced by 80%.

In some embodiments, the antibody is lyophilized. In some embodiments, the antibody is in a pharmaceutical composition of the present disclosure or those known in the art. In some embodiments, a composition comprising an antibody further comprises at least one excipient and/or carrier. In some embodiments, the antibody is in the animal feed. In some embodiments, the antibody is in the solid animal feed. In other embodiments, the antibody is in the liquid animal feed. In some embodiments, the antibody is in the drinking water or milk. In preferred embodiments, the antibody is orally consumed by a subject such that the antibody comes in contact with at least one methanogen present in the gut of the subject. In preferred embodiments, oral administration of the antibody reduces the number and/or type of at least one methanogen.

### Methods for Detection of Antibody

In certain embodiments, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In some embodiments, the antibody titer is produced or reached by 10 days following vaccination, by 20 days following vaccination, by 30 days following vaccination, by 40 days following vaccination, or by 50 or more days following vaccination. In some embodiments, the titer is produced or reached following a single dose of vaccine administered to the subject. In other embodiments, the titer is produced or reached following multiple doses, e.g., following a first and a second dose (e.g., a repeat dose.).

In certain aspects, antigen-specific antibodies are measured in units of pg/ml or are measured in units of IU/L (International Units per liter) or mIU/ml (milli International Units per ml). In some embodiments of the invention, an efficacious vaccine produces >0.5 pg/ml, >0.1 pg/ml, >0.2 pg/ml, >0.35 pg/ml, >0.5 pg/ml, >1 pg/ml, >2 pg/ml, >5 pg/ml or >10 pg/ml. In some embodiments, an efficacious vaccine produces >10 mIU/ml, >20 mIU/ml, >50 mIU/ml, >100 mIU/ml, >200 mIU/ml, >500 mIU/ml or >1000 mIU/ml.

Methods of detecting the presence of antibodies are well known in the art.

In some embodiments, antibody level or concentration is determined or measured by neutralization assay, e.g., neutralization of at least one methanogen.

Other exemplary methods include, but are not limited to, immunodiffusion, immunoelectrophoresis, radioimmunoassay (RIA), enzyme-linked immunosorbent assays (ELISAs; including variants such as competitive ELISA, sandwich ELISA, etc.), immunofluorescent assays, Western blotting, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991 which is incorporated by reference).

For example, ELISA and RIA procedures may be conducted such that a desired protein standard (e.g., an extracellular domain of at least one cell surface protein or a fragment thereof of at least one methanogen) is labeled (with a radioisotope such as ¹²⁵I or ³⁵S, or an assayable enzyme, such as horseradish peroxidase or alkaline phosphatase), and is brought into contact with a sample comprising the antibody, whereon the amount of the labeled protein standard bound to the antibody is measured.

Enzymatic and radiolabeling of a protein and/or the antibodies may be affected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzymes are non-specific (such as using formaldehyde), and will only yield a proportion of active enzyme.

It may be desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

### Milk

In certain aspects, provided herein are milk and derivatives thereof. Milk produced by vaccinated female subjects (e.g., dairy cows) comprises antibodies that bind at least one cell surface protein or a fragment thereof of at least one methanogen. Such milk can be orally consumed by subjects such that the antibodies therein can come in contact with at least one methanogen present in the gut of the subjects. Upon contact, the antibodies in the milk can neutralize the at least one methanogen and contribute to reducing CH₄ production by the subjects.

Thus, milk from vaccinated female subject can be used to treat breast-feeding animals, thereby reducing methane production and/or methanogen colonization in young animals. This can be especially important when vaccination of young animals occurs concurrently with weaning.

In some embodiments, the milk is pasteurized and/or homogenized. In some embodiments, the milk is lyophilized, filtered, concentrated, evaporated, or processed to form dry milk powder (e.g., boiling at low pressure at low temperature). In some embodiments, said processing may allow longer shelf life of the milk/milk product and the antibodies present therein. In some embodiments, the fat content is removed/reduced from the milk. Processing of milk and/or preparation of derivatives of milk are well known in the art.

Appropriate care is taken to preserve the structural and functional (e.g., binding a methanogen) aspects of the antibodies. For example, in some embodiments, high pressure (˜200 MPa) and low temperature (−4° C.) are used throughout the process as described at least by Kim et al. (2008) Journal of Dairy Science, 91:4176-4182. In other embodiments, milk may be pasteurized at low-temperature of 60° C. for 10 min. at standard pressure. These conditions may pasteurize milk without significantly altering the antibody function.

Alternatively, milk can be filtered to remove microorganisms instead of pasteurizing. Microfiltration is a process that replaces pasteurization and produces milk with fewer microorganisms and longer shelf life without a change in the quality of the milk. In this process, cream is separated from the skimmed milk and the skimmed milk is forced through ceramic microfilters that trap 99.9% of microorganisms in the milk (as compared to 99.999% killing of microorganisms in standard high temperature short time pasteurization).

Ultrafiltration uses finer filters than microfiltration, which allow lactose and water to pass through while retaining fats, calcium and protein. As with microfiltration, the fat may be removed before filtration and added back in afterwards. Ultrafiltered milk is used widely in the industry in cheese making.

Colostrum may similarly used in the milk embodiments disclosed herein.

### Animal Feed

Provided herein are animal feeds that are useful in reducing CH₄ production by a subject. Such animal feed may be used in combination with any one of vaccines, antibodies, milk, agents (e.g., an agent that reduces CH₄ production in a subject, a probiotic bacterial strain, a small molecule inhibitor, etc.), or other compositions of the present disclosure (e.g., those reducing CH₄ production in a subject). Animal feed comprises at least one feed additive, which reduces the CH₄ production in a subject.

In some embodiments, the animal feed comprises an antibody that binds at least one cell surface antigen or a fragment thereof of at least one methanogen.

In some embodiments, the animal feed comprises a composition comprising an antibody that binds at least one cell surface antigen or a fragment thereof of at least one methanogen. For example, the animal feed may comprise milk or derivatives thereof comprising said antibody.

In some embodiments, the animal feed is liquid (e.g., drinking water, milk). An antibody that binds at least one cell surface antigen or a fragment thereof of at least one methanogen or a composition comprising same may be added to the liquid animal feed (e.g., drinking water) before being given to the subject. In some embodiments, said antibody or a composition comprising same may be added to the drinking water. In other embodiments, the milk or derivatives thereof comprising said antibody may be given directly to the subject, or added to other liquid animal feed (e.g., drinking water).

In other embodiments, the animal feed is solid. In some such embodiments, the animal feed may comprise hay, straw, silage, compressed and pelleted feeds, oils and mixed rations, and sprouted grains and legumes. An antibody that binds at least one cell surface antigen or a fragment thereof of at least one methanogen or a composition comprising same may be added to the solid animal feed before being given to the subject.

In some embodiments, an animal feed may comprise fats and fatty acids that further aid in reducing CH₄ production in subjects. Based on a meta-analysis, fat supplementation reduced CH₄ by 3.77% in cattle and 4.30% in sheep per 1% dietary fats. Fat decreases CH₄ production (expressed as g/kg digestible dry matter (DM)) more from sheep than from cattle, which was attributed to the comparatively lower depression of DM digestion together with numerically larger depression of CH₄ production (g/kg DM) by fat in sheep. Among fatty acids, C12:0, C18:3 and other polyunsaturated fatty acids (PUFA) are more potent than saturated fatty acids. The CH₄-suppressing efficacy of fats generally persists, with persistent suppression being noted for 72 days and longer in cattle.

Fats supplemented up to 6% of the diet (DM) can also improve milk production while appreciably decreasing CH₄ emissions (15%) in cattle, but higher concentrations decreased production efficiency due to a reduction of feed digestion and fermentation. Medium-chain fatty acids (MCFA) and PUFA can lower abundance and metabolic activities of rumen methanogens and change their species composition. PUFA can also directly inhibit protozoa and serve as H₂ sink through biohydrogenation. Both MCFA and PUFA appear to damage the cell membrane, thereby abolishing the selective permeability of cell membrane, which is required for survival and growth of methanogens and other microbes. The inhibitory effect of fat on methanogenesis is more pronounced in cattle fed concentrate-based diets than in cattle fed forage-based diets. Because C12; and C14:0 is more inhibitory to M. ruminantium at pH 5 than at pH 7, the concentrate level-dependent anti-methanogenic efficacy of MCFA and PUFA is probably attributed to the lower pH associated with high-concentrate diets.

In some embodiments, the animal feed comprises fat and/or fatty acid. In some embodiments, the animal feed comprises fat and/or fatty acid that is at least about 1%, 2%, 3%, 4%, 5%, or 6% of the diet (e.g., diet based on dry matter).

Numerous animal feed and feed additives are known in the art. Any agent that reduces CH₄ production in a subject (e.g., small molecule inhibitors, e.g., Table 4, probiotic bacterial strain, etc.; see below) described herein or those known in the art may be used as a feed additive. Certain exemplary feed additives include: berberine, nitrate, eucalyptus oil, alliin, diallyl disulfide (DADS), flavanone glycoside (e.g., neohesperidin, isonaringin, poncirin, hesperidin), 3-nitrooxypropanol, rac-4-Phenylbutane-1,2-diyl dinitrate, 2-(hydroxymethyl)-2-(nitrooxymethyl)-1,3-propanediol, N-ethyl-3-nitro-oxy-propionic sulfonyl amide, 5-nitrooxy-pentanenitrile, 5-nitrooxy-pentane, 3-nitro-oxy-propyl propionate, 1,3-bis-nitrooxypropane, 1,4-bis-nitrooxybutane, 1,5-bis-nitrooxypentane, 3-nitro-oxy-propyl benzoate, 3-nitro-oxy-propyl hexanoate, 3-nitro-oxy-propyl 5-nitro-oxy-hexanoate, benzylnitrate, isosorbid-dinitrate, N-[2-(nitrooxy)ethyl]-3-pyridinecarboxamide, 3-nitrooxy propionic acid, methyl-3-nitrooxy propionate, ethyl-3-nitrooxy propionate, ethyl-4-nitrooxy butanoate, ethyl-3-nitrooxy butanoate, 5-nitrooxy pentanoic acid, ethyl-5-nitrooxy pentanoate, 6-nitrooxy hexanoic acid, ethyl-6-nitrooxy hexanoate, ethyl-4-nitrooxy-cyclohexylcarboxylate, 8-nitrooxy octanoic acid, ethyl-8-nitrooxy octanoate, 11-nitrooxy undecanoic acid, ethyl-11-nitrooxy undecanoate, 5-nitrooxy-pentanoic amide, 5-nitrooxy-N-methyl-pentanoic amide, lauric acid, and haloform (e.g., bromoform, chloroform, iodoform).

Agents that Reduce CH4 Production in Ruminants Combination Treatment

Vaccines, antibodies, milk, animal feed, and agents (e.g., an agent that reduces CH₄ production in a subject, a probiotic bacterial strain, etc.) may be administered to a subject in any combination. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent (vaccines, antibodies, milk, animal feed, agents that reduce CH₄ production in a subject) will be administered at a dose and/or on a time schedule determined for that agent. In some embodiments, the present disclosure encompasses the delivery of combinations that may improve immune response against at least one methanogen, and/or reduce CH₄ production by a subject.

The combinations can conveniently be presented for use in the form of a pharmaceutical formulation and thus pharmaceutical compositions comprising a combination as defined above together with a pharmaceutically acceptable diluent or carrier represent a further aspect of the invention.

The individual compounds of such combinations can be administered either sequentially or simultaneously in separate or combined pharmaceutical formulations.

It will further be appreciated that (vaccines, antibodies, milk, animal feed, agents that reduce CH₄ production in a subject) in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually. In some embodiments, the combinations, each or together may be administered according to the split dosing regimens described herein.

The term “conjoint” or “combination” administration, as used herein, refers to the administration of two or more agents that aid in reducing CH₄ production in a subject. The different agents comprising the combination may be administered concomitant with, prior to, or following the administration of one or more agents.

In certain embodiments, combination administration can demonstrate synergisms between the two or more agents resulting in a greater CH₄ reduction in subject than either agent alone.

Synergistic effects, also known as synergy, refer to the phenomenon where the combined effect of two or more factors or components is greater than the sum of their individual effects. In other words, when these factors or components interact, they create an amplified or enhanced effect that is greater than what would be expected based on their individual contributions.

In certain cases, the agents may have different modes of action or mechanisms by which they exert their effects, for example targeting different methanogens or different methanogen enzymes. When these agents are combined, their actions can complement each other, targeting different aspects of a problem or working on multiple pathways simultaneously. This complementary action enhances their overall effectiveness, resulting in a better outcome than either agent could achieve alone.

In certain cases, synergism can significantly enhance the efficacy of the agents involved. For example, the agents may interact in a way that enhances their absorption, distribution, or bioavailability, increasing their effectiveness in treating a particular condition.

In certain cases, one agent may enhance the effects of the other without contributing much individually. This is known as potentiation. The presence of one agent can increase the uptake, binding affinity, or sensitivity of the other, making it more potent and effective. The combined effect is greater than what would be achieved by either agent on its own.

In certain cases, one or more agents may have inherent weaknesses or face resistance from target organisms or systems. By combining them with another agent, the synergistic interaction can bypass or counteract these obstacles, leading to a more effective outcome. For example, antibodies generated via vaccination of a first vaccine may face resistance to ruminal proteases, thus, combining with protease inhibitors or one or more additional vaccine towards certain ruminal proteases may reduce the resistance of the first vaccine.

In certain cases, combining two agents can amplify the positive effects or benefits they provide individually. For example, combining a vaccine for a methanogen with a vaccine towards a separate microorganism that is syntropic with the methanogen can amplify the positive effects or benefits of the vaccine by further reducing the fitness of the methanogen.

The level of synergism achieved when combining agents depends on numerous factors and is typically assessed through experimental studies or empirical observations specific to the agents and desired outcomes. Generally, combination of agents can lead to different degrees of synergisms ranging from No synergism to Supra-additive synergism.

No Synergism: In some cases, the combined effect of two or more agents may simply be additive or even less than additive. This means that the combined effect is equal to the sum of their individual effects or even lower. In such instances, no synergism is observed, and the agents may not interact in a way that amplifies their effects.

Mild to Moderate Synergism: A common outcome when combining agents is a mild to moderate level of synergism. This implies that the combined effect is greater than the sum of their individual effects, but not dramatically so. The degree of synergism may vary depending on the specific agents and the conditions of their interaction.

Strong Synergism: In some cases, the combination of agents can lead to a strong synergistic effect. This means that the combined effect is significantly greater than the sum of their individual effects. Strong synergism often results in an amplified and more potent effect, exceeding what would be expected based on the additive effects of the individual agents.

Supra-additive Synergism: In rare instances, the combined effect of two or more agents can be supra-additive, meaning it surpasses even strong synergism. Supra-additive synergism results in an exceptionally powerful effect that far exceeds the sum of the individual effects. Such cases are usually considered highly beneficial, as they can provide remarkable outcomes in terms of efficacy, efficiency, or other desired parameters.

Any suitable degree of synergism (%_{Synergism}) can be demonstrated with a combination therapy comprising any two or more compositions or agents of the present disclosure (e.g., a vaccine and another agent), such as an improvement of at least 5, 10, 20, 30, 40, 50, 60, 70, 80, 90, 100, 200, 300, or 400% and/or not more than 10, 20, 30, 40, 50, 60, 70, 80, 90, 100, 200, 300, 400, 500, or 1000%, for example 5-1000%, preferably 10-500%, more preferably 30-300%. In certain embodiments, the improvement is measured in the amount of CH₄ reduced when administered a combination therapy as compared to either agent alone, for example %_{Synergism}=CH_{4,combo}*(CH_{4,vaccine})⁻¹. In certain embodiments, the %_{Synergism} is measured in a herd of at least 10, 20, 30, 40, 50, 60, 70, 80, 90, 100, 500, or 1000 animals and/or not more than 20, 30, 40, 50, 60, 70, 80, 90, 100, 500, 1000, or 2000 animals and the statistical significance, e.g., coefficient of variation of %_{Synergism} within the herd is at least 50, 60, 70, 75, 80, 85, 90, 95, 99, 99.5, or 100%.

In certain embodiments, the synergism resulting from the combinatorial therapy results in a prolonged efficacy of the treatment as compared to either alone. In certain embodiments, the combinatorial therapy is effective for at least 1, 2, 3, 4, 5, 6, 7, 8, 9, 10, 11, 12, 16, or 20 months and/or not more than 2, 3, 4, 5, 6, 7, 8, 9, 10, 11, 12, 16, 20, or 24 months, for example 1-24 months. In certain embodiments, the length of efficacy of the treatment is at least 5, 10, 20, 30, 40, 50, 60, 70, 80, 90, 100, 200, 300, or 400% and/or not more than 10, 20, 30, 40, 50, 60, 70, 80, 90, 100, 200, 300, 400, 500, or 1000% greater than either agent alone, for example 5-1000%, preferably 10-500%, more preferably 30-300%.

An exemplary combinatorial therapy includes vaccination with a first vaccine encoding one or more methanogen surface proteins in combination with at least one additional vaccine encoding one or more of different methanogen surface proteins.

Another exemplary combinatorial therapy includes vaccination with a vaccine encoding one or more methanogen surface proteins in combination with administration of a small molecule inhibitor of methanogenesis. Without wishing to be bound to theory, it is hypothesized that the small molecule inhibitor of methanogenesis may remove the plurality of ruminal methanogens, and antibodies generated from the vaccination prevent new methanogens for colonizing the methanogen-deficient rumen.

Accordingly, in certain aspects, a vaccine of the present disclosure is administered to a subject conjointly or in a combination with at least one inhibitor of CH₄ production described herein or those known in the art. In some embodiments, the at least one inhibitor is selected from Table 4.

In some embodiments, the at least one inhibitor is administered to a subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 times. In some embodiments, the at least one inhibitor is administered to a subject daily, semiweekly, weekly, biweekly (every 2 weeks), monthly, semiannually, or annually. In some embodiments, the at least one inhibitor is administered to a subject every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days. In some embodiments, the at least one inhibitor is administered to a subject for a duration of at least, about, or no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weeks. In some embodiments, the at least one inhibitor is administered to a subject for at least 1 week but no more than 1 month. In some embodiments, the at least one inhibitor is administered to a subject orally, intravenously, intramuscularly, or subcutaneously. In preferred embodiments, the at least one inhibitor is administered to a subject orally. In some embodiments, the at least one inhibitor is administered to a subject as a feed additive.

In some embodiments, the at least one inhibitor is administered to a subject concomitant with, prior to, or following the vaccination with a vaccine of the present disclosure. In some embodiments, the at least one inhibitor is administered to a subject on the same day as the subject is vaccinated. In some embodiments, a subject is administered with the at least one inhibitor one or more times to reduce the CH₄ production by the subject, and said subject is vaccinated as a maintenance regimen.

In some embodiments, the at least one inhibitor comprises 3NOP. In some embodiments, a subject is administered at least about or no more than 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 grams (g) of 3NOP per dose. In some embodiments, 3NOP is administered to a subject every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days.

In some embodiments, at least about 0.5 g but no more than 25 g of 3NOP is administered to a subject in a given day. In preferred embodiments, about 2.5 g of 3NOP is administered to a subject in a given day. In some embodiments, about 2.5 g of 3NOP is administered to a subject per day for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

TABLE 4
Exemplary methanogenesis inhibitor

| Chemical | Reference |
|---|---|
| 1,3-bis-nitrooxypropane | U.S. Pat. No. 10,154,981B2 |
| 1,3-bis-nitrooxypropane | U.S. Pat. No. 9,266,814B2 |
| 1,4-bis-nitrooxybutane | U.S. Pat. No. 10,806,706B2 |
| 1,4-bis-nitrooxybutane | U.S. Pat. No. 10,154,981B2 |
| 1,4-bis-nitrooxybutane | U.S. Pat. No. 9,266,814B2 |
| 1,5-bis-nitrooxypentane | U.S. Pat. No. 10,806,706B2 |
| 1,5-bis-nitrooxypentane | U.S. Pat. No. 10,154,981B2 |
| 1,5-bis-nitrooxypentane | U.S. Pat. No. 9,266,814B2 |
| 11-nitrooxy undecanoic acid | U.S. Pat. No. 9,365,489B2 |
| 11-nitrooxy undecanoic acid | U.S. Pat. No. 10,154,981B2 |
| 2 mercaptoethanesulfonate | EP2747181A1 |
| 2-bromoethane sulfonic acid (2-BESA) | EP2747181A1 |
| 2-bromoethane sulphonate | U.S. Pat. No. 6,251,879B1 |
| 2-chloroethanesulfonate | EP2747181A1 |
| 2-nitropropanol | EP2747181A1 |
| 3-nitro-oxy-propyl 5-nitro-oxy-hexanoate | U.S. Pat. No. 10,154,981B2 |
| 3-nitro-oxy-propyl benzoate | U.S. Pat. No. 10,154,981B2 |
| 3-nitro-oxy-propyl hexanoate | U.S. Pat. No. 10,154,981B2 |
| 3-nitro-oxy-propyl propionate | U.S. Pat. No. 10,154,981B2 |
| 3-nitrooxy propionic acid | U.S. Pat. No. 9,365,489B2 |
| 3-nitrooxy propionic acid | U.S. Pat. No. 10,154,981B2 |
| 3-nitrooxy-propyl 5-nitrooxy-hexanoate | U.S. Pat. No. 9,266,814B2 |
| 3-nitrooxy-propyl benzoate | U.S. Pat. No. 9,266,814B2 |
| 3-nitrooxy-propyl hexanoate | U.S. Pat. No. 9,266,814B2 |
| 3-nitrooxy-propyl propionate | U.S. Pat. No. 9,266,814B2 |
| 3-nitrooxypropanol | U.S. Pat. No. 10,806,706B2 |
| 3-nitrooxypropanol | U.S. Pat. No. 10,154,981B2 |
| 3-nitrooxypropanol | U.S. Pat. No. 9,266,814B2 |
| 5-nitrooxy hexanoic acid | U.S. Pat. No. 10,806,706B2 |
| 5-nitrooxy pentanoic acid | U.S. Pat. No. 10,806,706B2 |
| 5-nitrooxy pentanoic acid | U.S. Pat. No. 9,365,489B2 |
| 5-nitrooxy pentanoic acid | U.S. Pat. No. 10,154,981B2 |
| 5-nitrooxy-N-methyl-pentanoic amide | U.S. Pat. No. 9,365,489B2 |
| 5-nitrooxy-N-methyl-pentanoic amide | U.S. Pat. No. 10,154,981B2 |
| 5-nitrooxy-N-pentanoic amide | U.S. Pat. No. 9,365,489B2 |
| 5-nitrooxy-pentane | U.S. Pat. No. 10,154,981B2 |
| 5-nitrooxy-pentane | U.S. Pat. No. 9,266,814B2 |
| 5-nitrooxy-pentanenitrile | U.S. Pat. No. 10,154,981B2 |
| 5-nitrooxy-pentanenitrile | U.S. Pat. No. 9,266,814B2 |
| 5-nitrooxy-pentanoic amide | U.S. Pat. No. 10,154,981B2 |
| 6-nitrooxy hexanoic acid | U.S. Pat. No. 9,365,489B2 |
| 6-nitrooxy hexanoic acid | U.S. Pat. No. 10,154,981B2 |
| 8-nitrooxy octanoic acid | U.S. Pat. No. 9,365,489B2 |
| 8-nitrooxy octanoic acid | U.S. Pat. No. 10,154,981B2 |
| 9-nitrooxynonanol | U.S. Pat. No. 10,806,706B2 |
| abietic acid | U.S. Pat. No. 10,799,544B2 |
| acetylene | EP2747181A1 |
| ajoene | US20220256890A1 |
| Alcaligenes faecalis | US20140072535A1 |
| allicin | US20220256890A1 |
| alliin | US20220256890A1 |
| allylpropyl disulfide | US20220256890A1 |
| Asparagopsis armata | U.S. Pat. No. 10,881,697B2 |
| Bacillus amyloliquefaciens | WO2021211548A1 |
| Bacillus strain 300 (NRRL No. B-50943) | US20210360945A1 |
| Bacillus strain 86 (NRRL No. B-50944) | US20210360945A1 |
| Bacillus, Lactobacillus, Streptococcus, Candida and Pichia | WO2007072935A1 |
| bambermycin | AU2018229465A1 |
| benzylnitrate | U.S. Pat. No. 10,154,981B2 |
| berberine, nitrate, and eucalyptus oil | U.S. Pat. No. 10,440,975B2 |
| bis-(2-nitrooxyethyl) ether | U.S. Pat. No. 9,266,814B2 |
| bis(2-hydroxyethyl)amine dinitrate | U.S. Pat. No. 10,806,706B2 |
| bromochloromethane | U.S. Pat. No. 6,251,879B1 |
| Bromoforms | US20220175670A1 |
| chloroform | EP2747181A1 |
| Chloroforms | US20220175670A1 |
| Cysteine | U.S. Pat. No. 5,843,498A |
| dehydoabietic acid | U.S. Pat. No. 10,799,544B2 |
| diallyl disulfide | US20180093308A1 |
| diallyl disulphide | US20220256890A1 |
| diallyl trisulfide | US20180093308A1 |
| diallyl trisulfide | US20220256890A1 |
| egg powder from immunizing hens with CH₄-producing M. stadtmaniae | U.S. Pat. No. 7,820,171B2 |
| methanogens | |
| Eggs laid by hens inoculated with methanogen antigens | US20200121730A1 |
| essential oil | US20180001358A1 |
| ethyl 2 butynoate | EP2747181A1 |
| ethyl propionate | US20180093308A1 |
| ethyl-11-nitrooxy undecanoate | U.S. Pat. No. 9,365,489B2 |
| ethyl-11-nitrooxy undecanoate | U.S. Pat. No. 10,154,981B2 |
| ethyl-3-nitrooxy butanoate | U.S. Pat. No. 10,154,981B2 |
| ethyl-3-nitrooxy propionate | U.S. Pat. No. 9,365,489B2 |
| ethyl-3-nitrooxy propionate | U.S. Pat. No. 10,154,981B2 |
| ethyl-3-nitrooxy-butanoate | U.S. Pat. No. 9,365,489B2 |
| ethyl-4-nitrooxy butanoate | U.S. Pat. No. 10,154,981B2 |
| ethyl-4-nitrooxy-butanoate | U.S. Pat. No. 9,365,489B2 |
| ethyl-4-nitrooxy-cyclohexylcarboxylate | U.S. Pat. No. 9,365,489B2 |
| ethyl-4-nitrooxy-cyclohexylcarboxylate | U.S. Pat. No. 10,154,981B2 |
| ethyl-5-nitrooxy pentanoate | U.S. Pat No. 9,365,489B2 |
| ethyl-5-nitrooxy pentanoate | U.S. Pat. No. 1,0154,981B2 |
| ethyl-6-nitrooxy hexanoate | U.S. Pat. No. 9,365,489B2 |
| ethyl-6-nitrooxy hexanoate | U.S. Pat. No. 10,154,981B2 |
| ethyl-8-nitrooxy octanoate | U.S. Pat. No. 9,365,489B2 |
| ethyl-8-nitrooxy octanoate | U.S. Pat. No. 10,154,981B2 |
| ethylene | EP2747181A1 |
| eugenol | U.S. Pat. No. 7,763,273B2 |
| fluoroacetate | EP2747181A1 |
| fluvastatin | EP2747181A1 |
| halothane | U.S. Pat. No. 6,251,879B1 |
| hesperidin | EP2838376B1 |
| hop acid | U.S. Pat. No. 8,197,863B2 |
| hydrogen peroxide | WO2010071222A1 |
| hydroxycinnamic acid | WO2021038832A1 |
| Impatiens balsamina | NZ562783A |
| Iodoforms | US20220175670A1 |
| isonaringin | EP2838376B1 |
| isopimaric acid | U.S. Pat. No. 10,799,544B2 |
| isosorbid-dinitrate | U.S. Pat. No. 10,154,981B2 |
| isosorbid-dinitrate | U.S. Pat. No. 9,266,814B2 |
| laidlomycin | AU2018229465A1 |
| lasalocid | AU2018229465A1 |
| limonene | U.S. Pat. No. 7,7632,73B2 |
| lovastatin | EP2747181A1 |
| lumazine | EP2747181A1 |
| medium and long chain fatty acids | EP2747181A1 |
| Megasphaera sp. & Coprococcus catus | U.S. Pat. No. 10,961,559B2 |
| methyl chloride | EP2747181A1 |
| methyl fluoride | EP2747181A1 |
| methyl-3-nitrooxy propionate | U.S. Pat. No. 9,365,489B2 |
| methyl-3-nitrooxy propionate | U.S. Pat. No. 10,154,981B2 |
| mevastatin | EP2747181A1 |
| Monascus sp. | US20130011384A1 |
| monensin | AU2018229465A1 |
| N-[2-(nitrooxy)ethyl]-3-pyridinecarboxamide | U.S. Pat. No. 10,154,981B2 |
| N-[2-(nitrooxy)ethyl]-3-pyridinecarboxamide | U.S. Pat. No. 9,266,814B2 |
| N-ethyl-3-nitro-oxy-propionic sulfonyl amide | U.S. Pat. No. 10,154,981B2 |
| neoabietic acid | U.S. Pat. No. 10,799,544B2 |
| neohesperidin | EP2838376B1 |
| nitrate | EP2747181A1 |
| nitrite | EP2747181A1 |
| nitroethane | EP2747181A1 |
| palustric acid | U.S. Pat. No. 10,799,544B2 |
| Peroxide source, peroxidase | US20210227850A1 |
| phosphate | EP2747181A1 |
| pimaric acid | U.S. Pat. No. 10,799,544B2 |
| poncirin | EP2838376B1 |
| Propionibacterium | US20120276058A1 |
| propynoic acid | EP2747181A1 |
| protease-resistant bacteriocin | EP1673983A1 |
| R(OC₂H₄)n(OH); R = C12-15; n = 7 | AU2013288441B2 |
| R1(CH₂)nONO₂; n = 1-15; R1 = H, C1-C6alkyl, phenyl, —OH, —NH₂, | U.S. Pat. No. 10,806,706B2 |
| —CN, —COOH, —O(C═O)R8, —NHC(═O)R8, SO₂NHR8, or —ONO₂; | |
| R8 = C1-C6alkyl, phenyl, pyridyl; when n is >3 the hydrocarbon | |
| chain may be interrupted by —O— or —NH— | |
| rac-4-Phenylbutane-1,2-diyl dinitrate, 2-(hydroxymethyl)-2- | U.S. Pat. No. 1,015,4981B2 |
| (nitrooxymethyl)-1,3-propanediol | |
| red yeast rice | US20140322798A1 |
| s-allyleysteine | US20220256890A1 |
| saponin | US20180001358A1 |
| Saponin & medium chain fatty acid | U.S. Pat. No. 10,695,393B2 |
| sodium bromoethenesulfonate (BES) | EP2747181A1 |
| vinyldithiines | US20220256890A1 |
| ω-nitrooxyC3-10alkane-1-ol | US20210392922A1 |
| 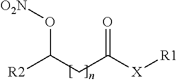 | U.S. 9,365,489B2 |
| 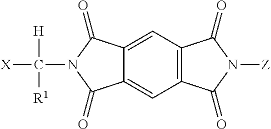 | WO2022103280A1 |
| 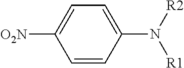 | US20160183564A1 |
| 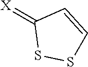 | EP3628169A1 |
| 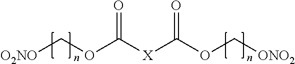 | US20210163397A1 |
| 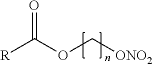 | WO2020221805A1 |
| 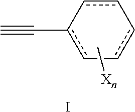 | AU2018383221A1 |
| 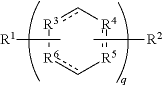 | US20030219467A1 |
| Asparagopsis taxiformis | USPP34607P3 |
| Lauric acid | |

*Each of the references in Table 4 is incorporated herein by reference.
Other exemplary inhibitors of CH₄ production are discussed below.

Probiotics that Reduce CH₄ Production in Animals

Probiotics are a class of beneficial active microorganisms or their cultures. Probiotics are useful in reducing CH₄ emissions in animals (Table 8C). There are many types of probiotics, and different strains have different inhibitory effects on CH₄ emissions. For example, the GA03 strain of Acetobacter is more effective at inhibiting CH₄ production than other isolated strains. Most probiotics reduce CH₄ production by influencing the activities of ruminal microorganisms, with no adverse effects on animals. In addition, probiotics enhance ruminal fermentation.

Lactic acid bacteria, which have been used as feed additives for a long time, not only reduce CH₄ emissions per unit volatile fatty acid (VFA) output, but also improve the fermentation quality and fiber digestibility of silage. In addition, the denitrifying bacterium Bacillus 79R4 could prevent NO-2-N poisoning and microbial ecosystems from impairing fermentation efficiency. Furthermore, Bacillus licheniformis reduces CH₄ production and increases feed energy and protein utilization.

TABLE 5
Exemplary probiotic bacteria that reduce CH₄ production

| | | Addition amount; maximum | |
|---|---|---|---|
| Types of probiotics | Inhibitory effect | suppression methane amount | Inhibition mechanism |
| Propionic acid bacillus | [symbol] (Most propionic bacteria) | 100 μL of the propionic acid | Unknown |
| | [symbol] (P. jensenii [symbol] MGT282 and P. | bacteria culture (2 × 10 [symbol] to 4 × 10 [symbol] ) | |
| | thoenii LMGT2827 or T159) | colony forming units), | |
| | | Propionibacteritum thoenii T159,20% | |
| Lactic acid bacteria | [symbol] | 5.3 lg cfu/g fresh weight, | Hydrogen consumption |
| | | Lactobacillus plantarum, 8.8 ml/g(72 h) | |
| Acetic acid bacteria | [symbol] | 1% Proteiniphilum acetatigenes | Reduced the number of |
| | | GA06; — | methanogens |
| Enterococcus faecium | [symbol] | 0.1%; 2.08 mM/mL | Alters microbial flora |
| SROD | | | |
| Probiotic products | [symbol] | 2 g probiotic products in | Reduce the number of |
| of Ruminococcus | | powder; 1.2 ml/g of dry matter | rumen protozoa |
| flavofaciens | | 10 ml probiotic products in liquid; | |
| | | 1.2 ml/g of dry matter | |
| Bacillus licheniformis | [symbol] | 2.5 × 10 [symbol] 2.7 L/d | Unknown |
| Saccharomyces | [symbol] (Pedrazo-Hernandez et al. 2019) | — | Affects rumen microbes |
| cerevisiae | [symbol] ( [symbol] et al., 2019) | | |

[symbol]

indicates data missing or illegible when filed

Prebiotics that Reduce CH₄ Production

Prebiotics are substances that are not easily digested or absorbed by the host. They selectively stimulate the growth and activity of one or several ruminal microorganisms with a positive effect on ruminal fermentation. Prebiotics suppress ruminal CH₄ production in subjects. Prebiotics mainly reduce rumen CH₄ production by altering the bacterial community structure, influencing the permeability of the cell walls of methanogenic archaea, and stimulating other bacteria to compete with methanogens for H₂ (Table 9).

The prebiotic chitosan can influence bacterial community structures by altering microbial population compositions, for example, by replacing fibrinolytic enzyme-producing microbes (Firmicutes and Fibrobacteres) with amylolytic enzyme-producing microbes (Bacteroides and Proteus); in turn, reducing CH₄ production. Chitosan could influence the ruminal fermentation process by altering VFA distributions and increasing propionic acid concentrations, which reduces CH₄ production in turn. However, the reduction in CH₄ is associated with the degree of chitosan deacetylation, which could alter the permeability of the methanogen cell wall. In addition, various yeast products could reduce CH₄ emissions by stimulating acetic acid-producing bacteria to compete with methanogens or metabolize H₂.

TABLE 6
Exemplary prebiotic bacteria that reduce CH₄ production

| Types of | Inhibitory | Addition amount; maximum | |
|---|---|---|---|
| prebiotics | effects | methane suppression amount | Inhibition mechanism |
| Chitosan | — | 3000 (molecular weights) | (1) Alters microbial |
| | | dry matter; 22.9% | community structure |
| | | ml/day (Tong et al., 2020) | (Tong et al., 2020) |
| | | 2% Chitosan + 21% of crude | (2) Alters fermentation |
| | | glycerin: 53.67% ( [symbol] , | pathway ( [symbol] , |
| | | Cherdthong [symbol] , 2020) | Cherdthong [symbol] |
| | | | [symbol] , 2020) |
| | | | (3) Influences |
| | | | methanogenic bacteria |
| | | | cell wall permeability |
| | | | ( [symbol] et al., 2018) |
| Yeast | — | 4 mg/l g dry matter; — | Indirect consumption of |
| products | | | hydrogen |

[symbol]

indicates data missing or illegible when filed

Other Agents that Reduce CH₄ in Animals

Among the CH₄ mitigation options, inhibiting the growth or the metabolic activity of methanogens is the most effective approach. Another strategy is to modulate rumen microbiome so that fermentation is shifted toward decreased H₂ production and increased production of reduced VFA (e.g., propionate). Provided herein are exemplary and non-exhaustive descriptions of anti-methanogenic compounds evaluated with a focus on their impact rumen methanogens.

### Coenzyme M Analogs

Methyl-CoM reductase (Mcr) mediates the final step of all the methanogenesis pathways and CoM (2-mercaptoethanesulfonic acid) is an essential cofactor serving as the methyl group carrier. Mcr reduces methyl-CoM to CH₄. CoM is found in all known methanogens but not in other archaea or bacteria. Several halogenated sulfonated compounds, including 2-bromoethanesulfonate (BBS), 2-chloroethanesulfonate (CBS), and 3-bromopropanesulfonate (BPS), are structural analogs of CoM, and they can competitively and specifically inhibit Mcr activity, lowering CH₄ production at relatively low concentrations. Different species of methanogens vary in sensitivity to these inhibitors. Of three species tested on BBS, Mbb. Ruminantium was the most sensitive, while Methanosarcina mazei was the least sensitive, with Methanomicrobium mobile being intermediate. All three species appeared to be resistant to BPS up to 250 μmol/L in pure cultures. The different sensitivity to these CoM analogs has been attributed to varying ability to uptake these inhibitors into the cells. Methanogens able to synthesize their own CoM are less dependent on external CoM and are thus less sensitive. Mbb. Ruminantium is the only ruminal methanogen that requires CoM synthesized by other methanogens.

Halogenated Aliphatic C₁-C₂ Hydrocarbon

Halogenated aliphatic compounds with 1 or 2 carbons, such as chloroform, bromochloromethane (BCM), bromoform, bromodichloromethane, dibromochloromethane, carbon tetrachloride, trichloroacetamide, and trichloroethyladipate, can lower ruminal CH₄ production. These halogenated compounds block the function of corrinoid enzymes and inhibit cobamide-dependent methyl group transfer in methanogenesis. These halogenated compounds also competitively inhibit CH₄ production by serving as terminal electron (e⁻) acceptors. Drenching chloroform to cattle inhibited methanogenesis substantially for up to 32 days without affecting feed digestion or basic rumen function. The addition of BCM depressed CH₄ production both in vitro and in vivo. In steers fed grain-based diets, BCM decreased CH₄ production by 50 to 60% with no signs of toxicity or residues in meat. It was also reported that the abundance of total bacteria and protozoa was not changed, but methanogenesis and growth of methanogens were drastically inhibited by BCM in both batch cultures and continuous fermenters. While the commercial use of chloroform, a recognized carcinogen, is not practical, it provides validation for the class of BCM compounds in reducing CH₄ production.

Some marine plants such as red seaweed, and algae, lichen, and fungi can contain high concentrations of organobromine compounds such as bromomethane and bromoform. A recent in vitro study showed that red seaweed Asparagopsis taxiformis lowered CH₄ production by 99% at a dose of 2% of organic matter substrate. No adverse effect on feed digestion or fermentation was noted at ≤5% (of dry matter) inclusion. Thus, red seaweed, and probably other organobromine-rich plants, may offer a potentially practical natural approach to mitigate CH₄ emission.

### Nitrooxy Compounds

3-Nitrooxypropanol (3NOP) and ethyl-3NOP, two new synthetic compounds, have been shown to have specific anti-methanogenic properties. 3NOP appears to inactive Mcr by competitively binding to the Mcr active site and then oxidizing the Ni¹⁺ that is required for Mcr activity. Feeding of 3NOP at a dose rate of 2.5 g/day/cow mixed in diets decreased CH₄ emission by 60% per kg of DM intake. In a study using beef cattle, 3NOP fed at 2.0 g/day/cow decreased CH₄ yield by 59%, and the inhibition persisted for up to 112 days without much effect on feed intake, nutrient digestibility or total VFA concentrations. In one recent study, 3NOP fed at 40-80 mg/kg feed DM in dairy cows decreased CH₄ production by about 30% persistently for up to 84 days. Similarly, 3NOP fed at 2.5 g/day/cow decreased CH₄ yield by 37% in dairy cows. In sheep, 3NOP at 0.5 g/day also decreased CH₄ production by 29% without adverse effect on digestion or rumen fermentation. However, when 3NOP was directly added to the rumen through rumen cannula at a daily dose of 0.50 or 2.5 g per cow (equivalent to 25 to 125 mg/kg feed dry matter), the degree of CH₄ Suppression declined to 7-10%. The later study suggests that 3NOP needs to be fed together with the diet to achieve higher efficacy. Thus, 3NOP could be used to lower CH₄ emission from cows and sheep without adverse effects on nutrient utilization or animal performance. It has been demonstrated that 3NOP indeed decreased methanogen abundance while increasing the abundance of protozoa.

TABLE 7
Exemplary nitrogen-containing compounds that reduce CH₄ production in animals

| | Inhibitory | Addition amount; maximum | |
|---|---|---|---|
| Types of nitrogenous compounds | effect | methane suppression amount | Inhibition mechanism |
| Nitrate | [symbol] | 20 mg/g dry matter: 21% | (1) Hydrogen consumption; |
| | | (Alvarez-Hess et al. 2019) | (2) Inhibits the proliferation |
| | | 5 mmol/L: 32.92% | of methanogens; reduces their |
| | | (Wu et al., 2019) | activity and abundance |
| | | 5 mM; 43.26% Liu et al., 2017) | |
| Encapsulated nitrate (EN) | [symbol] | 70 g/100 kg of body | Reduces methane reducing |
| | | weight: 18.5% CH₄/kg of | bacteria |
| | | forage dry matter intake | |
| | | 2.5%; 9.37 mM/d | |
| | | (Capelari et al., 2018) | |
| | | 2.5% 2.8 g/kg Dry matter intake | |
| | | ( [symbol] et al. 2019) | |
| Urea and nitrate mixture | [symbol] | 34 g/kg straw dry matter + 6 g/kg | Indirect consumption of hydrogen |
| | | dry matter of ammonium - | |
| | | nitrate; 10.2% (Zhang et al., 2019) | |
| | | Urea + ammonium nitrate (34 + | |
| | | 6 g/kg of dry matter, respectively); | |
| | | 3.1 mL/g dry matter (Zhang et al., | |
| | | 2018) | |
| Nitroethane (NE), 2-Nitroethanol | [symbol] | 10 mmol/L; 96.7% (NE), 96.7% | (1) Inhibits the activity |
| (NEOH), 2-Nitro-1-Propanol | | (NEOH), 41.7% (NPOH) | of methanogens; |
| (NPOH) | | | (2) Inhibits methyl- |
| | | | coenzyme M gene expression; |
| | | | (3) Reduces the content of |
| | | | coenzyme F420 and F430 |
| 3-Nitrooxypropanol (NOP) | [symbol] | 0.08 mg/g dry matter; 44% | Inhibits methyl-Coenzyme M |
| | | (Alvarez- [symbol] et al., 2019) | activity |
| | | 2.5 g/animal/day; 38%/kg | |
| | | dry matter intake (Martinez- | |
| | | Fernandez et al., 2018) | |
| | | 60 mg/kg of feed dry matter; | |
| | | 26%/day ( [symbol] et al., 2020) | |
| | | 1.6 g; 28%(roughage), 23% | |
| | | (concentrate pellet) (Van | |
| | | [symbol] et al., 2019) | |

[symbol]

indicates data missing or illegible when filed

### Pterin Compounds

Pterin is a group of structural analogs of deazaflavin (F₄₂₀), which is a coenzyme involved in two steps of the hydrogenotrophic methanogenesis pathway. Therefore, pterin compounds can competitively inhibit CH₄ production. In one study, CH₄ production by Mbb. Ruminantium, Ms. Mazei, and Mm. mobile was significantly decreased by lumazin (2,4-pteridinedione) in a dose-dependent manner from 0.06 to 0.24 mmol/L. As expected, pterin is much less efficacious in mixed rumen cultures than in pure methanogen cultures. It was suggested that lumazine could be degraded or transformed by some microbes in mixed cultures or adsorbed to solid particles where it becomes unavailable to methanogens. Some N-substituted derivatives of p-aminobenzoic acid, which are inhibitors of methanopterin synthesis in methanogens, decreased methanogenesis in ruminal cultures without inhibiting VFA production.

### Hydroxymethylglutaryl-CoA (HMG-S-CoA) Reductase Inhibitors

All archaea contain long-chain isoprenoid alcohols as the major component of their cell membrane. Isoprenoid alcohols are unique to archaea. They are synthesized from mevalonate that is formed by reduction of 3-hydroxy-3-methylglutaryl coenzyme A (HMG-S-CoA) catalyzed by HMG-S-CoA reductase. This enzyme is also used for the synthesis of the same precursor ultimately used in cholesterol synthesis in humans. As an inhibitor of HMG-S-CoA reductase, statins (e.g., lovastatin and mevastatin) can inhibit the growth of methanogens by inhibiting the synthesis of mevalonate. Lovastatin and mevastatin may also act as a potential inhibitor of F₄₂₀-dependent NADP oxidoreductase as shown in the model structure of that enzyme. In the earliest reported study, mevastatin at 5.6 μmol/L inhibited the growth of all three strains of rumen Methanobrevibacter, but not rumen bacteria in vitro. In studies using a rumen simulation technique (Rusitec), lovastatin (150 mg/L) reduced CH₄ production by approximately 42% without altering bacterial counts or nutrient fermentation.

The high cost of pure statins promoted a search for natural sources of statins as agents to mitigate CH₄ production. Lovastatin is a secondary metabolite of idiophase of several fungal species (e.g., Penicillium spp., Aspergillus terreus, Monascus purpureus, and Pleurotus ostreatus), and it can reach a concentration up to 2.8% of the dry weight of P. ostreatus (oyster mushrooms) and 261 mg/kg fermented rice straw. Lovastatin extracted from fermented rice straw significantly reduced total CH₄ production by rumen methanogens by nearly 28% after 48 h in vitro incubation. Extract from A. terreus-fermented rice straw containing lovastatin (97 mg/g dry mass) also significantly reduced CH₄ production and abundance of methanogens, especially Methanobacteriales, and aerobic fungi, but increased several fiber-degrading bacteria. Lovastatin also altered the morphology of M. smithii significantly, resulting in abnormal membrane formation and asymmetric cell divisions and increased HMG-S-CoA reductase gene expression. Fermented rice straw extract also modulated expression of several genes associated with methanogenesis, increasing expression of mtr, mta, and mcr while decreasing expression of hmd and fno. Supernatant fractions containing statins produced by Mortierella wolfii also appeared promising to inhibit methanogenesis without reducing overall fermentation. In another study using sheep, fermented rice straw containing metabolites (possibly pravastatin and mevastatin) produced by Monascus spp. Decreased CH₄ emission (by 30%), the abundance of methanogens, and ruminal acetate: propionate ratio compared with the unfermented rice straw.

Diallyl disulfide, the main ingredient of garlic oil, is known to inhibit HMG-S-CoA reductase. Garlic oil (300 mg/L) was more effective than lovastatin as an inhibitor of CH₄ production (by up to 91% reduction); however, garlic oil also inhibited bacterial growth, which likely reduces the availability of methanogenesis substrates. Garlic oil lowered CH₄ production in vitro and growth of methanogens, altered community structure of methanogens after 24 h incubation. Moreover, interestingly, the anti-methanogenic efficacy increased over time up to 18 days of incubation.

### Plant Secondary Metabolites

Plants secondary metabolites (PSM), such as saponins, tannins, flavonoids, organosulphur compounds, and essential oils, have anti-microbial activities against several types of microorganisms. Numerous PSM extracts have been recognized as potential inhibitors of rumen methanogens and CH₄ production. Some forage plants rich in tannins and saponins have also shown promise at mitigating CH₄ emission from animals.

TABLE 8
Exemplary plant extracts that reduce CH₄ production in animals

| | | Inhibitory | Addition amount; maximum | |
|---|---|---|---|---|
| Types of probiotics | | effects | suppression methane amount | Inhibition mechanisms |
| Plant | Corymbia citriodora leaf | [symbol] | 10 ml/calf/day; — | (1) Protozoa number reduced |
| extracts | extract | | | (1.84 × 10 [symbol] /ml) |
| | | | | (2) Ratios of volatile fatty |
| | | | | acids altered |
| | Aloe vera, Carica | [symbol] | 25 mg/L and 50 mg/L [symbol] | Unknown |
| | papaya, [symbol] | | indica, Carica papaya, Tithonia | |
| | indica, Moringa oleifera, | | diversifolia; 15%, Jatropha | |
| | Tithonia [symbol] , | | curcas and Moringa oleifera pods; | |
| | [symbol] curcas, and | | 30% ( [symbol] & Hassen, 2018) | |
| | Moringa oleifera pod | | Eragrostis substrate, 4 ml plant | |
| | extracts | | extracts 50% reduction in methane/ | |
| | | | Total gas production ( [symbol] , | |
| | | | Hassan [symbol] Adejaro, 2020) | |
| | Pomegranate peel | [symbol] | 2% of dry matter intake, | Unknown |
| | extract and Desert | | Punica granatum; 46% | |
| | teak extract | | Tecomella undulata; 42% | |
| | Rhus succedanea extract | [symbol] | 50 mg/L; lowest | Unknown |
| | Areca catechu and Acacia | [symbol] | 2% dry matter basis, Areca | Unknown |
| | nilotica extract | | Catechu; 21%; Acacia nilotica; 23% | |
| | Agoragapsis armata | [symbol] | 1.0% 67.2% (Roque et al., 2019) | Unknowa |
| | Garlic extract | [symbol] | 0.5% (Kim et al., 2018b) | Decreased abundance of |
| | | | 1 g the experimental mixture; | methanogenic archaea |
| | | | 6.9 ± 10.7 ml/d (Eger et al., 2018) | |
| | Plant extract; resveratrol | | 25 mg, high-forage diets; 60%; | Decreased abundance of |
| | | | high-concentrate diets; 41% | Methanobacter |
| | Plant extracts; caffeic | | 12 mM, Caffeic acid; 37.58%; | Unknown |
| | acid and p-coumaric acid | | p-coumaric acid; 28.33% | |

[symbol]

indicates data missing or illegible when filed

1. Tannins: Tannins decrease CH₄ production by directly inhibiting methanogens and indirectly decreasing H₂ production as a result of decreased fiber digestion and protozoal population in the rumen. The inhibitory activity of tannins extracted from Lotus pedunculatus was demonstrated on pure cultures of methanogens. The inhibition of methanogen populations was also shown by tannins in the rumen of goats fed diets containing tannins. Studies on structure-activity relationships have shown that types and molecular weights of tannins are important in determining their potency in lowering CH₄ production and abundance and diversity of rumen methanogens, with high molecular weight condensed tannins (CT) being more potent. Such structure-activity relationships have been demonstrated using members of Methanobacteriales including Methanobrevibacter.

2. Flavonoids: Flavonoids have not been extensively evaluated with respect to rumen methanogenesis. It was reported that inclusion of flavone, myricetin, naringin, rutin, quercetin, or kaempferol decreased in-vitro CH4 production by 5 to 9 mL/g DM. Their potency ranked as follows: myricetin≥kaempferol≥flavone>quercetin≥naringin>rutin≥catechin. Catechin decreased CH₄ production both in vitro and in vivo. All the flavonoids, when fed at 0.2 g/kg DM, noticeably decreased relative abundances of hydrogenotrophic methanogens, and citrus (Citrus aurantium) extract rich in mixed flavonoids and its pure flavonoid components, neohesperidin and naringin, appeared to result in the greatest inhibition. Methanosarcina spp. Were also inhibited by poncirin, neohesperidin, naringin and their mixture. Flavonoids directly inhibit methanogens and also likely acts as H₂ sinks via cleavage of ring structures (e.g., catechin) and reductive dihydroxylation.

3. Saponins: The effects of saponins on rumen fermentation, rumen microbial populations, and ruminant productivity have been examined extensively. Quillaja saponin at 1.2 g/L, but not at 0.6 g/L, lowered CH₄ production in vitro and the abundance of methanogens (by 0.2-0.3 log) and altered their composition. Ivy fruit saponin decreased CH₄ production by 40%, modified the structure of the methanogen community, and decreased its diversity. Saponins from Saponaria officinalis decreased CH₄ and abundance of both methanogens and protozoa in vitro. It is hypothesized that saponins lower H₂ production, thereby reduce CH₄ production.

4. Essential oils: The effects, mostly beneficial, of essential oils (EO) on rumen fermentation, microbial populations, and ruminant productivity have frequently been reviewed. Several EO compounds, either in pure form or in mixtures, are anti-methanogenic. The effects of EO on CH₄ production and methanogens are variable depending on dose, types, and diet. Five EO (clove, eucalyptus, peppermint, origanum, and garlic oil) that have different chemical structures in vitro at three different doses (0.25, 0.50 and 1.0 g/L) were tested for their effect on CH₄ production and archaeal abundance and diversity. Overall, all these EO suppressed CH₄ production and abundance of archaea and protozoa in a dose-dependent manner, but they differed in potency. Thyme oil or cinnamon oil fed to Holstein steers at 0.5 g/day decreased the relative abundance of total protozoa and methanogens. However, feeding beef cattle a blend of EO (CRINA®) did not affect CH₄ production, methanogen abundance or its diversity. Overall, methanogens may be directly inhibited or indirectly inhibited by Eos via inhibition of protozoa and H₂-producing bacteria in the rumen.

### Alternative Hydrogen Sinks

Compounds with a redox potential higher than CO₂ can thermodynamically outcompete CO₂ for reducing equivalents produced during rumen fermentation. These compounds, thus, can be used as alternative e⁻ acceptors to redirect e⁻ flux away from methanogenesis. The commonly evaluated alternative e⁻ acceptors are discussed below.

1. Nitrate and sulfate: Nitrate (NO₃¹⁻) decreased CH₄ production both in vitro and in vivo. Mechanistically, nitrate decreases CH₄ production by outcompeting CO₂ as an e⁻ acceptor, and its reduction intermediates, nitrite (NO₂¹⁻) and nitrous oxide (N₂O), also directly inhibit methanogens as well as some H₂ producers. Sulfate also lowers CH₄ production, but much less effectively than nitrate. Archaeal abundance declined in goats receiving nitrate. While nitrate is not toxic to methanogens, it is toxic to protozoa, fungi and to a lesser extent to select bacterial species, suggesting a more general toxicity of nitrate. Nitrate can replace a portion of the dietary nitrogen as it is reduced to ammonia.

2. Nitrocompounds: A few organic nitrocompounds have been evaluated for their efficacy to decrease methanogens and CH₄ production. These compounds can serve as e⁻ acceptors by some bacteria competing with methanogens for reducing equivalents. This is demonstrated by nitroethane that can be used as a terminal e⁻ acceptor by Dentitrobacterium detoxificans, thereby indirectly decreasing CH₄ production. Nitrocompounds may also inhibit methanogenesis by directly inhibiting the activity of formate dehydrogenase/formate hydrogen lyase and hydrogenase, all of which are involved in the early step(s) of the hydrogenotrophic methanogenesis pathway, or inhibiting e⁻ transfer between ferredoxin and hydrogenase. Nitrocompounds generally are quite effective in lowering CH₄ production, with 3-nitro-propionate, 2-nitropropanol, 2-nitroethanol and nitroethane being able to decrease CH₄ production by 57 to 98% in vitro. Using sheep, it was shown that nitroethane decreased CH₄ production by up to 45% and 69%, respectively, when orally administrated at 24 and 72 mg/kg body weight daily for 5 days. Although less effective than nitroethane, 2-nitropropanol also significantly lowered CH₄ production (by 37%) in steers.

3. Propionate and butyrate enhancers: Malate, acrylate, oxaloacetate, and fumarate are intermediates of carbohydrate fermentation. They can be converted to propionate or used in anabolism for the synthesis of amino acids or other molecules. They can accept reducing equivalents and thus stoichiometrically lower H₂ available for CH₄ production. When added at a concentration of 3.5 g/L, fumarate decreased CH₄ production by 38% in continuous fermenters with forages as a substrate. Types of forages and their combinations appeared to affect the anti-methanogenic efficacy of fumarate, ranging from 6 to 27% inhibition at 10 mmol/L. Acrylate also depresses CH₄ production in the rumen, but to a lesser extent than an equimolar level of fumarate. Malate was found to decrease CH₄ production by beef cattle in a dose-dependent manner, with a 16% decrease being noted when fed at 7.5% of DM intake, which corresponds to a 9% reduction per unit of DM intake. Different studies reported different anti-methanogenic potencies of this type of e⁻acceptors. Fumarate fed to goats at 10 g/day/goat was found to decrease the abundance of methanogens and CH₄ production only by 11.9% while increasing concentrations of total VFA, acetate and propionate. Some of the intermediates of pyruvate conversion to butyrate can act as e⁻ acceptors, which could also decrease CH₄ production.

4. Unsaturated organic acids: Unsaturated fatty acids can act as H₂ sinks during their biohydrogenation and thereby lower CH₄ production. Propynoic acid (an unsaturated analog of propionic acid), 3-butenoic acid and 2-butynoic acid (both unsaturated analogs of butyric acid), and ethyl 2-butynoate each at 6 to 18 mmol/L have been evaluated as alternative e⁻ sinks to lower methanogenesis in vitro. Only propynoic acid and ethyl 2-butynoate markedly lowered CH₄ production, by 65 to 76% and 24 to 79%, respectively. In another study, propynoic acid lowered CH₄ production by 67% and 78% at 6 and 12 mmol/L, respectively and decreased methanogen abundance. Propynoic acid and ethyl 2-butynoate are directly toxic to methanogens, and species of methanogens vary in their sensitivity to these two inhibitors, with Mbb. Ruminantium being most sensitive, Ms. Mazei least sensitive, and Mm. mobile intermediate.

### Inhibitors to Hydrogen-Producing Bacteria

1. Ionophores: Ionophores, such as monensin and lasalocid, are commonly used to improve rumen microbial metabolism. Being highly lipophilic ion carriers, they pass through the cell wall of Gram-positive bacteria and penetrate into the cell membrane. Therein, they serve as H⁺/Na⁺ and H⁺/K⁺ antiporters, dissipating ion gradients that are needed for ATP synthesis, nutrient transport, and other essential cellular activities and ultimately resulting in delayed cell division and even cell death. Ionophores preferentially inhibit Gram-positive bacteria, including members of class Clostridia, including Ruminococcus species that produce acetate and H₂. Ionophores can also inhibit some Gram-negative rumen bacteria, including bacteria that produce formate and H₂. Therefore, ionophores may lower CH₄ emission by decreasing H₂ production. For examples, monensin fed at 24-35 mg/kg diet lowered CH₄ production by up to 10% (g/kg DM intake), though no CH₄ suppression was observed at 10-15 ppm. In a recent in vivo study, however, monensin at 60 mg/day/cow did not lower CH₄ production by tropical cattle, though it decreased CH₄ production by about 30% when fed at 250 mg/day/cow. As repeatedly noted, at such high supplementation level, DM intake was lowered, which explains most of the observed decrease in CH₄ emission. Ionophores are not known to directly inhibit methanogens, but they can change the population dynamics of methanogen species. For example, monensin decreased the population of Methanomicrobium spp. While increasing that of Methanobrevibacter spp. Total methanogens were also decreased in cattle fed monensin. These can be explained by reduced availability of H₂ and differences in affinity for H₂ and growth kinetics among methanogen species.

2. Bacteriocins: Bacteriocins are proteins or peptides produced by bacteria and inhibit select microbial species in the rumen and other habitats. There are only a few studies investigating the effect of bacteriocins on CH₄ emission. Bovicin HC5, a bacteriocin produced by Streptococcus spp. From the rumen, was reported to suppress CH₄ by 50% in vitro. Nisin, a bacteriocin produced by Lactobacillus lactis subsp. Lactis, has also been shown to decrease CH₄ production in vitro by up to 40% depending upon its concentration. Similar to monensin, bacteriocins probably modulate rumen fermentation leading towards increased propionate, thereby decreasing CH₄ production.

Additional Agents that Reduce CH₄ in Animals

### Biochemical Pathways

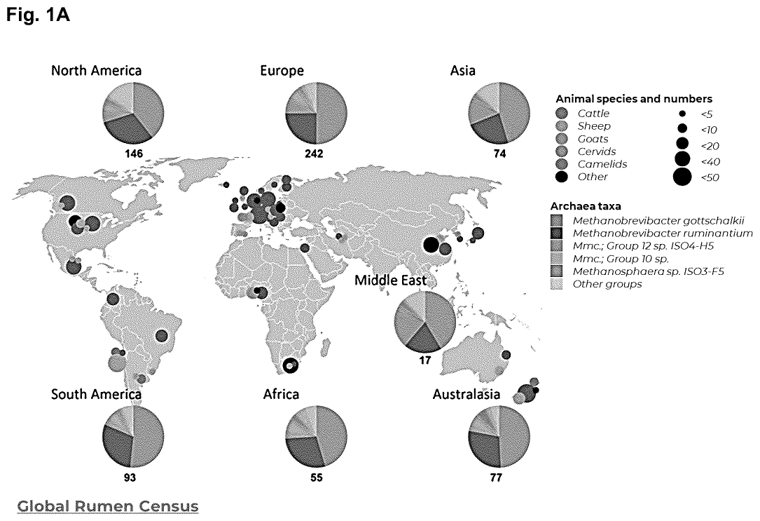

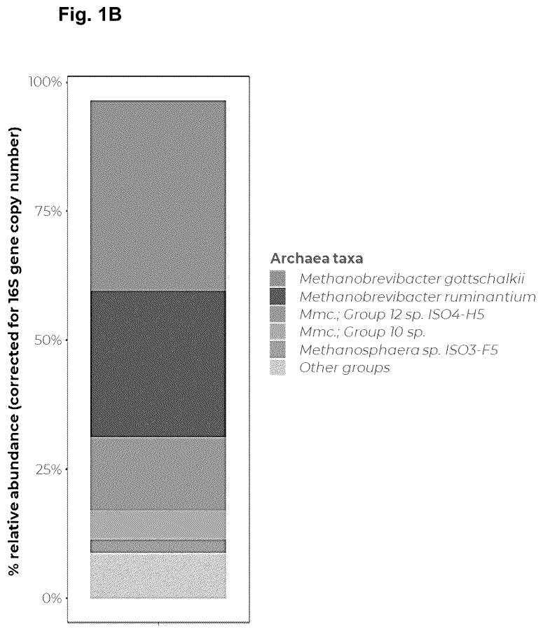

In certain embodiments, the one or more deleterious atmospheric gases and/or precursors thereof are microbially derived through one or more biosynthetic pathway. The deleterious atmospheric gas can be any suitable deleterious atmosphere gas, such as CO₂, CH₄, nitrous oxide, or a combination thereof. The deleterious atmospheric gas precursor can be any suitable precursor, such as acetate, H₂, CO₂, methanol, monomethylamine, dimethylamine, trimethylamine, nitric oxide, or a combination thereof. In preferred embodiments, the deleterious atmosphere gas comprises CO₂, H₂, or CH₄, more preferably CH₄. In certain embodiments, wherein the resultant deleterious atmospheric gas comprises CH₄, the one or more biosynthetic pathways include the acetoclastic, hydrogenotrophic, and methylotrophic pathways, which differ based on the starting substrates, i.e., precursor, (FIG. 1), more preferably the acetoclastic or hydrogenotrophic pathways, even more preferably the acetoclastic pathway.

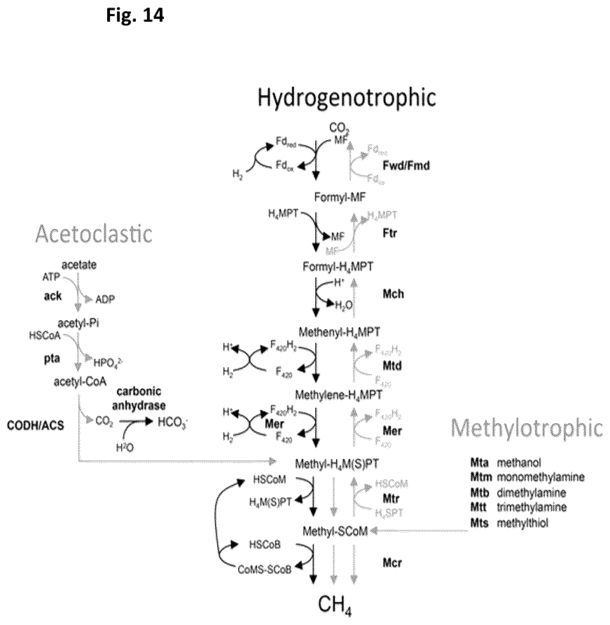

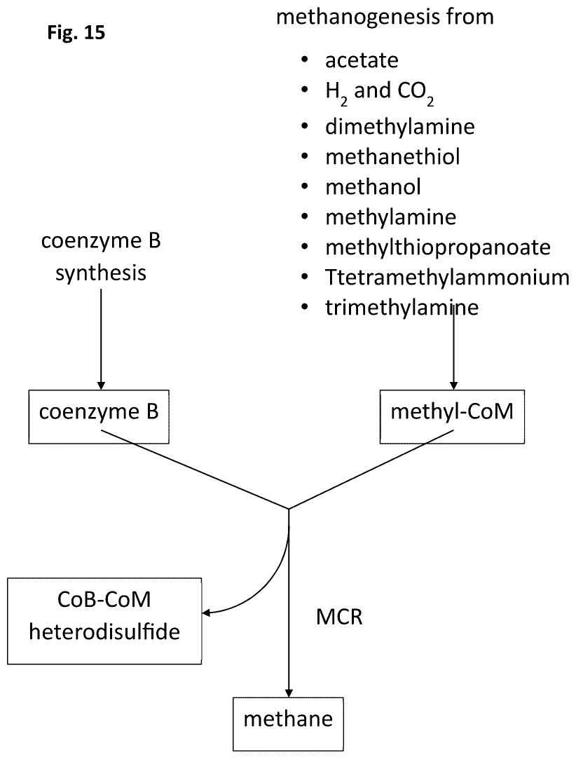

The acetoclastic pathway comprises a series of enzymes that convert the precursor acetate through a series of enzymatic conversions to CH₄. Starting from acetate, (1) acetate is converted to acetyl phosphate by acetate kinase (ack); (2) acetyl phosphate is converted to acetyl-CoA by phosphotransacetylase (pta); (3) the acetyl group from acetyla-CoA is transferred to a protein intermediate by acetyl-CoA decarbonylase; (4) the acetyl group is then transferred to tetrhydrosarcinapterin to form 5-methyl-tetrahydrosarcinapterin by methyltetrahydrosarcinapterin methyltransferase; (5) 5-methyl-tetrahydrosarcinapterin is converted to methyl-CoM by methyl-H₄SPT:CoM methyltransferase (Mtr); and (6) methyl-CoM is reduced to CH₄ by methyl-CoM reductase (Mcr) (FIG. 14 and FIG. 15).

The hydrogenotrophic pathway comprises a series of enzymes that convert the precursors H₂ and CO₂ to CH₄. Starting from CO₂ and H₂, (1) a formylmethanofuran dehydrogenase (Fwd/Fmd) produces a formylmethanofuran, (2) which is further converted into 5-formyl-tetrahydromethanopterin by a formylmethanofuran:H₄MPT formylatransfer (Ftr); (3) 5-formyl-tetrahydromethanopterin is further converted into 5,10-methenyltetrahydromethanopterin by methyl-H₄MPT cyclohydrolase (Mch); (4) 5,10-methenyltetrahydromethanopterin is converted to N₆-methyltetrahydromethanopterin by F₄₂₀-dependent methylene-H₄MPT reductase (Mer); (5) N₆-methyltetrahydromethanopterin is converted to methyl-CoM by methyl-H₄MPT:coenzyme M methyltransferase (Mtr); and (6) methyl-CoM is reduced to CH₄ by methyl-CoM reductase (Mcr) (FIG. 14 and FIG. 15).

The methylotrophic pathway comprises a series of enzymes that convert one or more of dimethylamine, methanethiol, methanol, methylamine, methylthiopropanoate, tetramethylammonium, and/or trimethylamine into methyl-CoM, wherein methyl-CoM is reduced to CH₄ by methyl-CoM reductase (Mcr) (FIG. 14 and FIG. 15).

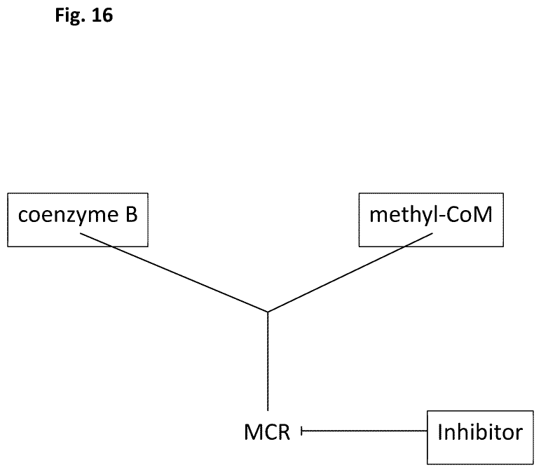

In certain embodiments, provided herein are compositions, methods, and/or kits comprising one or more small molecules that reduce the activity of one or more enzymes in one or more CH₄ biosynthetic pathways. The enzyme can be any suitable enzyme, such as 3-(methylthio)propanoate:coenzyme M methyltransferase, acetate kinase, acetyl-CoA decarbonylase, acetyl-CoA decarbonylase/synthase complex α₂ε₂, acetyl-CoA decarbonylase/synthase complex β, acetyl-CoA decarbonylase/synthase complex γδ, acetyl-CoA synthase, carbon monoxide dehydrogenase, carbonic anhydrase, Co-methyltransferase, coenzyme M reductase, cyclohydrolase, dehydrogenase, dimethylamine-[corrinoid protein] Co-methyltransferase, F₄₂₀-dependent methylene-H₄MPT reductase, F₄₂₀-dependent methylene-H₄SPT dehydrogenase, formylmethanofuran dehydrogenase, formylmethanofuran:H₄MPT formyltransferase, formylmethanofuran:H₄SPT formyltransferase, formyltransferase, H₂-forming methylene-H₄MPT dehydrogenase, methanol-5-hydroxybenzimidazolylcobamide Co-methyltransferase, methenyl-H₄MPT cyclohydrolase, methyl-coenzyme M reductase, methyl-H₄SPT:CoM methyltransferase, methylated [methylamine-specific corrinoid protein]:coenzyme M methyltransferase, methylcobamide:CoM methyltransferase, methylthiol:coenzyme M methyltransferase, methyltransferase, MtaC protein:coenzyme M methyltransferase, phosphotransacetylase, tetrahydromethanopterin S-methyltransferase, tetramethylammonium methyltransferase, trimethylamine-corrinoid protein Co-methyltransferase, or a combination thereof. In a preferred embodiment, the enzyme comprises methyl-CoM reductase (Mcr) (FIG. 16).

Compositions for Reducing Production of Deleterious Atmospheric Gases and/or Precursors Thereof

In certain embodiments provided herein are compositions. In certain embodiments, provided herein are compositions comprising one or more small molecules. In preferred embodiments, provided herein are compositions comprising one or more small molecules that reduce the production of one or more deleterious atmospheric gases and/or precursors thereof. The small molecule can be any suitable small molecule for reducing the production of one or more greenhouse gases and/or precursors thereof, for example a small molecule that interferes with the uptake and/or conversion of acetate, H₂, CO₂, methanol, monomethylamine, dimethylamine, trimethylamine, nitric oxide, or a combination thereof, and/or a small molecule that interfere with the production of CO₂, H₂ nitrous oxide, or a combination thereof. In preferred embodiments, the small molecule interferes with the uptake and/or conversion of acetate, H₂ and/or CO₂ and/or the production of CO₂ or CH₄, more preferably with the production of CH₄.

Small Molecules that Affect Production of Deleterious Atmospheric Gases and/or Precursors Thereof

In certain embodiments, provided herein is a composition for reducing emissions of deleterious atmospheric gasses and/or precursors thereof comprise: one or more small molecules that reduce the production of one or more deleterious atmospheric gasses and/or precursors thereof. The one or more small molecules that reduce the production of one or more deleterious atmospheric gasses and/or precursors can be any suitable molecule.

In certain embodiments, the one or more small molecules that reduce the production of one or more deleterious atmospheric gasses and/or precursors comprises a compound with the formula the formula R¹—[CH₂]n-ONO₂
wherein
n is an integer from 1 to 15;
R¹ is selected from the group consisting of H, C₁-C₆alkyl, phenyl, —OH, —NH₂, —CN,
—COOH, —O(C═O)R³, —NHC(═O)R³, SO₂NHR³, or —ONO₂, —SH and R³ is C₁-C₆alkyl, phenyl, pyridyl;
with the proviso that when n is >3 the hydrocarbon chain may be interrupted by —O— or —NH—.

In some embodiments, the one or more small molecules that reduce the production of one or more deleterious atmospheric gases and/or precursors comprises 3-nitrooxypropanol, 9-nitrooxynonanol, 5-nitrooxy pentanoic acid, 6-nitrooxy hexanoic acid, bis(2-hydroxyethyl)amine dinitrate, 1,4-bis-nitrooxybutane, 1,5-bis-nitrooxypentane, or any combination thereof. Preferably, the one or more small molecules is 3-nitrooxypropanol (3NOP).

In some embodiments, the composition comprises about 1 to about 25% by weight of the small molecule, about 5 to about 20% by weight of the small molecule, or about 5 to about 15% by weight of the small molecule.

### Solid Carriers

In certain embodiments, the composition further comprises one or more solid carriers. As used herein, the term “solid carrier” includes additives commonly used in the preparation of powderous formulations such as thickeners, for example gums or cellulose derivatives such as xanthan gum, karaya gum and/or ethylcellulose. The one or more solid carriers can be any agriculturally suitable carrier, such as attapulgite, kaolinite, fuller's earth, calcium carbonate, perlite, diatomaceous earth, calcium silicate, fly ash, a polysaccharide, a disaccharide, a monosaccharide, a gum, a natural or synthetic derivative thereof, or a combination thereof.

In certain embodiments, the one or more solid carriers comprises any carrier suitable for ingestion, such as a saccharide comprising cellulose, xantham gum, karaya gum, ethylcellulose, inositol, galactose, arabinose, lactose, lactulose, mannitol, mannose, sorbose, turanose, platinose, or a combination thereof.

In some embodiments, the carrier comprises attapulgite, kaolinite, fuller's earth, calcium carbonate, perlite, diatomaceous earth, calcium silicate, fly ash, a polysaccharide, a disaccharide, a monosaccharide, a gum, a natural or synthetic derivative thereof, or a combination thereof.

In other embodiments, the carrier comprises attapulgite, kaolinite, fuller's earth, calcium carbonate, perlite, diatomaceous earth, calcium silicate, fly ash, a polysaccharide, a disaccharide, a monosaccharide, a gum, silica, propylene glycol, hemp protein, biochar, montmorillonite, activated charcoal, lignin, wood flour, hemp protein, pea protein, soy protein, gelatin, casein, chitosan, talc, calcium phosphate, arginine, lysine, calcium carbonate, carbon black, glutamine, betaine, bismuth phosphate, bismuth citrate, iron phosphate, or any combination thereof.

In some embodiments, the carrier comprises the one or more solid carriers comprises a saccharide comprising cellulose, xanthan gum, karaya gum, ethylcellulose, inositol, galactose, arabinose, lactose, lactulose, mannitol, mannose, sorbose, turanose, platinose, or a combination thereof.

In some embodiments, the one or more solid carriers comprises a saccharide comprising cellulose, xanthan gum, karaya gum, ethylcellulose, inositol, galactose, arabinose, lactose, lactulose, mannitol, mannose, sorbose, turanose, platinose, carrageenan, cellulose acetate, hydroxypropyl cellulose, cellulose acteate phthalate, maltrodextran, dextran, inulin, corn starch, amylopectin, sodium starch glycolate, pentaerthritol, cyclodextrin, or a combination thereof.

In certain preferred embodiments, the solid carrier comprises silica and ethylcellulose, more particularly about 10% to about 50% by weight of the silica and about 50 to about 90% by weight of the ethylcelluose.

In other preferred embodiments, the solid carrier comprises silica and activated charcoal, particularly about 10% to about 90% by weight of the silica and about 10% to about 90% by weight of the activated charcoal.

In certain embodiments, the binder further comprises arginine, lysine, or both arginine and lysine. While not being bound by theory, it is believed that arginine and lysine are capable of forming hydrogen bonds with the small molecule, such as 3NOP, thereby altering the release rate.

In other preferred embodiments, the carrier comprises activated charcoal and ethylcellulose, particularly about 10% to about 50% by weight of the activated charcoal and about 40 to about 90% by weight of the ethylcellulose.

In some embodiments, the carrier further comprises about 1 to about 10% by weight of sodium lignosulfate. While not being bound by theory, it is believed that sodium lignosulfate improves coating adhesion to the tablet resulting in a reduction in release rate of the small molecule.

In other embodiments, the carrier comprises arginine and polycaprolactone, such as about 10 to about 60% by weight of the arginine and about 30 to about 90% by weight of the polycaprolactone.

In other preferred embodiments, the carrier comprises 25% silica, 66% polycaprolactone, such as about 10 to about 60% by weight of the silica and about 30 to about 90% by weight of the polycaprolactone.

In certain embodiments, the composition comprises a granular shape. The composition may comprise any suitable shape, such as a spherical-, square-, rectangular-, capsular-, cylindrical-, conical-, ovular-, triangular-, diamond-, disk-like shape, or a combination thereof. In certain embodiments, the shape of the particle affects the rate of dissolution of the particle.

The granular particle can comprise any suitable texture, for example hard or soft. In certain embodiments, the texture of the particle affects the rate of dissolution of the particle. In certain embodiments, the composition comprises a combination of differently textured pellets each of which release the small molecule at different rates.

In certain embodiments, the granular particles comprise a uniform size distribution, for example about ±20%, ±15%, ±10%, ±5%, ±2%, or ±1% size distribution in the median particle size. In certain embodiments, the granular particles comprise a non-uniform size distribution, for example greater than about ±20%. In certain embodiments, the granular particles comprise a plurality of differently sized populations of granular particles each of which comprise a uniform size distribution.

In certain embodiments, the one or more solid carrier dissolves and thereby releases the one or more small molecules that reduce the production of greenhouse gases and/or precursors thereof. In a preferred embodiment, the one or more solid carriers will dissolve in water.

### Extended and Delayed Release

It may be necessary to vary the rate of dissolution of the composition. For example, one may want to produce an extended-release formulation, wherein the composition releases the one or more small molecules over a period of time to maintain a suitable environmental concentration of the one or more small molecules. This can be beneficial to reduce the frequency of applications, for example to reduce labor costs and/or applications in rural and/or hard to reach environments. In certain embodiments, complete dissolution of the composition and full release of the one or more small molecules occurs over at least about 1, 2, 3, 4, 5, 6, 7, 14, 21, 28, 35, 42, 49, 56, 63, 70, 77, 84, 91, 98, 105, 112, 119, 126, 133, 140, and/or nor more than about 2, 3, 4, 5, 6, 7, 14, 21, 28, 35, 42, 49, 56, 63, 70, 77, 84, 91, 98, 105, 112, 119, 126, 133, 140, or 147 days, for example about 1 to about 147 days, preferably about 7 to about 63 days, more preferably about 7 to about 42 days even more preferably about 14 to about 42 days yet even more preferably about 14 to about 28 days.

In certain embodiments, the extended-release formula may comprise any suitable extended-release formula. In certain embodiments, the extended-release formula comprises one or more additives that reduce the rate of dissolution of the composition, one or more additives that reduce the rate of dissolution of the small molecule from the composition, one or more additives that comprise a membrane that dissolves over time, wherein the rate of dissolution of the membrane controls the rate of release of the one or more small molecules, a suitable alternative, or a combination thereof.

In certain embodiments, the population of granular particles comprises a plurality of populations of granular particles wherein each population comprises a different formulation and/or shape. In certain embodiments, the population of granular particles comprises a first population and a second population. In certain embodiments, the population of granular particles further comprises at least 1, 2, 3, 4, 5, 5, 6, 8, or 9 and/or no more than 4, 5, 6, 7, 8, 9, or 10 additional populations, for example a total of 3-10 additional populations, preferably 3-7 additional populations, more preferable 3-5 additional populations. In a preferred embodiment, each of the additional populations comprises a different formulation than the others.

In certain embodiments, the rate of dissolution of the granular particles is modulated by the size of the granular particle. In certain embodiments, smaller granular particles dissolve faster than larger granular particles, such that each successive larger population in the plurality of populations of differently size particles provides a delayed release compared to the smaller populations of particles. In certain embodiments, an increased proportion of larger to smaller granular particles in a population of granular particles results in slower rates of dissolution of the population of granular particles.

In certain embodiments, the first population of particles comprises an immediate release formulation. In certain embodiments, the second population comprises a delayed release formulation, wherein the second population dissolves and/or releases the one or more small molecules that reduce the production of one or more deleterious atmospheric gasses and/or precursors after the first population. In certain embodiments, each additional population comprises a delayed release formulation, wherein each population dissolves and/or releases the one or more small molecules that reduce the production of one or more deleterious atmospheric gasses and/or precursors at a different time than each of the other populations.

In some embodiments, an immediate release formulation releases the one or more small molecules that reduce the production of one or more deleterious atmospheric gases with about 1 day, 2 days, 3 days, 4 days, 5 days, or 6 days. In some embodiments, a delayed release formulation releases the one or small molecules after about 1 week or more, for example up to about 21 weeks.

In some embodiments, the first population of particles provides has a half-life for the one or more small molecules (such as about 1 to 12 hrs), the second population has a longer half-life (such as about 24 or more hrs), each additional population has a longer half-life than the previous population such that an effective amount of the one or more small molecules is maintained for weeks or months. Thus, the present compositions advantageously do not, in such embodiments, require repeated, frequent applications.

In certain embodiments, the extended release happens within the first 10% of the half-life and then the 1st delayed happens with 1-2 half lives, then the next with 1-2 of the delayed release.

In some embodiments, the particles have a size ranging from about 1 mm to about 20 mm, about 1 to about 15 mm, about 1 to about 10 mm, about 5 to about 20 mm, about 5 to about 15 mm, or about 5 to about 10 mm.

### Coatings

In certain embodiments, the composition may comprise a coating, for examples particles or tablet having a coating. The coating can comprise any suitable coating, such as a wax, a fat, or a synthetic polymer. In certain embodiments, the wax comprises organic compounds consisting of long alkyl chains, natural waxes (plant, animal) which are typically esters of fatty acids and long chain alcohols as well as synthetic waxes, which are long-chain hydrocarbons lacking functional groups. In certain embodiments, the fat comprises a wide group of compounds which are soluble in organic solvents and largely insoluble in water such as hydrogenated fats (or saturated fats) which are generally triesters of glycerol and fatty acids. Suitable fats can have natural or synthetic origin. In certain embodiment, the fat comprises glycerine monostearate, carnauba wax, candelilla wax, sugarcane wax, palmitic acid, stearic acid hydrogenated cottonseed oil, hydrogenated palm oil and hydrogenated rapeseed oil, or combinations thereof. Any suitable synthetic polymer can be used, such as poly-L-glutamic acid (PGA) and polylactic acid (PLA). In preferred embodiments, the synthetic polymer is at least partially water soluble.

The coating may be single layer or multiple layers, preferably two layers.

In some embodiments, the coating is selected from cellulose acetate phlalate, ethyl cellulose, hydroxypropyl cellulose, polycaprolactone,alginate, chitosan, polyethylene glycol, cellulose acetate, triacetin, propylene glycol, n-methyl-2-pyrollidone, and any combination thereof.

In certain preferred embodiments, the coating comprises two or more polyelectrolytes, such as polystyrene sulfonate, polyethyleneimine, sodium lignosulfate, polyglutamic acid and poly-L-lysine, poly-L-arginine, polyallylamine hydrochloride, polyacrylic acid, or any combination thereof.

In some preferred embodiments, the polyelectrolytes comprise polyallylamine hydrochloride and sodium lignosulfate.

In some preferred embodiments, the polyelectrolytes comprise polyallylamine hydrochloride and polystyrene suylfonate.

In other preferred embodiments, the polyelectrolytes comprise sodium lignosulfate and one of polyglutamic acid and poly-L-lysine, or poly-L-arginine, and sodium lignosulfate.

In still other preferred embodiments, the polyelectrolytes comprise polystyrene sulfonate and one of polyglutamic acid and poly-L-lysine, or poly-L-arginine.

The polyelectrolytes may, in certain embodiments, be chemically cross-linked with a cross-linking agent.

In certain embodiments, the composition comprises one or more coatings applied with minimal to no bubbles. Additionally or alternatively, the composition comprises one or more coatings that comprise a foam or a plurality of air bubbles. In certain cases, the foamed coating can temporarily alter the buoyancy of the composition. One such example includes a composition comprising a foamed coating that floats when initially applied, then, after a period of time, the air pockets in the foamed coating fill with water resulting in the composition sinking to the bottom.

Additives with a Density Greater than Water

In certain embodiments, the composition further comprises one or more additives with a density greater than water. For example, the additive may have a density greater than 1.1, preferably about 1.1 mg/mL to about 3 mg/mL, about 1.5 to about 3 mg/mL, about 1.5 to about 2.5 mg/mL, or about 1.5 to about 2 mg/mL. Suitable additives include silica, attapulgite, kaolinite, fuller's earth, calcium carbonate, perlite, diatomaceous earth, calcium silicate, fly ash, or a combination thereof. In certain embodiments, the one or more additives with a density greater than water result in the composition sinking below the surface. In certain embodiments, the one or more additives with a density greater than water result in the composition partially or completely sinking to the bottom. In preferred embodiments, the composition completely sinks to the bottom. In certain embodiments, the composition comprising the additive with a density greater than water has a density of at least 1.1, preferably about 1.1 mg/mL to about 3 mg/mL, about 1.5 to about 3 mg/mL, about 1.5 to about 2.5 mg/mL, or about 1.5 to about 2 mg/mL.

### Agriculturally Beneficial Additives

In certain embodiments, the composition further comprises one or more agriculturally beneficial additives. The agriculturally beneficial additive can be any suitable additive depending on the application, such a vitamin, a nutrient, an antibiotic, a fungicide, or a combination thereof.

In certain embodiments, the additive includes one or more suitable components that reduce methanogenesis by methanogens, such as, seaweed (e.g., Asparagopsis taxiformis), kelp, 3-nitrooxypropanol, anthraquinones, ionophores (e.g., monensin and/or lasalocid), polyphenols (e.g., saponins, tannins), organosulfurs (e.g., garlic extract), flavonoids (e.g., quercetin, rutin, kaempferol, naringin, and anthocyanidins; bioflavonoids from green citrus fruits, rose hips and black currants), carboxylic acid, terpenes (e.g., D-limonene, pinene and citrus extracts), or a combination thereof.

Methods for Reducing Production of Deleterious Atmospheric Gases and/or Precursors Thereof

In certain embodiments provided herein are methods. In certain embodiments, provided herein are methods for using one or more small molecules that reduce the production of one or more deleterious atmospheric gases and/or precursors thereof. In certain embodiments, provided herein are methods for applying one or more small molecules that reduce the production of one or more deleterious atmospheric gases and/or precursors thereof to any suitable environment. The suitable environment can comprise any suitable environment. In some embodiments, the suitable environment comprises an environment in which a ruminant (unvaccinated or vaccinated) lives or occupies, e.g., a habitat for a ruminant. In some embodiments, the suitable environment comprises the rumen.

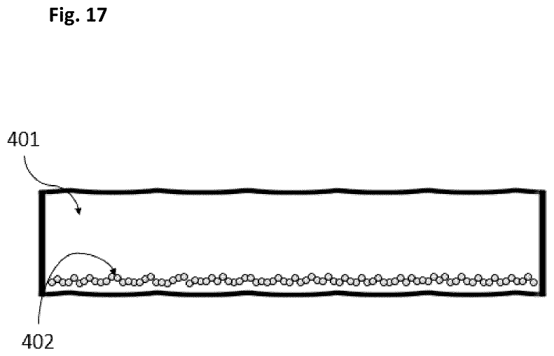

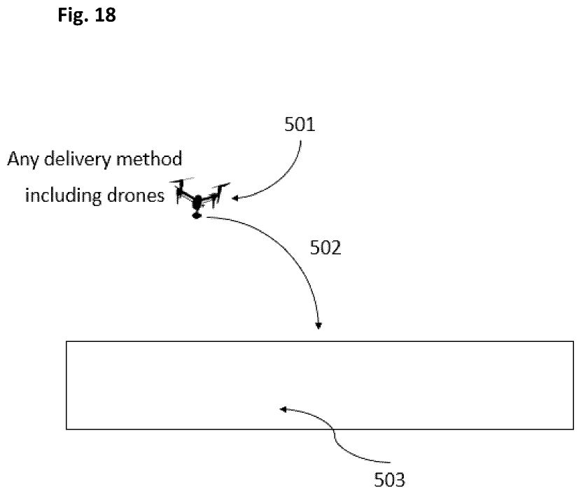

In certain embodiments, the method for reducing emissions of deleterious atmospheric gasses and/or precursors thereof comprises applying a composition comprising one or more small molecules that reduce the production of the deleterious atmospheric gasses and/or precursors thereof. The composition can comprise any suitable composition. In a preferred embodiment, the composition comprises any one of the compositions as described in the Compositions for reducing production of deleterious atmospheric gases and/or precursors thereof section above. In a more preferred embodiments, the composition comprises 3NOP. In certain embodiments, the composition is applied to a water source, such as a trough or a pond, from which a ruminant ingests the composition or a portion thereof from the water source into the rumen. For example as illustrated in FIGS. 17-18.

In certain cases, the composition needs to be reapplied periodically to maintain a suitable concentration of the one or more small molecules. In certain embodiments, the method further comprises reapplying after a period of time a composition comprising one or more small molecules that reduce the production of the deleterious atmospheric gasses and/or precursors thereof. In certain embodiments, the method further comprises, reapplying again after a period of time a composition comprising one or more small molecules that reduce the production of the deleterious atmospheric gasses and/or precursors thereof. Any suitable number of reapplications may be performed as needed to maintain an effective amount of the one or more small molecules. In some embodiments, the composition is reapplied after about 7 to about 28 days, about 7 to 46 days, about 7 to 92 days or about 7 to 147 days.

In certain embodiments, the composition is delivered to one or more water sources.

In certain cases, the concentration of the one or more small molecules can be measured to ensure the presence of a suitable concentration of the one or more small molecules. Any suitable method may be used to measure the concentration, such as a strip test, liquid chromatography, or thin layer chromatography. The method can be performed with or without human intervention.

Kits for reducing production of deleterious atmospheric gases and/or precursors thereof

In certain embodiments, provided herein are kits. In certain embodiments, the kit comprises any one of the compositions as described in the Compositions for reducing production of deleterious atmospheric gases and/or precursors thereof section. In certain embodiments, the kit further comprises a suitable container for shipping.

### Methods of Using Vaccines, Animal Feed, Antibodies, Milk

Provided herein are methods of using the vaccines, antibodies, milk, animal feed, agents (e.g., an agent that reduces CH₄ production in a subject, a probiotic bacterial strain, etc.), or any combination thereof.

In certain aspects, provided herein are methods of inducing an immune response against at least one methanogen in a subject, the method comprising administering to the subject the vaccines or pharmaceutical compositions of the present disclosure.

In some embodiments, the immune response comprises a B cell response (e.g., to produce the antibodies). The antibodies produced in response to the vaccine are transferred to the saliva of the subjects, which are swallowed by the subjects to enter the rumen. Once in the rumen, the antibodies come in contact with at least one methanogen to bind/neutralize said methanogen.

As used herein, the term “neutralization of a methanogen” encompasses any reduction in one or more activities that are normally carried out by the methanogen in the absence of the antibodies that bind the methanogen.

In some embodiments, the activity of a methanogen includes but is not limited to, the activity that aids in producing CH₄ gas. For example, binding of the antibodies to the methanogen may reduce the ability of the methanogen to carry out biochemical reactions that are necessary to produce CH₄, e.g., reduce the ability to convert H₂ and CO₂ or acetate into CH₄ and ATP. In some embodiments, the reduced ability to produce CH₄ may lower the fitness of methanogen in the rumen.

In some embodiments, the activity of a methanogen includes but is not limited to, the activity that aids in forming a granular colony with other bacteria. In some embodiments, such activity may be disrupted physically—e.g., antibodies binding to the methanogen would prevent physical association and/or film formation of the granular colony of bacteria. In some embodiments, a reduction in the activity of forming a granular colony may lead to the reduced ability of a methanogen to remain in the rumen. In some embodiments, such reduced ability may result in the reduction of the total number of methanogens inside the rumen.

In certain aspects, provided herein are methods of reducing the activity, number, and/or type of methanogens in the gut of a subject, the method comprising administering to the subject the vaccines or pharmaceutical compositions of the present disclosure.

In certain aspects, provided herein are methods of reducing the amount of CH₄ produced by a subject, the method comprising administering to the subject the vaccines or pharmaceutical compositions of the present disclosure.

In preferred embodiments, any one of the methods produces an antibody against at least one methanogen. In some embodiments, the antibody is an IgG or an IgA. In preferred embodiments, the antibody is an IgA. The IgA isoform, at least in cattle, may be more stable in the rumen. For example, IgA levels in cattle saliva were reduced by only 40% after 8 h exposure to rumen contents while IgG levels were reduced by 80%.

In some embodiments, the antibody is produced in an amount sufficient to: (a) carry the antibody to the gut; (b) reduce the number and/or type of methanogens in the gut; and/or (c) reduce the amount of CH₄ produced by the subject.

In some embodiments, the method reduces the CH₄ emission from the subject by at least about 1%, 5%, 10%, 20%, 30%, 40%, 50%, 60%, 70%, 80%, 90%, or 95% as compared with a control.

Additionally or alternatively, the method reduces the H₂ emission from the subject from at least about 1%, 5%, 10%, 20%, 30%, 40%, 50%, 60%, 70%, 80%, 90%, or 95% as compared with a control.

Additionally or alternatively, the method increases the feed conversion efficiency of the subject by about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, or 12% as compared with a control.

Additionally or alternatively, the method increases the concentration of one or more volatile fatty acids (e.g., propionate, butyrate, acetate) in the rumen of the subject by about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, or 12% as compared with a control.

Additionally or alternatively, the method increases the average daily gain (ADG) of the subject by about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, or 12% as compared with a control.

Additionally or alternatively, the method increases the dry matter intake (DMI) of the subject by about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, or 12% as compared with a control.

Additionally or alternatively, the method increases the milk production of the subject by about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, or 12% as compared with a control.

In some embodiments, the control is an accepted reference, or the amount of CH₄ production in a subject that has not been vaccinated.

Notably, CH₄ emission or production of CH₄ by a subject can occur at any part of its intestinal track, which includes, e.g., a rumen and a lower bowel (lower intestinal track). The rumen accounts for 90% of all CH₄ production. The rumen has no adaptive immune response. Thus, to be effective in reducing the level of CH₄ production, the rumen or a methanogen therein must be exposed to neutralizing antibodies that bind and inactivate the methanogen. By contrast, the lower bowel, which accounts for 10% of all CH₄ production, has adaptive immune response such that any immune response due to a vaccine can be amplified in the lower bowel. Accordingly, in preferred embodiments, the result of immune response from a vaccine is exposed to the lower bowel of a subject, which then further amplifies the effect of the vaccine. In other words, in preferred embodiments, a vaccine of the present disclosure or the immune response it elicits is exposed to a methanogen in a lower bowel of the subject. Thus, the present disclosure encompasses a method of reducing (i) CH₄ production and/or (ii) activity, number, and/or type of methanogens in the lower intestinal track of a subject, the method comprising administering to the subject a vaccine comprising at least one methanogen cell surface protein or a fragment thereof.

As described herein, in some embodiments, the vaccine or the pharmaceutical composition is administered to the subject via a route selected from intramuscular administration, intradermal administration, subcutaneous administration, and nasal administration.

In some embodiments, the subject is administered with at least one dose of the vaccine or pharmaceutical composition.

In some embodiments, the subject is administered with at least one or two repeat doses of the vaccine or pharmaceutical composition (e.g., booster dose).

In some embodiments, the subject is administered with the repeat dose of the vaccine or pharmaceutical composition after at least about 2 weeks, 1 month, 6 months, or 12 months from the time the subject is administered with the preceding dose of the vaccine.

In some embodiments, the subject is administered with the repeat dose of the vaccine or pharmaceutical composition no more than about 3 months, 6 months, 12 months, or 24 months from the time the subject is administered with the preceding dose of the vaccine.

In some embodiments, the subject receives the repeat dose of the vaccine after at least about 2 weeks and no more than about 18 months from the time the subject is administered with the preceding dose of the vaccine.

In some embodiments, the subject receives a repeat dose of the vaccine after at least about 4 weeks and no more than about 12 months from the time the subject is administered with the preceding dose of the vaccine.

In certain embodiments, the methods of the present disclosure further comprises administering to the subject at least one agent (e.g., at least one additional agent) that reduces the level of CH₄ produced by the subject.

The vaccine composition may be administered before, concurrently with, or after, any agent, milk, antibody, animal feed, or any composition of the present disclosure.

In some embodiments, the at least one agent is selected from 3-Nitrooxypropanol (3NOP), ethyl-3NOP, 2-bromoethanesulfonate (BES), 2-chloroethanesulfonate (CES), 3-bromopropanesulfonate (BPS), bromochloromethane (BCM), bromoform, bromodichloromethane, dibromochloromethane, carbon tetrachloride, trichloroacetamide, trichloroethyladipate, lumazin (2,4-pteridinedione), p-aminobenzoic acid, lovastatin, mevastatin, pravastatin, diallyl disulfide, garlic oil, saponins, tannins, flavonoids, nitrate, nitroethane, -nitro-propionate, 2-nitropropanol, 2-nitroethanol, malate, acrylate, oxaloacetate, fumarate, propynoic acid, 3-butenoic acid, 2-butynoic acid, ethyl 2-butynoate, monensin, lasalocid, bovicin HC5, nisin, and any combination thereof.

In preferred embodiments, the agent is 3NOP or ethyl-3NOP.

In certain aspects, provided herein are methods of reducing CH₄ production in a subject, the method comprising orally administering to and/or feeding the subject the antibody, the milk and/or the derivatives thereof, and/or the animal feed of the present disclosure.

In certain aspects, the methods of the present disclosure relate to a subject. In some embodiments, the subject is selected from a cow, a bull, a bison, a yak, a buffalo, an antelope, a goat, a sheep, a deer, a giraffe, a caribou, a gazelle, a macropod, a llama, a camel, and an alpaca.

In some embodiments, the subject is an offspring (e.g., calf) of the vaccinated female subject that received the milk comprising an antibody that binds at least one methanogen.

In some embodiments, the subject is an adult subject. In other embodiments, the subject is a young subject (e.g., a calf). In some embodiments, a young subject includes a subject from birth to weaning. In some embodiments, a young subject includes a subject from birth up to two years of age, such from birth up to 3, 4, 5, 6, 7, 8, 9, 10, 11, or 12 months. Accordingly, in some embodiments, a young subject may be at least, about, or no more than 1, 2, 3, 4, 5, 6, 7, 8, 9, 10, 11, 12, 13, 14, 15, 16, 17, 18, 19, 20, 21, 22, 23, or 24 months in age. In preferred embodiments, a young subject is administered with a vaccine, antibodies, milk, animal feed, agent (e.g., an agent that reduces CH₄ production in a subject, a probiotic bacterial strain, a small molecule inhibitor, etc.), or other composition of the present disclosure prior to establishment of methanogens. In some such embodiments, a lower dose of vaccine or other agents may be required.

In some embodiments, a subject is a subject born from a vaccinated parent(s). In some embodiments, a subject is a subject born from a vaccinated mother such that the subject received a high level of methanogen-neutralizing antibodies in the colostrum and milk fed to the subject at birth. Such a subject or a subject who received an early treatment may have low initial methanogen establishment, thereby enhancing a long term performance of the vaccines, antibodies, milk, animal feed, agents (e.g., an agent that reduces CH₄ production in a subject, a probiotic bacterial strain, a small molecule inhibitor, etc.), or other compositions of the present disclosure.

In some embodiments, a subject is vaccinated with each change in hands and/or environment, such as from one owner to another, one ranch to another, or one geographical area to another. Typical ranch operations will vaccinate a newly arrived animal to manage disease, and vaccinating with a vaccine of the present disclosure (e.g., a vaccine that targets at least one methanogen cell surface protein) will fall into this standard agronomic practice.

In some embodiments, a subject is vaccinated with a vaccine of the present disclosure (e.g., a vaccine that targets at least one methanogen cell surface protein) at a time when the subject is subject to at least one other vaccination. For example, a subject (e.g., a domesticated ruminant, a dairy cow, a beef cow) is subject to vaccination against infectious bovine rhinotracheitis (IBR), bovine virus diarrhea (BVD), parainfluenza-3 (PI3), bovine respiratory syncytial virus (BRSV), clostridia, E. Coli mastitis, leptospirosis, mannheimia hemolytica, brucella, vibriosis, campylobacter, trichomonas, trichomoniasis, rotavirus, foot and mouth disease virus (FMDV), coronavirus, and/or respiratory disease. An exemplary vaccination regimen for a ruminant throughout the life cycle is shown in Table 9.

Additionally or alternatively, CH₄ reducing vaccine compositions may be combined with one or more additional antigens configured to target infectious bovine rhinotracheitis (IBR), bovine virus diarrhea (BVD), parainfluenza-3 (PI3), bovine respiratory syncytial virus (BRSV), clostridia, E. Coli mastitis, leptospirosis, mannheimia hemolytica, brucella, vibriosis, campylobacter, trichomonas, trichomoniasis, rotavirus, foot any mouth disease virus (FMDV) coronavirus, and/or respiratory disease to form a multivalent vaccine composition, which is then administered to the subject as disclosed herein.

TABLE 9
Exemplary administration schedule of USDA-approved vaccinations for a ruminant

| Animal | Life Stage | Exemplary Vaccinations |
|---|---|---|
| Dairy | Adult | 4-way viral-IBR/BVD/PI3/BRSV |
| | Dry-off (e.g., 45-60 days pre-calving) | Leptospirosis (5-way) |
| Animal | Life Stage | Exemplary Vaccinations |
| | | Clostridial (7-way) |
| | | J-5 (mastitis) |
| Dairy | Adult | 4-way viral-IBR/BVD/PI3/BRSV |
| | Pre-breeding (e.g., 30 days prior to | Leptospirosis (5-way) |
| | breeding season) | |
| Dairy | Calf | 4-way viral-IBR/BVD/PI3/BRSV |
| | Weaning (e.g., 6-12 weeks) | Leptospirosis (5-way) |
| | | Clostridial (7-way) |
| | | Mannheimia hemolytica |
| Dairy | Calf | 4-way viral-IBR/BVD/PI3/BRSV |
| | Yearling (i.e., 12-24 mo) | Leptospirosis (5-way) |
| | | Clostridial (7-way) |
| | | Mannheimia hemolytica |
| | | Brucella |
| Beef | Adult | 4-way viral-IBR/BVD/PI3/BRSV |
| | Pre-breeding (e.g., 30 days prior to | Leptospirosis (5-way) |
| | breeding season) | Vibriosis (Campylobacter)-natural |
| | | mating |
| | | Trichomonas-natural mating |
| Beef | Adult | 4-way viral-IBR/BVD/PI3/BRSV |
| | Pregnancy examination (e.g., 30-45 days | Leptospirosis (5-way) |
| | post-breeding season) | Clostridial (7-way) |
| | | Scour vaccines-E. coli, possibly |
| | | combination with rota and coronavirus |
| Beef | Calf | Clostridial vaccines |
| | Newborn (e.g., 0-3 mo) | Possibly respiratory vaccines, if there is |
| | | a history of disease in very young calves |
| Beef | Calf | 4-way viral-IBR/BVD/PI3/BRSV |
| | Weaning (e.g., 6-8 mo) | Leptospirosis (5-way) |
| | | Clostridial (7-way) |
| | | Mannheimia hemolytica |
| | | Brucella |
| Beef | Adult | 4-way viral-IBR/BVD/PI3/BRSV |
| | Arrival at stocker operation (e.g., 6-8 mo) | Mannheimia hemolytica |
| Beef | Adult | 4-way viral-IBR/BVD/PI3/BRSV |
| | 14-21 d post-arrival at stocker operation | Clostridial (7-way) |
| | | Mannheimia hemolytica |
| | | Leptospirosis (5-way) (optional) |
| Beef | Adult | 4-way viral-IBR/BVD/PI3/BRSV |
| | Arrival at feedlot operation (e.g., 9-11 mo) | Mannheimia hemolytica |
| Beef | Adult | 4-way viral-IBR/BVD/PI3/BRSV |
| | 14-21 d post-arrival at feedlot operation | Clostridial (7-way) |
| | | Mannheimia hemolytica |
| | | Leptospirosis (5-way) (optional |

Vaccinating a large number of animals (e.g., in a ranch, e.g., herding ruminants) is a challenging process. Thus, in preferred embodiments, the administration of a vaccine of the present disclosure is incorporated into a pre-existing vaccination program that an animal is already subject to. Such a method reduces the cost, time, and efforts in administering a vaccine of the present disclosure to an animal.

### Methods for Detection of CH4 Production

Various methods for detecting the level of CH₄ produced by animals are known in the art and can be applied to the methods of the present disclosure.

In some embodiments, portable respiration hoods for tethered and non-tethered animals (Garnsworthy et al. (2012) J. Dairy Sci. 95:3166-3180; Garnsworthy et al. (2019) Animals 9:837; Zimmerman and Zimmerman WO2011130538; each of which is incorporated herein by reference) directly measure the gas concentration of incoming and exhaust air from individual animals.

In some embodiments, tracer-ratio gas releases from the animal (Johnson et al. (1994) Environ. Sci. Technol., 28, 359-362, which is incorporated herein by reference), such as Sulfur hexafluoride (SF6) (Grainger et al. (2007) J. Dairy Sci., 90:2755-2766; Vechi et al. (2022) Agriculture, Ecosystems and Environment 330:107885; each of which is incorporated herein by reference), assumes that the tracer gas and the emitted CH₄ have similar transport paths, so that a tracer measurement can establish the CH₄ emission rate.

In some embodiments, micrometeorological techniques are typically considered a herd-scale measurement, where the emission rate is calculated from the measurement of enhanced gas concentrations downwind of an animal herd (Harper et al. (2011) Anim. Feed Sci. Tech., 166-167, 227-239, which is incorporated herein by reference), and these include the mass balance technique (Laubach et al. (2008) Aust. J. Exp. Agr., 48:132-137; Lockyer and Jarvis (1995) Environ. Pollut. 90:383-390; each of which is incorporated by reference), eddy covariance (Dengel et al. (2011) Glob. Change Biol., 17:3524-3533; Felber et al. (2015) Biogeosciences, 12:3925-3940; each of which is incorporated herein by reference), and inverse dispersion techniques (Flesch et al., (2005) Atmos. Environ., 39:4863-4874; Todd et al. (2014) J. Environ. Qual., 43:1125-1130; Bai et al. (2021) Atmos. Meas. Tech., 14:3469-3479; each of which is incorporated herein by reference). The main advantage of micrometeorological techniques is that they do not interfere with the animals or the environment.

There are also devices that measure the level of CH₄ (see e.g., Rey et al. (2019) Animals 9:563, Mapfumo et al. (2018) Pastoralism: Research, Policy and Practice 8:15; each of which is incorporated herein by reference). For example, the laser CH₄ detector (LMD) is a handheld open path laser measuring device (e.g., LaserMethaneMini (Tokyo Gas Engineering Co., Ltd. Anritsu Devices Co., Ltd., Tokyo, Japan)). The principle of the LMD measuring technology is described (Chagunda et al. (2013) Animal, 7:394-400; Garnsworthy et al. (2012) J. Dairy Sci. 95:3166-3180; and Chagunda et al. (2009) Comput. Electron. Agric. 68:157-160; each of which is incorporated herein by reference). Briefly, this device is based on infrared absorption spectroscopy using a semiconductor laser for CH₄ detection. The device must be pointed towards the nostrils of the cow from a fixed distance. Then, the LMD measures the density of the air column between the device and the animal's nostrils. The reflected laser beam is detected by the device, and its signal is processed and converted to the cumulative CH₄ concentration along the laser path in ppm-m. The LMD is connected to a tablet (Samsung Galaxy Tab A6, New Jersey, USA) running GasViewer app (Tokyo Gas Engineering Solutions, Tokyo, Japan) via Bluetooth connection for exporting and storing the data in real time at 0.5 s intervals. The effect of atmospheric ambient CH₄ concentration from the measurements is discounted using the offset function of the LMD.

The non-dispersive infrared analyzer CH₄ analyzer (NDIR) (Guardian NG Edinburg Instruments Ltd., Livinstong, UK) is one of the so-called sniffer methods that measure CH₄ concentration (ppm) in breath or exhaled air. These methods have been previously used (e.g., by Garnsworthy et al. (2012) J. Dairy Sci. 95:3166-3180) to assess the CH₄ production of dairy cows at commercial farms. Briefly, a gas sampling tube from the front of a cow's head to a gas analyzer to continuously measure CH₄ concentration in the cow's breath is used. Then, air is drawn through the instrument by an integral pump between the gas inlet port and analyzer. The device can have a range of 0 to 10,000 ppm, and air can be sampled continuously at a rate of 1 L/min through an 8 mm polyamide tube, using approximately 2 m of tube from the analyzer to cow's nostrils. CH₄ concentration can be recorded at 1 s intervals and stored in a datalogger (Data Recorder SRD-99; Simex Sp. Z o.o, Gdansk, Poland). Baseline or ambient CH₄ concentration can be calculated as mean CH₄ concentration before starting the measurements and subtracted from the measured data. Each day before starting measurements, the NDIR analyzer should be verified using standard mixtures of CH₄ in N₂ (0.0%, 0.25%, 0.50%, 0.75% and 1.0%; MESA International Technologies INC, Santa Ana, CA, USA).

Certain methods and devices are described further below and in Table 10.

TABLE 10
Features of exemplary methods for measuring CH₄ output by individual animals

| | Purchase | Running | | | Behaviour | |
|---|---|---|---|---|---|---|
| Method | Cost ² | Costs ² | Labour ² | Repeatability | Alteration ³ | Throughput |
| Respiration chamber | High | High | High | High | High | Low |
| SF₆ technique | Medium | High | High | Medium | Medium | Medium |
| Breath sampling during | Low ⁴ | Low | Low | Medium | None | High |
| milking and feeding | | | | | | |
| GreenFeed | Medium | Medium | Low | Medium | Low | Medium |
| Laser methane detector | Low | Low | High | Low | Low-Medium | Medium |

As indicated above and shown in Table 10, exemplary methods include respiration chambers, the sulfur hexafluoride (SF6) tracer technique, breath sampling during milking or feeding, the GreenFeed system, and the laser CH₄ detector. Each method measures different components of CH₄ output. Only respiration chambers measure total emissions from the animal via the oral, nasal and anal routes; all other methods ignore emissions via the anus and only measure CH₄ emitted in breath. Breath measurements are justified because 99% of CH₄ is emitted from the mouth and nostrils, and only 1% via the anus. The SF6 technique samples breath over 24 h, whereas other techniques use spot samples of breath over periods of min. throughout the day, so diurnal variation has to be considered. The majority of CH₄ (87%) is released by eructation, which provides a clear signal for sample processing.

### Respiration Chamber

Respiration chambers for open- or closed-circuit indirect calorimetry are considered the ‘Gold Standard’, and were used extensively in nutrition studies when establishing the Metabolizable Energy system. A single animal (or occasionally more) is confined in a chamber for between 2 and 7 days. Concentration of CH₄ (and other gases if required) is measured at the air inlet and outlet vents of the chamber. The difference between outlet and inlet concentrations is multiplied by airflow to indicate CH₄ emissions rate. In most installations, a single gas analyzer is used to measure both inlet and outlet concentrations, often for two or more chambers. This involves switching the analyzer between sampling points at set intervals, so concentrations are actually measured for only a fraction of the day.

Respiration chambers vary in construction materials, size of chamber, gas analysis equipment and airflow rate, all of which can influence results. Validation of 22 chambers at six UK research sites revealed an uncertainty of 25.7% between facilities, which was reduced to 2.1% when correction factors were applied to trace each facility to the international standard for CH₄. The main sources of uncertainty were stability and measurement of airflow, which are crucial for measuring CH₄ emission rate. It was concluded, however, that chambers were accurate for comparing animals measured at the same site. It is an added challenge, when benchmarking alternative methods against respiration chambers, that the respiration chambers themselves have not been benchmarked against respiration chambers at other facilities.

For large-scale evaluation of CH₄ emissions by individual animals, respiration chambers are challenging, with only a single study in growing Angus steers and heifers exceeding 1000 animals, which found CH₄ production to be moderately heritable h²=0.27±0.07. Installation costs and running costs are high, and only one animal can be measured at a time. If the monitoring time is three days per animal, and chambers are run continuously, then maximum throughput would be approximately 100 animals per chamber per year. In practice, throughput is likely to be 30 to 50 animals per year. Cows are social animals, and confinement in a chamber may ultimately influence their feeding behavior, resulting in less feed being consumed and in a different meal pattern compared with farm conditions. Altered feeding patterns or levels is not a problem for metabolic studies evaluating feeds, but can be a problem when evaluating individual animals. Furthermore, the representativeness of respiration chambers to grazing systems has been called into question. However, promising developments have led to more animal friendly respiration chambers constructed from cheaper, transparent materials. These lower the cost and reduce the stress of confinement with minimal disruptions to accuracy, precision and no drop in feed intake of the cows (Hellwing et al. (2012) J. Dairy Sci. 95:6077-6085, which is incorporated herein by reference).

### The SF₆ Method

The SF₆ tracer gas technique was developed in an attempt to measure CH₄ emissions by animals without confinement in respiration chambers. Air is sampled near the animal's nostrils through a tube attached to a halter and connected to an evacuated canister worn around the animal's neck or on its back. A capillary tube or orifice plate is used to restrict airflow through the tube so that the canister is between 50 and 70% full after approximately 24 h. A permeation tube containing SF₆ is placed into the rumen of each animal. The pre-determined release rate of SF₆ is multiplied by the ratio of CH₄ to SF₆ concentrations in the canister to calculate CH₄ emission rate.

Many research centers have used the SF₆ technique with variations in design of sampling and collection equipment, permeation tubes, and gas analysis. Reliable results depend on following standard protocols, with greatest variation coming from accuracy of determining SF6 release rate from permeation tubes and control of sampling rate. With capillary tubes, sampling rate decreases as pressure in the canister increases, whereas an orifice plate gives a steadier sampling rate over 24 h. A source of error that has not been evaluated is that animals might interact and share CH₄ emissions when the sampling tube of one animal is near the head of another animal. There is good agreement between CH₄ emissions measured by the SF6 technique and respiration chambers, although results from the SF₆ technique are more variable.

For large-scale evaluation of CH₄ emissions by individual animals, the SF₆ technique is more useful than respiration chambers. Animal behavior and intake might be affected by wearing the apparatus, and by daily handling to exchange canisters, but the technique is considerably less intrusive than respiration chambers, because cows remain in the herd. Labor and monetary costs for changing canisters each day and for lab analysis are high. Throughput is limited by the number of sets of apparatus available, handling facilities, labor, and the capacity of the lab for gas analysis. Animals need to be measured for 5 to 7 days, and it is recommended that group size should be less than 15 animals, so maximum throughput would be about 750 animals per year. Heritability has been estimated for CH₄ production in grazing Holstein cows at h²=0.33±0.15.

### Breath Sampling During Milking and Feeding

Several research groups have developed methods to measure CH₄ concentration in breath of cows during milking and/or feeding. These are often referred to as ‘sniffer methods’ because they use devices originally designed to detect dangerous gas leaks. Air is sampled near the animal's nostrils through a tube fixed in a feed bin and connected directly to a gas analyzer. The feed bin might be in an automatic milking station or in a concentrate feeding station. Different research centers use different gas analyzers (Nondispersive Infrared (NDIR), Fourier-transform infrared (FTIR) or photoacoustic infrared (PAIR)) and different sampling intervals (1, 5, 20 or 90-120 s). CH₄ concentration during a sampling visit of typically between 3 and 10 min may be specified as the overall mean, or the mean of eructation peaks. Some centers use CO₂ as a tracer gas and calculate daily CH₄ output according to ratio of CH₄ to CO₂ and daily CO₂ output predicted from performance of the cow. Repeatability and rank correlations were higher for eructation peaks than for mean concentrations, and were higher for eructation peaks than for CH₄ to CO₂ ratio. However, all methods show good repeatability.

For large-scale evaluation of CH₄ emissions by individual animals, breath-sampling methods have significant advantages compared with other methods. Breath-sampling methods are non-invasive because, once installed, animals are unaware of the equipment and are in their normal environment. Animals follow their normal routine, which includes milking and feeding, so no training of animals, handling, or change of diet is required. Equipment is relatively cheap, although more expensive gas analyzers are available, and running costs are negligible.

The compromise for non-invasiveness of breath-sampling is that concentrations of gasses in the sampled air are influenced by cow head position relative to the sampling tube. The use of head position sensors and data filtering algorithms can remove the effects when the cow's head is completely out of the feed bin, but not within the feed bin. Consequently, sniffer measurements are more variable than flux methods, with factors like variable air flow in the barn increasing measurement error (imprecision), and head position, a highly repeatable characteristic, inflating between-cow variability.

Using CO₂ as a tracer gas partly addresses the issue but, because CO₂ arises from metabolism as well as rumen fermentation, variability of CO₂ emissions has to be considered. A further consideration is diurnal variation in breath concentrations of CH₄ and CO₂ because animals are spot-sampled at different times of day and night. Diurnal variation can be accounted for either by fitting a model derived from the whole group of animals, or by including time of measurement in the statistical model.

The number of observations per analyzer is limited only by number of cows assigned to one automatic milking station or concentrate feeding station and length of time equipment is installed. Typically, each analyzer will record 40 to 70 animals 2 to 7 times per day for 7 to 10 days, although the number of sampling stations per analyzer can be increased by using an automatic switching system. Throughput per analyzer is likely to be 2000 to 3000 animals per year. Estimates of heritability for CH₄ production measured using this method range from h²=0.12 to 0.45 over multiple studies.

GreenFeed GreenFeed (C-Lock Inc., Rapid City, SD, USA) is a sophisticated sniffer system where breath samples are provided when animals visit a bait station. As with other sniffer systems, GreenFeed samples breath from individual animals several times per day for short periods (3 to 7 min). GreenFeed is a portable standalone system used in barn and pasture applications, and incorporates an extractor fan to ensure active airflow and head position sensing for representative breath sampling. Measurements are pre-processed by the manufacturer, and data are available in real time through a web-based data management system. As GreenFeed captures a high proportion of emitted air and measures airflow, which can be calibrated using a tracer gas, CH₄ emission is estimated as a flux at each visit. Providing visits occur throughout the 24 h, CH₄ emission can be estimated directly as g/day.

A limitation of the GreenFeed system Is that animals require training to use the system, although animals which have been trained to use the system will readily use it again. However, some animals will not use the system or will use it infrequently, and frequency of visits is affected by diet. This can be a challenge when screening commercial herds for CH₄ emission under genetic evaluation.

The manufacturer recommends 15 to 25 animals per GreenFeed unit, and recordings are made typically for 7 days. If all animals visit the unit adequately, throughput per unit is likely to be 750 to 1250 animals per year.

### Laser CH₄ Detector (LMD)

The laser CH₄ detector (LMD) is a highly responsive, hand-held device that is pointed at an animal's nostrils and measures CH₄ column density along the length of the laser beam (ppm.m). In the first implementation of LMD on a farm, measurements for each cow were taken over periods of 15 to 25 s between eructation events, and could detect CH₄ emitted each time the animal breathed out. In a later study with sheep and beef cattle, monitoring periods of 2 to 4 min allowed authors to separate breathing cycles from eructation events. Typically, animals are restrained either manually or in head yokes at a feed fence for the required length of time. The operator has to stand at the same distance (1 to 3 m) from each animal every time and must be careful to keep the laser pointed at the animal's nostrils throughout the measurement period.

The LMD can be used in the animal's normal environment, although for consistency restraint is required during measurement. Because the LMD measures CH₄ in the plume originating from the animal's nostrils, results can be affected by factors such as: distance from the animal; pointing angle; animal's head orientation and head movement; air movement and temperature in the barn; adjacent animals; and operator variation. Operator variation is likely to be one of the biggest factors, because the operator controls distance and pointing angle, and is responsible for ensuring that the laser remains on target. The structure of the barn and the resulting ventilation conditions and wind speed at the location of the measurement are also considerable sources of variation in recorded CH₄.

Assuming operator fatigue does not limit measurements, each LMD could record up to 10 animals per hr. If each animal is recorded 3 times (on 3 consecutive days, for example), throughput is likely to be up to 1000 animals per year.

Methods for Producing of Recombinant Protein and/or Antibodies

The terms “expression vector” mean the vehicle by which a DNA or RNA sequence (e.g., a foreign gene) can be introduced into a host cell, so as to transform the host and promote expression (e.g., transcription and translation) of the introduced sequence. Thus, a further object of the invention relates to a vector comprising a nucleic acid of the present invention.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell can be used. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, gPenv-positive cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present invention relates to a cell which has been transfected, infected or transformed by a nucleic acid and/or a vector according to the invention. The term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.”

The nucleic acids may be used to produce a recombinant polypeptide of the invention in a suitable expression system. The term “expression system” means a host cell and compatible vector under suitable conditions, e.g., for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL 1662, hereinafter referred to as “YB2/0 cell”), and the like. The YB2/0 cell is preferred, since ADCC activity of chimeric or humanized antibodies is enhanced when expressed in this cell.

The present invention also relates to a method of producing a recombinant host cell expressing an antibody or a polypeptide of the invention according to the invention, said method comprising the steps consisting of (i) introducing in vitro or ex vivo a recombinant nucleic acid or a vector as described herein into a competent host cell, (ii) culturing in vitro or ex vivo the recombinant host cell obtained and (iii), optionally, selecting the cells which express and/or secrete said antibody or polypeptide. Such recombinant host cells can be used for the production of antibodies and polypeptides of the invention.

Antibodies and fragments thereof, immunoglobulins, and polypeptides of the present invention may be produced by any technique known in the art, such as, without limitation, any chemical, biological, genetic or enzymatic technique, either alone or in combination.

Knowing the amino acid sequence of the desired sequence, one skilled in the art can readily produce said antibodies or polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies and other polypeptides of the present invention can be synthesized by recombinant DNA techniques as is well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

In particular, the present invention further relates to a method of producing an antibody or a polypeptide of the invention, which method comprises the steps consisting of: (i) culturing a transformed host cell according to the invention under conditions suitable to allow expression of said antibody or polypeptide; and (ii) recovering the expressed antibody or polypeptide.

Antibodies and other polypeptides of the present invention are suitably separated from the culture medium by conventional immunoglobulin purification procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Chimeric antibodies (e.g., mouse-ruminant chimeras, or one ruminant-another ruminant (e.g., goat-cow) chimeras, or ruminant-human chimeras) of the present invention can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell. The CH domain of a human chimeric antibody can be any region which belongs to human immunoglobulin, such as the IgG class or a subclass thereof, such as IgG1, IgG2, IgG3 and IgG4. Similarly, the CL of a human chimeric antibody can be any region which belongs to Ig, such as the kappa class or lambda class. Chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al.

International Patent Publication PCT/US86/0226; Akira et al. European Patent Application 184,18; Taniguchi, M. European Patent Application 171,49; Morrison et al. European Patent Application 173,49; Neuberger et al. PCT Application WO 86/0153; Cabilly et al. U.S. Pa. No. 4,816,56; Cabilly et al. European Patent Application 125,02; Better et al. (1988) Science 24:1041-104; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison, S. L. (1985) Science 229:1202-1207; Oi et al. (1986) Biotechniques:21; Winter U.S. Pat. No. 5,225,53; Jones et al. (1986) Nature 32:552-52; Verhoeyan et al. (1988) Science 239:1534; and Beidler et al. (1988) J. Immunol. 141:4053-4060.

In addition, methods for producing antibody fragments are well-known. For example, Fab fragments of the present invention can be obtained by treating an antibody which specifically reacts with a ganglioside with a protease such as papain. Also, Fabs can be produced by inserting DNA encoding Fabs of the antibody into a vector for prokaryotic expression system, or for eukaryotic expression system, and introducing the vector into a procaryote or eucaryote (as appropriate) to express the Fabs.

Similarly, F(ab′)2 fragments of the present invention can be obtained treating an antibody which specifically reacts with a ganglioside with a protease, pepsin. Also, the F(ab′)2 fragment can be produced by binding Fab′ described below via a thioether bond or a disulfide bond.

Fab′ fragments of the present invention can be obtained treating F(ab′)2 which specifically reacts with a ganglioside with a reducing agent, dithiothreitol. Also, the Fab′ fragments can be produced by inserting DNA encoding a Fab′ fragment of the antibody into an expression vector for prokaryote, or an expression vector for eukaryote, and introducing the vector into a prokaryote or eukaryote (as appropriate) to perform its expression.

In addition, scFvs of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv.

### Kits

The present invention also encompasses kits. For example, the kit can comprise a vaccine of the present disclosure, any one of pharmaceutical compositions described herein, at least one additional agent that reduces CH₄ production in a subject described herein, or any combination thereof, packaged in a suitable container and can further comprise instructions for using such reagents. The kit may also contain other components, such as administration tools packaged in the same or separate container.

### Production of Low Carbon Animal Products

In some aspects, the vaccine compositions and methods disclosed herein may be used to produce low carbon animal products. The production of low carbon animal products may involve administering a vaccine composition to an animal, determining greenhouse gas emissions from the vaccinated animal, harvesting an animal product, and determining the carbon intensity of the harvested product compared to a product from an unvaccinated animal.

In some embodiments, a method of producing a low carbon animal product may comprise:
(a) Administering to an animal a vaccine composition comprising cells and/or cell parts of at least one methanogen, as described in any of the embodiments herein. The animal may be a mammal, such as a ruminant.
(b) Determining an amount of emissions of at least one greenhouse gas from the vaccinated animal following administration until animal product harvesting. The greenhouse gas may include, but is not limited to, CO₂, CH₄, N₂O, and/or H₂. In some cases, the emissions may be measured using a GreenFeed system or other suitable emissions measurement technique.
(c) Harvesting an animal product from the vaccinated animal. The animal product may include, but is not limited to, meat, milk, wool, or other products derived from the animal.
(d) Determining an amount of the harvested animal product.
(e) Determining a first carbon intensity as a ratio of the amount of emissions from the vaccinated animal and the amount of harvested animal product.
(f) Determining a second carbon intensity as a ratio of an amount of carbon emissions from an untreated animal and the same amount of harvested animal product.
(g) Determining the difference between the first carbon intensity and the second carbon intensity.

In some embodiments, the method may further comprise administering to the animal at least one agent that reduces methane production, in addition to the vaccine composition. This may provide a synergistic effect in reducing greenhouse gas emissions.

In some aspects, the method may include certifying the animal product as a low carbon intensity product based on the determined difference between the first carbon intensity and the second carbon intensity. This certification may provide added value to the animal product in markets where low carbon products are desired.

The methods described herein may allow for the production of animal products with a reduced carbon footprint compared to conventional production methods. This may be beneficial for meeting sustainability goals, reducing environmental impact, and potentially commanding premium prices for low carbon animal products in certain markets.

### Exemplary Embodiments

1. A vaccine composition comprising cells and/or cell parts of at least one hydrogenotroph or at least one methanogen, optionally wherein the at least one hydrogenotroph or at least one methanogen is selected from the hydrogenotrophs or methanogens in Table A and/or Table B.

2. The vaccine composition of embodiment 1, wherein the vaccine comprises at least one methanogen. 3. The vaccine composition of embodiment 1 or 2, wherein the at least one methanogen is of a genus Methanobrevibacter.

4. The vaccine composition of any one of embodiments 1-3, wherein the at least one methanogen comprises Methanobrevibacter ruminantium, optionally Methanobrevibacter ruminantium M1.

5. The vaccine composition of any one of embodiments 1-4, wherein the at least one methanogen comprises Methanobrevibacter gottschalkii, optionally Methanobrevibacter gottschalkii DSM11977.

6. The vaccine composition of any one of embodiments 1-5, wherein the vaccine composition comprises at least 2, 3, 4, 5, 6, 7, 8, 9, or 10 methanogens, preferably at least 8 methanogens.

7. The vaccine composition of any one of embodiments 1-6, wherein the vaccine composition comprises two methanogens.

8. The vaccine composition of embodiment 7, wherein the ratio of the two methanogens is at least about 1:9, 2:8, 3:7, 4:6, 5:5, 6:4, 7:3, 8:2, or 9:1 by cell number.

9. The vaccine composition of embodiment 7 or 8, wherein the two methanogens are Methanobrevibacter ruminantium and Methanobrevibacter gottschalkii.

10. The vaccine composition of embodiment 9, wherein the ratio of Methanobrevibacter ruminantium to Methanobrevibacter gottschalkii is at least about 1:9, 2:8, 3:7, 4:6, 5:5, 6:4, 7:3, 8:2, or 9:1 by cell number.

11. The vaccine composition of any one of embodiments 1-10, wherein at least about 50%, 55%, 60%, 65%, 70%, 75%, 80%, 85%, 90%, 95%, 96%, 97%, 98%, 99%, or 100% of the cells and/or cell parts are cells (i.e., whole cells).

12. The vaccine composition of any one of embodiments 1-11, wherein the vaccine composition comprises at least about 10⁶, 10⁷, 10⁸, 10⁹, or 10¹⁰ cells per mL; and/or no more than about 10⁷, 10⁸, 10⁹, 10¹⁰, 10¹¹, or 10¹² cells per mL.

13. The vaccine composition of any of embodiments 1-12, wherein the vaccine composition comprises:
(a) at least about 10⁶ but no more than about 10¹² cells per mL;
(b) at least about 10⁸ but no more than about 10¹² cells per mL;
(c) at least about 10⁸ but no more than about 10¹¹ cells per mL; or
(d) at least about 10⁸ but no more than about 10¹⁰ cells per mL.

14. The vaccine composition of embodiment 12 or 13, wherein the cells per mL is equivalent to events per mL.

15. The vaccine composition of any one of embodiments 1-14, wherein the vaccine composition comprises:
(a) at least about 0.2, 0.4, 0.6, 0.8, 1, 1.2, 1.4, 1.6, 1.8, 2, or 4 mg of total proteins; and/or
(b) no more than about 0.4, 0.6, 0.8, 1, 1.2, 1.4, 1.6, 1.8, 2, 4, 6, 8, or 10 mg of total proteins.

16. The vaccine composition of any one of embodiments 1-15, wherein the vaccine composition comprises:
(a) at least about 0.2 but no more than about 10 mg of total protein; or
(b) at least about 0.6 but no more than about 5 mg of total protein.

17. The vaccine composition of any one of embodiments 1-16, wherein the cells and/or cell parts are killed, fixed, and/or irradiated. 18. The vaccine composition of embodiment 17, wherein the cells and/or cell parts are:
(a) killed by heat, steam, freezing, or sonicated;
(b) fixed by formaldehyde or formalin; and/or
(c) irradiated by UV or gamma irradiation.

19. The vaccine composition of any one of embodiments 1-18, wherein the cells and/or cell parts are lyophilized.

20. The vaccine composition of any one of embodiments 1-19, wherein the vaccine composition is a pharmaceutical composition.

21. The vaccine composition of embodiment 20, wherein the pharmaceutical composition comprises at least one carrier, at least one excipient, at least one cryoprotectant, and/or at least one inactive ingredient.

22. The vaccine composition of any one of embodiments 1-21, wherein the vaccine composition comprises at least one adjuvant.

23. The vaccine composition of embodiment 22, wherein the at least one adjuvant comprises:
(a) inorganic salts, preferably aluminum and calcium salts;
(b) an oil emulsion;
(c) saponins;
(d) lipid or liposomes
(e) an immune-stimulating complex;
(f) a carrier protein (e.g., keyhole limpet hemocyanin (KLH), optionally conjugated to cells and/or cell parts);
(g) a bacterial product or derivatives thereof (e.g., bacterial toxins, lipopolysaccharide, etc.);
(h) a cytokine; or
(i) any combination of two or more selected from (a)-(h).

24. The vaccine composition of embodiment 22 or 23, wherein the at least one adjuvant comprises Complete Freund's adjuvant, Incomplete Freund's adjuvant, Montanide ISA70, Montanide ISA61, Saponin, chitosan thermogel, lipid (e.g., monophosphoryl lipid A), a lipid nanoparticle/cationic liposome adjuvant, Emulsigen-D, Emulsigen, Emulsigen-P, Polygen, ENABL 06, Montainde ISA 201, Montanide Gel 02, or any combination of two or more thereof.

25. The vaccine composition of embodiment 24, wherein the at least one adjuvant comprises Complete Freund's adjuvant or Incomplete Freund's adjuvant.

26. The vaccine composition of any one of embodiments 1-25, wherein the vaccine composition induces immune response against at least one cell surface protein or a fragment thereof of the at least one methanogen.

27. The vaccine composition of embodiment 26, wherein the at least one cell surface protein or a fragment thereof is selected from an adhesin-like protein, adhesin-like protein with cysteine protease domain, tetrahydromethanopterin S-methyltransferase subunit, a fragment thereof, and/or any combination thereof.

28. A method of treating a disease in a subject, the method comprising administering to the subject a vaccine composition of any one of embodiments 1-27.

29. The method of embodiment 28, wherein the disease is a periodontal disease, Inflammatory Bowel Disease (IBD), gingivitis, bloat, and/or liver abscess.

30. The method of embodiment 28, wherein the disease is associated with elevated, increased, or severe lactic acidosis.

31. A method of inducing an immune response against at least one methanogen in a subject, the method comprising administering to the subject the vaccine of any one of embodiments 1-27.

32. The method of embodiment 31, wherein the immune response comprises a B cell response and/or a T cell response, preferably a B cell response.

33. A method of reducing (i) lactate in a digestive tract and/or (ii) increasing pH in a digestive tract in a subject, the method comprising administering to the subject the vaccine of any one of embodiments 1-27.

34. A method of reducing the activity, number, and/or type of methanogens in a digestive tract of a subject, the method comprising administering to the subject the vaccine composition of any one of embodiments 1-27.

35. The method of embodiment 33 or 34, wherein the digestive track comprises rumen, reticulum, omasum, abomasum, stomach, small intestine, large intestine, and/or rectum, preferably rumen.

36. A method of reducing the amount of methane (CH₄) and/or hydrogen (H₂) emitted by a subject, preferably eructated and/or exhaled, the method comprising administering to the subject the vaccine composition of any one of embodiments 1-27.

37. The method of embodiment 36, wherein the amount of methane (CH₄) and/or hydrogen (H₂) is reduced by about 5-100%, preferably by about 10-100%, compared to a control, optionally wherein the amount of methane (CH₄) and/or hydrogen (H₂) is reduced by at least about 5%, 10%, 15%, 20%, 25%, 30%, 35%, 40%, 45%, 50%, 55%, 60%, 65%, 70%, 75%, 80%, 85%, 90%, 95%, or 100% compared to a control. 38. The method of embodiment 36 or 37, wherein the amount of methane (CH₄) and/or hydrogen (H₂) is reduced by about 20-100%, preferably by about 30-100%, compared to a control.

39. The method of any one of embodiments 36-38, wherein the amount of methane (CH₄) is reduced by (a) about 1 kg-about 50 kg within 8 weeks from the time of first vaccination, or (b) about 5 kg-about 300 kg within a year from the time of first vaccination, compared to a control.

40. The method of any one of embodiments 36-39, wherein the amount of methane (CH₄) normalized to an amount of CO₂ emitted by the subject (i.e., CH₄/CO₂) is reduced by about 5-100%, preferably by about 10-100%, compared to a control, optionally wherein the amount of methane (CH₄) normalized to the amount of CO₂ is reduced by at least about 5%, 10%, 15%, 20%, 25%, 30%, 35%, 40%, 45%, 50%, 55%, 60%, 65%, 70%, 75%, 80%, 85%, 90%, 95%, or 100% compared to a control.

41. The method of embodiment 40, wherein the amount of methane (CH₄) normalized to the amount of CO₂ is reduced by about 20-100%, preferably by about 30-100%, compared to a control.

42. The method of any one of embodiments 36-41, wherein the amount of hydrogen (H₂) is reduced by (a) about 10 g-about 500 g within 8 weeks from the time of first vaccination, or (b) about 50 g-about 3 kg within a year from the time of first vaccination, compared to a control.

43. A method of increasing the amount of carbon dioxide (CO₂) emitted by a subject, preferably eructated and/or exhaled, the method comprising administering to the subject the vaccine composition of any one of embodiments 1-27.

44. The method of embodiment 43, wherein the amount of carbon dioxide (CO₂) is increased by about 1-100%, preferably by about 1-20%, compared to a control, optionally wherein the amount of CO₂ is increased by at least about !%, 5%, 10%, 15%, 20%, 25%, 30%, 35%, 40%, 45%, 50%, 55%, 60%, 65%, 70%, 75%, 80%, 85%, 90%, 95%, or 100% compared to a control.

45. The method of embodiment 43 or 44, wherein the amount of carbon dioxide (CO₂) is increased by about 3-10%, preferably by about 3-20%, compared to a control.

46. The method of any one of embodiments 43-45, wherein the amount of carbon dioxide (CO₂) is increased by (a) about 29.8 kg-about 1,490 kg within 8 weeks from the time of first vaccination, or (b) about 149 kg-about 8,940 kg within a year from the time of first vaccination, compared to a control.

47. The method of any one of embodiments 37-42 and 44-46, wherein the control is:
(a) an accepted reference;
(b) the amount of methane, CO₂-normalized methane, hydrogen, or carbon dioxide emitted by an unvaccinated subject; or
(c) the amount of methane, C02-normalized methane, hydrogen, or carbon dioxide emitted by the vaccinated subject prior to vaccination.

48. The method of any one of embodiments 28-47, wherein the subject produces an antibody against at least one methanogen.

49. The method of embodiment 48, wherein the antibody is an IgG, IgM, or an IgA, preferably an IgA or IgM.

50. The method of embodiment 48 or 49, wherein the antibody is produced in an amount sufficient to:
(a) be detectable in the digestive tract;
(b) reduce the number and/or alter the relative abundances of methanogens in the digestive tract;
(c) reduce the amount of methane, the CO₂-normalized methane, and/or hydrogen emitted by the subject; and/or
(d) increase the amount of CO₂ emitted by the subject.

51. The method of any one of embodiments 28-50, wherein the vaccine composition is administered to the subject via a route selected from intramuscular administration, intradermal administration, subcutaneous administration, and nasal administration.

52. The method of any one of embodiments 28-51, wherein the vaccine composition is administered to the subject via intramuscular administration or subcutaneous administration, preferably subcutaneous administration.

53. The method of any one of embodiments 28-52, wherein the subject is administered with at least one repeat dose of the vaccine composition of any one of embodiments 1-27.

54. The method of embodiment 53, wherein the subject is administered with at least two repeat doses of the vaccine composition.

55. The method of embodiment 53 or 54, wherein the subject is administered with the vaccine composition at least 3 times per year.

56. The method of any one of embodiments 53-55, wherein the at least one repeat dose comprises the same dose or a different dose (e.g., number or amount of cells and/or cell parts) compared to the preceding dose of the vaccine composition.

57. The method of any one of embodiments 53-56, wherein the at least one repeat dose comprises the same adjuvant or a different adjuvant compared to the preceding dose of the vaccine composition.

58. The method of any one of embodiments 53-57, wherein the subject is administered with the repeat dose of the vaccine composition after at least about 2 weeks, about 3 weeks, about 1 month, about 6 months, or about 12 months from the time the subject is administered with the preceding dose of the vaccine composition.

59. The method of any one of embodiments 53-58, wherein the subject is administered with the repeat dose of the vaccine composition no later than about 1 month, about 2 months, about 3 months, 6 months, 12 months, 18 months, or 24 months from the time the subject is administered with the preceding dose of the vaccine composition.

60. The method of any one of embodiments 53-59, wherein the subject receives the repeat dose of the vaccine after at least about 2 weeks and no more than about 18 months from the time the subject is administered with the preceding dose of the vaccine.

61. The method of any one of embodiments 53-60, wherein the subject is administered with at least two repeat doses of the vaccine composition, and the subject receives:
(a) the first repeat dose (“boost 1”) after about 2 weeks to about 4 weeks from the time the subject is administered with the initial vaccine dose (“prime”); and/or
(b) the second repeat dose (“boost 2”) after about 3 months to about 6 months from the time the subject is administered with the initial vaccine dose (“prime”).

62. The method of any one of embodiments 28-61, wherein the subject is administered with a dosage of between about 10³ cells per vaccine dose per kg of animal body weight and 10⁹ cells per vaccine dose per kg of animal body weight of the vaccine composition each time of vaccination.

63. The method of any one of embodiments 28-62, further comprising administering to the subject (a) at least one agent that reduces the level of methane and/or hydrogen produced by the subject; and/or (b) at least one agent that increases production efficiency.

64. The method of embodiment 63, wherein the at least one agent is administered to a subject concomitant with, prior to, or after the vaccination.

65. The method of embodiment 63 or 64, wherein the at least one agent is administered to a subject after the vaccination.

66. The method of any one of embodiments 63-65, wherein the at least one agent is administered to a subject daily, semiweekly, weekly, biweekly (every two weeks), or monthly.

67. The method of any one of embodiments 63-66, wherein the at least one agent is administered to a subject for a duration of at least 1 week but no more than 1 month.

68. The method of any one of embodiments 63-67, wherein the at least one agent comprises:
(a) an agent selected from the agents listed in Tables 4-8;
(b) 3-Nitrooxypropanol (3NOP), ethyl-3NOP, 2-bromoethanesulfonate (BES), 2-chloroethanesulfonate (CES), 3-bromopropanesulfonate (BPS), bromochloromethane (BCM), bromoform, bromodichloromethane, dibromochloromethane, carbon tetrachloride, trichloroacetamide, trichloroethyladipate, lumazin (2,4-pteridinedione), p-aminobenzoic acid, lovastatin, mevastatin, pravastatin, diallyl disulfide, garlic oil, saponins, tannins, flavonoids, nitrate, nitroethane, -nitro-propionate, 2-nitropropanol, 2-nitroethanol, malate, acrylate, oxaloacetate, fumarate, propionic acid, 3-butenoic acid, 2-butynoic acid, ethyl 2-butynoate, monensin, lasalocid, bovicin HC5, nisin, or any combination thereof; or
(c) monensin (Rumensin®), Optigrid® 45 (ractopamine hydrochloride), Amprolium (Corid), Bacitracin (Albac, BMD), Bambermycin (GainPro), Decoquinate (Deccox), Fenbendazole (Safe-Guard), Laidlomycin (Cattlyst), Lasalocid (Bovetec), Melengestrol Acetate (MGA), Methoprene (Altosid), Morantel (Rumatel), Poloxalene (Bloat Guard), Ractopamine (Optaflexx, Actogain), Tetraclovinphos (Rabon), or any combination thereof.

69. The method of any one of embodiments 63-68, wherein the at least one agent comprises 3NOP or ethyl-3NOP.

70. The method of embodiment 69, wherein the subject is administered with at least about 0.5 g but no more than 25 g of 3NOP per day.

71. The method of embodiment 69 or 70, wherein the subject is administered with at least about 1 g but no more than 5 g of 3NOP per day.

72. The method of any one of embodiments 69-71, wherein the subject is administered with about 2.5 g of 3NOP per day.

73. The method of any one of embodiments 69-72, wherein the subject is administered with 3NOP for a duration of at least 1 week but no more than 1 month.

74. The method of any one of embodiments 63-73, wherein the at least one agent comprises Monensin (Rumensin®).

75. The method of any one of embodiments 63-74, wherein the at least one agent is formulated in animal feed.

76. The method of any one of embodiments 63-75, wherein the at least one agent is a composition comprising one or more small molecules that reduce the production of one or more deleterious atmospheric gases and/or precursors thereof and one or more agriculturally suitable carriers.

77. The method of embodiment 76, wherein the one or more agriculturally suitable carriers comprises a solid carrier.

78. The method of embodiment 77, wherein the one or more solid carriers comprises attapulgite, kaolinite, fuller's earth, calcium carbonate, perlite, diatomaceous earth, calcium silicate, fly ash, a polysaccharide, a disaccharide, a monosaccharide, a gum, a natural or synthetic derivative thereof, or a combination thereof.

79. The method of embodiment 77, wherein the one or more solid carriers comprises attapulgite, kaolinite, fuller's earth, calcium carbonate, perlite, diatomaceous earth, calcium silicate, fly ash, a polysaccharide, a disaccharide, a monosaccharide, a gum, silica, propylene glycol, hemp protein, biochar, montmorillonite, activated charcoal, lignin, wood flour, hemp protein, pea protein, soy protein, gelatin, casein, chitosan, talc, calcium phosphate, arginine, lysine, calcium carbonate, carbon black, glutamine, betaine, bismuth phosphate, bismuth citrate, iron phosphate, or any combination thereof.

80. The method of any one of embodiments 77-79, wherein the one or more solid carriers comprises a saccharide comprising cellulose, xanthan gum, karaya gum, ethylcellulose, inositol, galactose, arabinose, lactose, lactulose, mannitol, mannose, sorbose, turanose, platinose, or a combination thereof.

81. The method of any one of embodiments 77-79, wherein the one or more solid carriers comprises a saccharide comprising cellulose, xanthan gum, karaya gum, ethylcellulose, inositol, galactose, arabinose, lactose, lactulose, mannitol, mannose, sorbose, turanose, platinose, carrageenan, cellulose acetate, hydroxypropyl cellulose, cellulose acteate phthalate, maltrodextran, dextran, inulin, corn starch, amylopectin, sodium starch glycolate, pentaerthritol, cyclodextrin, or a combination thereof.

82. The method of any one of embodiments 77-81, wherein the solid carrier comprises silica and ethylcellulose.

83. The method of embodiment 82, wherein the carrier comprises about 10% to about 50% by weight of the silica and about 50 to about 90% by weight of the ethylcelluose.

84. The method of any one of embodiments 76-83, wherein the carrier comprises silica and activated charcoal.

85. The method of any one of embodiments 76-84, wherein the carrier comprises about 10% to about 90% by weight of the silica and about 10% to about 90% by weight of the activated charcoal.

86. The method of any one of embodiments 85, wherein the carrier further comprises arginine, lysine, or both arginine and lysine.

87. The method of any one of embodiments 76-86, wherein the carrier comprises activated charcoal and ethylcellulose.

88. The method of embodiment 87, wherein the carrier comprises about 10% to about 50% by weight of the activated charcoal and about 40% to about 90% by weight of the ethylcellulose.

89. The method of embodiment 87 or 88, wherein the carrier further comprises about 1 to about 10% by weight of sodium lignosulfate or about 1 to about 10% by weight of hydroxyethyl cellulose.

90. The method of any one of embodiments 76-89, wherein the carrier comprises arginine and polycaprolactone.

91. The method of embodiment 90, wherein the carrier comprises about 10 to about 60% by weight of the arginine and about 30 to about 90% by weight of the polycaprolactone.

92. The method of any one of embodiments 76-91, wherein the carrier comprises silica and polycaprolactone.

93. The method of embodiment 92, wherein the carrier comprises about 10 to about 60% by weight of the silica and about 30 to about 90% by weight of the polycaprolactone.

94. The method of any one of embodiments 77-93, wherein the one or more solid carriers is inert.

95. The method of any one of embodiments 77-94, wherein the one or more solid carriers is water soluble.

96. The method of any one of embodiments 77-95, further comprising one or more additives with a density greater than water and/or one or more additives that reduces the rate of dissolution of the composition in water.

97. The method of embodiment 96, wherein the composition has a density of at least 1.1, preferably about 1.1 mg/mL to about 3 mg/mL, about 1.5 to about 3 mg/mL, about 1.5 to about 2.5 mg/mL, or about 1.5 to about 2 mg/mL.

98. The method of embodiment 96 or 97, wherein the one or more additives with a density greater than water comprises silica, attapulgite, kaolinite, fuller's earth, calcium carbonate, perlite, diatomaceous earth, calcium silicate, fly ash, or any combination thereof.

99. The method of any one of embodiments 96-98, wherein the one or more additives that reduces the rate of dissolution of the composition further reduces a rate of release of the one or more small molecules into water.

100. The method of embodiment 99, wherein the composition dissolves over at least about 1, 2, 3, 4, 5, 6, 7, 14, 21, 28, 35, 42, 49, 56, 63, 70, 77, 84, 91, 98, 105, 112, 119, 126, 133, 140, and/or nor more than about 2, 3, 4, 5, 6, 7, 14, 21, 28, 35, 42, 49, 56, 63, 70, 77, 84, 91, 98, 105, 112, 119, 126, 133, 140, or 147 days, about 1 to about 147 days, more preferably 7-63 days, more preferably about 7-42 days, even more preferably 14-42 days yet even more preferably 14-28 days.

101. The method of any one of embodiments 76-100, wherein about 40 to about 80% of the small molecules that reduce the production of one or more deleterious atmospheric gases and/or precursors thereof is released in water after 15 days.

102. The method of any one of embodiments 76-101, wherein the composition comprises particles having a uniform size distribution.

103. The method of any one of embodiments 76-102, wherein the composition comprises particles having a non-uniform size distribution.

104. The method of embodiment 102 or 103, wherein the particles comprise a spherical-, square-, rectangular-, capsular-, cylindrical-, conical-, ovular-, triangular-, diamond-, or disk-like shape.

105. The method of any one of embodiments 76-104, wherein the particles have a size ranging from about 1 mm to about 20 mm, about 1 to about 15 mm, about 1 to about 10 mm, about 5 to about 20 mm, about 5 to about 15 mm, or about 5 to about 10 mm.

106. The method of any one of embodiments 76-105, wherein the particles further comprise a coating.

107. The method of embodiment 106, wherein the coating comprises at least two layers.

108. The method of embodiment 106 or 107, wherein the coating is selected from cellulose acetate phlalate, ethyl cellulose, hydroxypropyl cellulose, polycaprolactone, alginate, chitosan, polyethylene glycol, cellulose acetate, triacetin, propylene glycol, n-methyl-2-pyrollidone, and any combination thereof.

109. The method of embodiment 106 or 107, wherein the coating comprises two or more polyelectrolytes.

110. The method of embodiment 109, wherein the polyelectrolytes comprise polystyrene sulfonate, polyethyleneimine, sodium lignosulfate, polyglutamic acid and poly-L-lysine, poly-L-arginine, polyallylamine hydrochloride, polyacrylic acid, or any combination thereof.

111. The method of embodiment 109 or 110, wherein the polyelectrolytes comprise polyallylamine hydrochloride and sodium lignosulfate.

112. The method of embodiment 109 or 110, wherein the polyelectrolytes comprise polyallylamine hydrochloride and polystyrene sulfonate.

113. The method of embodiment 109 or 110, wherein the polyelectrolytes comprise sodium lignosulfate and one of polyglutamic acid and poly-L-lysine, or poly-L-arginine, and sodium lignosulfate.

114. The method of embodiment 109 or 110, wherein the polyelectrolytes comprise polystyrene sulfonate and one of polyglutamic acid and poly-L-lysine, or poly-L-arginine.

115. The method of any one of embodiments 109-114, wherein the two or more polyelectrolytes are crosslinked.

116. The method of any one of embodiments 76-115, wherein the one or more small molecules comprise a molecule that interferes with the uptake and/or conversion of acetate, H₂, CO₂, methanol, monomethylamine, dimethylamine, trimethylamine, nitric oxide, or a combination thereof.

117. The method of any one of embodiments 76-116, wherein the one or more small molecules that reduce the production of one or more deleterious atmospheric gases and/or precursors interact with an enzyme selected from the group consisting of 3-(methylthio)propanoate:coenzyme M methyltransferase, acetate kinase, acetyl-CoA decarbonylase, acetyl-CoA decarbonylase/synthase complex α₂ε₂, acetyl-CoA decarbonylase/synthase complex β, acetyl-CoA decarbonylase/synthase complex γδ, acetyl-CoA synthase, carbon monoxide dehydrogenase, carbonic anhydrase, Co-methyltransferase, coenzyme M reductase, cyclohydrolase, dehydrogenase, dimethylamine—[corrinoid protein] Co-methyltransferase, F₄₂₀-dependent methylene-H₄MPT reductase, F₄₂₀-dependent methylene-H₄SPT dehydrogenase, formylmethanofuran dehydrogenase, formylmethanofuran:H₄MPT formyltransferase, formylmethanofuran:H₄SPT formyltransferase, formyltransferase, H₂-forming methylene-H₄MPT dehydrogenase, methanol-5-hydroxybenzimidazolylcobamide Co-methyltransferase, methenyl-H₄MPT cyclohydrolase, methyl-coenzyme M reductase, methyl-H₄SPT:CoM methyltransferase, methylated [methylamine-specific corrinoid protein]:coenzyme M methyltransferase, methylcobamide:CoM methyltransferase, methylthiol:coenzyme M methyltransferase, methyltransferase, MtaC protein:coenzyme M methyltransferase, phosphotransacetylase, tetrahydromethanopterin S-methyltransferase, tetramethylammonium methyltransferase, trimethylamine—corrinoid protein Co-methyltransferase, and any combination thereof.

118. The method of embodiment 117, wherein the one or more small molecules that reduce the production of one or more deleterious atmospheric gases and/or precursors interact with methyl-coenzyme M reductase (MCR).

119. The method of any one of embodiments 76-118, wherein the one or more small molecules that reduce the production of one or more deleterious atmospheric gases and/or precursors comprise a compound having the formula R¹—[CH₂]ₙ—ONO₂ wherein
n is an integer from 1 to 15;
R¹ is selected from the group consisting of H, C₁-C₆alkyl, phenyl, —OH, —NH₂, —CN,
—COOH, —O(C═O)R³, —NHC(═O)R³, SO₂NHR³, or —ONO₂, —SH and R³ is C₁-C₆alkyl, phenyl, pyridyl;
with the proviso that when n is >3 the hydrocarbon chain may be interrupted by —O— or —NH—.

120. The method of embodiment 119, wherein the one or more small molecules that reduce the production of one or more deleterious atmospheric gases and/or precursors is selected from 3-nitrooxypropanol, 9-nitrooxynonanol, 5-nitrooxy pentanoic acid, 6-nitrooxy hexanoic acid, bis(2-hydroxyethyl)amine dinitrate, 1,4-bis-nitrooxybutane, 1,5-bis-nitrooxypentane, and any combination thereof.

121. The method of embodiment 119, wherein the one or more small molecules that reduce the production of one or more deleterious atmospheric gases and/or precursors comprises 3-nitrooxypropanol (3NOP).

122. The method of any one of embodiments 76-121, wherein the composition comprises about 1 to about 25% by weight of the small molecule, about 5 to about 20% by weight of the small molecule, or about 5 to about 15% by weight of the small molecule.

123. The method of any one of embodiments 76-122, wherein the composition comprises a plurality of populations of particles, wherein each population or particles comprises a different formulation, a different shape, and/or a different size distribution.

124. The method of embodiment 123, wherein the plurality of populations of particles comprises a first population and a second population.

125. The method of embodiment 124, wherein the population of granular particles further comprises at least 1, 2, 3, 4, 5, 5, 6, 8, or 9 and/or no more than 4, 5, 6, 7, 8, 9, or 10 additional populations, for example 3-10 additional populations, preferably 3-7 additional populations, more preferably 3-5 additional populations.

126. The method of embodiment 124 or 125, wherein the first population comprises an immediate release formulation.

127. The method of any one of embodiments 124-126, wherein the second population comprises a delayed release formulation.

128. The method of any one of embodiments 125-127, wherein each additional population comprises a delayed release formulation, wherein each population dissolves and/or releases the one or more small molecules that reduce the production of one or more deleterious atmospheric gases and/or precursors at a different time than each of the other populations.

129. An antibody produced by the method of any one of embodiments 28-128, or a fragment thereof.

130. The antibody of embodiment 129, wherein the antibody is a monoclonal antibody.

131. The antibody of embodiment 129 or 130, wherein the antibody is an IgM, an IgG or an IgA, preferably an IgA or an IgM.

132. The antibody of any one of embodiments 129-131, wherein the antibody is lyophilized.

133. The antibody of any one of embodiments 129-132, wherein the antibody is in a pharmaceutical composition comprising at least one excipient and/or carrier.

134. Milk and/or a derivative thereof produced by the subject of any one of embodiments 28-128.

135. The milk and/or a derivative thereof of embodiment 134, wherein the milk and/or derivatives thereof comprises an antibody that binds at least one methanogen.

136. The milk and/or a derivative thereof of embodiment 134 or 135, wherein the milk and/or derivatives thereof is pasteurized and/or homogenized.

137. The milk and/or a derivative thereof of any one of embodiments 134-136, wherein the milk and/or derivatives thereof is lyophilized or evaporated to form dry milk powder (e.g., boiling at low pressure at low temperature).

138. The milk and/or a derivative thereof of any one of embodiments 134-137, further comprising at least one agent that reduces methane and/or hydrogen production in a subject, optionally wherein the at least one agent is selected from the agents in Tables 4-8.

139. An animal feed comprising:
(a) the antibody of any one of embodiments 129-133;
(b) at least one agent that reduces methane and/or hydrogen production in a subject, optionally wherein the at least one agent is selected from the agents in Tables 4-8;
(c) the milk of any one of embodiments 134-138; or
(d) any combination of two or more of (a)-(c).

140. The animal feed of embodiment 139, wherein the animal feed is liquid (e.g., drinking water, milk) or solid (e.g., fodder).

141. The animal feed of embodiment 139 or 140, wherein the animal feed comprises fat and/or fatty acid, optionally wherein the animal feed comprises fat and/or fatty acid that is at least about 1%, 2%, 3%, 4%, 5%, or 6% of the diet.

142. A method of reducing methane and/or hydrogen production in a subject, the method comprising orally administering to and/or feeding the subject the antibody of any one of embodiments 129-133, the milk and/or a derivative thereof of any one of embodiments 134-138, the animal feed of any one of embodiments 139-141, or any combination of two or more thereof.

143. The method of embodiment 142, further comprising administering at least one agent that reduces methane and/or hydrogen production in a subject, optionally wherein the at least one agent is selected from the agents in Tables 4-8.

144. The method of embodiment 142 or 143, further comprising administering the subject with the at least one vaccine composition of any one of embodiments 1-27, optionally according to the method of any one of embodiments 28-128.

145. The method of any one of embodiments 28-128 and 142-144, wherein the subject is a mammal, a human, or a ruminant.

146. The method of embodiment 145, wherein the ruminant is selected from a cow, a bull, a bison, a yak, a buffalo, an antelope, a goat, a sheep, a deer, a giraffe, a caribou, a gazelle, a macropod, a llama, a camel, and an alpaca.

147. The method of any one of embodiments 28-128 and 142-146, wherein the subject is cattle.

148. The method of embodiment 147, wherein the cattle is selected from a pregnant cow, heifer, bull, and steer.

149. The method of any one of embodiments 28-128 and 142-148, wherein the subject is an adult.

150. The method of embodiment 149, wherein the subject is adult cattle selected from:
(a) a cow that is about 6-8 months to about 2-7 years old;
(a) a beef adult that is about 1.5 years to about 2.5 years old; and
(b) a dairy adult that is about 4 years to 7 years old.

151. The method of any one of embodiments 28-128 and 142-148, wherein the subject is a young subject (e.g., before weaning or below 2 years of age).

152. The method of embodiment 151, wherein the subject is young cattle selected from:
(a) a newborn calf and a pre-weaned calf that is about 0 month to about 3 months old; and
(b) a weaned cow that is about 3 months to 8 months old.

153. The method of any one of embodiments 28-128 and 142-152, wherein the subject is a pregnant female subject.

154. The method of any one of embodiments 28-128 and 142-153, wherein the subject is an offspring (e.g., calf) of the vaccinated female subject that received the milk comprising an antibody that binds at least one methanogen.

155. The method of any one of embodiments 28-128 and 142-154, wherein the vaccine is administered to a subject as a part of a pre-existing vaccination program to which the subject is subject (e.g., Table 9).

156. The method of any one of embodiments 28-128 and 142-155, wherein the vaccine is administered to a subject when the subject is subject to or receives at least one other vaccine, wherein the at least one other vaccine is against infectious bovine rhinotracheitis (IBR), bovine virus diarrhea (BVD), parainfluenza-3 (PI3), bovine respiratory syncytial virus (BRSV), clostridia, E. Coli mastitis, leptospirosis, mannheimia hemolytica, brucella, vibriosis, campylobacter, trichomonas, trichomoniasis, rotavirus, coronavirus, and/or respiratory disease.

157. The method of any one of embodiments 28-128 and 142-156, wherein the vaccine is administered to a subject when the subject changes in hands and/or a changes in environment.

158. The method of any one of embodiments 28-128 and 142-157, wherein the vaccine reduces methane and/or hydrogen production in the lower intestinal track (lower bowel) or the rumen of the subject.

159. The method of embodiment 158, wherein the method results in at least about 1%, 2%, 3%, 4%, 5%, 6%, 7%, 8%, 9%, 10%, 11%, 12%, 13%, 14%, 15%, 16%, 17%, 18%, 19%, or 20% reduction in the level of methane and/or produced by the subject, optionally wherein the reduction in the level of methane and/or hydrogen is compared to an untreated subject.

160. A kit comprising the vaccine composition of any one of embodiments 1-27.

161. The kit of embodiment 160, wherein the vaccine composition comprises no more than one methanogen.

162. The kit of embodiment 160, wherein the vaccine composition comprises at least two methanogens.

163. The kit of embodiment 160, wherein the kit comprises at least two vaccine compositions comprising same or different methanogens.

164. The kit of embodiment 163, wherein the at least two vaccine compositions comprise different methanogens, and the different methanogens are in separate containers.

165. The kit of any one of embodiments 160-164, wherein the kit comprises about or at least about 1, 2, 3, 4, 5, 10, 20, 50, 100, 250, 500, 1000, 1500, 2000, 2500, or 3000 doses of the vaccine composition.

166. The kit of embodiment 165, wherein
(a) all doses are in a single container;
(b) each dose is in an individual container; or
(c) about or at least about 2, 3, 4, 5, 10, 25, 50, or 100 doses are pooled in a single container.

167. The kit of any one of embodiments 160-166, further comprising at least one adjuvant.

168. The kit of embodiment 167, wherein the vaccine composition and the at least one adjuvant are in separate containers.

169. The kit of embodiment 167 or 168, wherein the kit comprises at least about 1, 2, 3, 4, 5, 10, 20, 50, 100, 250, 500, 1000, 1500, 2000, 2500, or 3000 doses; and
(a) all doses are in a single container, and the at least one adjuvant for all doses are in a separate single container;
(b) each dose is in an individual container, and the at least one adjuvant for all doses are in a separate single container;
(c) each dose is in an individual container, and the at least one adjuvant for each dose is in a separate individual container; or
(d) about or at least about 2, 3, 4, 5, 10, 25, 50, or 100 doses are pooled in a single container, and the pooled amount of at least one adjuvant for the pooled doses are in a separate single container.

170. The kit of any one of embodiments 167-169, wherein the kit comprises at least two adjuvants that are different.

171. A system comprising a growth chamber (e.g., bioreactor), wherein the growth chamber comprises a growth medium under an anaerobic atmosphere comprising less than 80% H₂, optionally wherein the system further comprises at least one hydrogenotroph.

172. The system of embodiment 171, wherein the anaerobic atmosphere comprises less than 50% H₂. 173. The system of embodiment 171 or 172, wherein the anaerobic atmosphere comprises at least 0.5% but no more than 20% H₂, preferably at least 0.5% but no more than 4% H₂.

174. The system of any one of embodiments 171-173, wherein the anaerobic atmosphere comprises at least 1% but no more than 4% H₂, preferably at least 2% but no more than 4% H₂.

175. The system of any one of embodiments 171-174, wherein the anaerobic atmosphere comprises CO₂.

176. The system of embodiment 175, wherein the anaerobic atmosphere comprises at least about 1 part of CO₂ for every 4 parts of H₂.

177. The system of any one of embodiments 171-176, wherein the anaerobic atmosphere comprises inert gas.

178. The system of embodiment 177, wherein the inert gas is N₂.

179. The system of any one of embodiments 171-178, wherein the anaerobic atmosphere consists essentially of H₂, CO₂, and inert gas, preferably wherein the inert gas is N₂.

180. The system of any one of embodiments 171-179, wherein the at least one hydrogenotroph is under the pressure of less than about 180 kPa, about 150 kPa, about 125 kPa, about 120 kPa, about 110 kPa, or about 105 kPa.

181. The system of any one of embodiments 171-180, wherein the growth chamber is at least about 0.1 L, 0.5 L, 1 L, 5 L, 10 L, 50 L, 100 L, 250 L, 500 L, or 1000 L in volume.

182. The system of any one of embodiments 171-181, wherein a volume of the growth medium is at least about 0.1 L, 0.5 L, 1 L, 5 L, 10 L, 50 L, 100 L, 250 L, 500 L, or 1000 L.

183. The system of any one of embodiments 171-182, wherein the growth medium comprises
(a) at least one component listed in Table D or Table E;
(b) at least 10, 20, 30, or 40 components listed in Table D;
(c) at least 10, 20, 30, 40, or 50 components listed in Table E;
(d) all components listed in Table D or Table E;
(e) medium according to Table D or Table E (e.g., having an exemplary concentration of at least one component, or having a preferred range of concentrations (or a value or a sub-range of concentrations therein) of at least one component);
(f) BY medium; or
(g) SD0001 medium.

184. The system of any one of embodiments 171-183, wherein the growth medium does not comprise a non-hydrogenotrophic additive (e.g., acetate, methanol, ethanol).

185. The system of any one of embodiments 171-184, wherein the system comprises at least one auxiliary instrument.

186. The system of embodiment 185, wherein the at least one auxiliary instrument is selected from gas supply, gas inlet, gas outlet, gas mixer upstream of the growth chamber, temperature control, gas flow control, H₂ sensor, CO₂ sensor, CH₄ sensor, spectrophotometer, turbimeter, fluorometer, pH sensor, oxygen sensor, liquid flow control, pressure sensor, foam sensor, H₂S sensor, scale, flow meter, camera, redox sensor, contamination sensor, motor, magnetic stirrer, shaker, agitator, impeller, sparger, sampling port (e.g., cells, gas, media), methane conversion system, and any one or more of the instruments listed in Table C.

187. The system of any one of embodiments 171-186, wherein the system further comprises at least one hydrogenotroph.

188. The system of embodiment 187, wherein the at least one hydrogenotroph comprises:
(a) at least one hydrogenotroph selected from the hydrogenotrophs in Table A and/or Table B;
(b) at least one methanogen;
(c) at least one methanogen, wherein the at least one methanogen comprises a methanogen of a genus Methanobrevibacter; and/or
(d) at least one methanogen, wherein the at least one methanogen comprises Methanobrevibacter ruminantium and/or Methanobrevibacter gottschalkii.

189. A method of growing at least one hydrogenotroph, the method comprising incubating the at least one hydrogenotroph in the system of any one of embodiments 171-188.

190. A method of growing at least one hydrogenotroph, the method comprising the steps of:
(a) purging a growth chamber (e.g., bioreactor) with an anaerobic gaseous mixture comprising less than 80% H₂, optionally less than 50% H₂;
(b) incubating the at least one hydrogenotroph in growth medium in the growth chamber purged in step (a).

191. The method of embodiment 190, wherein the anaerobic gaseous mixture comprises at least 0.5% but no more than 20% H₂, preferably at least 0.5% but no more than 4% H₂.

192. The method of embodiment 190 or 191, wherein the anaerobic gaseous mixture comprises at least 1% but no more than 4% H₂, preferably at least 2% but no more than 4% H₂.

193. The method of any one of embodiments 190-192, wherein the anaerobic gaseous mixture comprises CO₂.

194. The method of embodiment 193, wherein the anaerobic gaseous mixture comprises at least about 1 part of CO₂ for every 4 parts of H₂.

195. The method of any one of embodiments 190-194, wherein the anaerobic gaseous mixture comprises inert gas.

196. The method of embodiment 195, wherein the inert gas is N₂.

197. The method of any one of embodiments 190-196, wherein the anaerobic gaseous mixture consists essentially of H₂, CO₂, and inert gas, preferably wherein the inert gas is N₂.

198. The method of any one of embodiments 190-197, wherein the gases of the anaerobic gaseous mixture are pre-mixed before entering the growth chamber.

199. The method of any one of embodiments 190-197, wherein the gases of the anaerobic gaseous mixture are mixed in a gas mixer upstream of the growth chamber.

200. The method of any one of embodiments 190-197, wherein the gases of the anaerobic gaseous mixture enter the growth chamber individually, and the gases are mixed in the growth chamber.

201. The method of any one of embodiments 190-200, wherein the anaerobic gaseous mixture is sparged into the growth media.

202. The method of any one of embodiments 190-201, wherein the anaerobic gaseous mixture is supplied continuously during the culturing of the at least one hydrogenotroph.

203. The method of any one of embodiments 190-202, wherein the gases of the anaerobic gaseous mixture is supplied from a single gas tank.

204. The method of any one of embodiments 190-202, wherein the gases of the anaerobic gaseous mixture is supplied from at least two gas cylinders, each of which comprises a different gas or gas mixture.

205. The method of any one of embodiments 190-204, wherein the at least one hydrogenotroph is under the pressure of less than about 180 kPa, about 150 kPa, about 125 kPa, about 120 kPa, about 110 kPa, or about 105 kPa.

206. The method of any one of embodiments 190-205, wherein the pressure of the supplied gas at the gas inlet to the growth chamber is at least about 102 kPa but no more than about 125 kPa.

207. The method of any one of embodiments 190-206, wherein the growth chamber is at least about 0.1 L, 0.5 L, 1 L, 5 L, 10 L, 50 L, 100 L, 250 L, 500 L, or 1000 L in volume.

208. The method of any one of embodiments 190-207, wherein a volume of the growth medium is at least about 0.1 L, 0.5 L, 1 L, 5 L, 10 L, 50 L, 100 L, 250 L, 500 L, or 1000 L.

209. The method of any one of embodiments 190-208, wherein the growth medium comprises
(a) at least one component listed in Table D or Table E;
(b) at least 10, 20, 30, or 40 components listed in Table D;
(c) at least 10, 20, 30, 40, or 50 components listed in Table E; or
(d) all components listed in Table D or Table E;
(e) medium according to Table D or Table E (e.g., having an exemplary concentration of at least one component, or having a preferred range of concentrations (or a value or a sub-range of concentrations therein) of at least one component);
(f) BY medium; or
(g) SD0001 medium.

210. The method of any one of embodiments 190-209, wherein the growth medium does not comprise a non-hydrogenotrophic additive (e.g., acetate, methanol, ethanol).

211. The method of any one of embodiments 190-210, wherein the growth chamber further comprises at least one auxiliary instrument.

212. The method of embodiment 211, wherein the at least one auxiliary instrument is selected from gas supply, gas inlet, gas outlet, gas mixer upstream of the growth chamber, temperature control, gas flow control, H₂ sensor, CO₂ sensor, CH₄ sensor, spectrophotometer, turbimeter, fluorometer, pH sensor, oxygen sensor, liquid flow control, pressure sensor, foam sensor, H₂S sensor, scale, flow meter, camera, redox sensor, contamination sensor, motor, magnetic stirrer, shaker, agitator, impeller, sparger, sampling port (e.g., cells, gas, media), methane conversion system, and any one of the instruments listed in Table C.

213. The method of embodiment 211 or 212, wherein the at least one auxiliary instrument comprises a methane conversion system, wherein the gases from the growth medium are circulated back into the growth chamber.

214. The method of any one of embodiments 190-213, wherein the at least one hydrogenotroph comprises at least one hydrogenotroph selected from the hydrogenotrophs in Table A and/or Table B.

215. The method of embodiment 214, wherein the at least one hydrogenotroph comprises:
(a) at least one methanogen;
(b) at least one methanogen, wherein the at least one methanogen is of a genus Methanobrevibacter; and/or
(c) at least one methanogen, wherein the at least one methanogen comprises Methanobrevibacter ruminantium and/or Methanobrevibacter gottschalkii.

216. A culture medium comprising
(a) at least one component listed in Table D or Table E;
(b) at least 10, 20, 30, or 40 components listed in Table D;
(c) at least 10, 20, 30, 40, or 50 components listed in Table E;
(d) all components listed in Table D or Table E; or
(e) medium according to Table D or Table E (e.g., having a preferred range of concentrations (or a value or a sub-range therein) of at least one component).

217. A method of reducing CH₄ emissions in a ruminant comprising administering to the ruminant a vaccine composition comprising a cell and/or cell part of at least one methanogen, wherein the CH₄ emissions are reduced by at least about 10, 11, 12, 13, 14, 15, 16, 17, 18, 19, or 20% as compared to an untreated control ruminant.

218. A method of reducing H₂ emissions in a ruminant comprising administering to the ruminant a vaccine composition comprising a cell and/or cell part of at least one methanogen, wherein the H₂ emissions are reduced by at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50% as compared to an untreated control ruminant.

219. A method of increasing the productivity of a ruminant comprising administering to the ruminant a vaccine composition comprising a cell and/or cell part of at least one methanogen, wherein the productivity is increased by at least about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15% as compared to an untreated control ruminant.

220. The method of any one of embodiments 217-219, wherein the vaccine composition comprises the vaccine composition of any one of embodiments 1-27.

221. The method of any one of embodiments 217-220, wherein the ruminant is cattle.

222. An animal injected subcutaneously with the vaccine composition of any one of embodiments 1-27, wherein the vaccine composition comprises about 10⁷, 10⁸, 10⁹, 10¹⁰, 10¹¹, 10¹², 10¹³, or 10¹⁴ methanogen cells, for example about 10⁷-about 10¹⁴ methanogen cells. 223. A method of producing a low carbon animal product, the method comprising:
(a) administering to an animal (e.g., a mammal, a ruminant) a vaccine composition of any one of embodiments 1-27;
(b) determining an amount of emissions of at least one greenhouse gas (e.g., CO₂, CH₄, N₂O, and/or H₂) from the animal of (a) following administration until animal product harvesting;
(c) determining a first carbon intensity as a ratio of a first amount of emissions from the vaccinated animal in (b) and the amount of harvested animal product;
(d) determining a second carbon intensity as a ratio of a second amount of carbon emissions from an untreated animal and the same amount of harvested animal product; and
(e) determining the difference between the first carbon intensity and the second carbon intensity.

224. The method of embodiment 223, wherein determining the amount of emissions comprises measuring the emissions using a GreenFeed system.

225. The method of embodiment 223, wherein the animal product is selected from the group consisting of meat, milk, and wool.

226. The method of embodiment 223, further comprising administering to the animal at least one agent that reduces methane production.

227. The method of embodiment 223, further comprising certifying the animal product as a low carbon intensity product based on the determined difference between the first carbon intensity and the second carbon intensity.

### Examples

### Example 1: Preparation of Compositions Comprising at Least One Methanogen of a Genus Methanobrevibacter

This example demonstrates a method to formulate an exemplary vaccine composition comprising at least one methanogen, which is of a genus Methanobrevibacter. Production of the vaccine components and formulation of the vaccines is described herein.

Briefly, M. ruminantium and M. gottschalkii, two representative species of the genus Methanobrevibacter, were grown in a heterologous growth medium, hereinafter referred to as BY medium (formulated as described in Table 11A) in a H₂-rich (80% H₂, 20% CO₂), anaerobic environment prior to fixation. Cells were harvested, exchanged and concentrated into storage buffer, and frozen for later use as described below.

BY medium recipes are listed in Tables 11A-G below. Briefly, at the time of BY medium preparation, rumen fluid aliquots (Bar Diamond, Parma, ID; stored at −20° C. in conical centrifuge tubes) were thawed at room temperature. Thawed rumen fluid was then centrifuged at 15,000×g for 60 min. to clarify. Clarified rumen fluid was then transferred via serological pipette to a fresh vessel without disturbing the pellet. All additional components of BY medium, except for cysteine and BY medium vitamins, were combined into the vessel and autoclaved for 25-60 min. at 121° C. to sterilize. Once cooled, the vessel was transferred into a glove box, and left open in the glove box for at least 2 hrs to degas. Finally, the cysteine and BY medium vitamins were added.

TABLE 11A
Exemplary recipe for BY medium

| Component | Amount | |
|---|---|---|
| BY Medium Solution A (see Table 11B) | 170 | mL/L |
| BY Medium Solution B (see Table 11C) | 170 | mL/L |
| Sodium hydrogen carbonate (VWR, Radnor, PA) | 5 | g/L |
| 0.1% resazurin (w/v) (see Table11G) | 400 | μL/L |
| Yeast extract (VWR, Radnor, PA) | 1 | g/L |
| Rumen fluid (Bar Diamond, Parma, ID) | 300 | mL/L |
| BY Medium Trace Elements (see Table 11F) | 10 | mL/L |
| Cysteine hydrochloride (Fisher Scientific, Waltham, MA) | 500 | mg/L |
| BY Medium Vitamins (see Table 11D) | 11.11 | mL/L |

TABLE 11B
Exemplary recipe for BY medium solution A

| Component | Amount | |
|---|---|---|
| Sodium chloride (VWR, Radnor, PA) | 6 | g/L |
| Potassium dihydrogen phosphate (VWR, Radnor, PA) | 3 | g/L |
| Calcium chloride dihydrate (VWR, Radnor, PA) | 0.79 | g/L |
| Magnesium sulfate heptahydrate (VWR, Radnor, PA) | 1.2 | g/L |
| Ammonium sulfate (VWR, Radnor, PA) | 1.5 | g/L |

TABLE 11C
Exemplary recipe for BY medium solution B

| Component | Amount |
|---|---|
| Dipotassium hydrogen phosphate (VWR, Radnor, PA) | 6 g/L |

TABLE 11D
Exemplary recipe for BY medium vitamins

| Component | Amount | |
|---|---|---|
| Pyridoxine hydrochloride (VWR, Radnor, PA) | 9.52 | mg/L |
| Ascorbic acid (VWR, Radnor, PA) | 4.76 | mg/L |
| Calcium pantothenate (VWR, Radnor, PA) | 4.76 | mg/L |
| Lipoic acid (Fisher Scientific, Waltham, MA) | 4.76 | mg/L |
| Nicotinamide (VWR, Radnor, PA) | 4.76 | mg/L |
| Nicotinic acid (Niacin) | 4.76 | mg/L |
| 4-Aminobenzoic acid (VWR, Radnor, PA) | 4.76 | mg/L |
| Pyridoxal hydrochloride (VWR, Radnor, PA) | 4.76 | mg/L |
| Riboflavin (vitamin B2) (VWR, Radnor, PA) | 4.76 | mg/L |
| Thiamine hydrochloride (VWR, Radnor, PA) | 4.76 | mg/L |
| D-(+)-Biotin (VWR, Radnor, PA) | 1.9 | mg/L |
| Folic acid (VWR, Radnor, PA) | 1.9 | mg/L |
| Vitamin B12 (cyanocobalamin) (VWR, Radnor, PA) | 0.095 | mg/L |
| BY Medium Reducing Agent (see Table 11E) | 10.5 | mL/L |

TABLE 11E
Exemplary recipe for BY medium reducing agent

| Component | Amount |
|---|---|
| Cysteine hydrochloride (Fisher Scientific, | 12.195 g/L |
| Waltham, MA) | |
| Sodium sulfide nonahydrate (Fisher Scientific, | 12.195 g/L |
| Waltham, MA) | |

TABLE 11F
Exemplary recipe for BY medium trace elements

| Component | Amount | |
|---|---|---|
| Nitrilotriacetic acid (VWR, Radnor, PA) | 1.5 | g/L |
| Magnesium sulfate heptahydrate (VWR, Radnor, PA) | 5.43 | g/L |
| Manganese(II) sulfate monohydrate (VWR, Radnor, PA) | 0.5 | g/L |
| Sodium chloride (VWR, Radnor, PA) | 1 | g/L |
| Iron(II) sulfate heptahydrate (VWR, Radnor, PA) | 0.1 | g/L |
| Cobalt (II) chloride hexahydrate (VWR, Radnor, PA) | 0.1 | g/L |
| Calcium chloride dihydrate (VWR, Radnor, PA) | 1.32 | g/L |
| Zinc sulfate heptahydrate (VWR, Radnor, PA) | 0.1 | g/L |
| Copper (II) sulfate pentahydrate (VWR, Radnor, PA) | 10 | mg/L |
| Aluminium potassium sulfate dodecahydrate | 10 | mg/L |
| (VWR, Radnor, PA) | | |
| Boric acid (VWR, Radnor, PA) | 10 | mg/L |
| Sodium molybdate(VI) dihydrate (VWR, Radnor, PA) | 10 | mg/L |
| Nickel (II) sulfate hexahydrate (VWR, Radnor, PA) | 30 | mg/L |
| Sodium selenite (VWR, Radnor, PA) | 20 | mg/L |
| Sodium tungstate dihydrate (VWR, Radnor, PA) | 20 | mg/L |

TABLE 11G
Exemplary recipe for 0.1% resazurin (weight/volume)

| Component | Amount |
|---|---|
| Resazurin, sodium salt (Fisher Scientific, Waltham, MA) | 1 g/L |

### Exemplary Growth Protocol for M. Ruminantium

Briefly, 1,300 mL of heterologous BY medium (prepared as described above) was inoculated with 130 mL of actively growing M. ruminantium (Cat. No. 1093, DSMZ, Braunschweig, Germany) seed culture, e.g., inoculum, in an anaerobic environment. The seed was well mixed into the BY medium. 350 mL of the inoculated medium was then transferred to four separate culture vessels, e.g., 1 L Duran pressure bottles affixed with GL45 caps.

Next, the four culture vessels were affixed with rubber stopper-equipped septum caps, removed from the anaerobic chamber, and pressurized to 12 psi with an 80% H₂, 20% CO₂ gas mix delivered through the bottle septum using a needle. The four culture vessels were then incubated for approximately 10 days at 38° C. in an incubator equipped with an orbital shaking platform. Periodically, the headspace of each bottle was exchanged with fresh 80% H₂, 20% CO₂ gas mix and re-pressurized to 12 psi.

After the incubation period, the M. ruminantium cells and/or cell parts were harvested and processed into 5% DMSO stocks for cryopreservation. Briefly, the four culture vessels were combined and centrifuged at 3,200×g for 10 min. in 500 mL centrifuge bottles to pellet the M. ruminantium cells and/or cell parts. The resulting pellets were then combined in 200 mL of PBS and divided across four 50 mL Falcon tubes which were spun down at 6,000×g for 20 min. to wash away the BY medium. The resulting pellets were then combined again in 100 mL of PBS across two 50 mL Falcon tubes and centrifuged for 20 min. at 6,000×g. The supernatant was then poured off, and each pellet was resuspended in 25 mL of PBS. Finally, DMSO was added to each 50 mL Falcon tube to a final concentration of 5% to cryoprotect the M. ruminantium cells and/or cell parts, and tubes were stored at −80° C.

### Exemplary Growth Protocol for M. Gottschalkii

Briefly, growth of 3.3 L cultures of M. gottschalkii (Cat. No. 11977, DSMZ, Braunschweig, Germany) in heterologous BY medium (prepared as described above) was carried out in culture vessels, e.g., 10 L Torpedo Kegs (D794,899, Brewmaster Wholesale, Pittsburgh, CA) under anaerobic conditions. The BY medium was inoculated with actively growing M. gottschalkii seed culture, e.g., inoculum, aseptically while simultaneously preserving the anaerobic environment of both the M. gottschalkii inoculum and the BY medium. Following inoculation, the culture vessels were affixed to rotating platforms in a fume hood, outfitted with a temperature-controlled blanket set to 38° C., and incubated for approximately 2 weeks. Upon inoculation and periodically throughout the incubation period, the headspace of the culture vessels was exchanged by pushing an oxygen-free 80% H₂, 20% CO₂ gas mix through a gas input conduit while allowing headspace turnover outlet conduit, e.g., a pressure relief valve, (vented into the fume hood). After this turnover the culture vessels were pressurized to 60 psi with the same 80% H₂, 20% CO₂ gas mix.

At the end of the incubation period, the culture vessels were opened to the aerobic environment and the M. gottschalkii cells and/or cell parts were processed into a 5% DMSO stocks for cryopreservation. Briefly, the cultures were transferred to 500 mL centrifuge tubes and centrifuged at 3,200×g for 10 min. to pellet the M. gottschalkii cells and/or cell parts. The resulting pellets were then combined and resuspended in 200 mL of PBS, and divided across four 50 mL Falcon tubes. Then, the four 50 mL Falcon tubes were centrifuged at 6,000×g for 20 min. to wash away the BY medium. Next, the resulting pellets were combined and resuspended in 100 mL of PBS across two 50 mL Falcon tubes. Then, the two 50 mL Falcon tubes were centrifuged for 20 min. at 6,000×g, and each resultant pellet was resuspended in 25 mL PBS. Finally, DMSO was added to each 50 mL Falcon tube to a final concentration of 5% to cryoprotect the M. gottschalkii cells and/or cell parts, and tubes were stored at −80° C.

### Fixation of Methanogens

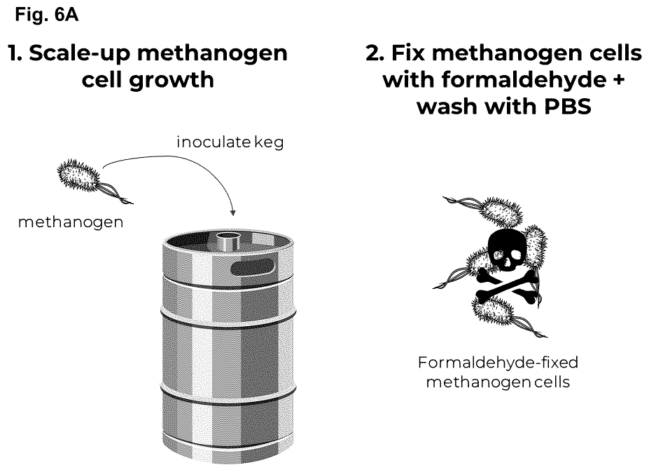

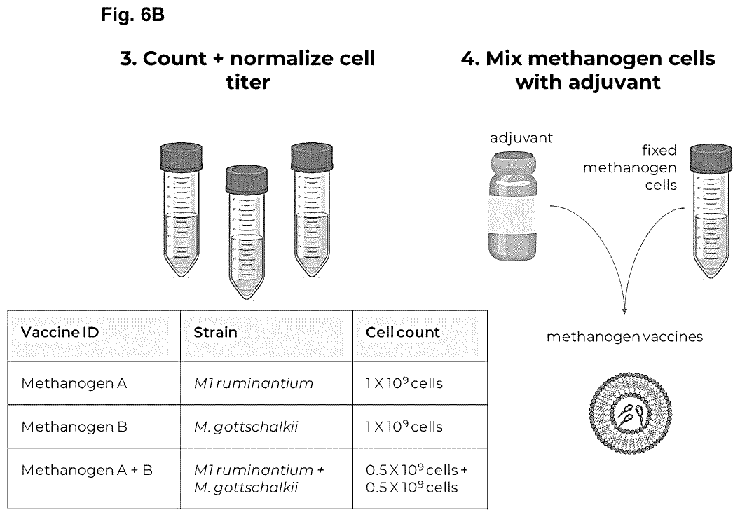

Fixed cells and/or cell parts of at least one methanogen of a genus Methanobrevibacter, e.g., Methanobrevibacter ruminantium and/or Methanobrevibacter gottschalkii, were prepared following procedures described by Wright et al. (2004) Vaccine 22(29-30):3976-85 and Wedlock et al. (2010) N Z Vet J 58(1):29-36, each of which is incorporated herein by reference. Exemplary reagents and materials for the fixation and storage of Methanobrevibacter cells and/or cell parts are described below in Table 12 and FIG. 6A.

Specifically, two 50 mL Falcon tubes of frozen, concentrated M. ruminantium and/or M. gottschalkii cells and/or cell parts were thawed at room temperature. The 50 mL Falcon tubes were centrifuged at 5° C. 14,000×g for 10 min., and the supernatant was then discarded. Each resultant pellet was then resuspended in 35 mL of fixative (the fixative comprising 25.45 mL of 10% formalin combined with 9.54 mL of DPBS). Next, the suspension of cells and/or cell parts in fixative were incubated for 1 hr at room temperature in the fume hood. Then, to pellet the fixed cells and/or cell parts, the suspension was centrifuged at 5° C. 10,000×g for 10 min. and the supernatant discarded. Next, the resultant pellet was resuspended in 35 mL of DPBS, then centrifuged at 5° C. 7,000×g for 10 min. and the supernatant was discarded; this resuspension and centrifugation step was repeated two additional times to wash away any remaining volume of fixative. Then, for each Methanobrevibacter strain, the resultant pellets were resuspended and combined in 100 mL of DPBS in the 150 mL sterile bottles from the Fisherbrand™ disposable PES filter units. The density of the Methanobrevibacter cells was determined using flow cytometry, using the intrinsic fluorescence of cofactor F₄₂₀, a protein expressed by methanogens, at 420 nm to detect and record the Methanobrevibacter cells as single particles identified by the instrument, e.g., events. The number of events per mL of the analyzed solution, as measured by the flow cytometry instrument, corresponds to the reported number of cells per mL.

100 mL of fixed M. ruminantium and/or M. gottschalkii cells and/or cell parts was prepared at 10⁹ cells per mL in DPBS by diluting the appropriate quantity of fixed cells in DPBS. Such a density of Methanobrevibacter cells, e.g., 10⁹ cells per mL, is significantly greater than that observed in the rumen of ruminants. Finally, 100 mL resuspensions were split into two equal aliquots of approximately 50 mL each and stored at −80° C. for cryopreservation.

1 mL aliquots of fixed M. ruminantium and/or M. gottschalkii cells and/or cell parts were prepared by thawing one or more previously frozen 50 mL tubes (10⁹ cells per mL; stored at −80° C.) at room temperature and transferring 1 mL of thawed, fixed cells and/or cell parts into 2 mL threaded cryogenic vials, e.g., cryotubes. Fixed stocks of M. ruminantium and/or M. gottschalkii cells and/or cell parts were transferred parts using a disposable 30 mL sterile syringe through a 23G BD PrecisionGlide needle to minimize clogging during subsequent vaccine formulation and administration. Finally, cryotube lids were closed tightly, wrapped with parafilm, and cryotubes were stored at −80° C.

This example demonstrates a method to grow cells of at least one methanogen, which is of a genus Methanobrevibacter, e.g., M. ruminantium and/or M. gottschalkii, in an oxygen-free environment, e.g., 80% H₂, 20% CO₂ gas mix, with a heterologous growth medium, e.g., BY medium. Such conditions enable the propagation Methanobrevibacter cells to a density not observed in the rumen of ruminants. Furthermore, this example demonstrates a method to grow, fix, and store cells and/or cell parts of at least one methanogen of a genus Methanobrevibacter, e.g., M. ruminantium and/or M. gottschalkii, in a composition suitable for vaccination.

TABLE 12
Exemplary reagents and consumables for fixation and storage
of Methanobrevibacter cells and/or cell parts

| Description | Cat. No. |
|---|---|
| Formalin (diluted 1:10) (Fisher Scientific, Waltham, MA) | 305-510 |
| Dulbecco's Phosphate Buffered Saline (dPBS) | 14190-326 |
| (ThermoFisher, Waltham, MA) | |
| Automatic Setup Beads Kit (SONY, Tokyo, Japan) | LE-B3001 |
| SPHERO Flow Cytometry Standard Size Kit | PPS-1 2.0 μm |
| (Spherotech, Lake Forest, IL) | |
| Microcentrifuge tubes, 1.5 mL (Pierce, Waltham, MA) | 1615-5510 |
| Conical tube, 50 mL (Falcon, Glendale, AZ) | 352098 |
| Fisherbrand ™ Disposable PES Filter Unit, 150 mL | FB12566500 |
| (Fisher Scientific, Waltham, MA) | |
| 5 mL polypropylene tubes (Falcon, Glendale, AZ) | 352063 |
| Tris-EDTA (Fisher Scientific, Waltham, MA) | BP2473-100 |
| Threaded cryogenic storage vials, 2 mL | 1050026 |
| (Fisher Scientific, Waltham, MA) | |
| BD Luer Slip Syringe sterile, single use, 30 mL | 302833 |
| 23G BD PrecisionGlide Needle | 305194 |

### Example 2: Vaccination of Animals (e.g., Cattle), with Cells and/or Cell Parts of Methanobrevibacter Ruminantium and/or Methanobrevibacter Gottschalkii, and Subsequent Reductions in Emitted CH₄, H₂, and/or CO₂ Normalized CH4 and/or Increase in Emitted CO₂

This example demonstrates a method of administering a vaccine composition to an animal. The vaccine composition used in this study contains cells and/or cell parts of Methanobrevibacter ruminantium and/or Methanobrevibacter gottschalkii, which upon vaccination to animals resulted in reduction in the animal emitted CH₄, H₂, and/or CO₂ normalized CH₄; and/or increase emitted CO₂.

The present study demonstrated a statistically significant >10% reduction of animal emitted CH₄, >10% reduction of animal emitted H₂, and/or >10% reduction of animal emitted CO₂ normalized CH₄; and/or >5% increase of animal emitted CO₂. Moreover, these changes were sustained for 5-7 weeks in animals treated with the vaccine compositions compared to the control. Further, there was no noticeable change in the weight of the vaccinated animals at the end of the study compared to the control, demonstrating the safety of the vaccines.

### Formulation and Administration of an Exemplary Vaccine Composition

Formulation and administration of vaccine compositions comprising Methanobrevibacter ruminantium and/or Methanobrevibacter gottschalkii cells and/or cell parts were performed as described below. Briefly, frozen, fixed aliquots of methanogen cells and/or cell parts as prepared in Example 1 were combined with a suitable adjuvant. Animals were inoculated with the resulting vaccine compositions by veterinary-trained staff.

For the treatment groups, 1 mL aliquots of fixed Methanobrevibacter cells and/or cell parts (10⁹ cells per mL) as prepared in Example 1 were thawed on wet ice and mixed with 1 mL of the appropriate adjuvant (1:1 volume ratio) using a vortexer, for a final volume of 2 mL. For the control groups, 1 mL aliquots of DPBS were mixed with 1 mL of the appropriate adjuvant (1:1 volume ratio) using a vortex, for a final volume of 2 mL.

The vaccine compositions used in this study were prepared using Freund's complete adjuvant (ThermoFisher, Waltham, MA) for primary injections, and Freund's incomplete adjuvant (ThermoFisher, Waltham, MA) for subsequent booster injections; however, any suitable adjuvant may be used for the preparation of the vaccine compositions as disclosed herein. Beneficial properties of an adjuvant can include enhanced antigen presentation, improved antigen stability, and/or service as immunomodulators. Exemplary adjuvants are known in the art (e.g., Spickler, A. R., & Roth, J. A. Adjuvants in veterinary vaccines: modes of action and adverse effects. Journal of veterinary internal medicine, 17(3), 273-281 (2003), which is incorporated herein by referenced).

Following formulation, the vaccine compositions were kept at 4° C. or on wet ice until injection into animals. Full and abbreviated descriptions of the vaccine compositions used in this study are described in Table 13.

TABLE 13
Description of vaccine compositions

| Abbreviated | |
|---|---|
| vaccine | |
| description | Full vaccine description |
| Buffer control | 1 mL of DPBS + 1 mL of Freund's adjuvant |
| M. ruminantium | 1 mL (10⁹ cells/mL) of fixed M. ruminantium + |
| | 1 mL of Freund's adjuvant |
| M. gottschalkii | 1 mL (10⁹ cells/mL) of fixed M. gottschalkii + |
| | 1 mL of Freund's adjuvant |
| Blend | 0.5 mL (1 × 10⁹ cells/mL) of fixed M. ruminantium + |
| | 0.5 mL (1 × 10⁹ cells/mL) of fixed M. gottschalkii + |
| | 1 mL of Freund's adjuvant |

Prior to initiation of the study, cattle were transferred to the animal study site (Beef Cattle Systems, 10724 FM 50, Somerville, TX 77879) for a covariate period; the covariate period included time to adapt to the study diet and environment, as well as to learn behaviors associated with GrowSafe systems (GrowSafe Systems, Calgary, AB, CA) which record individual animal dry matter intake and GreenFeed (C-Lock, Rapid City, SD) systems which record individual animal CH₄, H₂ and CO₂ emissions. The covariate period lasted approximately two weeks.

Only healthy, weaned Angus Cross steers were enrolled in this study. All steers selected were within 100 lbs of each other and adapted to a grower's diet consisting of 40% rolled corn, 25% dried distillers grains, 2.5% mineral premix, 7.5% molasses and 25% Sudan hay.

Vaccines were administered on day 0 (d0) (Freund's complete adjuvant), d21 (Freund's incomplete adjuvant), d56 (Freund's incomplete adjuvant), and d77 (Freund's incomplete adjuvant) in this study to animals according to the treatment assignments in Table 14.

TABLE 14
Treatment assignments for ruminants,
e.g., cattle, enrolled in Trial 2

| | Abbreviated |
|---|---|
| Animal ID | vaccine description |
| 245K | M. ruminantium |
| 327K | M. ruminantium |
| 396K | M. gottschalkii |
| 398K | M. gottschalkii |
| 432K | Blend |
| 442K | Blend |
| 444K | M. gottschalkii |
| 466K | Buffer control |
| 477K | M. ruminantium |
| 487K | M. ruminantium |
| 520K | Buffer control |
| 522K | M. gottschalkii |
| 534K | Buffer control |
| 564K | Blend |
| 614K | Buffer control |
| 620K | Blend |
| 634K | Blend |
| 884K | M. gottschalkii |
| 937K | M. ruminantium |
| 945K | Buffer control |

Total blood, saliva and rumen fluid samples were collected periodically throughout the study for subsequent processing and analysis, the frequency of which is described in Table 15.

TABLE 15
Sample collection schedule for Trial 2

| | | Total blood | Saliva | Rumen fluid |
|---|---|---|---|---|
| Collection date | Vaccination | draw | collection | collection |
| d0 | X | X | X | X |
| d21 | X | X | | |
| d28 | | X | X | X |
| d42 | | X | | |
| d49 | | X | X | X |
| d56 | X | X | | |
| d63 | | X | X | X |
| d77 | X | X | | |
| d84 | | X | X | X |
| d98 | | X | X | X |
| d112 (Buffer | | X | X | X |
| control + | | | | |
| M. gottschalkii | | | | |
| only) | | | | |
| d126 (Buffer | | X | X | X |
| control + | | | | |
| M. gottschalkii | | | | |
| only) | | | | |
| d140 (Buffer | | X | X | X |
| control + | | | | |
| M. gottschalkii | | | | |
| only) | | | | |

### Sera Collection and Confirmation of Sera Antibody Binding to Methanogens

Total blood samples were collected under the supervision of veterinary-trained staff. Samples were collected via jugular vein needle puncture into blood tubes. Collected blood samples were then clarified via ultracentrifugation within 24 hrs of collection in order to harvest serum. Clarified serum samples were aliquoted, labeled and stored at ≤−20° C.

Specifically, jugular blood draws were accomplished by first restraining the animal in a squeeze chute with a head gate. The neck area was then cleaned by wiping the area with isopropyl alcohol, e.g., rubbing alcohol, and gauze to remove any superficial dirt and debris. The jugular vein was occluded by applying pressure at the base of the jugular groove in order to visualize the raised vein. After a sufficient volume of blood was collected, the needle was removed and disposed of in an approved sharps container.

### Confirmation of Sera Antibody Binding to Methanogens Using A Methanogen ELISA

In order to determine a humoral immune response to the vaccine in the immunized animals, a methanogen ELISA was developed and utilized to assess sera antibody binding to at least one methanogen of a genus Methanobrevibacter, e.g., M. ruminantium and/or M. gottschalkii.

Exemplary reagents used for the preparation of methanogen ELISA microplates, and subsequent execution of the methanogen ELISA protocol, are described below in Table 16. Specifically, each well of a 96-well high binding ELISA plate, e.g., plate, was coated with 50 μL of 0.001% polylysine. The plate was then stored at 4° C. for at least 10 hrs, and up to 2 weeks. Fixed methanogen cells and/or cell parts were prepared following procedures described in Example 1 and diluted in DPBS to an empirically determined dilution for each plate preparation (typically 1:10). Next, the volume of 0.001% polylysine was removed from the plate, and 50 μL of diluted, fixed methanogen cells and/or cell parts was added to each well. The plate was then centrifuged for 5 min. at 900×g to facilitate binding of the methanogen cells and/or cell parts. Next, 50 μL of 0.1% glutaraldehyde was added to each well, mixed well, and incubated for 20 min. at room temperature. The liquid from each well was removed, and 200 μL of 5% goat serum in PBST (blocking solution) was added to each well. The plate was then incubated for 1 hr at room temperature to prevent non-specific binding of the detection antibodies in subsequent steps, also referred to as ‘blocking’.

The clarified sera collected from the animals enrolled in this study as described above was diluted in 5% goat serum in PBST, e.g., blocking solution. Next, the blocked plates were washed twice by adding 200 μL of PBST to each well, and then removing the liquid. 50 μL of PBST-diluted sera was added to each well and incubated for 1 hr at room temperature. The plate was then washed three times with 200 μL of PBST per well. 50 μL of 1:1,000 rabbit anti-bovine-HRP conjugate was added to each well and incubated for 1 hr at room temperature. The plate was then washed three times with 200 μL of PBST per well. Next, 50 μL of TMB solution was added to each well, incubated for 20 min. at room temperature, to develop. Finally, 50 μL of 2 M H₂SO₄ was added to each well to stop the reaction. OD₄₅₀ was measured for each well using a Synergy plate reader (BioTek, Winooski, VT).

TABLE 16
Exemplary list of reagents for the preparation
and execution of a methanogen ELISA

| Description | Cat. No. |
|---|---|
| 20X PBST (ThermoFisher, Waltham, MA) | 28352 |
| OneStep TMB ELISA (ThermoFisher, Waltham, MA) | 34024 |
| 2M H₂SO₄ (Fisher Scientific, Waltham, MA) | SA818 |
| 25% glutaraldehyde (Sigma-Aldrich, St. Louis, MO) | G6257 |
| Polylysine (Sigma-Aldrich, St. Louis, MO) | P8920 |
| High Binding ELISA 96 W plate (Greiner Bio-One, | 655085 |
| Kremsmünster, Austria) | |
| DPBS (ThermoFisher, Waltham, MA) | 14190-326 |

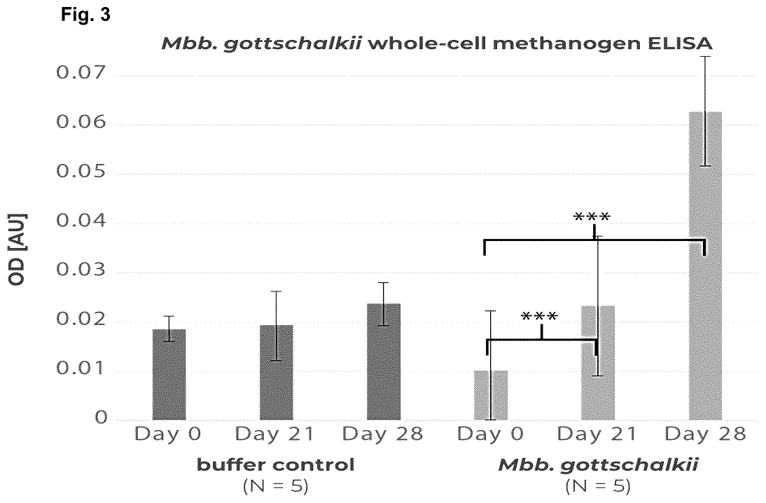

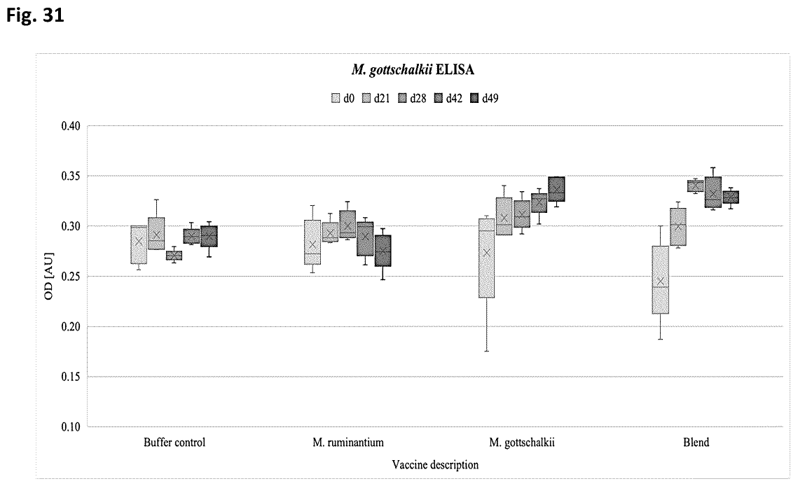

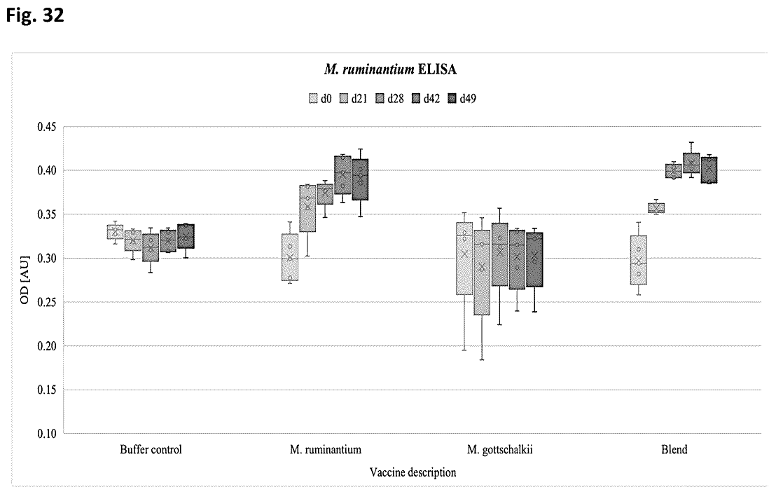

FIG. 3, FIG. 31 and FIG. 32 show whole-cell methanogen ELISA results using M. ruminantium or M. gottschalkii affixed to the ELISA plates. Sera collected from animals vaccinated with M. gottschalkii cells and/or cell parts show specific and increasing antibody titer to M. gottschalkii cells (FIG. 31) and little to no binding to M. ruminantium cells. Sera collected from animals vaccinated with M. ruminantium cells and/or cell parts show specific and increasing antibody titer to M. ruminantium cells (FIG. 31) and little to no binding to M. gottschalkii cells. Cells vaccinated with both show specific and increasing antibody titer to both (FIG. 31 and FIG. 32). Animals vaccinated with buffer control showed little to no specific binding to either M. gottschalkii or M. ruminantium (FIG. 31 and FIG. 32). The statistically significant increase in sera binding to the methanogen to which the animals were vaccinated, for example at d21 as compared to d0 of the study, was observed, thereby demonstrating an immune response to the vaccine. The statistically significant increase in binding of the antibody to the methanogen was specific to the methanogen to which the animals were vaccinated. The sera did not bind to a methanogen to which the animals were not vaccinated. For example, an increase in binding to M. ruminantium ELISA plates was observed with sera samples collected from animals vaccinated against M. ruminantium while a similar increase in binding to M. gottschalkii ELISA plates was not observed with the same set of sera samples. This example demonstrates a confirmation of a humoral immune response in animals, e.g., cattle, immunized with the vaccine composition.

### Confirmation of Sera Antibody Binding to Methanogens Using FACS

In order to confirm the development of a humoral immune response to the vaccine in animals immunized with a vaccine composition comprising M. gottschalkii cells and/or cell parts, fluorescence-activated cell sorting (FACS) was utilized to observe sera antibody binding to M. gottschalkii.

Specifically, 5 μL of fixed M. gottschalkii cells and/or cell parts prepared following procedures described in Example 1 was added to 50 μL of PBS-T in an Eppendorf tube. Then, where applicable, 1 μL of clarified sera collected from the animals enrolled in this study as described above, was added to the relevant Eppendorf tube and mixed well. A detection solution was prepared by adding 150 μL of PBST to 1.5 μL of rabbit anti-bovine IgG biotin and 1.5 μL of SA-PECy7 and mixing well. Once prepared, 50 μL of detection solution was added to sample in Eppendorf tube(s) and mixed well. Sample in Eppendorf tubes, including detection solution, was incubated for 30 min. at room temperature. Finally, 5 μL of each previously incubated sample was added to 1 mL of DPBS immediately before running on SONY SH800 (FSC gain: 5; threshold: 0.04%; all other settings: default).

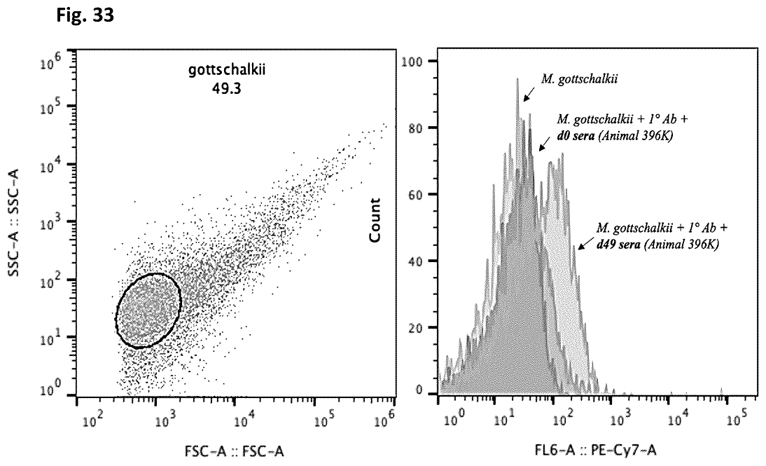

FIG. 33 displays the FACS-based sera binding to fixed M. gottschalkii cells. The sera harvested from Animal 396K before (d0) and after (d49) immunization with a vaccine comprising M. gottschalkii cells and/or cells parts and Freund's adjuvant were analyzed.

The statistically significant increase of >200% in sera binding, as measured by shift in mean fluorescence of the PE-Cy7 channel of the gated M. gottschalkii population, to fixed M. gottschalkii cells was observed for d49 as compared to d0 of the study. This example demonstrates a confirmation of a humoral immune response in animals, e.g., cattle, immunized with the vaccine composition.

### Confirmation of Sera Antiboding Binding to Methanogens Using Western Blot

In order to confirm the development of a humoral immune response to the vaccine in animals immunized with the vaccine composition, a methanogen Western blot was utilized to observe the sera antibody binding to the methanogen(s) to which the animals were vaccinated. The Western blot analysis detects the interaction of (a) the sera antibodies previously collected from the animals enrolled in this study as described above, and (b) methanogen protein antigens resolved on an SDS-PAGE gel.

Specifically, between 5 and 10 mL of actively growing M. ruminantium and/or M. gottschalkii cultures grown as described in Example 1 was centrifuged for 5 min. at 3,000×g to pellet the methanogen cells and/or cell parts. Next, the resultant supernatant was discarded and the pellet was resuspended in 1 to 2 mL of PBS and again centrifuged for 5 min. at 3,000×g. The resultant supernatant was discarded and the pellet was resuspended in 100 to 200 μL of 1× SDS sample buffer. Next, the resuspended methanogen cells and/or cell parts were boiled for 5 min. at 100° C. to obtain methanogen cell and/or cell parts lysate (methanogen lysate).

Between 5 and 10 μL of methanogen lysate in 1× SDS sample buffer was loaded into individual wells of a 4-15% SDS-PAGE gel. Next, the SDS-PAGE gel was run for 10 min. at 100 V, and then 30 min. at 200 V. The proteins were then transferred using wet transfer technique for 1 hr at 100 V onto a 0.22 μm PVDF membrane. Next, the 0.22 μm PVDF membrane was incubated in SuperBlock T20 TBS buffer solution (blocking solution) for 1 hr at room temperature to prevent non-specific binding of the detection antibodies in subsequent steps. The blocked 0.22 μm PVDF membrane was then exposed to various dilutions of clarified sera samples, previously collected from the animals enrolled in this study as described above, in blocking solution for 1 hr at room temperature. Next, the 0.22 μm PVDF membrane was washed three times (5 min. for each wash) in TBS-T20 to remove any unbound sera and/or antibodies. The 0.22 μm PVDF membrane was then exposed to HRP-conjugated anti-bovine IgG secondary antibody in SuperBlock T20 TBS buffer solution for 1 hr at room temperature to detect any bound bovine sera antibodies. Next, the 0.22 μm PVDF membrane was washed three times (10 min. for each wash) in TBS-T20 to remove any unbound HRP-conjugated anti-bovine IgG antibodies. Finally, the 0.22 μm PVDF membrane was developed using chromogenic TMB blotting solution, or an appropriate chemiluminescence substrate.

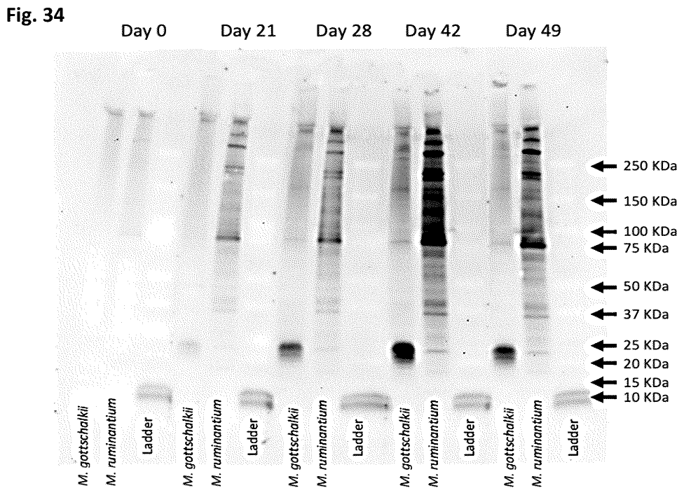

FIG. 34 displays the sera binding to M. ruminantium and M. gottschalkii proteins resolved on the Western blot, where the sera was harvested from Animal 432K before (d0) and after (d21, d28, d42 and d49) immunization with a vaccine composition containing a blend of M. ruminantium cells and/or cell parts and M. gottschalkii cells and/or cells parts, and Freund's adjuvant.

The sera binding to M. ruminantium and M. gottschalkii proteins was observed following the administration of the vaccine containing both methanogens to the animals (see, e.g., d49 sera compared to d0 sera of the study). Moreover, an increased binding of the sera antibodies to the methanogen proteins was observed with the progression of time (see, e.g., d49 sera compared to d21 sera of the study). This example confirms a humoral immune response to the vaccine in immunized animals, e.g., cattle.

### Saliva Collection

Saliva samples were collected using a suction tube in the oral cavity, into a conical flask or directly into a sterile Eppendorf tube. Equipment contacting saliva, e.g., collection flask or plastic tubing, was changed out after each animal's collection; either single-use or autoclavable materials were used. Sample were immediately put on wet ice, and then transported to the lab for storage at ≤−80° C.

### Rumen Fluid Collection

First, the ruminant, e.g., steer, was restrained in a squeeze chute with a head gate and nose tucked down toward the chest. Then, a long flexible plastic tube was inserted into the animal's nostril and down into the stomach. A hand-operated rumen fluid pump, attached to the flexible plastic tube, is then used to harvest 20-50 mL of rumen fluid per collection. Harvested rumen fluid samples were stored in conical vials and transferred to the lab. Finally, within 24 hrs of collection, rumen fluid samples were strained through three layers of cheesecloth, and the liquid phase was aliquoted into 50 mL conical vials and snap-frozen in liquid N₂ for cryostorage at ≤−20° C.

### Recording and Analysis of Dry Matter Intake

This example describes a method to record and analyze daily dry matter intake of vaccinated animals in order to determine whether the administration of a vaccine has an adverse effect on individual daily feed intake. Furthermore, such a record may be used to normalize inter-animal, e.g., ruminant, for example, cattle, comparisons of daily emitted CH₄.

Animals were fed once daily in the morning and had access to a water source at all times for the duration of the study. Feed intake was recorded continuously using GrowSafe systems (GrowSafe Systems, Calgary, AB, CA). Briefly, an individual animal's electronic identification tag, e.g., EID, which is typically affixed to the ear, is read by the GrowSafe feed bunk when the animal's head is in sufficiently close proximity to the bunk, e.g., when the animal is feeding from the bunk. During recorded feed events, the animal's EID is associated with the amount of feed that animal consumes from the bunk, which is automatically measured using scales equipped within the bunk. A comparable method for tracking individual dry matter intake may be used where GrowSafe systems are unavailable.

### Recording and Analysis of Animal Body Weight

This example demonstrates a method of recording individual animal body weight (lb) in order to confirm that administration of a vaccine composition does not have an adverse effect on the rate of individual animal weight gain.

Animal body weight was recorded periodically, at the time of total blood draws, throughout the study. Body weight was recorded using a livestock scale. Specifically, animals to be weighed were walked through a pipe alley from their housing area to the holding pens. After being weighed, they were released to return to their holding pen with other animals from the same pen. At the end of the weighing, animals were then walked back to their assigned pen.

TABLE 15A
Animal body weights recorded for d 0-d 49

| Animal ID | Treatment description | d 0 | d 14 | d 21 | d 28 | d 42 | d 49 |
|---|---|---|---|---|---|---|---|
| 245K | M. ruminantium | 1210 | 1230 | 1260 | 1255 | 1275 | 1275 |
| 327K | M. ruminantium | 1180 | 1235 | 1270 | 1285 | 1285 | 1275 |
| 396K | M. gottschalkii | 1180 | 1260 | 1265 | 1240 | 1270 | 1270 |
| 398K | M. gottschalkii | 1155 | 1200 | 1210 | 1220 | 1250 | 1250 |
| 432K | Blend | 1215 | 1265 | 1295 | 1300 | 1335 | 1315 |
| 442K | Blend | 1135 | 1155 | 1170 | 1165 | 1200 | 1170 |
| 444K | M. gottschalkii | 1115 | 1135 | 1160 | 1165 | 1200 | 1200 |
| 466K | Buffer control | 1190 | 1225 | 1225 | 1255 | 1275 | 1250 |
| 477K | M. ruminantium | 1160 | 1200 | 1215 | 1230 | 1295 | 1285 |
| 487K | M. ruminantium | 1185 | 1210 | 1225 | 1235 | 1265 | 1260 |
| 520K | Blend | N/A | 1090 | N/A | 1075 | | |
| 522K | Buffer control | 1195 | 1250 | 1250 | 1285 | 1125 | 1095 |
| 534K | M. gottschalkii | 1215 | 1255 | N/A | 1285 | 1285 | 1300 |
| 564K | Buffer control | 1200 | 1230 | 1260 | 1260 | 1315 | 1280 |
| 614K | Blend | 1190 | 1270 | 1270 | 1270 | 1285 | 1325 |
| 620K | Buffer control | 1130 | 1195 | 1215 | 1235 | 1300 | 1290 |
| 634K | Blend | 1210 | 1250 | 1290 | 1270 | 1290 | 1295 |
| 884K | Blend | 1240 | 1280 | 1315 | 1310 | 1335 | 1320 |
| 937K | M. gottschalkii | 1210 | 1220 | 1250 | 1250 | 1345 | 1340 |
| 945K | M. ruminantium | N/A | 1235 | 1250 | 1300 | 1260 | 1220 |

TABLE 15B
Animal body weights recorded for d 56-d 140

| | | | | | | | |
|---|---|---|---|---|---|---|---|
| Animal ID | Treatment description | d 56 | d 63 | d 77 | d 84 | d 98 | d 112 |

| | | | |
|---|---|---|---|
| 245K | M. ruminantium | 1300 | 1305 |
| 327K | M. ruminantium | 1305 | 1325 |
| 396K | M. gottschalkii | 1285 | 1240 |
| 398K | M. gottschalkii | 1250 | 1285 |
| 432K | Blend | 1335 | 1340 |
| 442K | Blend | 1185 | 1175 |
| 444K | M. gottschalkii | 1200 | 1215 |
| 466K | Buffer control | 1280 | 1305 |
| 477K | M. ruminantium | 1300 | 1290 |
| 487K | M. ruminantium | 1320 | 1305 |
| 520K | Buffer control | 1105 | 1110 |
| 522K | M. gottschalkii | 1305 | 1290 |
| 534K | Buffer control | 1330 | 1320 |
| 564K | Blend | 1315 | 1310 |
| 614K | Buffer control | 1305 | 1275 |
| 620K | Blend | 1315 | 1325 |
| 634K | Blend | 1325 | 1320 |
| 884K | M. gottschalkii | 1355 | 1350 |
| 937K | M. ruminantium | 1265 | 1270 |
| 945K | Buffer control | 1295 | 1325 |

There were no noticeable reductions in weight gain at the end of the study in the vaccinated animals compared to the control.

Measurement of Animal Emitted CH₄, H₂ and CO₂

The amount of animal-emitted CH₄, H₂, and CO₂ was measured using GreenFeed systems (C-Lock, Inc., Rapid City, SD). Briefly, the GreenFeed system uses a pelletized feed to incentivize animals to visit multiple times per day. When an animal visits a GreenFeed system to consume the dispensed feed pellets, its individual animal's electronic identification tag, e.g., EID, which is typically affixed to the ear, is read by GreenFeed and a measurement period associated with that animal is recorded. During the measurement period, GreenFeed records the amount of animal-emitted CH₄, H₂, and CO₂.

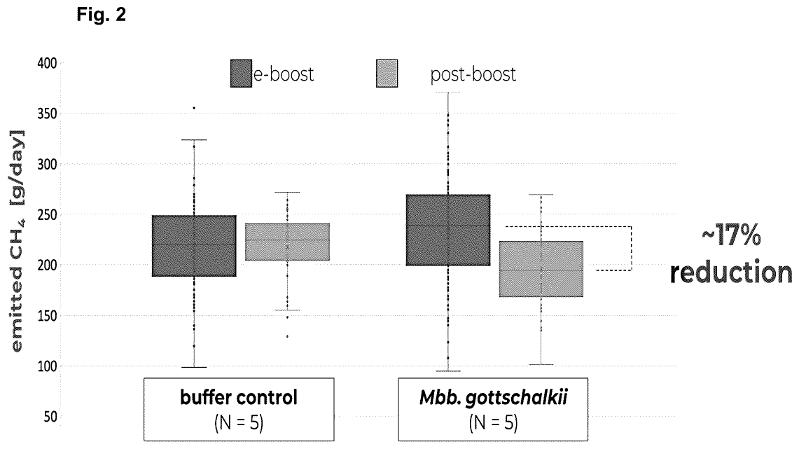

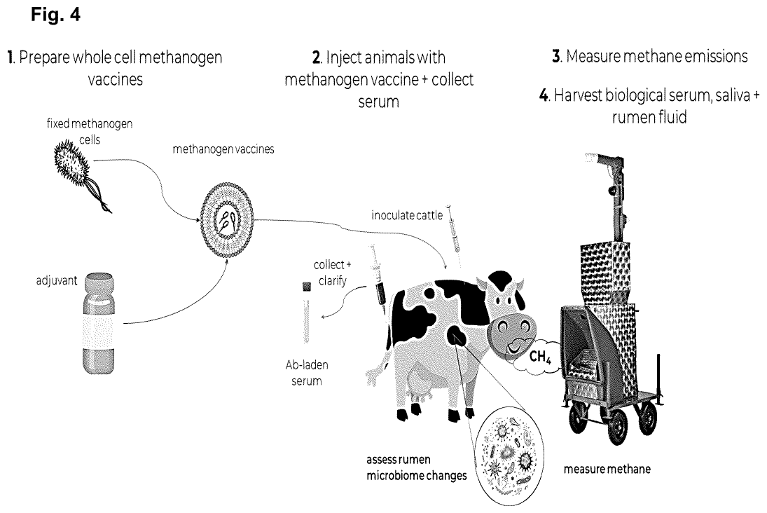

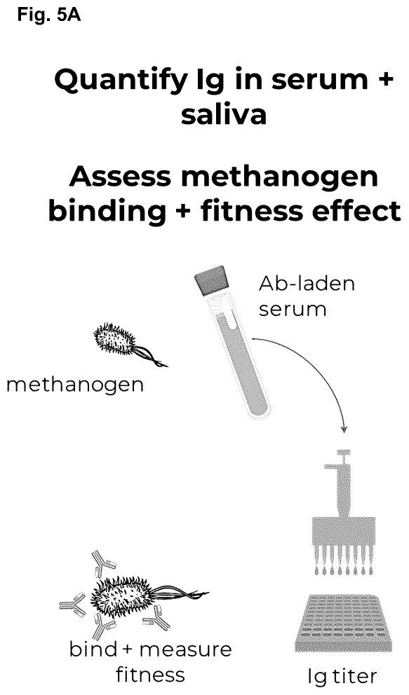

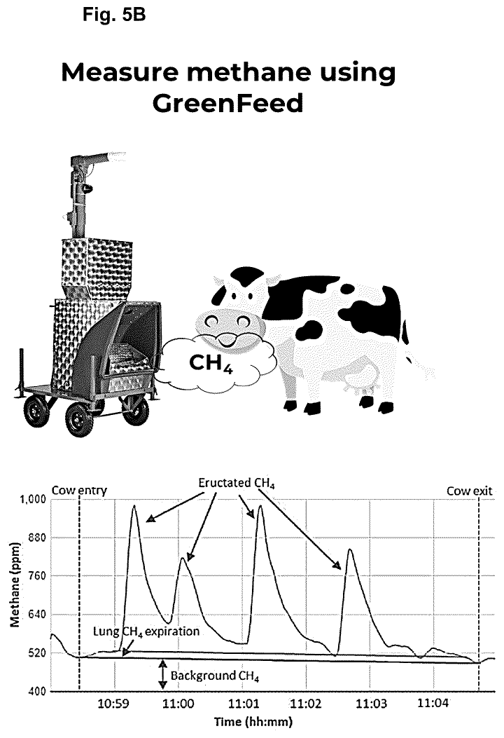

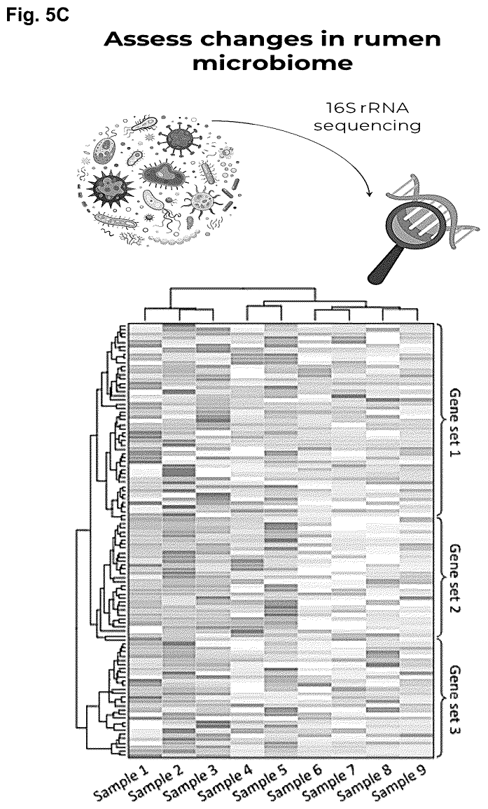

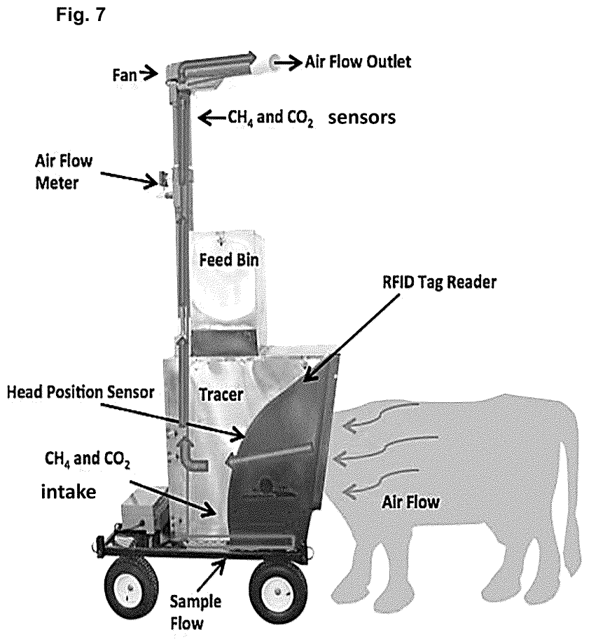

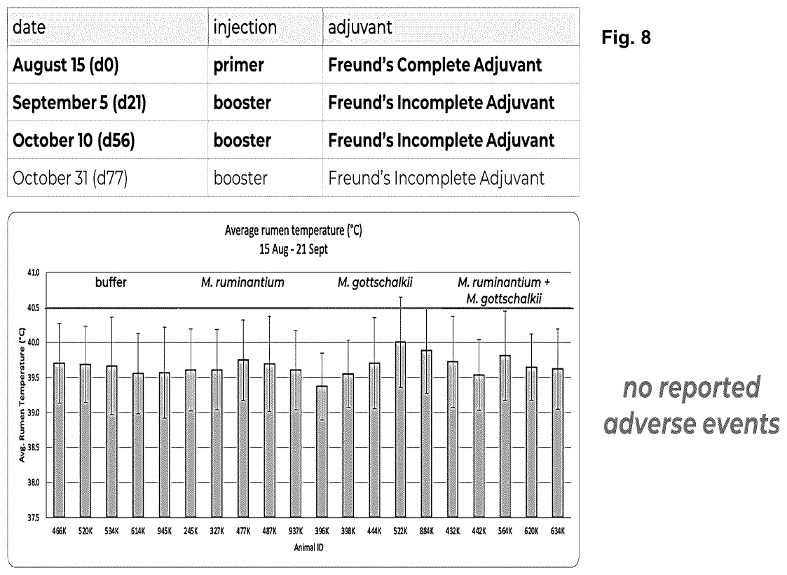

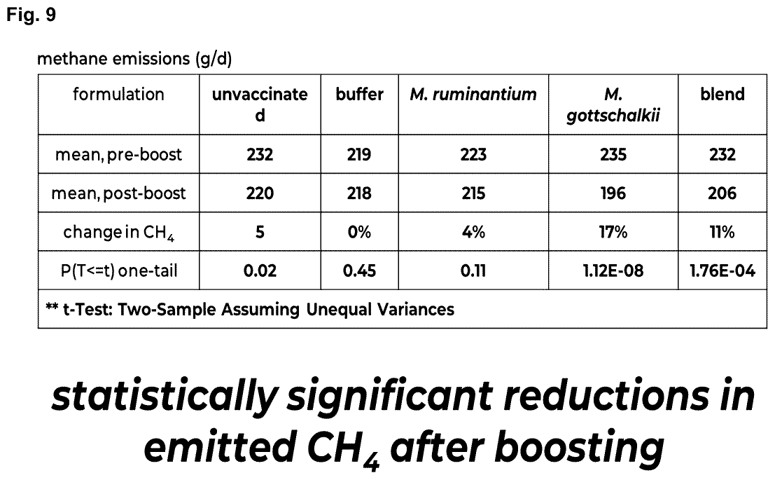

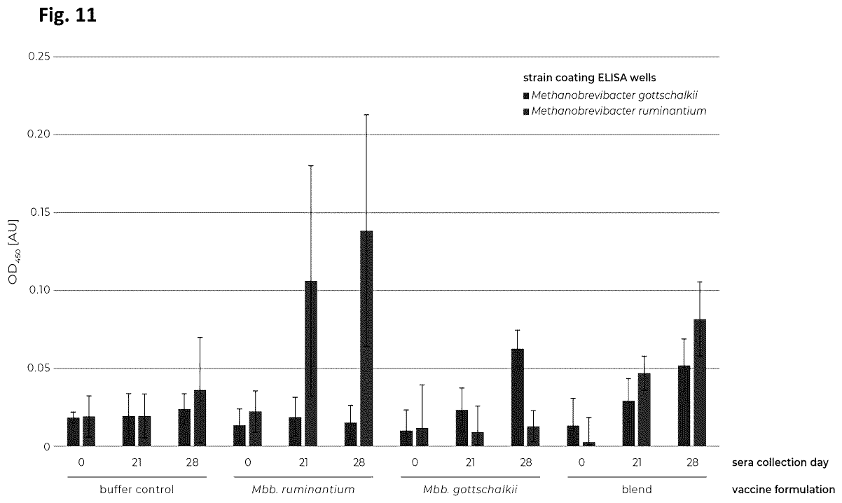

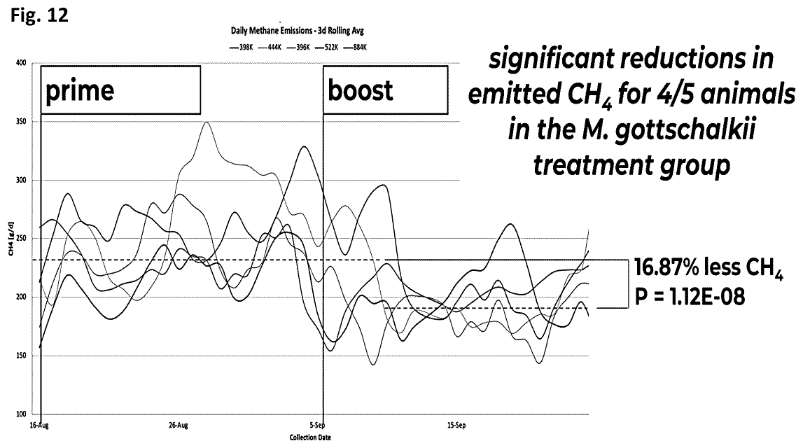

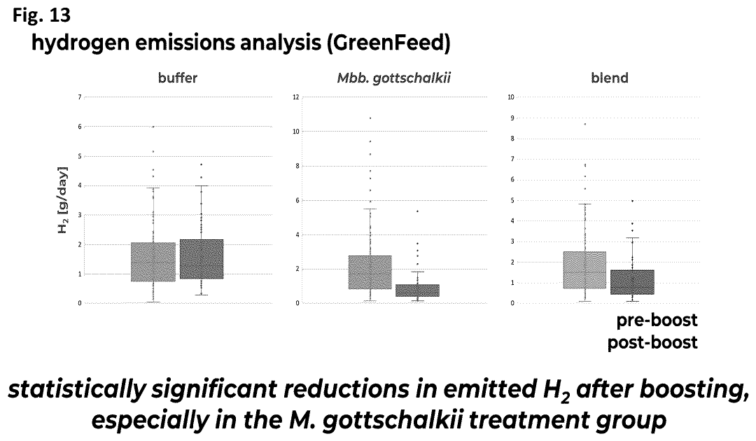

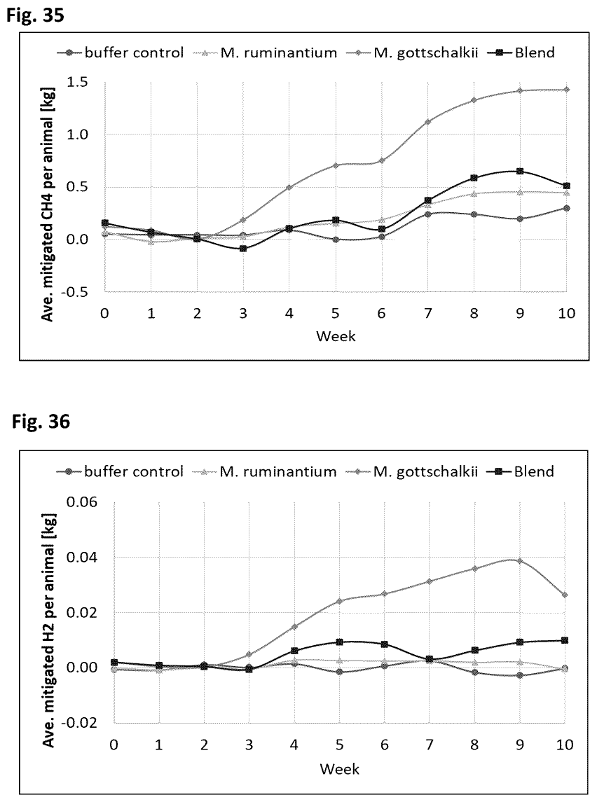

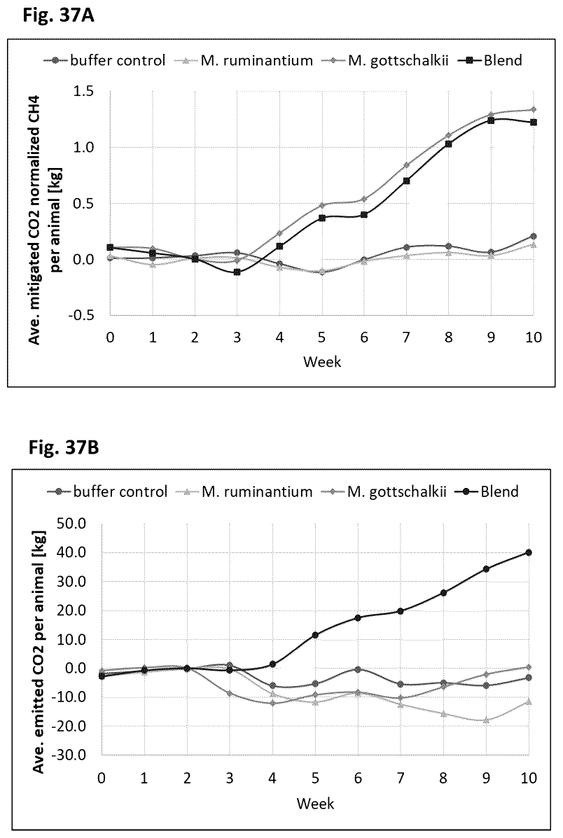

Representative results are summarized in FIGS. 2, 9A, 9B, 11, 12 and FIG. 35-FIG. 37, which demonstrate a statistically significant >10% reduction of animal emitted CH₄, >10% reduction of animal emitted H₂, and/or >10% reduction of animal emitted CO₂ normalized CH₄, and/or >5% increase of animal emitted CO₂. Surprisingly, these changes were sustained for at least 5-7 weeks in animals immunized with the vaccine compositions. Illustrative schematics of vaccination and measurements are shown in FIG. 4-7.

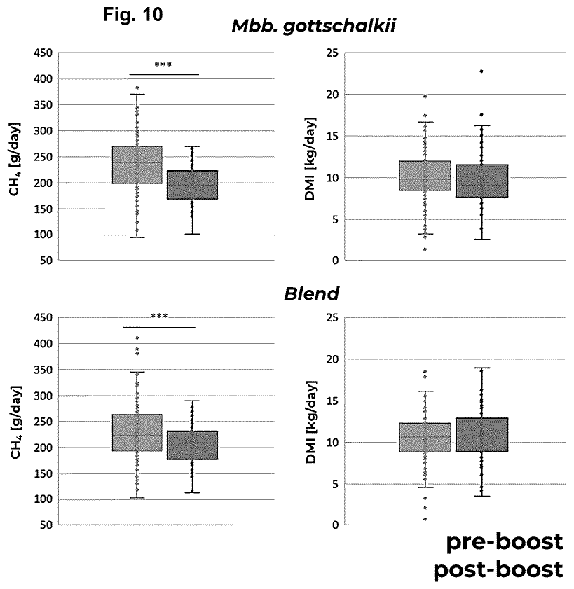

Specifically, FIG. 2 shows emitted CH₄ in units of g/day for buffer control and Methanobrevibacter gottschalkii vaccinated animals before (dark) and after (light) boost vaccine (i.e., second vaccination). Buffer control animals showed no change in emitted CH₄ following boost. Methanobrevibacter gottschalkii vaccinated animals showed a significant 17% reduction in emitted CH₄ following vaccination. This is further supported by a significant increase in Methanobrevibacter gottschalkii-specific antibodies following vaccination as measured by antigen-specific ELISA as shown in FIG. 3. There was a similar reduction in emitted CH₄ for animals vaccinated with the blend formulation as shown in FIG. 10. Surprisingly and unexpectedly, animals vaccinated with Methanobrevibacter gottschalkii and the blended vaccine showed a significant reduction (˜50% and ˜30%, respectively) in emitted H₂ following boost. This effect is contrary to the effects of small molecule inhibitors of methanogenesis which see increases in H₂ emissions following treatment, suggesting that vaccines of the present disclosure act through different mechanisms on the rumen microbiota. The concomitant reduction is CH₄ and H₂ emissions and lack of reduction in ADG and DMI suggest that there may be productivity improvements in vaccinated animals.

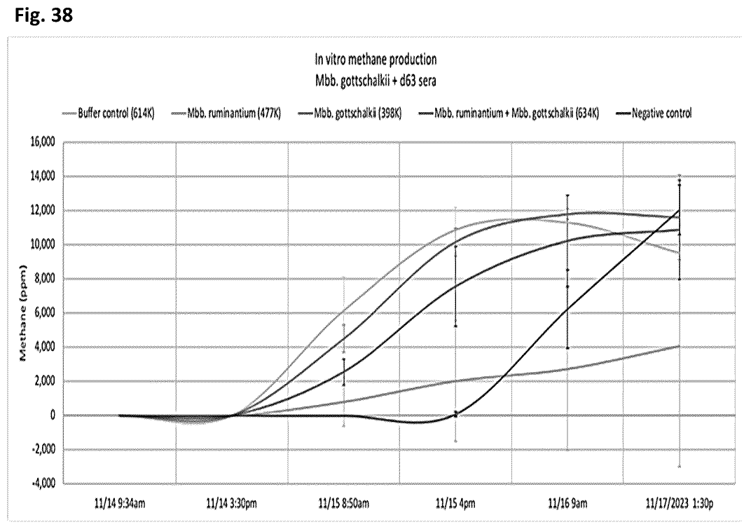

Methanogen In Vitro Inhibition Assay Protocol FIG. 38 shows in vitro CH₄ production following treatment with sera antibodies. A significant reduction in CH₄ production is present in cultures treated with sera containing antibodies from vaccinated cattle.

A three day seed culture of methanogen strain M. gottschalkii was diluted 1:100 into Hungate tubes containing BY medium with the addition of either (1) sera from cattle prior to vaccination (AN396 DO, 4% v/v), (2) sera from vaccinated cattle (AN396 D84, 4% v/v), (3) commercially available adult bovine serum (ABS, 4% v/v, Gibco), (4) metronidazole (met, 32 pg/ml, Thermo Scientific Chemicals), or (5) no addition as a growth control. These tubes had the headspace exchange and charged to 20 psi with an 80% H₂, 20% CO₂ gas mix. The tubes were incubated at an angle with shaking at 200 rpm at 38° C. The optical density of the cultures and CH₄ concentration in the headspace were monitored over 75 hrs using a spectrophotometer (Spectronic20, Bausch & Lomb) and CH₄ detector (Gazomat Gazoscan), respectively.

The CH₄ concentration and optical density for each replicate was plotted versus time and then the area under the curve was calculated (GraphPad Prism). A one-way ANOVA with Tukey's multiple comparison correction was performed to determine if there was a significant difference between the conditions (GraphPad Prism).

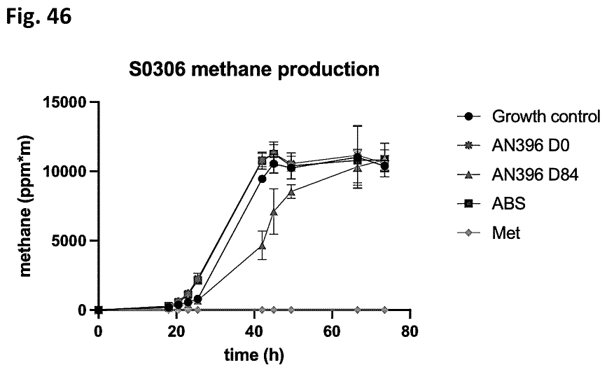

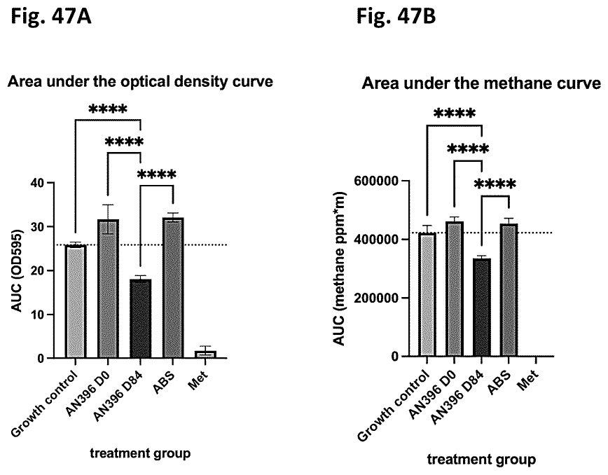

FIG. 46 demonstrates a significant reduction in CH₄ production in vitro following treatment with sera from an animal before treatment (DO; asterisks) and 84 days after treatment (D84; triangles). An untreated growth control is shown in circles; cultures treated with commercially available bovine serum (ABS) is shown in squares, and metronidazole treated cultures is shown in diamonds. FIG. 47 A and B show a bar chart of growth as measured by optical density (OD₅₉₅) and CH₄ production following the treatments as described for FIG. 46. This example demonstrates that treated animals have significant amounts of sera antibody titers that inhibit CH₄ production of methanogens in monoculture.

This example demonstrates that sera antibodies collected from cattle treated with vaccines as disclosed herein have the ability to affect methanogen growth and CH₄ production in vitro. The antibodies themselves have utility for the in vitro testing as well as potentially for use as a treatment or supplement. This further suggests that milk containing antibodies and/or milk antibodies agenerated through the treatment with vaccines as disclosed herein would prove efficacious in providing anti-methanogen activity to a subject ingesting that milk, e.g., a calf.

### Example 3: Immunization

This example further describes a method to vaccinate an animal.

Eighteen 5-month-old male Holstein-Friesian calves are used. All animals are grazed on pasture with water ad libitum.

Eighteen calves are allocated randomly into 3 treatment groups. A vaccine with an adjuvant Montanide ISA61 (n=6) is administered subcutaneously in the anterior region of the neck. A second group receives the vaccine without adjuvant (n=6) administered in a similar manner. A control group (n=6) is not vaccinated. Animals are re-vaccinated at 3 weeks after the first vaccination with the same vaccine. Blood samples are collected by jugular venipuncture, with serum clarified and stored at ≤−20° C. according to methods described above in Example 2 or to comparable methods known to skilled artisan in the field. Saliva samples are collected using a cotton swab or suction tube placed in the oral cavity. If a cotton swab is used, the swab is then placed in a salivette saliva collection tube (Sarstedt, Germany), centrifuged at 2,000×g for 10 min. and the flow-through material harvested is stored at ≤−20° C. If a suction tube is used, the saliva sample is processed following methods described above in Example 2. Rumen content samples are collected using a stomach tube, centrifuged at 10,000×g for 15 min and the supernatant, e.g., rumen fluid, stored at ≤−20° C. Blood, saliva and rumen fluid samples are collected at weeks 0, 3, 6, and 8. Week 0 samples (n=18) are used to determine level of total and/or antigen-specific bovine Ig in serum, saliva and rumen fluid samples prior to vaccination. Weeks 3, 6, and 8 samples are used to determine level of total and/or antigen-specific bovine Ig in serum, saliva and rumen fluid samples follow vaccination.

Throughout the study period, animal emitted CH₄, H₂ and CO₂ is monitored using at least one GreenFeed system (C-Lock, Inc., Rapid City, SD) or using a suitable alternative strategy, for example as described below in Example 5, as determined by a skilled artisan in the field.

### Example 4: Measurement of Bovine Antibodies Using ELISA

### Total Bovine IgA and IGG ELISA

ELISA plates (Maxisorp, Nunc™, Thermo Fisher Scientific, Denmark) are coated overnight at 4° C. with sheep anti-bovine IgA (AbD-serotec, UK) for total IgA or sheep anti-bovine IgG (AbD-serotec, UK) for total IgG at a protein concentration of 1 μg/mL in PBS. The plates are then washed three times with PBS containing 0.05% w/v Tween-20 (PBST) and blocked for 2 hrs at room temperature with 150 μL/well of normal sheep serum in PBS (0.1% w/v for IgA and 1% w/v for IgG). 100 μL/well of saliva (dilution range 1:10²-1:10⁵; IgG-about 1:1,000; IgA-about 1:10,000), serum (dilution range 1:103-1:107; IgG-about 1:250,000; IgA-about 1:500), rumen fluid (dilution range 1:10-1:103) or 2-fold serial dilutions of standards in PBS are added to wells induplicate and plates are incubated for 2 hrs at room temperature. The plates are washed three times in PBST and incubated for 1 hr at room temperature with 100 μL/well of either horseradish peroxidase (HRP)-conjugated sheep anti-bovine IgA (AbD-serotec, UK) diluted 1:10,000 or HRP-conjugated sheep anti-bovine IgG (AbD-serotec, UK) diluted 1:30,000 in blocking buffer for IgA and IgG determination, respectively. The plates are washed three times in PBST and incubated for 10 min. at room temperature in the dark with 100 μL/well of tetramethyl benzidine (TMB) substrate (BD OptEIA™, BD Biosciences, USA). The reactions are stopped by addition of 50 μL/well of 0.05M H₂SO₄ and read at 450 nm. A linear standard curve is fitted to the 2-fold serial dilution of the standards (100 ng/mL to 1.56 ng/mL) and used for the calculation of antibody concentration (μg/mL). Bovine IgA standard is obtained from purification of cattle saliva using anion-exchange chromatography followed by size exclusion gel filtration whereas bovine IgG standard was obtained commercially (Bethyl Laboratories, USA).

### Antigen-Specific IgA and IGG ELISA

ELISA assays are developed to monitor a mtr-specific IgA and mtr-specific IgG responses in serum, saliva and rumen content. ELISA plates are coated overnight with 50 μL/well of mtr (4 ug/mL) in PBS at 4° C. The plates are washed three times in PBST and then blocked for 1 hr at room temperature with 150 μL/well of 1% casein in PBS. Then, 100 μL/well of saliva (dilution range 1:10-1:104), serum (dilution range 1:10-1:105), rumen fluid (dilution range 1:2-1:20) or 2-fold serial dilutions of the positive serum control in PBS are added to duplicate wells and plates incubated for 2 h at room temperature. The plates are washed three times in PBST and incubated for 1 hr at room temperature with 100 μL/well of either HRP-conjugated sheep anti-bovine IgA (AbD-serotec, UK) or HRP-conjugated sheep anti-bovine IgG (AbD-serotec, UK) diluted 1:5,000 in blocking buffer for mtr-IgA and mtr-IgG determination, respectively. The plates are washed three times in PBST and incubated for 10 min. at room temperature in the dark with 100 μL/well of TMB substrate. The reactions are stopped by addition of 50 μL/well of 0.05 M H₂SO₄ and read at 450 nm. Mtr specific IgA and IgG levels of the unknown samples are assigned a unit value based on a positive control (serum obtained from the animal with the highest antibody responses at week 8). A linear curve is fitted to the 2-fold serial dilution of the positive serum control (1:100 to 1:6400 for mtr-IgA and 1:100,000 to 1:6,400,000 for mtr-IgG) and then used for the calculation of antibody concentration (units/mL) on every ELISA plate. Based on the curves, the mtr-specific IgA concentration in the positive serum control (1:100 dilution) is assigned a value of 6,400 units/mL, while the mtr-specific IgG concentration in the positive serum (1:100,000 dilution) is assigned a value of 6,400,000 units/mL. For saliva and rumen fluid samples, mtr-specific IgA and mtr-specific IgG are standardized by total IgA or total IgG to control for animal variation in saliva flow rates and rumen content compositions. Data are presented as units mtr-specific IgA/mg of total IgA or mtr-specific IgG/mg of total IgG.

### Example 5: Measurement of CH4 Produced by Animals, e.g., Cattle

The LMD hand-held open path laser measuring device is used. The NDIR, a sniffer method that measures CH₄ concentration (ppm) in breath or exhaled air, is also used.

The records are measured in 29 Holstein and Brown Swiss dairy cows. Cows are 18.5% of the 1st parity, 52% of the 2nd parity, 18.5% of the 3rd parity and 11% of the 4th parity. Average days in milk at the beginning of the experiment is 102±88 d. Cows are offered a partial mixed ration consisting of corn silage, grass silage and straw ad libitum, and a mean of 5 kg of concentrate supplied in the automated milking systems (AMS).

Breath CH₄ concentration is measured on six different days during four months. Measurements are performed after morning unified distribution between 10:00 and 14:00. Animals are restrained and CH₄ is measured simultaneously with the two devices during a 5 min. sampling period, obtaining a total of 164 paired measurements.

An operator points the LMD at a cow's nostril at a fixed distance of 1 meter and trying to maintain the angle from which the LMD is pointed to the cow. Data are recorded every 0.5 sec. in an electronic tablet. Another operator simultaneously places NDIR sampling tube on the cow's nostrils in order to measure CH₄ concentration in exhaled air with the NDIR and data are recorded every 1 sec. in a datalogger.

### Example 6: Metagenomic Analysis of the Ruminal Microbiome

This example demonstrates that two methanogen strains, Methanobrevibacter ruminantium and Methanobrevibacter gottschalkii, comprise ˜65% of the methanogen load in the ruminal microbiome, and, thus, are high value targets for vaccine development. Ruminal 16S sequencing datasets were used to analyze the microbial community composition of rumen samples from 394 cattle across 26 countries (Henderson (2015) Scientific Reports). The average relative abundance value of each archaeal species was pulled from the dataset and corrected for 16S gene copy number (GCN). Methanogen genomes can contain multiple copies of the 16S rRNA gene, so correcting for the GCN enables the capture of a more accurate picture of archaeal community structure. The 16S GCN values for each taxon was determined using the rrnDB, which is a database that documents rRNA operon copy number variation in Bacteria and Archaea. All taxa within the dataset were represented in the rrnDB2. The resulting abundance analysis is shown in FIG. 1A and FIG. 1B. Specifically, FIG. 1A and FIG. 1B show the relative abundance of the archaeal species that composed >1% of the 16S GCN-corrected data. The methanogenic taxa Methanobrevibacter, Methanosphaera, and Methanomethylophiceae compose the vast majority of the archaeal community. Methanobrevibacter gottschalkii and Methanobrevibacter ruminantium are the dominant species, composing ˜65% of the ruminal microbiome across all 394 cattle when combined. In addition, the study found that within the rumen microbial community, the archaeal community in general is stable across host species, diet, and geographic region, as bacterial taxa are the main drivers of most observed variation. This knowledge builds confidence that these rumen dominant methanogen species, either alone or in combination, and/or the antigen(s) present in one or both of these rumen dominant methanogen species will be relevant on a global scale.

### Vaccination and Sample Collection

Healthy, weaned, Angus Cross steers are weaned for a minimum of sixty days and received pre-weaned vaccinations a minimum of sixty days prior to initiation of the study. Animals are transferred to the study site at least fourteen days prior to vaccination to allow for acclimation to the study diet and environment. Each vaccine formulation is administered by intramuscular (IM) injection into three animals. In total, 120 animals are vaccinated with a vaccine comprising methanogen cells and/or cell parts; and 3 animals are vaccinated with a negative control (saline). Intramuscular injections are delivered into the cow's right neck muscle by veterinary-trained staff using an 18-gauge 1-1.5″ needle. Animals are boosted with a second vaccine administered on day 21. Total blood samples (50-100 mL) are collected from the jugular vein by veterinary-trained staff and processed by ultracentrifugation within 24 hrs of sample collection to clarify serum. Clarified serum samples are aliquoted, labeled and stored in cryogenic tubes at ≤−20° C. until use.

### Ruminal Microbiome Analysis

This example demonstrates a method for isolating DNA and then amplifying 16S rRNA to assess changes in bacterial and/or archaeal populations within the rumen microbiome following vaccination with the vaccines of the present disclosure.

First, 20-50 mL of rumen contents is collected. Briefly, the ruminant, e.g., steer, is restrained in a squeeze chute with a head gate and nose tucked down toward the chest. Then, a long flexible plastic tube is inserted into the animal's nostril and down into the stomach. A hand-operated rumen fluid pump, attached to the flexible plastic tube, is then used to harvest 20-50 mL of rumen fluid per collection. Harvested rumen fluid samples is then stored in conical vials and transferred to the lab. Finally, within 24 hrs of collection, rumen fluid samples are strained through three layers of cheesecloth, and the liquid phase is aliquoted into 50 mL conical vials, appropriately labeled and snap-frozen in liquid N₂ for cryostorage at ≤−20° C.

Next, isolation of DNA from at least one rumen fluid sample follows one of the methods defined in Henderson, G. et al. Effect of DNA Extraction Methods and Sampling Techniques on the Apparent Structure of Cow and Sheep Rumen Microbial Communities. PLOS One 8, e74787 (2013), which is incorporated herein by reference, or uses the DNEasy Power Biofilm Kit (Qiagen, Hilden, Germany), with preference given to methods involving both phenol-chloroform and mechanical lysis steps (PCQI, PCBB, PCSA). RBBC is the preferred extraction method if the use of phenol-chloroform poses an issue.

Next, PCR amplification of the hypervariable V6-V8 regions of the 16S rRNA gene is performed using the archaea-specific Ar915aF/Ar1386R primer set with Illumina adapters as defined in Table 1 of Kittelmann 2015 (Kittelmann et al. Buccal swabbing as a noninvasive method to determine bacterial, archaeal, and eukaryotic microbial community structures in the rumen. Appl Environ Microbiol 81:7470-7483 (2015), which is incorporated herein by reference) and PCR cycle conditions as defined in Kittelmann 2013 (Kittelmann et al. Simultaneous Amplicon Sequencing to Explore CoOccurrence Patterns of Bacterial, Archaeal and Eukaryotic Microorganisms in Rumen Microbial Communities. PLOS One 8(2): e47879 (2013), which is incorporated herein by reference, each of which is incorporated herein by reference.

PCR products are then used for purification, quality control (QC), library preparation, and 16S rRNA sequencing PCR samples should be purified and QC'ed according to any method well known in the art. PCR samples are then used to generate at least one 16S rRNA amplicon sequencing library using, preferably, the NextFlex DNA-Seq kit (PerkinElmer, Waltham, MA), or a comparable alternative kit selected by a skilled artisan. Finally, the 16S rRNA amplicon sequence library is sequenced on the MiSeq v3 600-cycle platform (2×300 bp; Illumina, San Diego, CA) according to the manufacturer's protocol.

Following the completion of 16S rRNA sequencing, the resulting data is analyzed for methanogen abundance. Methanogen abundance is reduced by ˜10-80% after treatment with cell-based vaccine composition of the present disclosure as compared to pre-vaccination.

### CH₄ Production Measurements

Animal enteric CH₄ production is monitored using GreenFeed Systems (C-Lock, Inc., Rapid City, SD). Briefly, feed intake is recorded continuously throughout the study period using GrowSafe Systems, or a comparable method for tracking individual dry matter intake, as selected by a skilled artisan. Animals are fed once daily in the morning and have access to a water source at all times. Body weight is recorded periodically. Enteric CH₄, H₂, and CO₂ emissions are measured ad libitum throughout the study period using GreenFeed Systems (C-Lock, Inc., Rapid City, SD). Animals have free access to the GreenFeed System throughout the study period; animals are not physically coerced to use the system. CH₄ yield and intensity is calculated using dry matter intake for each measurement period separately. CH₄ production is reduced by ˜10-80% when treated with cell-based vaccine composition of the present disclosure as compared to pre-vaccination.

### Example 7: Pregnant and Postpartum Animal Vaccination

A vaccine is used to vaccinate a pregnant animal (e.g., ruminant). The unborn fetus is exposed to the antibodies against methanogen generated by the mother. After birth, the offspring receives milk and/or colostrum laden with the antibodies against the at least one methanogen surface antigen. The offspring is vaccinated with at least one vaccine of the present disclosure prior to weaning. The offspring may be vaccinated with a cell-based vaccine that is the same or different from the cell-based vaccine used to vaccinate the mother.

### Example 8: Combinatorial Therapies

A subject is treated with a combinatory therapy, which comprises any two or more selected from a vaccine, antibodies, milk, animal feed, an agent (e.g., an agent that reduces CH₄ production in a subject, a probiotic bacterial strain, a small molecule inhibitor, etc.), and other composition of the present disclosure (e.g., those reducing CH₄ production in a subject). Any one of the combinatory therapies may be given in any order, i.e., before, concurrently with, or after any other combinatory therapy. Here, a reduction in CH₄ production that is greater than the use of a single therapy alone is achieved.

### Example 9: Synthesis of 3NOP

Adapted from “Structure-Based Design, Synthesis, and Biological Evaluation of Indomethacin Derivatives as Cyclooxygenase-2 Inhibiting Nitric Oxide Donors” (J. Med. Chem. 2007, 50, 6367-6382).

All reagents purchased from Sigma-Aldrich and Fisher Scientific and used as received.

3-Bromopropanol (78.0 g, 0.56 mol) in acetonitrile (300 mL) was added dropwise within min. to a solution of silver nitrate (145.9 g, 0.86 mol) in acetonitrile (600 mL) and stirred at room temperature for 24 hr. The solution was protected from light by being covered with aluminum foil. After 24 hr, 5:1 excess of brine was added to the reaction mixture and stirred for 1 hr. Silver halide was filtered through Celite and filtrate was extracted with diethyl ether (300 mL×3). The organic layer was washed with brine (300 mL×3). Dried over sodium sulfate, and concentrated (59 g, 86% yield). Product confirmed via NMR (>97% purity based on HPLC).

3NOP pH and Temperature Stability: Confirmed via HPLC over a 1-month time period at temperature between 4-30° C. and pH 4-10.

3NOP Volatility:500 mg of 3NOP in a 5 dram vial was left open to air at ˜18-24° C. at 15-30% RH over the course of 6 days. Average mass loss is ˜0.25% per day.

### Compaction Study

The viability of different binders for densification were examined. 0.5 g of powder was tableted under 20 kN in a 13 mm pellet die. Potential binders are summarized in Table 16A.

TABLE 16A
Potential binders ranked based on densification.

| | | | 2: compacts, |
|---|---|---|---|
| | | | no strength |
| | | 3: powdery break, | 1: compacts, |
| 5: Unbreakable | 4: clean braking, | retains moderate | disintegrates |
| tablet by hand | no dust | strength | 0: no compaction |
| Xanthan gum | Lignin | Pea protein | Chitosan |
| k-carrageenan | Inulin | Soy protein | Hydroxyethyl cellulose |
| Sodium alginate | Wood flour | Gelatin | Talc |
| Cellulose acetate | Corn Starch | Casein | Sodium starch glycolate |
| Ethyl cellulose | Amylopectin | | Pentaerythritol |
| Hydroxypropyl cellulose | Hemp protein | | Calcium phosphate |
| Cellulose acetate | | | Diatomaceous earth |
| phthalate | | | (Celite) |
| Maltodextrin | | | Biochar |
| Dextran | | | |

### Tablets for Encapsulation of 3NOP

A multilayer encapsulation system was employed for extended-release formulations consisting of 3 main parts:
1) Adsorbent—inert, non-toxic, solid-support for 3NOP (i.e., silica, biochar, activated charcoal, talc, arginine, lysine, calcium carbonate, carbon black, cyclodextrin, calcium phosphate, celite (diatomaceous earth), glutamine, betaine, bismuth phosphate, bismuth citrate, iron phosphate, etc.)
2) Binder/matrix—allow for granulation of adsorbent to form solid core (i.e., starches, flours, sugars, proteins, gums, gelatin, cellulose and derivatives, etc.)
3) Coating—single to multilayer coatings to decrease water penetration and modify release kinetics

All materials purchased from Sigma-Aldrich and Fisher Scientific and used as received unless otherwise specified.

### Tablet Preparation

3NOP was adsorbed onto the corresponding adsorbent at a given weight percent (noted in formulations) by dropwise addition to adsorbent stirring on a hot plate. The mixture was left to stir for more than 30 min. until a free-flowing powder was obtained. The resulting mixture was speed mixed with the corresponding binder for 1 minute followed by compaction of in a carver press using an 8-13 mm pellet die under various forces of 10-60 kN.

### Coated Tablet Preparation

Selected coating materials were dissolved in various solvents at 5-20 wt %. Tablets were then dip coated multiple times allowing for drying in between to give the desired coating weight.

### 3NOP Release Quantification and Corresponding Tablet Formulations

13 mm, 0.5 g tablets were placed in a sealed 5 dram vial with 10 mL of distilled water and incubated at 70° F. until full dissolution of 3NOP. 1 mL aliquots were taken every few days and refreshed with 1 mL of distilled water. Aliquots were filtered through a 0.22 μm PVDF filter before being quantified via HPLC.

### Uncoated Tablets

All formulation percentages are based on weight. 2 replicates of 13 mm disc-shaped, 0.5 g tablets were compacted under 20 kN. The uncoated formulations had the following compositions:
AH-01-7.5% 3NOP, 12.5% propylene glycol, 60% Cargill hemp protein
AH-02-8.57% 3NOP, 22.86% Celite, 68.57% hemp protein
AH-03-8.57% 3NOP, 22.86% Wakefield Biochar, 68.57% Cargill hemp protein
AH-04-8.57% 3NOP, 22.86% K10 montmorillonite, 68.57% Cargill hemp protein
AH-05-8.57% 3NOP, 22.86% activated charcoal, 68.57% Cargill hemp protein The 3NOP release of the uncoated tablets after 12 hrs is shown in Table 17.

TABLE 17
3NOP release after 12 hrs

| | Total Average Concen- | Standard |
|---|---|---|
| Formula | tration of 3NOP (mM) | deviation (mM) |
| AH-01 | 24.56 | 2.09 |
| AH-02 | 24.50 | 1.76 |
| AH-03 | 19.61 | 0.17 |
| AH-04 | 33.10 | 0.10 |
| AH-05 | 18.58 | 0.07 |

### Coated Tablets

Tablets for coating experiments consist of 10% 3NOP, 20% Wakefield biochar, and 70% cellulose acetate (Mn:50,000). Tablets were then coated with ˜0.2 wt % of the corresponding coating material, as shown in the table in Table 18. The release profile over periods of 4, 6 and 11 days for each formula is also shown.

TABLE 18
3NOP release profile of coated tablets over 4 days.

| | Time | Total Ave. | St. dev. |
|---|---|---|---|
| | (days) | Conc. (mM) | (mM) |
| Cellulose Acetate Phthalate (CAP) | 4 | 34.21 | 2.23 |
| CAP | 6 | 30.65 | 0.04 |
| CAP | 11 | 32.38 | 0.24 |
| Ethyl cellulose (EC) | 4 | 29.92 | 0.06 |
| EC | 6 | 31.07 | 0.01 |
| EC | 11 | 31.50 | 0.26 |
| Hydroxy propyl cellulose (HPC) | 4 | 31.70 | 0.05 |
| HPC | 6 | 30.35 | 0.01 |
| HPC | 11 | 32.53 | 0.4 |
| Polycaprolactone (CAPA 6800) | 4 | 32.08 | 0.29 |
| CAPA 6800 | 6 | 31.00 | 0.14 |
| CAPA 6800 | 11 | 31.99 | 0.23 |
| Alignate (layer 1) Chitosan (layer 2) | 4 | 30.30 | 0.17 |
| Alignate (layer 1) Chitosan (layer 2) | 6 | 30.22 | 0.17 |
| Alignate (layer 1) Chitosan (layer 2) | 11 | 31.90 | 0.10 |
| Polycaprolactone Mn: 2000 | 4 | 33.68 | 0.01 |
| Polycaprolactone Mn: 2000 | 6 | 32.25 | 0.18 |
| Polycaprolactone Mn: 2000 | 11 | 33.23 | 0.09 |
| PEG Mn: 1500 | 4 | 32.74 | 0.22 |
| PEG Mn: 1500 | 6 | 31.88 | 1.30 |
| PEG Mn: 1500 | 11 | 33.48 | 0.29 |

Further formulations were prepared and tested. 3 replicates of 13 mm disc-shaped, 0.5 g tablets were compacted under 60 kN. All formulas consist of 23.8% 3NOP, 28.6% silica, and 47.6% ethyl cellulose. Tablets were then coated with 60-80 wt % of the corresponding coating material. Control tablet (CTRL) is uncoated. “R” denotes replicate number. CAP coating solution was 18 wt % in 70% ethanol/water. Cellulose acetate (CA) coating was 6 wt % in 70% ethanol/ethyl acetate with an additional 5 wt % triethyl citrate, 5 wt % propylene glycol, and 5 wt % N-methyl-2-pyrollidone. The release profiles were analyzed at 1, 4, 8, 11, and 15 days, as summarized in Table 19.

TABLE 19
3NOP release profile of coated tablets over 15 days.
Total concentration of 3NOP is listed in mM.

| | | | | | |
|---|---|---|---|---|---|
| CAP R1 | 25.74 | 47.79 | 59.39 | 61.05 | 59.68 |
| CAP R2 | 18.72 | 43.71 | 52.56 | 53.74 | 53.37 |
| CAP R3 | 23.12 | 50.66 | 57.23 | 59.23 | 58.75 |
| AVG | 22.53 | 47.39 | 56.39 | 58.01 | 57.26 |
| STD | 3.54 | 3.49 | 3.49 | 3.81 | 3.41 |
| CA R1 | 50.90 | 75.98 | 73.80 | 66.97 | 61.48 |
| CA R2 | 71.29 | 78.41 | 73.11 | 66.66 | 60.18 |
| CA R3 | 63.86 | 69.38 | 65.56 | 61.28 | 57.00 |
| AVG | 62.01 | 74.59 | 70.82 | 64.97 | 59.55 |
| STD | 10.32 | 4.67 | 4.57 | 3.20 | 2.31 |
| CTRL R1 | 45.80 | 80.64 | 80.33 | 73.64 | |
| CTRL R2 | 45.80 | 84.41 | 83.39 | 76.22 | 69.25 |
| CTRL R3 | 45.80 | 77.15 | 75.92 | 70.11 | 62.74 |
| AVG | 45.80 | 80.73 | 79.88 | 73.32 | 66.00 |
| STD | 0.00 | 3.63 | 3.76 | 3.07 | 4.60 |

### Coated Tablets

All formulation percentages are based on weight. 3 replicates of 13 mm disc-shaped, 0.5 g tablets were compacted under 60 kN. All formulas consist of 23% 3NOP, 30% silica, and 47% ethyl cellulose. Tablets were then coated with 60-80 wt % of the corresponding coating material. Control tablet (CTRL) is uncoated. V1 coating solution was 20 wt % cellulose acetate Mn: 50000 in 70% acetone/ethyl acetate with BYK coating additives. V2 coating solution was 20 wt % in 70% acetone/ethyl acetate cellulose acetate Mn: 50000 with BYK coating additives. Ethyl cellulose coating solution was 20 wt % in 70% ethanol/water with 15 wt % triacetin. CAP coating solution was 18 wt % in 70% ethanol/water with 10 wt % propylene glycol. The 3NOP release profile of the coated tablets over 18 days is shown in Table 20.

TABLE 20
3NOP release profile of coated tablets over 18 days.
Total concentration of 3NOP is listed in mM.

| Time | 1 | 5 | 8 | 12 | 18 |
|---|---|---|---|---|---|
| V1 R1 | 33.04 | 70.32 | 70.57 | 66.04 | 60.88 |
| V1 R2 | 57.54 | 77.54 | 77.42 | 72.65 | 67.44 |
| V1 R3 | 61.42 | 71.21 | 70.08 | 65.71 | 61.19 |
| AVG | 50.67 | 73.02 | 72.69 | 68.13 | 63.17 |
| STD | 15.39 | 3.93 | 4.10 | 3.91 | 3.70 |
| V2 R1 | 31.15 | 66.47 | 65.62 | 61.03 | 56.78 |
| V2 R2 | 32.55 | 66.81 | 67.61 | 63.67 | 59.55 |
| V2 R3 | 53.59 | 69.37 | 71.28 | 67.53 | 63.01 |
| AVG | 39.10 | 67.55 | 68.17 | 64.08 | 59.78 |
| STD | 12.57 | 1.59 | 2.87 | 3.27 | 3.12 |
| EC R1 | 17.99 | 39.67 | 45.39 | 47.12 | 50.00 |
| EC R2 | 15.84 | 38.15 | 43.53 | 45.83 | 49.27 |
| EC R3 | 21.21 | 52.28 | 58.64 | 60.42 | 63.56 |
| AVG | 18.35 | 43.37 | 49.19 | 51.13 | 54.28 |
| STD | 2.70 | 7.75 | 8.24 | 8.08 | 8.05 |
| CAP R1 | 24.07 | 57.62 | 63.10 | 69.28 | 68.61 |
| CAP R2 | 23.24 | 56.30 | 61.85 | 60.67 | 59.42 |
| CAP R3 | 37.58 | 63.57 | 71.80 | 69.89 | 70.48 |
| AVG | 28.30 | 59.17 | 65.58 | 66.61 | 66.17 |
| STD | 8.05 | 3.87 | 5.42 | 5.15 | 5.92 |
| CTRL R1 | 61.62 | 97.29 | 89.52 | 81.26 | 74.20 |
| CTRL R2 | 45.40 | 82.69 | 79.42 | 73.16 | 67.07 |
| CTRL R3 | 53.75 | 83.30 | 76.77 | 70.18 | 64.12 |
| AVG | 53.59 | 87.76 | 81.90 | 74.87 | 68.47 |
| STD | 8.11 | 8.26 | 6.73 | 5.74 | 5.18 |

### Bilayer Coated Tablets

All formulation percentages are based on weight. 3 replicates of 13 mm disc-shaped, 0.5 g tablets were compacted under 60 kN. All formulas consist of 15% 3NOP, 20% silica, and 65% ethyl cellulose. Bilayer coatings were applied with ˜10 wt % of the first coating material and ˜20 wt % of the second coating material. CAP/EC denoted first and second coating material. The 3NOP release profile of bilayer coated tablets over 27 days is shown in Table 21.

TABLE 21
3NOP release profile of bilayer coated tablets over
27 days. Total concentration of 3NOP is listed in mM.

| Time (days) | 1.25 | 2.5 | 5.5 | 8 | 10 | 14 | 17 | 21 | 27 |
|---|---|---|---|---|---|---|---|---|---|
| CAP/EC R1 | 6.27 | 8.80 | 15.97 | 18.49 | 20.41 | 24.15 | 24.59 | 24.46 | 26.21 |
| CAP/EC R2 | 4.39 | 7.77 | 15.50 | 17.78 | 19.54 | 22.90 | 23.44 | 23.74 | 25.94 |
| CAP/EC R3 | 5.63 | 8.43 | 15.44 | 17.84 | 19.29 | 20.36 | 20.94 | 21.18 | 22.65 |
| AVG | 5.43 | 8.33 | 15.64 | 18.04 | 19.75 | 22.47 | 22.99 | 23.13 | 24.93 |
| STD | 0.96 | 0.52 | 0.29 | 0.39 | 0.59 | 1.93 | 1.87 | 1.73 | 1.98 |
| EC/CAP R1 | 9.75 | 14.58 | 22.35 | 23.09 | 23.76 | 26.40 | 26.04 | 26.12 | 26.82 |
| EC/CAP R2 | 10.72 | 17.13 | 24.89 | 26.10 | 26.78 | 29.51 | 28.89 | 28.52 | 29.31 |
| EC/CAP R3 | 9.84 | 15.34 | 22.33 | 24.08 | 25.21 | 28.03 | 27.91 | 27.56 | 28.17 |
| AVG | 10.10 | 15.68 | 23.19 | 24.42 | 25.25 | 27.98 | 27.61 | 27.40 | 28.10 |
| STD | 0.54 | 1.31 | 1.47 | 1.54 | 1.51 | 1.56 | 1.45 | 1.21 | 1.25 |
| CTRL R1 | 26.11 | 28.76 | 36.26 | 37.49 | 36.75 | 35.77 | 33.69 | 31.26 | 32.66 |
| CTRL R2 | 26.12 | 28.43 | 38.45 | 39.57 | 38.58 | 37.45 | 34.91 | 32.16 | 33.30 |
| CTRL R3 | 25.23 | 29.94 | 40.65 | 41.23 | 45.02 | 42.91 | 39.10 | 27.92 | −2.04 |
| AVG | 25.82 | 29.05 | 38.45 | 39.43 | 40.12 | 38.71 | 35.90 | 30.45 | 21.31 |
| STD | 0.51 | 0.79 | 2.19 | 1.87 | 4.34 | 3.73 | 2.84 | 2.24 | 20.22 |

### Adsorbent Study

Various adsorbents were assessed for potential binding affinity and adsorptive capacity of 3NOP. Activated carbon/charcoal and silica were identified as the most suitable adsorbents based on their high binding affinity and adsorptive capacity.

Briefly, 0.2 g of adsorbent was added to 4 mL of 2.5% 3NOP/H₂O stock solution or 10 mL of 1% 3NOP/H₂O stock solution. The solution was then placed in an ultrasonic bath for ˜4 hr and then allowed to equilibrate over 24 hr. All materials were purchased from Sigma-Aldrich and used as received. After 24 hr, adsorbents were tested for their binding affinity and adsorptive capacity by looking at the reduction of 3NOP concentration via HPLC at 230 nm.

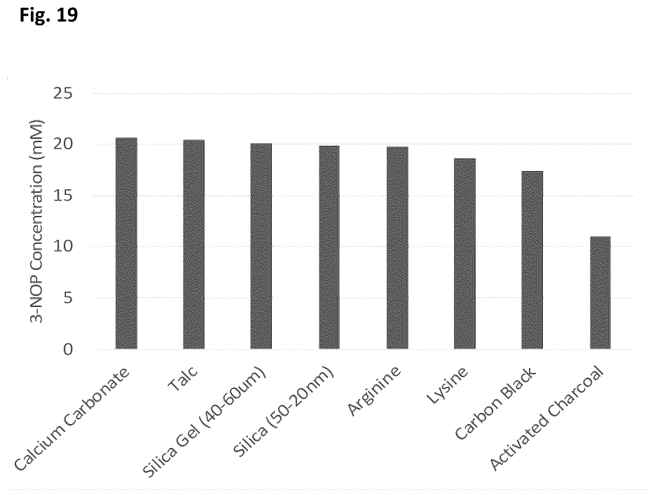

The following adsorbents were tested and integrated areas were converted to mM concentrations of 3NOP based on calibration standards of known 3NOP concentration in distilled water, as seen in Table 22. FIG. 19 is graph showing the concentration of 3NOP (mM) v. adsorbent (20 mM stock solution).

TABLE 22
Change in concentration vs. control of 0.2
g of adsorbent in 4 mL of 2.5% 3NOP/H₂O

| Adsorbent | 3NOP concentration (mM) |
|---|---|
| Control | 20.649 |
| Calcium Carbonate | 20.649 |
| Talc | 20.463 |
| Silica Gel (40-60 um) | 20.043 |
| Silica (5-20 nm) | 19.920 |
| Arginine | 19.782 |
| Lysine | 18.583 |
| Carbon Black | 17.422 |
| Activated Charcoal | 11.063 |

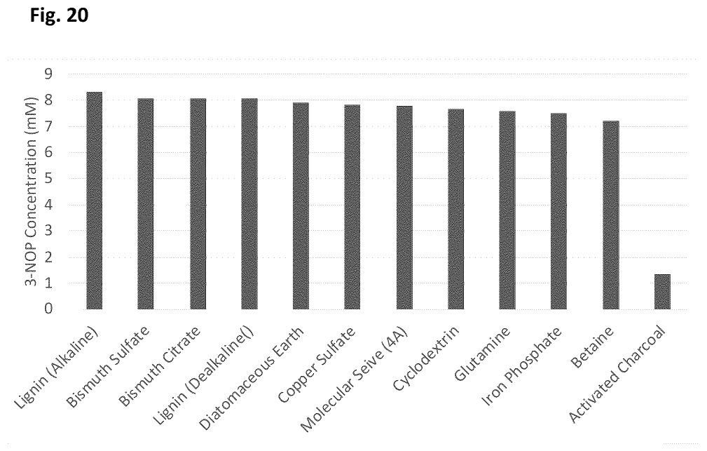

Table 23 shows the change in concentration v. control of 0.2 g of adsorbent in 10 mL of 1% 3NOP/H₂O, and FIG. 20 shows 3NOP concentration (mM) v. adsorbent (˜8 mM stock solution).

TABLE 23
Change in concentration vs. control of 0.2
g of adsorbent in 10 mL of 1% 3NOP/H₂O

| Adsorbent | 3NOP concentration (mM) |
|---|---|
| Control | 8.258 |
| Lignin (Alkaline) | 8.326 |
| Bismuth Sulfate | 8.079 |
| Bismuth Citrate | 8.072 |
| Lignin (Dealkaline) | 8.056 |
| Diatomaceous Earth | 7.895 |
| Copper Sulfate | 7.814 |
| Molecular Sieve (4Å) | 7.771 |
| Cyclodextrin | 7.676 |
| Glutamine | 7.588 |
| Iron Phosphate | 7.503 |
| Betaine | 7.202 |
| Activated Charcoal | 1.358 |

Activated carbon shows strong binding affinity for 3NOP filtering ˜45 wt % in concentrated (20 mM) conditions with ˜80 wt % in more dilute concentrations (8 mM). It appears that the adsorbent pore size should be greater than 4 Å for adsorption of 3NOP.

Mesoporous adsorbents with high surface area such as silica and activated carbon show high adsorptive capacity with no visible wetting seen until ˜75 wt %. Layer-by-Layer

### Polyelectrolytes Coatings

Formulated tablets containing 3NOP, an adsorbent, and a binder can be coated with sequential layers of oppositely charged polyelectrolytes. While not being bound by theory, it is believed that ionic crosslinking between the layers can result in a controlled release of 3NOP out of the tablets. Examples of polyelectrolyte pairs that can be used include polyglutamic acid and polylysine, polyallylamine hydrochloride and polyacrylic acid, and polyallylamine hydrochloride and polystyrene sulfonate. An exemplary formulated tablet includes an adsorbent of silica, arginine, lysine, and activated charcoal (30-45%) and a binder of ethyl cellulose and hydroxypropyl cellulose (40-55%).

TABLE 24
Formulation examples of 3NOP tablets. Tablet weight
varied from 0.2-0.5 g
Tablet Formulation

| | Part per | | Part per |
|---|---|---|---|
| | formulation | | formulation |
| Adsorbent | (wt %) | Binder | (wt %) |
| Silica, Arginine, Lysine, | 30-45 | Ethyl Cellulose, | 40-55 |
| Activated Charcoal | | Hydroxypropyl | |
| | | Cellulose | |

The release data of 3NOP tablets coated with multilayers of some polyelectrolytes is shown in Table 25.

TABLE 25
Release (%) of 3NOP out of tablets formulated with 15% 3NOP, 30% silica,
and 55% ethyl cellulose. Weight of tablets ~0.5 g. Calculations
are based on a comparison to control tablets with no coating.

| | Release Compared to Control Tablets (%) | | | | | | | |
|---|---|---|---|---|---|---|---|---|
| PE Pair | Day 1 | Day 3 | Day 6 | Day 8 | Day 14 | Day 16 | Day 21 | Day 30 |
| PLK20 + | 58.1 | 56.6 | 61.3 | 62.6 | 64.7 | 63.6 | 63.8 | 63.2 |
| PRE20 | | | | | | | | |
| PAH + PAA | 61.5 | 62.5 | 67.1 | 70.2 | 70.4 | 71.4 | 71.7 | 71.6 |
| PAH + PSS | 61 | 66.5 | 74.7 | 76 | 77.5 | 76.6 | 76.7 | 74.8 |

### 3NOP to Adsorbent Ratio Study

Optimal loading of 3NOP onto adsorbents was determined by varying ratios of 3NOP to adsorbent. 3NOP and adsorbent were speed mixed at 2000 rpm for 1 min. at the following ratios by weight: 1:1.5, 1:2, 1:2.5. The adsorbed 3NOP powder mixture was then placed in distilled water at 1% 3NOP loading. The solution was allowed to equilibrate for 24 hr and then an aliquot was taken for HPLC to determine the reduction in 3NOP concentration. Table 26 shows the average integrates are of 3NOP in solution at various adsorbent ratios.

TABLE 26
integrated area of 3NOP in solution at varying 3NOP:adsorbent ratios

| | 3NOP:Adsorbent | | | | | |
|---|---|---|---|---|---|---|
| | 1:1.5 | | 1:2 | | 1:2.5 | |
| | Average | | Average | | Average | |
| | Integrated | Standard | Integrated | Standard | Integrated | Standard |
| | Area | Deviation | Area | Deviation | Area | Deviation |
| 3NOP | 2566.4 | 51.3 | 2566.4 | 51.3 | 2566.4 | 51.3 |
| Silica | 2499.4 | 293.2 | 2321.9 | 99.3 | 2340.9 | 30.3 |
| Activated | 1751.2 | 29.6 | 1581.9 | 8.5 | 1403.7 | 14.5 |
| charcoal | | | | | | |
| Lysine | 2336.2 | 61.7 | 2308.9 | 23.9 | 2266.6 | 56.1 |
| Arginine | 2406.0 | 113.2 | 2372.8 | 61.2 | 2435.2 | 230.2 |
| Glutamic acid | 2445.9 | 149.9 | 2452.5 | 78.9 | 2446.3 | 85.0 |

Higher ratios of activated carbon to 3NOP result in higher adsorptive capacity. At ratio of 1:2.5 an adsorption capacity of ˜45% is observed and at a ratio of 1:1.5 an adsorption capacity of ˜30% is observed.

### Activated Carbon Tablets

Based on the 3NOP:adsorbent ratio study, the same ratios were tested for tablet integrity, friability, porosity, etc. to determine the optimal 3NOP loading. 500 mg, 13 mm tablets were compacted under 60 kN.

High ratios of activated carbon (1:2.5 3NOP:adsorbent) resulted in tablets that absorb more water and coating solutions. This water/solvent absorption of non-wetted charcoal causes difficulty in producing uniform, defect free coatings. High loadings of activated charcoal >25% in tablets result in poor tablet integrity characterized by loss in hardness, increased porosity, tablet spilling, and poor shape retention. The preferred loading for tablets with poor disintegration was 1:1.5 3NOP:adsorbent.

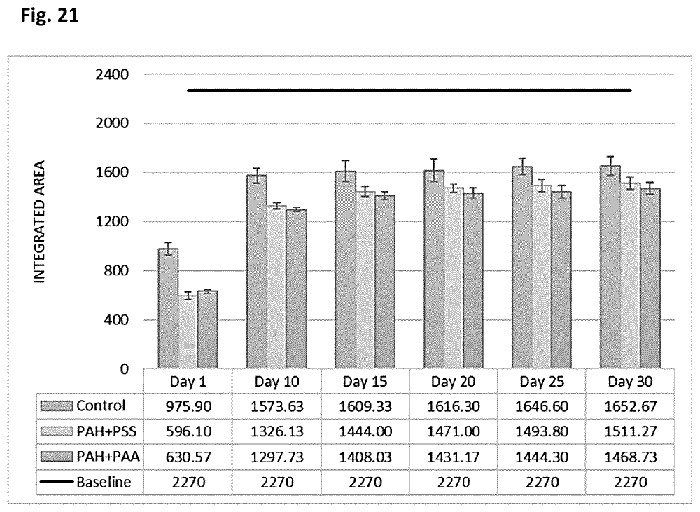

FIG. 21 shows a multilayer tablet formulation above consisting of 10 wt % 3NOP, 15 wt % activated carbon, and 75 wt % ethyl cellulose. The first day release of coated tablets is ˜40% less than uncoated tablets and after 30 days, ˜30% 3NOP remains adsorbed on activated carbon.

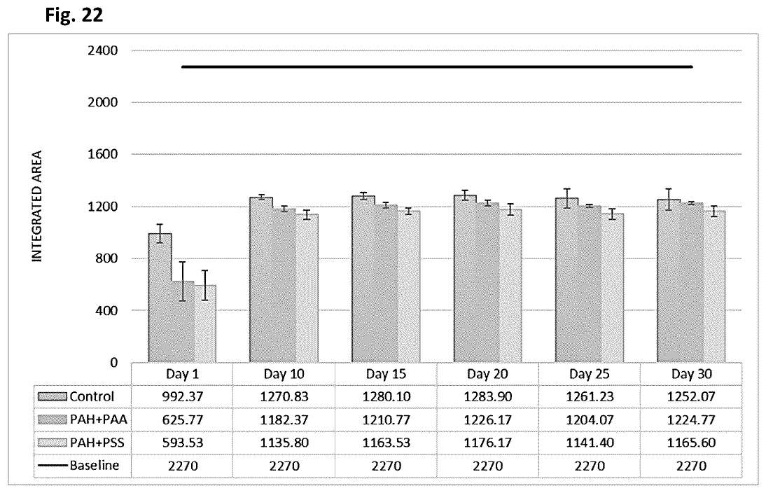

FIG. 22 shows a multilayer tablet formulation consisting of 10 wt % 3NOP, 25 wt % activated carbon, and 75 wt % ethyl cellulose. After 30 days, ˜50% 3NOP remains adsorbed on activated carbon. These adsorptive capacities are in-line with what was observed during the 3NOP:adsorbent ratio study.

FIG. 23 shows a multilayer formulation consisting of 10 wt % 3NOP, 15 wt % activated carbon, 5 wt % sodium lignosulfonate, and 70 wt % ethyl cellulose. The addition of 5 wt % sodium lignosulfonate improves coating adhesion to the tablet resulting in ˜30% reduction in the first day 3NOP release. Over time, the release rate remains similar to previous formulations with a residual 30% 3NOP remaining adsorbed to activated carbon.

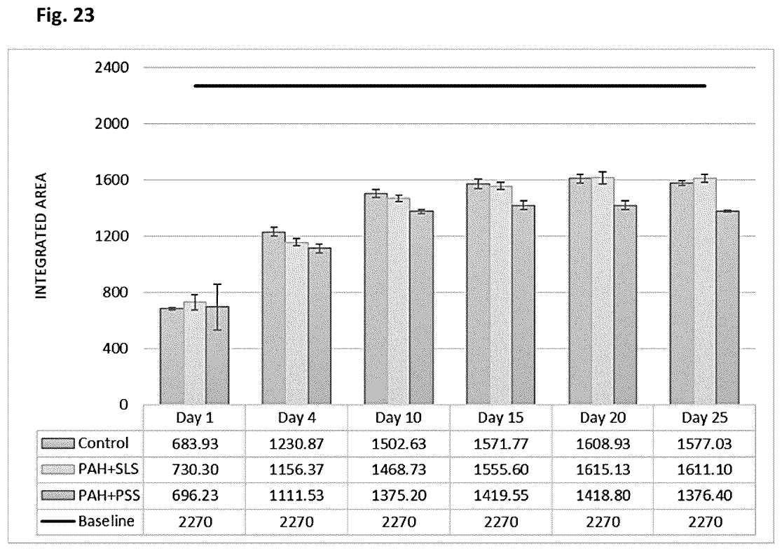

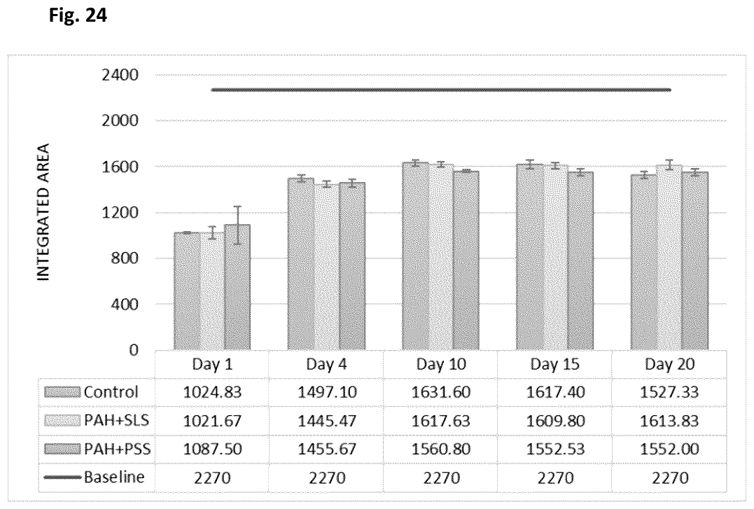

FIG. 24 shows a multilayer formulation consisting of 10 wt % 3NOP, 15 wt % activated carbon, 5 wt % hydroxypropyl cellulose, and 70 wt % ethyl cellulose. The addition of 5 wt % hydroxypropyl cellulose, a hydrophilic water-soluble binder, results in increased release between day 4 and day 10. After 30 days, ˜30% 3NOP remains adsorbed to activated carbon.

### Silica V. Activated Carbon Controls to Assess Binding Affinity of 3NOP

Various control experiments were conducted to confirm the adsorbed 3NOP to activated carbon after 30 days. Activated carbon was compared to silica as preferred adsorbents.

500 mg of 1:1.5 3NOP:adsorbent were speed mixed at 2000 rpm for 1 min. The resulting powder mixture was placed in 10 mL of distilled water and 10 mL of acetonitrile (ACN) separately. The solutions were placed in a sonic bath for ˜5 hr, removed, and allowed to equilibrate for 24 hr.

Aliquots were filtered through a 0.22 μm PTFE filter, and taken for HPLC to measure the 3NOP concentration in solution.

The same trend was observed in the adsorbent study. However, 3NOP is recoverable by washing the activated carbon with acetonitrile or a similar organic solvent with high 3NOP solubility (i.e., ethanol, acetone, ethyl acetate, tetrahydrofuran, chloroform).

TABLE 27
Silica powder release vs. activated carbon release
in distilled water and acetonitrile.

| | 24 hr in H₂O | 24 hr in ACN |
|---|---|---|
| | Integrated Area | Integrated Area |
| Si-1 | 8415.7 | 8847 |
| Si-2 | 8507.3 | 8822.2 |
| Average | 8461.5 | 8834.6 |
| AC-1 | 4940.8 | 8103.9 |
| AC-2 | 5577.5 | 8082.8 |
| Average | 5259.2 | 8093.4 |

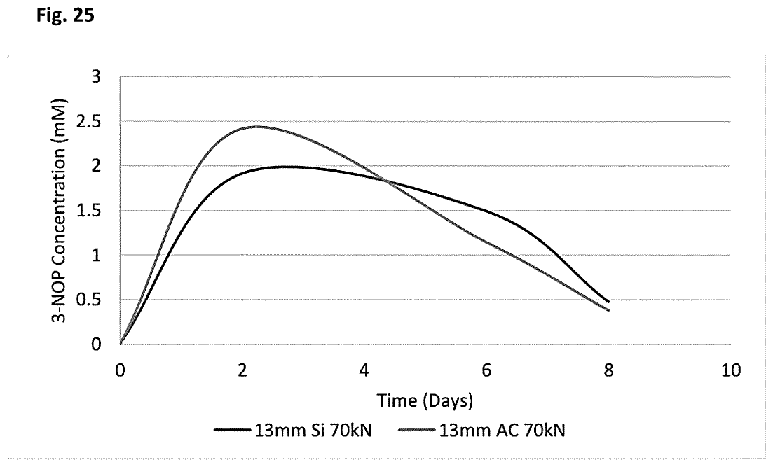

After 24 hr in water, less than 5 wt % 3NOP remained adsorbed to silica compared to the powder mix placed in acetonitrile. After 24 hr in water, ˜35 wt % 3NOP remained adsorbed to activated carbon compared to the powder mix placed in acetonitrile. By washing the activated carbon in acetonitrile, the adsorbed 3NOP can be recovered in solution proving that the remaining 30-50% 3NOP that is seen in FIG. 17-FIG. 19 remains adsorbed. Further studies need to be conducted to determine the driving forces for 3NOP desorption from activated carbon under different environmental conditions to utilize adsorptive capacity of activated carbon. This study also confirms that from previous tablet formulations, diffusion is the primary driver for 3NOP release of unbound 3NOP. This can be seen by very similar release profiles between silica-based adsorbent tablets and activated carbon-based adsorbent tablets. FIG. 25 compares a silica-based tablet and activated carbon-based tablet over 8 days. Small differences in release profiles could be due to differences in porosity between tablets using silica or activated carbon.

### Compounding of 3NOP

3NOP was compounded with a variety of matrix materials using a DSM Xplore MC15 conical twin-screw extruder. The extrudate was pelletized and used as is or further compression molded into tablets as a suitable 3NOP extended-release form.

3NOP was adsorbed onto the corresponding adsorbent and speed mixed with binder prior to compounding is a DSM Xplore MC15 conical twin-screw extruder at 100° C. and 50 rpm. The strand was then pelletized, heated, and molded into a 13 mm disc-shaped tablet using a carver press under 40 kN of compaction force. Formula consists of 8.3% 3NOP, 25.0% arginine, 66.7% CAPA 6800.

TABLE 28
3NOP release profile of compounded tablets over 24
days. Total concentration of 3NOP is listed in mM.

| Time | 1 | 3 | 7 | 10 | 14 | 20 | 24 |
|---|---|---|---|---|---|---|---|
| Arg R1 | 8.97 | 16.37 | 22.55 | 23.42 | 24.11 | 23.07 | 24.56 |
| Arg R2 | 12.49 | 15.85 | 20.43 | 21.45 | 21.60 | 19.33 | 19.97 |
| Arg R3 | 9.90 | 17.78 | 24.52 | 24.50 | 24.36 | 22.60 | 22.33 |
| AVG | 10.45 | 16.67 | 22.50 | 23.13 | 23.36 | 21.67 | 22.29 |
| STD | 1.82 | 1.00 | 2.04 | 1.55 | 1.52 | 2.04 | 2.29 |

Formulas consists of 11.1% 3NOP, 13.9% silica, 75.0% CAPA 6800 denoted “11PCL” and 19.4% 3NOP, 24.2% silica, 56.5% CAPA 6800 denoted “19PCL.”

TABLE 29
3NOP release profile of compounded tablets over 24
days. Total concentration of 3NOP is listed in mM.

| Time | 1 | 3 | 7 | 13 | 17 |
|---|---|---|---|---|---|
| 11PCL R1 | 9.79 | 18.77 | 24.31 | 29.15 | 27.93 |
| 11PCL R2 | 10.34 | 19.81 | 24.99 | 30.02 | 29.49 |
| 11PCL R3 | | 19.05 | 22.51 | 27.14 | 26.95 |
| AVG | 10.06 | 19.21 | 23.94 | 28.77 | 28.12 |
| STD | 0.39 | 0.54 | 1.28 | 1.48 | 1.28 |
| 19PCL R1 | 23.55 | 40.56 | 49.99 | 55.00 | 3.48 |
| 19PCL R2 | 24.19 | 40.59 | 51.03 | 53.46 | 51.64 |
| 19PCL R3 | 25.05 | 42.76 | 52.21 | 57.93 | 55.77 |
| AVG | 24.26 | 41.30 | 51.08 | 55.47 | 36.96 |
| STD | 0.75 | 1.26 | 1.11 | 2.27 | 29.07 |

### Compounded Polyelectrolyte Complexes for Extended Release of 3NOP

Solid polyelectrolyte complexes can be processible when plasticized with aqueous electrolytes to act as a matrix for 3NOP molecules. An example of a polyelectrolyte pair for compounding is polydiallyldimethylammonium chloride (PDADMAC), as a cationic polyelectrolyte, and polystyrene sulfonate (PSS), as an anionic polyelectrolyte. At no salt conditions, the complex is brittle and not ideal for processing and the addition of salt, such as sodium chloride, can help with the plasticization and further processing. A trial formulation used for compounding includes mixing of equal charge stoichiometry of PDADMAC and PSS solutions with a concentration of 100 mM, with respect to each polymer, and a total volume of 200 mL (as per preparation method described in ACS Appl. Mater. Interfaces 2015, 7, 895-901). The polyelectrolyte complex can be separated, removed from the supernatant phase, and soaked in salt water with a concentration of 1M for 24 h. The polyelectrolyte complex should be decanted and ready for compounding. A typical formulation used for compounding is summarized in Table 30.

TABLE 30
Compounding formulation with polyelectrolyte complexes for extended
release of 3NOP. Total materials used 20 g. 3NOP was mixed with
arginine (as in solid powder) overnight at 30° C.
PEC Compounding Formulation

| | Part per | | Part per |
|---|---|---|---|
| | formulation | | formulation |
| 3NOP Complex | (wt %) | PEC Complex | (wt %) |
| 3NOP:Arginine | 25 | PDADMAC:PSS | 75 |
| (ratio 1:3, wt %) | | (Equimolar with respect | |
| | | to the monomer) | |

Table 31 shows release (%) of 3NOP out of compounded PEC formulated as per Table 21. The compounded PECs were cut into 5-10 mm pellets and soaked in water (10 mL) for the release study.

TABLE 31
Release percentages
Release (%)

| Day 1 | Day 4 | Day 6 | Day 12 |
|---|---|---|---|
| 22.4 | 34.4 | 35 | 35.7 |

### Compounded Pellets

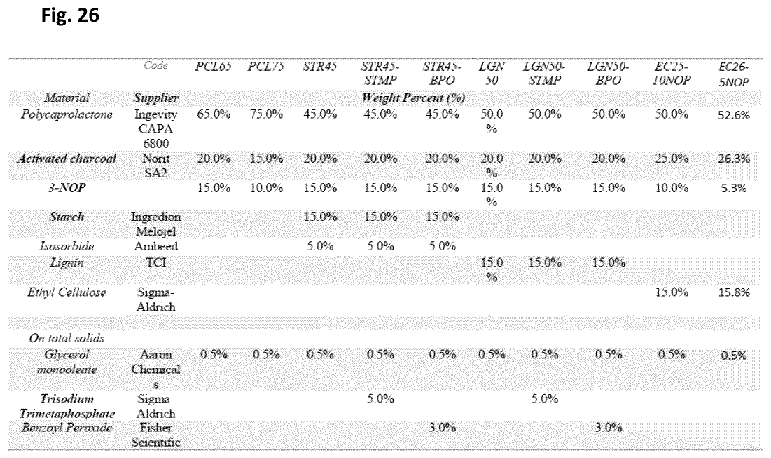

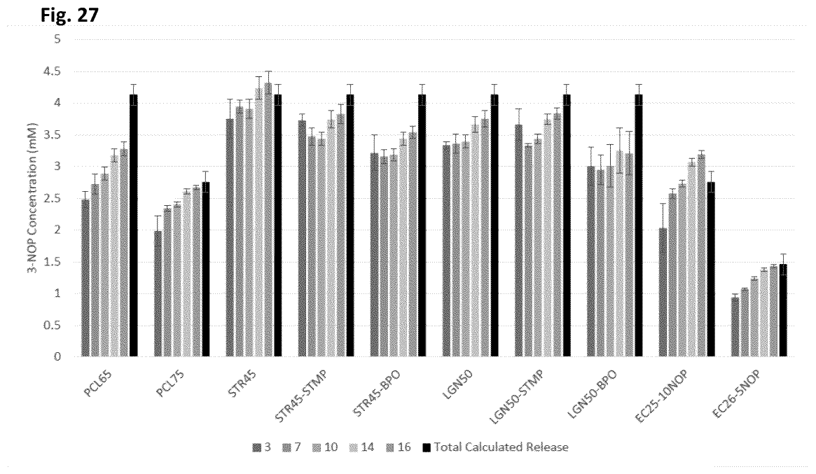

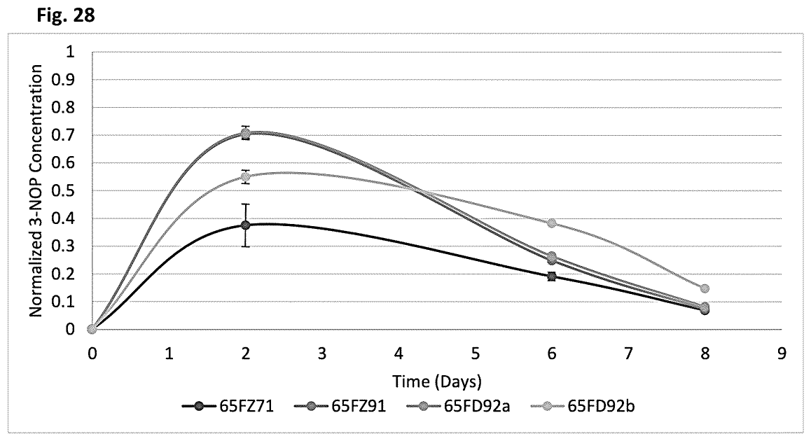

3NOP was adsorbed onto the corresponding adsorbent and speed mixed with binder prior to compounding is a DSM Xplore MC15 conical twin-screw extruder at temperatures of 110-130° C. and 100 rpm. The extrudate was then pelletized forming ˜2 mm pellets. Material suppliers are listed in Table 30. Polybutylene succinate grades FZ71, FZ91, FD92 were supplied by Mitsubishi Chemical. Polycaprolactone-based formulations are summarized in the Table in FIG. 26. FIG. 27 shows 3NOP release in mM for polycaprolactone-based formulations.

TABLE 32
Polybutylene succinate-based formulations

| | | | | |
|---|---|---|---|---|
| Code | 65FZ71 | 65FZ91 | 65FD92a | 65FD92b |

Material

| | | | | |
|---|---|---|---|---|
| Polybutylene succinate | 65.0% | 65.0% | 65.0% | 65.0% |
| Activated charcoal | 20.0% | 20.0% | 20.0% | 20.0% |
| 3NOP | 15.0% | 15.0% | 15.0% | 15.0% |

On total solids

| | | | | |
|---|---|---|---|---|
| Glycerol monooleate | 0.5% | 0.5% | 0.5% | 0.0% |
| Compounding Temperature | 120° C. | 120° C. | 130° C. | 110° C. |

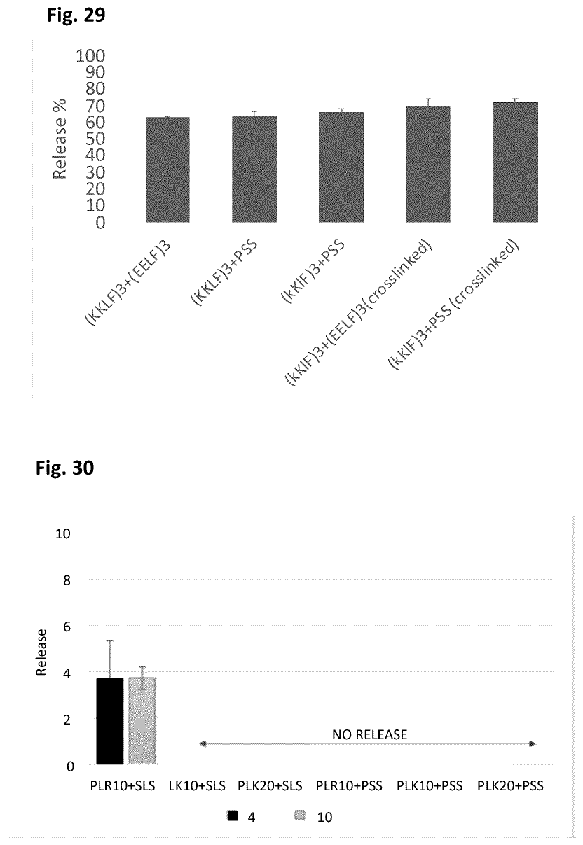

FIG. 29 is chart showing the normalized 3NOP concentration v. Release time in days of polybutylene succinate-based formulations. Lower 3NOP loadings (<10%) have a less pronounced burst release over the first 7 days of monitored release. Glycerol monooleate was identified at a potent processing aid at loadings <1%. High levels <25 wt % activated carbon can easily be processed unlike silica which leads to high viscosity and screw torque. Cross-linked systems show slower release in comparison to un-crosslinked systems. High shear conditions of compounding appear to destroy activated carbon structure and adsorption capacity is lost.

### Microcapsules for the Encapsulation of 3NOP

Polyelectrolyte complexes (PECs) are used to form microcapsules and encapsulate 3NOP. PECs are formed through attractive forces such as ionic interactions, hydrogen bonding, hydrophobic, and pi-interactions between the polyelectrolytes. Polyelectrolytes are polymers with ionic groups bonded to counter ions and can dissociate in a solution to make positively or negatively charged polymers. Herein, PEC microcapsules refer to interconnecting networks of polyelectrolytes formed upon the interaction between oppositely charged polymers.

Examples of polyelectrolytes used in this invention include synthetic polyelectrolytes such as polystyrene sulfonate (PSS), polyacrylic acid (PAA), and polyallylamine hydrochloride (PAH). Further examples include naturally occurring polyelectrolytes such as chitosan and alginate or ionic biopolymers such as proteins, enzymes, and charged polypeptides.

Examples of Polyelectrolytes (charged polymers with positively or negatively charged repeating units) Used:

### Synthetic Polyelectrolytes

Polystyrene sulfonate (PSS), molecular weight ˜200,000 Da, Sigma Aldrich
Sodium Lignosulfonate (SLS), molecular weight ˜52000 Da, Sigma Aldrich
Polyethyleneimine (PEI), molecular weight ˜2000 Da, Polysciences
Polyallylamine hydrochloride (PAH), molecular weight ˜17,500 Da, Sigma Aldrich
Commercially Available Peptide Sequences
   Poly (L-e), degree of polymerization: 10, molecular weight: 1900 Da, Alamanda Polymers
Examples of Crosslinker Used:
   Glutaraldehyde, 25% solution in water, Sigma Aldrich
   EDC (1-ethyl-3-(3-dimethylaminopropyl) carbodiimide hydrochloride), Thermo Fisher.

### PEC Microcapsules Preparation

Equal stoichiometry of oppositely charged polyelectrolytes is sequentially added to water or water-3NOP solutions followed by vortexing for 10 sec., after the addition of each component, to form PEC microcapsules. Some samples are chemically crosslinked (glutaraldehyde is an example of the crosslinker used). The PEC microcapsules may also be formed with non-stoichiometric ratios of polyelectrolytes. While stoichiometric ratios of polyelectrolytes provide almost a neutral microcapsule, microcapsules prepared with non-stoichiometric ratios are positively or negatively charged. All polyelectrolyte solutions are prepared in water and pH adjusted. The concentration of solutions is based on the monomer charge. The stock solution of 3NOP is prepared in water with a concentration of 100 mM and pH adjusted to 8.

High-performance liquid chromatography (HPLC) is used to determine the concentration of 3NOP in PEC microcapsules and calculate the release of 3NOP from the microcapsules. Samples are centrifuged to separate the supernatant phase from the complex phase. The supernatant phase is then removed carefully by using a micropipette and transferred to a 2 mL glass vial for further analysis. The 3NOP release of some PEC microcapsules prepared at neutral pH conditions and after 96 hr of preparation is summarized in Table 31.

TABLE 33
3NOP release (%) from the PEC microcapsules. Samples are prepared
with a 3NOP concentration of 100 μM. Final pH solution
~7. K, L, F, and E refer to lysine, leucine, phenylalanine,
and glutamic respectively. PSS refers to sulfonate.

| | Release (%) | |
|---|---|---|
| | at 96 h | 3NOP Concentration in |
| Polyelectrolyte Pair | timepoint | Microcapsules (μM) |
| (KKLF)₃ + (EELF)₃ | 63 | 37 |
| (KKLF)₃ + PSS | 64 | 36 |
| (kKlF)₃ + PSS | 66 | 34 |
| (kKlF)₃ + (EELF)3(crosslinked) | 70 | 30 |
| (kKlF)₃ + PSS (crosslinked) | 72 | 28 |

FIG. 29 is a graph showing 3NOP release (%) from the PEC microcapsules. Samples are prepared with a 3NOP concentration of 100 μM. Final pH solution ˜7. K, L,F, and E refer to lysine, leucine, phenylalanine, and glutamic acid, respectively. PSS refers to polystyrene sulfonate.

Examples of release profiles of some microcapsules made of positively charged polypeptides with either polystyrene sulfonate (PSS) or sodium lignosulfonate (SLS) are shown in FIG. 30 (3NOP release (%) from the PEC microcapsules. Samples are prepared with a 3NOP concentration of 100 μM. Final pH solution ˜7. PLR refers to poly (L-arginine), and PLK refers to poly (L-lysine). SLS and PSS refer to sodium lignosulfonate and polystyrene sulfonate).

The encapsulation efficiency of some PEC microcapsules prepared at neutral pH conditions is summarized in Table 34. Table 35 shows the encapsulation efficiency of samples prepared at pH˜8.

TABLE 34
Encapsulation efficiency of the PEC microcapsules. Samples are prepared
with a 3NOP concentration of 100 μM. Final pH solution ~7. Upper and lower
cases in (kKlF)₃ sequences represent L and D-chirality, respectively.

| | Total Concentration | | 3NOP Concentration |
|---|---|---|---|
| | (monomer charge basis) | Encapsulation | in Microcapsules |
| Polyelectrolyte Pair | (mM) | Efficiency (%) | (μM) |
| PLK20 + PRE20 | 5 | 7.4 | 7.4 |
| PLR10 + PRE20 | 5 | 0 | 0 |
| (KKLF)₃ + (EELF)₃ | 5 | 100 | 100 |
| (kKlF)₃ + (EELF)₃ | 5 | 22.2 | 22.2 |
| (KKLF)₃ + PSS | 5 | 37 | 37 |
| (kKlF)₃ + PSS | 5 | 100 | 100 |
| PAH + PSS | 5 | 30.9 | 30.9 |

TABLE 35
Encapsulation efficiency of the PEC microcapsules. Samples are prepared with a 3NOP
concentration of 2.5 mM. Final pH solution ~8. Upper and lower cases in (kKlF)₃
sequences represent L and D-chirality of the amino acid residue, respectively.

| | Total Concentration | | 3NOP Concentration |
|---|---|---|---|
| | (monomer charge basis) | Encapsulation | in Microcapsules |
| Polyelectrolyte Pair | (mM) | Efficiency (%) | (mM) |
| PLR10 + PRE20 | 5 | 50 | 1.25 |
| PLK20 + PRE20 | 5 | 53.8 | 1.34 |
| (kKlF)₃ + PRE20 | 5 | 50.2 | 1.26 |

Physical Crosslinking of 3NOP with Small Molecules

3NOP molecules can form hydrogen bonds with small molecules such as amino acids to make a larger size conjugate resulting in enhanced encapsulation efficiency. For example, an equal stoichiometry of 3NOP and two selective amino acids, arginine and lysine, were reacted in aqueous solution for 24 hr, and the solution was used for encapsulation studies. The results of such encapsulation with some PEC microcapsules are shown in Table 36.

TABLE 36
Encapsulation efficiency of the PEC microcapsules. Samples are prepared
with a 3NOP concentration of 125 μM. Final pH solution ~7.

| | Encapsulation | | 3NOP Concentration in | |
|---|---|---|---|---|
| | Efficiency (%) | | Microcapsules (μM) | |
| | Arginine- | Lysine- | Arginine- | Lysine- |
| Polyelectrolyte Pair | 3NOP | 3NOP | 3NOP | 3NOP |
| PLR10 + PRE20 | 4.6 | 19.1 | 5.75 | 23.9 |
| PLK20 + PRE20 | 34.3 | 28.4 | 42.9 | 35.5 |
| (KKLF)₃ + (EELF)₃ | 27.4 | 34.8 | 34.25 | 43.5 |
| PAH + PAA | 30.3 | 47.1 | 37.9 | 58.9 |
| PAH + PSS | 67.4 | 67.6 | 84.3 | 84.5 |

### Example 10: Growth of Methanobrevibacter Gottschalkii on a Continuous Flow of 4% H₂, 10% CO₂, and 86% N₂

This example demonstrates a representative method for growing a methanogen (e.g., Methanobrevibacter gottschalkii) on a continuous flow of H₂, CO₂, and N₂ mixed gas.

All components of BY medium are detailed in Tables 11A-11G, except that cysteine and BY medium Vitamins were mixed in a 1 L bottle. Rumen fluid aliquots (stored at −20° C. in conical centrifuge tubes) were thawed at room temperature. Thawed rumen fluid was spun down at 15,000×g for 60 min. The supernatant was then transferred via serological pipette to a fresh bottle without disturbing the pellet and the appropriate volume was used in media preparation according to Tables 11A-11G. The bottle was autoclaved for 25 min at 250° F., then made anaerobic by alternating vacuuming and replacement of the headspace with O₂-free CO₂. The bottle was then transferred into an anaerobic chamber (Coy 7150000 Anaerobic Chamber, Type A, Vinyl).

300 mL of the anaerobic solution above was transferred into a 500 mL Duran pressure plus bottle (DWK Life Sciences catalog #1175926) in the anaerobic environment. The required quantity of cysteine hydrochloride was dissolved in anaerobic water within the anaerobic chamber, and sterilized by filtration through an 0.2 μm syringe filter prior to addition to the bottle described above. BY Medium Vitamins were added to the mixture, completing the BY medium.

The bottle was then inoculated 1% with an actively growing M. gottschalkii culture, which had been grown using methods described previously (Joblin K. N. 2005. Methanogenic archaea. Methods in Gut Microbial Ecology for Ruminants). The inoculated 300 mL was then split evenly into two 500 mL Duran Pressure Plus bottles.

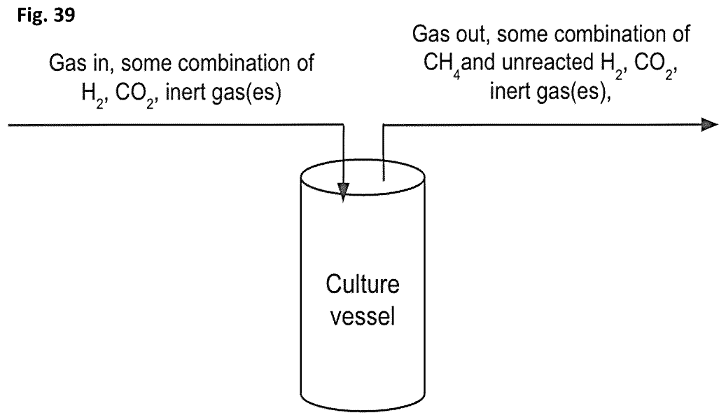

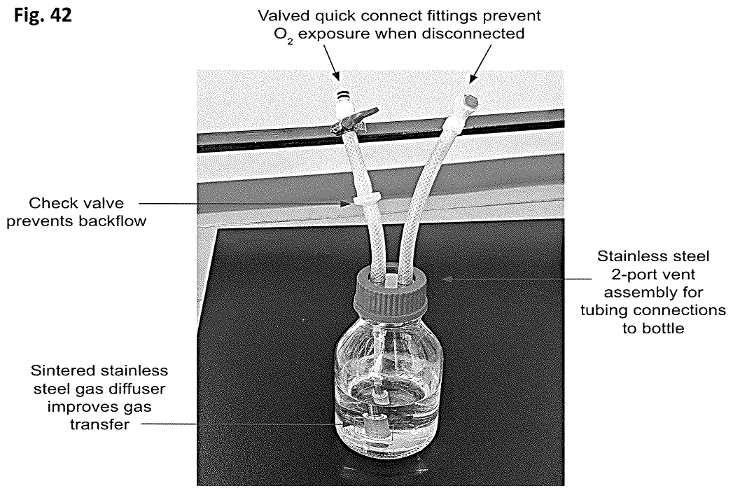

One bottle containing 150 mL inoculated culture was sealed with a Duran Grey Bromobutyl Rubber Stopper (Cat No. 09-841-311). The headspace was then fully exchanged and pressurized with 80% H₂, 20% CO₂ to 15 psi overpressure. This bottle is labeled “control” in FIG. 39-FIG. 43, as it more closely resembles standard culturing protocols for hydrogenotrophic bacteria (e.g., methanogens). This bottle was incubated at 38° C. with 200 rpm shaking.

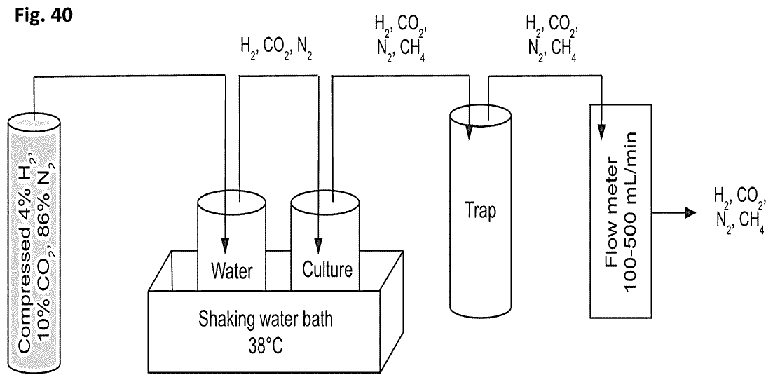

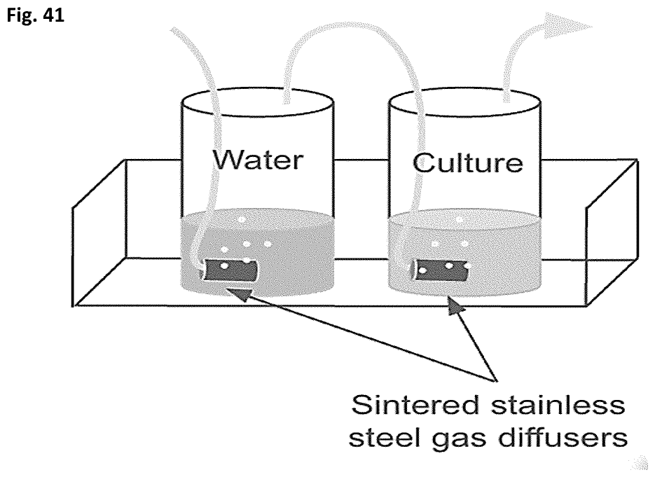

The other bottle containing 150 mL inoculated culture was attached to a continuous gas flow apparatus, a process flow diagram of which is shown in FIG. 39. Flow rate was controlled to ˜100-500 mL/min of non-flammable 4% H₂, 10% CO₂, and 86% N₂ (Middlesex Gases Part #34832) using a flow meter (Siargo Ltd. MF5700). Prior to sparging into the culture bottle, gas was flowed through sterile, anaerobic water (500 mL Duran Pressure Plus bottle containing approximately 300 mL sterile anaerobic water) heated to 38° C., to prevent evaporation of the culture. In both the anaerobic water gas humidifying bottle and in the culture bottle, gassing was performed through a sintered stainless steel gas diffuser at the base of the bottle (removed from Ferroday MN0005 keg lid). Braided silicone tubing was used for all flow (5/32″ ID-⅜″ OD Food Grade Silicone), and tubing was connected to bottles through a INTLLAB 316 Stainless Steel Small 2-Port Vent Assembly (Amazon part #GL45_S2). FIG. 40 depicts an example of a gas diffusion system at the base of the bottles, as well as a humidifying bottle upstream of the culture bottle to prevent volume loss. This volume loss prevention could be achieved by other operations, e.g., a condenser affixed on top of the culture vessel. FIG. 41 shows a labeled image of the setup for water and culture bottles. Further, both bottles were incubated to approximately 38° C. in an actively shaking water bath (Precision Reciprocal Shaking Bath). To prevent fouling of the flow meter, which was downstream of both bottles, a trap (250 mL bottle with inlet and outlet tubing for which neither reached the bottom of the bottle) was placed between the culture bottle and the flow meter. Valved quick connect fittings at the culture bottle gas inlet and outlet prevent contamination by oxygen while the bottle is handled in the anaerobic chamber.

Throughout growth, both bottles were measured for CH₄ production at multiple time points using a Gazomat Gazoscan, to confirm that they were still actively growing. To do so, the continuous flow bottle was detached from the continuous flow to allow CH₄ to accumulate, whereas the control bottle could be measured immediately. Both bottles were then transferred to the anaerobic chamber. 1 mL was sampled from each and frozen at −80° C. until the end of the experiment. After sampling, the control bottle headspace was fully exchanged and pressurized with 80% H₂, 20% CO₂ to 15 psi overpressure, and placed back in the 38° C. shaking incubator. The continuous flow bottle was reattached to the continuous flow apparatus.

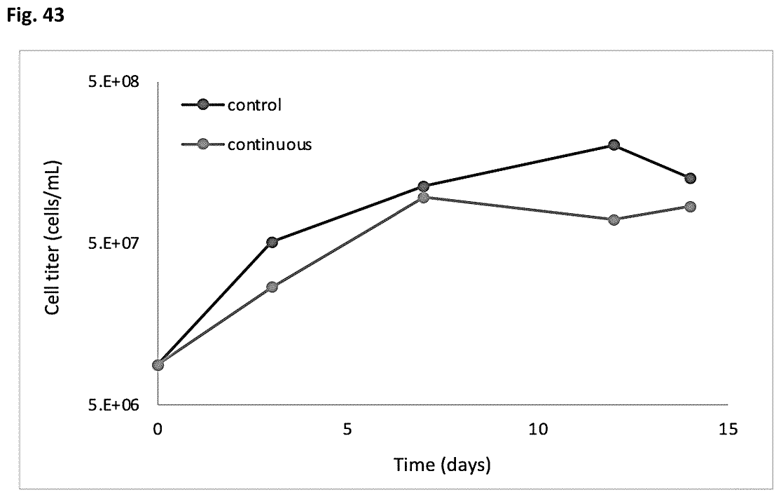

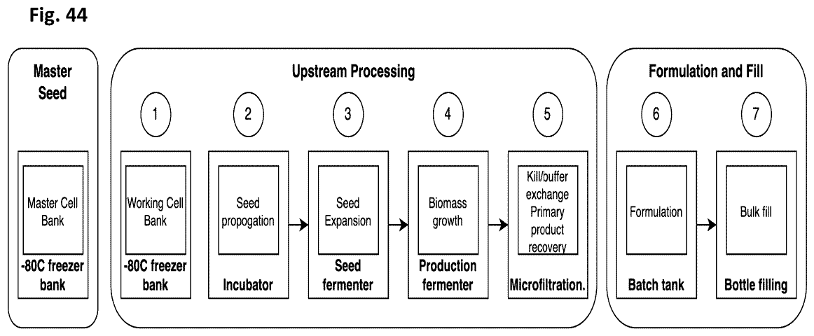

At the end of the experiment, all samples were thawed at room temperature and quantified for cell titer in the following way. Undiluted SONY calibration beads (LE-B3001) were analyzed with a sample pressure of 4 on a SONY SH800 using a 100 μm SONY chip. The 3 μm sized beads were identified, and 25,000 3 μm events were captured. The time to record those events was documented. All samples throughout the M. gottschalkii growth study were then analyzed at the same sample pressure, and the time required to reach 25,000 events was recorded. At the same sample pressure, the cell titer can be calculated by comparing the time required to record 25,000 cell events to the time required to record the calibration standard beads, which are at a known density of 2.5×10⁶ beads/mL. The growth curves are shown in FIG. 43, and demonstrate that M. gottschalkii was able to grow on a continuous flow of non-flammable H₂, CO₂, N₂ mix.

This shows the ability to grow methanogens using non-explosive low pressure gas. This is the first example showing this and was able to achieve cell titers similar to or at least as good as typical higher pressure growth. These results are surprising and unexpected as it was previously not believed capable to grow hydrogenotrophic methanogens without the use of high pressure, explosive gas mixes, especially without the use of one or more substrates in a non-hydrogenotrophic biochemical pathway, for example acetate, ethanol, or methanol.

### Example 11: Additional Protocols and Methods for Vaccination of Animals (e.g., Cattle), with Cells and/or Cell Parts of Methanobrevibacter

This example demonstrates additional protocols and methods of administering a vaccine composition to an animal. The vaccine composition used in this study contains cells and/or cell parts of Methanobrevibacter ruminantium and/or Methanobrevibacter gottschalkii, which upon vaccination to animals resulted in reduction in the animal emitted CH₄, H₂, and/or CO₂ normalized CH₄; increase emitted CO₂, and/or effect on lactic acidosis in ruminants (e.g., reducing rumen lactate and increasing pH).

Trial 2—Species: Cattle—USDA Breeders: No—Unused Offspring: 0—Research Animals: 20

Trial 2 was used to (1) assess the total immune response generated following vaccination with various killed methanogen cell vaccine preparations, using total IgG and IgA quantitation; (2) assess modulation of the rumen microbiome following vaccination with various killed methanogen cell vaccine preparations using 16S rRNA sequencing; (3) assess modulation of CH₄ emissions following vaccination with various killed methanogen cell vaccine preparations using GreenFeed systems (C-Lock, Rapid City, SD); and (4) harvest serum and PBMCs from immunized cattle, in support of method development for in vitro characterization.

### Experimental Description

Animals were inoculated with killed whole methanogen cells to generate bovine anti-methanogen antibodies (Abs) which (1) bind methanogens and (2) modulate ruminal microbiome function. Vaccines were prepared according to methods described in the art (Wedlock, D. et al. Development of a vaccine to mitigate greenhouse gas emissions in agriculture: Vaccination of sheep with methanogen fractions induces antibodies that block methane production in vitro. New Zeal Vet J 58, 29-36 (2010); Wright, A. D. G. et al. Reducing methane emissions in sheep by immunization against rumen methanogens. Vaccine 22, 3976-3985 (2004) summarized in Select Procedures (described below)). Animals were inoculated with a four-dose series, administered at Days 0, 21, 56 and 77. Each vaccine dose contained approximately 10⁹ methanogen cells. Volumes of antigen preparations, were prepared and mixed at a 1:1 ratio with the appropriate adjuvant immediately prior to inoculation to formulate the final vaccine. Each vaccine formulation was inoculated into five animals and administered through the subcutaneous (SQ) route into the anterior region of the neck. The injection site was alternated between subsequent injections, e.g., right neck, left neck, right neck. A buffer-only control, which included the appropriate vaccine adjuvant, was inoculated into five animals following the same vaccination protocol as the treatment groups.

Total blood, saliva and rumen fluid samples were collected periodically throughout the duration of the immune monitoring period (90 days), according to Table 37, for subsequent processing. Whole blood were initially processed for sera and/or PBMCs on-site. Sera samples were analyzed on-site using commercially available bovine IgG and/or IgA quantitative test kits, e.g., ELISA assays.

TABLE 37
Study schedule

| | Vaccina- | Total blood | PBMC | Saliva | Rumen |
|---|---|---|---|---|---|
| Date | tion | draw | Isolation | collection | sample |
| Day 0 | X | X (400 mL) | X | X | X |
| | | | (Group B + C | | |
| | | | only) | | |
| Day 21 | X | X (100 mL) | | | |
| Day 28 | | X (100 mL) | | X | X |
| Day 42 | X | X (100 mL) | | | |
| Day 49 | | X (100 mL) | | X | X |
| Day 63 | X | X (100 mL) | | | |
| Day 70 | | X (400 mL) | X | X | X |
| | | | (Group B + C | | |
| | | | only) | | |
| Day 90 | | X (100 mL) | | X | X |

TABLE 38
Vaccination groups

| Number of | | |
|---|---|---|
| Animals | Vaccination Group | Description |
| 5 | A | Buffer Control |
| 5 | B | Methanogen A |
| | | Preparation:Whole cell |
| 5 | C | Methanogen B |
| | | Preparation:Whole cell |
| 5 | D | Methanogen A + B (1:1 mix) |
| | | Preparation:Whole cell |

Additionally, remaining sera and saliva sample volumes were processed for subsequent sample analysis and retention. Isolated PBMCs were also further analyzed.

CH₄, H₂ and CO₂ emissions were measured daily throughout the study using GreenFeed (GF) systems. Prior to initialization of the study, cattle were acclimated with and trained to use the GrowSafe System (Calgary, AB, CA). The study period included a 105 d immune monitoring period, which began after primary vaccination, in addition to a 2-3 week GreenFeed training period prior to study initiation. At least five additional animals beyond the number required for this study (20) were enrolled in the GreenFeed training period. Animals which did not learn the desirable behaviors associated with GreenFeed systems within a reasonable period of time were disenrolled from the study. The training period was designed for acclimatization to the herd diet, as well as the GrowSafe and GreenFeed Systems.

Rumen fluid samples were collected via the esophagus periodically throughout the study, according to Table 37. Samples were strained through three layers of cheesecloth, with the liquid phase aliquoted into 50 mL conical vials. All samples were snap-frozen in liquid N₂ and stored at −20° C. for subsequent processing. Samples shall be sequenced, using 16S rRNA sequencing techniques summarized in Select Procedures (described below), in order to establish whether ruminal methanogen populations were affected by the immunization protocol.

Previously frozen rumen samples were thawed on wet ice, and further processed. Isolation of the total DNA from liquid phase rumen samples followed methods defined in Wang, P. et al. Isolation of high-quality total RNA from rumen anaerobic bacteria and fungi, and subsequent detection of glycoside hydrolases. Can J Microbiol 57, 590-598 (2011), according to the methods defined in Select Procedures (described below).

Only healthy, weaned steers (Angus cross) were utilized for this study. All calves were weaned for a minimum of 60 days and received pre-weaned vaccinations a minimum of 60 days prior to initiation of the study. In addition, steers demonstrated no clinical signs of health concerns (i.e., dull or sunken eye, depression, signs of scours, listlessness, weakness, or raspy breathing). All calves selected were within 50 lbs of each other and were adapted to a growing calf diet. Rumen boluses were installed by veterinary-trained staff during the GreenFeed training period, no less than five days prior to the scheduled primary vaccination, and used to quantitatively monitor changes in rumen temperature and pH.

Animals were retained for up to six months after the study's completion. Animals were not under scheduled study monitoring during this period.

Special attention was paid to animal products generated during and after vaccine studies, as they were discarded and/or withheld from processing for a given period. No animals underwent processing for at least twenty-one days post-vaccination.

### Select Procedures

### Vaccine Preparation

Antigen preparations were provided frozen to the study site. Each antigen preparation was labeled with the appropriate treatment group ID. Antigen preparations were packaged as individual aliquots in single 5 mL cryotubes. Antigen preparations were provided at 2× appropriate dosing concentration, and mixed with the appropriate adjuvant at 1:1 volume ratio to prepare the final vaccine formulation immediately prior to injection.

Vaccines were prepared for final injection volumes, including adjuvant, of 2 mL+/− 50%. Exact dosage volumes were provided at the time of study.

Adjuvants were supplied as a bulk solution. Primary injections utilized Freund's Complete Adjuvant (Sigma, St. Louis, MO), whereas all booster injections utilized Freund's Incomplete Adjuvant (Sigma, St. Louis, MO).

### Subcutaneous Injection

Vaccines were administered, formulated with the appropriate adjuvant. Subcutaneous injections were delivered into the animal's neck by veterinary-trained staff using an 18-gauge 0.5-0.75″ needle. Injection sites were alternated between scheduled injections, e.g., right shoulder, left shoulder. New sterile needles and syringes were used for each injection, loaded from the appropriate bulk vaccine formulation solution.

Antigen preparations were provided frozen and immediately store at −80° C. Immediately prior to injection, antigen preparations were thawed on wet ice (4° C.), and mixed at a 1:1 volume ratio with the appropriate adjuvant to prepare the final vaccine formulation.

### Serum Isolation & Analysis

Total blood samples shall be collected under the supervision of veterinary-trained staff; sample volume and collection frequency are defined in Table 37. Samples were collected via jugular vein needle puncture into blood tubes or collection bags. Collected blood samples were processed via ultracentrifugation for serum within 2 hrs of collection. Isolated serum samples were aliquoted, labeled and stored at −20° C. or lower. Estimated total isolated serum volume was expected to be roughly half of the total blood draw volume. 25-50 mL aliquots were prepared for further processing.

Serum sample aliquots of sufficient volume were retained for total IgA and IgG analyses and results compiled according to the schedule defined in Deliverables below.

All necessary materials for sample analysis by ELISA were sourced and available at the study's initiation. At least 3 additional ELISA plates were kept on hand, in the case that sample analysis needed to be repeated.

Remaining serum sample aliquots, not scheduled for analysis, were appropriately labeled with animal ID, treatment group and collection date, frozen and provided on dry ice, within the Deliverables schedule defined below.

Serum samples used for analysis were retained for one month after the final Study Report. At such a time, samples were discarded or available for further studies.

### PBMC Isolation

PBMCs were isolated from Group B+C serum samples collected at Day 0 and 70. All available PBMCs were harvested from 150 mL of serum, with the aim of harvesting at least 5×10₇ PBMCs. Once isolated, PBMCs were immediately treated with RNA later, flash-frozen and stored at −80° C. Frozen PBMCs can we further analyzed within 5 days of harvest.

Exact protocols for PBMC isolation are subject to additional modifications. Method development utilized untreated bovine serum from selected animals.

### Saliva Collection & Analysis

Saliva samples were collected using a suction tube in the oral cavity, into a conical flask or directly into a sterile Eppendorf tube. Equipment contacting saliva (e.g., collection flask, plastic tubing) was changed out after each animal's collection; either single-use or autoclavable materials were chosen. Samples were immediately stored on wet ice (4° C.), and then transported to the lab for storage under −80° C.

Saliva sample aliquots of sufficient volume were retained to support total IgA and IgG analysis and results shared electronically at a later time, according to the schedule defined in Deliverables below.

All necessary materials for sample analysis by ELISA were sourced and available at the study's initiation.

Remaining saliva sample aliquots, not scheduled for analysis, were appropriately labeled with animal ID, treatment group and collection date, frozen and provided on dry ice, within the deliverables schedule defined below.

Samples used for analysis were retained for one month after the final Study Report has been shared with ArkeaBio. At such a time, samples shall be discarded or available for further studies.

### Dry Matter Intake and Cow Body Weight

Feed intake were recorded daily throughout the study period using GrowSafe Systems. Animals were fed once daily in the morning and had access to a water source at all times. Body weight were recorded periodically, at the time of total blood draws.

### Enteric CH₄ Emissions Measurement

Enteric CH₄, H₂ and CO₂ emissions were measured daily throughout the study period using GreenFeed Systems (C-Lock, Inc., Rapid City, SD). Animals had free access to the GreenFeed System throughout the study period; animals were not coerced to use the system. CH₄ yield and intensity were calculated using dry matter intake for each measurement period separately.

### Rumen Sampling by Esophageal Tubing

Fresh rumen samples were collected by esophageal tubing, with 50 mL collections expected, and retained in conical vials. Samples were strained through three layers of cheesecloth, with the liquid phase aliquoted into 50 mL conical vials. All samples were snapped-frozen in liquid N₂ and stored at −20° C. for subsequent processing.

Samples were processed within twenty-four hrs of collection. Sample aliquots were labeled with animal number, collection method, collection date and time.

### DNA Isolation

Isolation of DNA from the rumen followed one of the methods defined in Henderson, G. et al. Effect of DNA Extraction Methods and Sampling Techniques on the Apparent Structure of Cow and Sheep Rumen Microbial Communities. Plos One 8, e74787 (2013), with preference given to methods involving both phenol-chloroform and mechanical lysis steps (PCQI, PCBB, PCSA). RBBC was the preferred extraction method should the use of phenol-chloroform be an issue.

PCR amplification of the hypervariable V6-V8 regions of the 16S rRNA gene was performed using the archaea-specific Ar915aF/Ar1386R primer set with Illumina adapters as defined in Table 1 of Kittelmann et al. Buccal swabbing as a noninvasive method to determine bacterial, archaeal, and eukaryotic microbial community structures in the rumen. Appl Environ Microbiol 81:7470-7483 (2015), and PCR cycle conditions as defined in Kittelmann et al. Simultaneous Amplicon Sequencing to Explore CoOccurrence Patterns of Bacterial, Archaeal and Eukaryotic Microorganisms in Rumen Microbial Communities. PLoS ONE 8(2): e47879 (2013).

### Exemplary Deliverables

1. Animal Descriptors Database: An electronic database was provided of the enrolled animals' information, collected during the study period, as defined below. Database was provided within fourteen days of study initiation except for final body weight, which may be included as part of the final Study Report. Data included: Animal descriptors, Breed, Age, Body weight, at study initiation, Body weight, at study completion, Environment prior to transportation to AgriLife Research Facilities, Environment at AgriLife Research Facilities, Herd diet contents

2. Study Report: A written report was provided summarizing the study results, as well as a database of all sample analysis results. The Study Report was provided within thirty days of the study's completion. The Study Report included analysis and interpretation for the following endpoints: Copy of animal descriptors database, Sera and salivary antibody titer, including total IgG and total IgA, for all collected timepoints, Dataset to include standard curve and technical replicates for all ELISA plates, Methods summary, including Materials details, Daily CH₄ emissions, corrected for dry matter intake.

3. Serum samples: Results of serum sample analysis were provided within the timelines below: (a) Serum samples collected on Days 0, 21 and 28 were analyzed, and results provided within +14 days of Day 28 collection; (b) Serum samples collected on Days 42 and 49 were analyzed, and results provided within +14 days of Day 49 collection; (c) Serum samples collected on Days 63 and 70 were analyzed, and results provided within +14 days of Day 70 collection; (d) Serum samples collected on Day 90 were analyzed, and results provided within +14 days of Day 90 collection; (e) Serum sample aliquots were processed, labeled, and provided within the following schedule. For each collection, transferred aliquots totaled at least 70 mL; except for Days 49 and 70, which totaled at least 250 mL; (f) Serum samples collected on Days 0, 21 and 28 were provided within +7 days of Day 28 collection; (g) Serum samples collected on Days 42 and 49 were provided within +7 days of Day 49 collection; (h) Serum samples collected on Days 63 and 70 were provided within +7 days of Day 70 collection; (i) Serum samples collected on Day 90 were provided within +7 days of Day 90 collection; (j) Sample aliquots were labeled with animal ID, treatment group, collection date, and, if multiple aliquots were provided, aliquot number. Samples were provided in a tube rack, grouped by treatment group.

4. PBMCs: PBMC samples were harvested, labeled, and provided within the following schedule: (a) PBMC samples collected on Day 0 were provided within +5 days of Day 0 collection; (b) PBMC samples collected on Day 70 were provided within +5 days of Day 70 collection.

5. Saliva samples: Results of saliva sample analysis were provided within the timelines below: (a) Saliva samples collected on Days 0 and 28 were analyzed, and results provided within +14 days of Day 28 collection; (b) Saliva samples collected on 49 and 70 were analyzed, and results provided within +14 days of Day 70 collection; (c) Saliva samples collected on Day 90 were analyzed, and results provided within +14 days of Day 90 collection; (d) Saliva sample aliquots were processed, labeled, and provided within the following schedule; (e) Saliva samples collected on Days 0 and 28 were provided within +7 days of Day 28 collection; (f) Saliva samples collected on Day 49 were provided within +7 days of Day 49 collection; (g) Saliva samples collected on Day 70 were provided within +7 days of Day 70 collection; (h) Saliva samples collected on Day 90 were provided within +7 days of Day 90 collection; (i) Sample aliquots were labeled with animal ID, treatment group, collection date, and, if multiple aliquots were provided, aliquot number. Samples were provided in a tube rack, grouped by treatment group.

Upon injection of the final vaccination (d77) on Oct. 31, 2023, all animals were moved from the GrowSafe pens to the ‘receiving pens’ at Beef Cattle Systems (College Station, TX) for completion of the study period (d104; Nov. 27, 2023). Some animals may be released prior to d104. Upon transfer to the receiving pens, CH₄ data ceased to be collected (d77.

Following completion of the study period (d104), animals enrolled in Treatment Groups A (5) and C (5) were retained for an additional 36 days of observation and sample collection. The study period for these animals (10) ended on d140 (Jan. 2, 2024).

Observations from animals enrolled in Treatment Groups B (5), D (5) and Control (4) were considered complete after sample collection on d98 (Nov. 21, 2023); animals were released from the study.

Total blood, saliva and rumen fluid samples were collected periodically throughout the duration of the extended immune monitoring period, according to Table 39, for subsequent processing. Whole blood was initially processed for sera on-site. Rumen fluid samples were collected via the esophagus periodically throughout the study, according to Table 39. Samples were strained through three layers of cheesecloth, with the liquid phase aliquoted into 50 mL conical vials. All rumen fluid samples were snapped-frozen in liquid N₂ and stored at −20° C. for subsequent storage.

TABLE 39
Study schedule, Trial 2b

| | | | PBMC | Saliva | Rumen |
|---|---|---|---|---|---|
| Date | Vaccination | Blood draw | Isolation | collection | sample |
| d 77 | X * | X (100 mL) * | | | |
| Oct. 31, 2023 | | | | | |
| d 84 | | X (300 mL) * | X * | X * | X * |
| Nov. 7, 2023 | | | Group B + C | | |
| d 98 | | X (100 mL) * | | X * | X * |
| Nov. 21, 2023 | | | | | |

d 99 - Group B + D animals may be released from study

| | | | |
|---|---|---|---|
| d 112 | X (100 mL) | X | X |
| Dec. 5, 2023 | Group A + C | Group A + C | Group A + C |
| d 126 | X (100 mL) | X | X |
| Dec. 19, 2023 | Group A + C | Group A + C | Group A + C |
| d 140 | X (100 mL) | X | X |
| Jan. 2, 2024 | Group A + C | Group A + C | Group A + C |

* denotes vaccination and/or sample collections previously contracted in Trial 2.

TABLE 40
Vaccination groups, extension period

| Number of | Vaccination | |
|---|---|---|
| Animals | Group | Description |
| 5 | A | Buffer Control |
| 5 | C | Methanogen B |
| | | Preparation:Whole cell |

Sera, saliva and rumen fluid sample volumes were provided for subsequent sample analysis and retention.

Trial 2 enrolled only healthy, weaned steers (Angus cross). All calves were weaned for a minimum of sixty days and received pre-weaned vaccinations a minimum of sixty days prior to initiation of the study. In addition, steers demonstrated no clinical signs of health concerns (i.e., dull or sunken eye, depression, signs of scours, listlessness, weakness, or raspy breathing).

Special attention was paid to animal products generated during and after vaccine studies, as they were discarded and/or withheld from processing for a given period. No animals underwent processing for at least twenty-one days post-vaccination.

### Deliverables

1. Animal Descriptors Database (Trial 2):An electronic database was provided of the enrolled animals' information, collected during the study period, as defined below. Database was provided within fourteen days of study initiation except for final body weight, which may be included as part of the final Study Report. Data included: Animal descriptors: Breed, Age, Body weight, at study initiation, Body weight, at study completion, Environment prior to transportation to AgriLife Research Facilities, Environment at AgriLife Research Facilities, Herd diet contents

2. Study Report, Extension Period: A biweekly report of animal body weight was provided.

3. Serum samples: Serum sample aliquots were processed, labeled and provided within the following schedule: (a) Serum samples collected on Days 77 and 84 were provided within +7 days of Day 84 collection; (b) Serum samples collected on Day 98 were provided within +7 days of Day 98 collection; (c) Serum samples collected on Day 112 were provided within +7 days of Day 112 collection; (d) Serum samples collected on Day 126 were provided within +7 days of Day 126 collection; (e) Serum samples collected on Day 140 were provided within +7 days of Day 140 collection; (f) Sample aliquots were labeled with animal ID, treatment group, collection date, and, if multiple aliquots were provided, aliquot number. Samples were provided in a tube rack, grouped by treatment group.

4. PBMCs (Trial 2): PBMC samples were harvested, labeled, and shipped within the following Schedule: (a) PBMC samples collected on Day 84 were provided within +5 days of Day 84 collection.

5. Saliva samples: Saliva sample aliquots were processed, labeled, and shipped within the following Schedule: (a) Serum samples collected on Day 84 were provided within +7 days of Day 84 collection; (b) Serum samples collected on Day 98 were provided within +7 days of Day 98 collection; (c) Serum samples collected on Day 112 were provided within +7 days of Day 112 collection; (d) Serum samples collected on Day 126 were provided within +7 days of Day 126 collection; (e) Serum samples collected on Day 140 were provided within +7 days of Day 140 collection; (f) Sample aliquots were labeled with animal ID, treatment group, collection date, and, if multiple aliquots were provided, aliquot number. Samples were shipped in a tube rack, grouped by treatment group.

6. Rumen fluid simples: Rumen fluid simples were processed, labeled, and stored at or below −20° C. for up to two months following the study's termination (d140).

### Adverse Reactions

Any adverse reactions to vaccine adjuvants, e.g., Freund's Complete Adjuvant, are monitored and recorded and may include, local reactions, such as those at the injection site, and generalized reactions. Generalized reactions may include, but are not limited to, elevated body temperature, loss of appetite, anaphylaxis, abortion and/or death. Adverse reactions can be monitored through daily clinical health evaluations, and performed under the supervision of trained veterinary staff.

Any adverse reactions to be reported include the following details: (1) Animal(s) involved, including number vaccinated and number reacting; (2) Product(s) involved including, lot/serial numbers; (3) Contact information of those reporting the event; (4) Details of the event.

If an animal death occurs during the study or animal product holding period, a necropsy is to be performed by the appropriate authority, as required by applicable law or regulation. A full detailed report on the necropsy's findings shall be submitted where appropriate.

### Exemplary Protocol

The data below was generated following vaccination with a formalin-inactivated whole cell methanogen vaccine. Specifically, this data set looks at animals vaccinated with M. gottschalkii on d0, d21, d56, and d77 (but d77 was after this sample collection). Fresh rumen fluid from Angus-crossbred (Brangus) steers was collected on day 63 (d63) of Trial 2, immediately prior to this study. To collect the fresh rumen fluid, animals were first restrained in a hydraulic squeeze chute with a head gate. Next, a long flexible plastic tube was inserted into the animal's stomach by way of the esophagus. Fresh rumen fluid samples were then collected, via the esophageal tubing, using a vacuum pump, strained through three (3) layers of cheesecloth, and divided into liquid and solid phases. The liquid phase was retained in pre-warmed thermoses for transportation to the lab, and immediately utilized for this study.

Liquid phase samples (approximately 2 mL each) were immediately analyzed for volatile fatty acid (VFA) concentration using high-performance liquid chromatography, specifically a GOW-MAC Series 580 Isothermal Gas Chromatograph (Shimadzu Scientific Instruments, Columbia, MD), following modified procedures by Weimer, P. J., Y. Shi, and C. L. Odt. 1991, A segmented gas/liquid delivery system for continuous culture of microorganisms on insoluble substrates and its use for growth of Ruminococcus flavefaciens on cellulose. Appl. Microbiol. Biotechnol. 36:178-183.

### Results

Fresh rumen fluid was collected on d63 of Trial 2 from Brangus steers vaccinated on d0, d21, d56, and d77 with either a formalin-inactivated whole cell M. gottschalkii vaccine (Group C) or a placebo control (Group A); note that d77 booster vaccination was administered after the sample collection used in this study.

As depicted in Table 41, it was observed that lactic acid concentration was about half that in vaccinated animals as that in non-vaccinated animals (p=0.02). Furthermore, rumen pH was slightly higher in vaccinated animals, as compared to non-vaccinated animals (p=0.81).

Vaccinated animals (i.e., M. gottschalkii): Lactic acid concentration of 0.071 mmol pH 6.57; Collection date: d63

Non-vaccinated animals (i.e., placebo control); Lactic acid concentration of 0.139 mmol; pH 6.50; Collection date: d63

The ability of a methanogen-targeted vaccine to reduce lactic acid concentrations in the rumen has not been previously demonstrated. Moreover, it was surprising and unexpected that vaccination with a formalin-inactivated whole cell methanogen vaccine had such significant effects on lactic acidosis (e.g., reducing rumen lactate and increasing pH).

TABLE 41
Rumen fluid pH and volatile fatty acid concentrations of a grower-
type diet from non-vaccinated (A) and vaccinated (C) steers.

| Item | Treatment¹ | | | |
|---|---|---|---|---|
| VFA, mmol | A | C | SEM | P-value |
| Succinic | 0.046 | 0.035 | 0.0040 | 0.07 |
| Formic | 0.018^{b} | 0.038^{a} | 0.0042 | <0.01 |
| Acetic | 55.831 | 57.404 | 2.7201 | 0.68 |
| Propionic | 28.771 | 23.617 | 1.8151 | 0.05 |
| Isobutyric | 0.778^{b} | 0.992^{a} | 0.0721 | 0.04 |
| Butyric | 10.962 | 12.415 | 0.9730 | 0.30 |
| Isovaleric | 1.504^{b} | 1.849^{a} | 0.0820 | <0.01 |
| Valeric | 1.384 | 1.276 | 0.1013 | 0.45 |
| Lactic | 0.139^{a} | 0.071^{b} | 0.0195 | 0.02 |
| Total | 99.431 | 97.703 | 5.0490 | 0.81 |
| Acetate:Propionate | 2.133^{b} | 2.524^{a} | 0.1104 | 0.02 |
| pH | 6.50 | 6.57 | 0.216 | 0.81 |
| Redox, mV | −302.24 | −256.94 | 42.916 | 0.48 |

¹A, rumen fluid from non-vaccinated steers; C, rumen fluid from vaccinated steers.
^{a-b}Least-squares means with different superscripts within a row differ at P < 0.05.

### Example 12: Growth of Methanobrevibacter Gottschalkii on by Medium and SD0001

This example demonstrates the growth of M. gottschalkii in BY and SD0001 media in batch.

BY medium was prepared by combining all BY medium components (listed in Table D above) except for L-cysteine and BY vitamins. Rumen fluid aliquots (taken from storage at −20° C.) were thawed at room temperature. Thawed rumen fluid was centrifuged at 15,000×g for 60 min. The supernatant was then carefully decanted to minimally disturb the pellet. The appropriate amount of rumen fluid was added to the mixture as described above. The bottle was actively degassed by alternating between vacuuming and replacing headspace with O₂-free CO₂ for 15 min. The bottle was then transferred to an anaerobic chamber (Coy 7150000 Anaerobic Chamber, Type A, Vinyl).

The bottle was opened, and the appropriate amount of L-cysteine was added to the bottle. Media was then aliquoted across 28 mL Hungate tubes (Chemglass, anaerobic culture tubes [18×150 mm], CLS-4209-10), filling with 10 mL of media. The Hungate tubes were then capped with a 20 mm rubber stopper (Chemglass, Blue Butyl Rubber [20 mm], CLS-4209-14) and crimped with an aluminum band (Chemglass, Aluminum seal [20 mm], CLS-4209-12) with an automated crimper (PerkinElmer, 20 mm Electronic Universal Crimper, N9304501). Once tubes were sealed, they were autoclaved for 25 min. at 250° F. Tubes were stored at 4° C. until needed.

SD0001 media was prepared by combining all SD0001 components except for sodium sulfide, L-cysteine, and fatty acid solution (pre-prepared isobutyric acid, DL-2-methylbutyric acid, valeric acid, and isovaleric acid solution stored at −20° C.). The bottle was actively degassed by alternating between vacuuming and replacing headspace with O₂-free CO₂ for 15 min. The pre-prepared fatty acid solution was thawed at room temperature in a fume hood. The appropriate amount of sodium sulfide was measured in an analytical balance under a fume hood and poured into a capped, airtight 15 mL falcon tube (Fisher Scientific, Falcon™ 15 mL Conical Centrifuge Tubes, 14-959-70C). The bottle, thawed fatty acid solution, measured sodium sulfide and L-cysteine were transferred to an anaerobic chamber (Coy 7150000 Anaerobic Chamber, Type A, Vinyl).

The bottle was opened in the glove box, and the appropriate amount of fatty acid solution was added to the mixture. Measured sodium sulfide and cysteine were added to the mixture. The bottle was gently swirled to dissolve and mix the fatty acid solution, sodium sulfide, and L-cysteine. Media was adjusted to pH 6.5+/−0.1 with 37% HCl or 10N NaOH. Clean 28 mL Hungate tubes (Chemglass, anaerobic culture tubes [18×150 mm], CLS-4209-10) were filled with 10 mL of media each. The Hungate tubes were capped with a 20 mm rubber stopper (Chemglass, Blue Butyl Rubber [20 mm], CLS-4209-14) and crimped with an aluminum band (Chemglass, Aluminum seal [20 mm], CLS-4209-12) with an automated crimper (PerkinElmer, 20 mm Electronic Universal Crimper, N9304501). Once tubes were sealed, they were autoclaved for 25 min. at 250° F. Tubes were stored at 4° C. until needed.

On the day of inoculation, 2 tubes of BY medium and 2 tubes of SD0001 were taken from 4° C. storage and placed in the anaerobic chamber. 1 tube containing the appropriate amount of BY medium vitamins was taken out of −20° C. and thawed at room temperature before placing in an anaerobic chamber. Prior to inoculation, the appropriate amount of BY medium vitamins were added to each tube. Live M. gottschalkii culture which had been grown using methods previously described (Joblin K. N. (2005) Methanogenic archaea. Methods in Gut Microbial Ecologyfor Ruminants) was used to inoculate the tubes with an inoculation ratio of 1% via syringe (BD, BD Disposable Syringes with Luer-Lok™ Tips, BD 309628) with a 23G luer-lock needle attachment (BD, BD General Use and PrecisionGlide Hypodermic Needles 23G, BD 305193). The inoculated tubes were gently inverted twice to mix.

The tubes were taken out of the anaerobic chamber. The tubes were pressurized to −25 psi overpressure with 80% H₂ and 20% CO₂. Tubes were placed in a 38° C. shaking incubator (New Brunswick Scientific, innova 4230 refrigerated incubator shaker) in a tube rack at an approximately 450 angle.

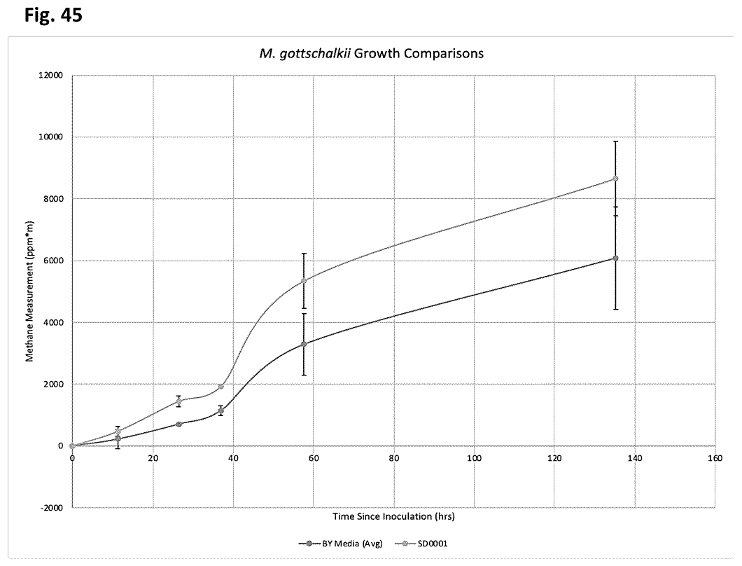

The tubes were sampled with a Gazomat Gazoscan by placing them in a proprietary tube and Gazomat Gazoscan holder to measure the CH₄ in the tube headspace. CH₄ reading was recorded after a steady reading (3 steady and continuous readings). CH₄ measurements were made once daily during weekdays. The tubes were grown for 1 week. Additional 80% H₂ and 20% CO₂ was injected into tubes every 3 days to 25 psi overpressure. FIG. 45 shows CH₄ production by M. gottschalkii in BY medium (gray) and in SD0001 media (yellow).

### REFERENCES

Costa, K. C., et al. 2013. H₂-Independent Growth of the Hydrogenotrophic Methanogen Methanococcus maripaludis. mBio. 4:2.
Long, F., et al. 2017. A Flexible System for Cultivation of Methanococcus and Other Formate-Utilizing Methanogens. Archaea.
Weimar, M. R., et al. 2017. Development of Multiwell-Plate Methods Using Pure Cultures of Methanogens To Identify New Inhibitors for Suppressing Ruminant Methane Emissions. Appl Environ Microbiol. 83:15.
Buan, N. R. 2018. Methanogens: pushing the boundaries of biology. Emerg Top Life Sci. 2:4.
Enzmann, F., et al. 2018. Methanogens: biochemical background and biotechnological applications. AMB Express. 8:1.
Eecke, H. C. V., et al. 2012. Hydrogen-limited growth of hyperthermophilic methanogens at deep-sea hydrothermal vents. PNAS. 109:34.
Leigh, J. A. 2011. Growth of methanogens under defined hydrogen conditions. Methods Enzymol. 494:111-8.
Lackner, N., et al. 2018. Hydrogenotrophic Methanogenesis and Autotrophic Growth of Methanosarcina thermophila. Archaea.
Feldewert, C., et al. 2020. The hydrogen threshold of obligately methyl-reducing methanogens. FEMS Microbiol Lett. 367:17.
Kato, S., et al. 2020. An iron corrosion-assisted H2-supplying system: a culture method for methanogens and acetogens under low H2 pressures. Scientific Reports. 10.
Lovely, D. R., Klug, M. J. 1983. Methanogenesis from Methanol and Methylamines and Acetogenesis from Hydrogen and Carbon Dioxide in the Sediments of a Eutrophic Lake. Appl Environ Microbiol. 45:4.
Zhang, G., et al. 2008. Methanogenesis from Methanol at Low Temperatures by a Novel Psychrophilic Methanogen, “Methanolobus psychrophilus” sp. nov., Prevalent in Zoige Wetland of the Tibetan Plateau. Appl Environ Microbiol. 74:19.
Welander, P. V., Metcalf, W. W. 2005. Loss of the mtr operon in Methanosarcina blocks growth on methanol, but not methanogenesis, and reveals an unknown methanogenic pathway. PNAS. 102:30
Lackner, N., et al. 2018. Hydrogenotrophic Methanogenesis and Autotrophic Growth of Methanosarcina thermophila. Archaea
Joblin K. N. 2005. Methanogenic archaea. Methods in Gut Microbial Ecology for Ruminants
Wedlock, D. N., et al. 2010. Development of a vaccine to mitigate greenhouse gas emissions in agriculture: Vaccination of sheep with methanogen fractions induces antibodies that block methane production in vitro. New Zealand Veterinary Journal. 58:1.
Wright, A. D. G. et al. Reducing methane emissions in sheep by immunization against rumen methanogens. Vaccine 22, 3976-3985 (2004).
Wang, P. et al. Isolation of high-quality total RNA from rumen anaerobic bacteria and fungi, and subsequent detection of glycoside hydrolases. Can J Microbiol 57, 590-598 (2011).
Henderson, G. et al. Effect of DNA Extraction Methods and Sampling Techniques on the Apparent Structure of Cow and Sheep Rumen Microbial Communities. Plos One 8, e74787 (2013).
Kittelmann et al. Buccal swabbing as a noninvasive method to determine bacterial, archaeal, and eukaryotic microbial community structures in the rumen. Appl Environ Microbiol 81:7470-7483 (2015).
Kittelmann et al. Simultaneous Amplicon Sequencing to Explore CoOccurrence Patterns of Bacterial, Archaeal and Eukaryotic Microorganisms in Rumen Microbial Communities. PLoS ONE 8(2): e47879 (2013).
Weimer, P. J., Y. Shi, and C. L. Odt. 1991, A segmented gas/liquid delivery system for continuous culture of microorganisms on insoluble substrates and its use for growth of Ruminococcus flavefaciens on cellulose. Appl. Microbiol. Biotechnol. 36:178-183.

### Ranges

Where ranges are given, endpoints are included. Furthermore, it is to be understood that unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1-227. (canceled)

228. A vaccine composition comprising cells and/or cell parts of at least one methanogen, wherein the at least one methanogen comprises Methanobrevibacter gottschalkii.

229. The vaccine composition of claim 228, wherein the cells and/or cell parts are killed, fixed, and/or irradiated.

230. The vaccine composition of claim 228, wherein the cells and/or cell parts are:
(a) killed by heat, steam, freezing, or sonicated;
(b) fixed by formaldehyde or formalin; and/or
(c) irradiated by UV or gamma irradiation.

231. The vaccine composition of claim 228, wherein at least about 50%, 55%, 60%, 65%, 70%, 75%, 80%, 85%, 90%, 95%, 96%, 97%, 98%, 99%, or 100% of the cells and/or cell parts are cells.

232. The vaccine composition of claim 228, wherein the vaccine composition comprises at least about 10⁶, 10⁷, 10⁸, 10⁹, or 10¹⁰ cells per mL; and/or no more than about 10⁷, 10⁸, 10⁹, 10¹⁰, 10¹¹, or 10¹² cells per mL.

233. The vaccine composition of claim 228, wherein the vaccine composition comprises:
(a) at least about 10⁶ but no more than about 10¹² cells per mL;
(b) at least about 10⁸ but no more than about 10¹² cells per mL;
(c) at least about 10⁸ but no more than about 10¹¹ cells per mL; or
(d) at least about 10⁸ but no more than about 10¹⁰ cells per mL.

234. The vaccine composition of claim 228, wherein the vaccine composition comprises:
(a) at least about 0.2, 0.4, 0.6, 0.8, 1, 1.2, 1.4, 1.6, 1.8, 2, or 4 mg of total proteins; and/or
(b) no more than about 0.4, 0.6, 0.8, 1, 1.2, 1.4, 1.6, 1.8, 2, 4, 6, 8, or 10 mg of total proteins.

235. The vaccine composition of claim 228, wherein the vaccine composition comprises:
(a) at least about 0.2 mg but no more than about 10 mg of total protein; or
(b) at least about 0.6 mg but no more than about 5 mg of total protein.

236. The vaccine composition of claim 228, wherein the cells and/or cell parts are lyophilized.

237. The vaccine composition of claim 228, wherein the vaccine composition is a pharmaceutical composition.

238. The vaccine composition of claim 237, wherein the pharmaceutical composition comprises at least one carrier, at least one excipient, at least one cryoprotectant, and/or at least one inactive ingredient.

239. The vaccine composition of claim 228, wherein wherein the vaccine composition comprises at least one adjuvant.

240. The vaccine composition of claim 239, wherein the at least one adjuvant comprises:
(a) inorganic salts, preferably aluminum and calcium salts;
(b) an oil emulsion;
(c) saponins;
(d) lipid or liposomes
(e) an immune-stimulating complex;
(f) a carrier protein (e.g., keyhole limpet hemocyanin (KLH), optionally conjugated to cells and/or cell parts);
(g) a bacterial product or derivatives thereof (e.g., bacterial toxins, lipopolysaccharide, etc.);
(h) a cytokine; or
(i) any combination of two or more selected from (a)-(h).

241. The vaccine composition of claim 239, wherein the at least one adjuvant comprises Complete Freund's adjuvant, Incomplete Freund's adjuvant, Montanide ISA70, Montanide ISA61, Saponin, chitosan thermogel, lipid, monophosphoryl lipid A, a lipid nanoparticle/cationic liposome adjuvant, Emulsigen-D, Emulsigen, Emulsigen-P, Polygen, ENABL 06, Montainde ISA 201, Montanide Gel 02, or any combination of two or more thereof.

242. The vaccine composition of claim 228, wherein the at least one methanogen further comprises Methanobrevibacter ruminantium.

243. The vaccine composition of claim 242, wherein the ratio of Methanobrevibacter ruminantium to Methanobrevibacter gottschalkii is at least about 1:9, 2:8, 3:7, 4:6, 5:5, 6:4, 7:3, 8:2, or 9:1 by cell number.

244. The vaccine composition of claim 242, wherein the at least one methanogen consists of Methanobrevibacter gottschalkii and Methanobrevibacter ruminantium.

245. The vaccine composition of claim 228, wherein Methanobrevibacter gottschalkii is Methanobrevibacter gottschalkii DSM11977.

246. The vaccine composition of claim 242, wherein Methanobrevibacter ruminantium is Methanobrevibacter ruminantium M1.

247. A method of inducing an immune response against at least one methanogen in a subject, the method comprising administering to the subject the vaccine of claim 228.

248. A method of reducing the activity, number, and/or type of methanogens in a digestive tract of a subject, the method comprising administering to the subject the vaccine composition of claim 228.
